SEQUENCE LISTING (19 sequences)

SEQ ID NO: 1 - DNA, 28 bp, Artificial
  PCR primer
cgagaatatgatagattatatgaagaat

SEQ ID NO: 2 - DNA, 30 bp, Artificial
  PCR primer
tggtttaatgctgttcatacgtttgtcaat

SEQ ID NO: 3 - DNA, 76 bp, Artificial
  PCR primer
gggacaagtttgtacaaaaaagcaggctacgaaggagatatacatatgcgagaatatgatagattatatgaagaat

SEQ ID NO: 4 - DNA, 54 bp, Artificial
  PCR primer
attaaaccaggaggaggaggaggaggatgcttcagtttcataatgcctcctgct

SEQ ID NO: 5 - DNA, 26 bp, Artificial
  PCR primer
ctagtggaatgtttactaccaaatgg

SEQ ID NO: 6 - DNA, 26 bp, Artificial
  PCR primer
gttcaatgcatgctgtttaattgtgt

SEQ ID NO: 7 - DNA, 63 bp, Artificial
  PCR primer
gggggaatttccggtggtggtggtggaattatggtactagtggaatgtttactaccaaatgga

SEQ ID NO: 8 - DNA, 56 bp, Artificial
  PCR primer
agctccgtgatggtgatggtgatgtgctccgttcaatgcatgctgtttaattgtgt

SEQ ID NO: 9 - DNA, 61 bp, Artificial
  PCR primer
gggaccactttgtacaagaaagctgggtttaagctccgtgatggtgatggtgatgtgctcc

SEQ ID NO: 10 - DNA, 42 bp, Artificial
  PCR Primer
gctagcatgcgagaatatgatagattatatgaagaatatacc

SEQ ID NO: 11 - DNA, 45 bp, Artificial
  PCR primer
gcctccaccacctccgcctggtttaatgctgttcatacgtttgtc

SEQ ID NO: 12 - DNA, 42 bp, Artificial
  PCR primer
tactagtccgcctccaccacctccgcctccaccacctccgcc

SEQ ID NO: 13 - DNA, 54 bp, Artificial
  PCR primer
actgaagcatcctcctcctcctcctcctggtttaatgctgttcatacgtttgtc

SEQ ID NO: 14 - DNA, 57 bp, Artificial
  PCR primer
agctccgtgatggtgatggtgatgtgctccagatctgtagtctttccgaactgtgtg

SEQ ID NO: 15 - DNA, 45 bp, Artificial
  PCR primer
tcctcctcctcctcctcctggtttaatgctgttcatacgtttgtc

SEQ ID NO: 16 - DNA, 26 bp, Artificial
  PCR primer
atgccccctggggtggactgccccat

SEQ ID NO: 17 - DNA, 26 bp, Artificial
  PCR primer
ctactgcctgttgtctttggacacgt

SEQ ID NO: 18 - DNA, 53 bp, Artificial
  PCR primer
attaaaccaggaggaggaggaggaggaccccctggggtggactgccccatgga

SEQ ID NO: 19 - DNA, 56 bp, Artificial
  PCR primer
agctccgtgatggtgatggtgatgtgctccctgcctgttgtctttggacacgttgt

(19) United States
(12) Patent Application Publication
(10) Pub. No.: US 2010/0311714 A1
(43) Pub. Date: Dec. 9, 2010

(54) 1H-IMIDAZO[4,5-c]QUINOLINONE COMPOUNDS

(76) Inventors: Pascal Furet, Basel (CH); Patricia Imbach, Basel (CH); Robert Mah, Basel (CH); Frédéric Stauffer, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/792,187

(22) Filed: Jun. 2, 2010

Related U.S. Application Data
(60) Provisional application No. 61/217,836, filed on Jun. 4, 2009.

(51) Int. Cl. A61K 31/437; C07D 471/04; A61K 31/506; A61K 31/497; A61P 35/04

(52) U.S. Cl. 514/210.21; 546/82; 514/293; 544/331; 514/275; 544/361; 514/253.03

(57) ABSTRACT

The invention relates to the use of 1H-imidazo[4,5-c]quinolinone compounds and salts thereof in the treatment of protein and/or lipid kinase dependent diseases and for the manufacture of pharmaceutical preparations for the treatment of said diseases; 1H-imidazo[4,5-c]quinolinone compounds for use in the treatment of protein and/or lipid kinase dependent diseases; a method of treatment against said diseases, comprising administering the 1H-imidazo[4,5-c]quinolinone compounds to a warm-blooded animal, especially a human; pharmaceutical preparations comprising an 1H-imidazo[4,5-c]quinolinone compounds, especially for the treatment of a protein and/or lipid kinase dependent disease; novel 1H-imidazo[4,5-c]quinolinone compounds; and a process for the preparation of the novel 1H-imidazo[4,5-c]quinolinone compounds.

### BACKGROUND OF THE INVENTION

The invention relates to imidazoquinolinones, salts and prodrugs thereof, processes for their preparation, their use in the treatment of protein or lipid kinase dependent diseases and in particular phosphatidylinositol-3-kinase (PI3K) dependent diseases, their use, either alone or in combination with at least one additional therapeutic agent and optionally in combination with a pharmaceutically acceptable carrier, for the manufacture of pharmaceutical preparations, use of the pharmaceutical preparations for the treatment of protein or lipid kinase dependant diseases and in particular PI3K dependent diseases, and a method of treatment of said diseases, comprising administering the imidazoquinolinones to a warm-blooded animal, especially a human. The invention also relates to pharmaceutical preparations comprising an imidazoquinolinone of the invention, either alone or in combination with at least one additional therapeutic agent, and optionally in combination with a pharmaceutically acceptable carrier.

### SUMMARY OF THE INVENTION

The phosphatidylinositol-3-kinases superfamily comprises 4 different PI3K related lipid or protein kinases. Class I, II and III are lipid kinases that differ from their substrate specificities whereas class IV PI3Ks (also called PIKKs) are protein kinases. Class I phosphatidylinositol-3-kinases comprise a family of lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP_{S}) that, in turn, act as second messengers in signaling cascades by docking proteins containing pleckstrin-homology, FYVE, Phox and other phospholipid-binding domains into a variety of signaling complexes often at the plasma membrane ((Vanhaesebroeck et al., Annu. Rev. Biochem 70:535 (2001); Katso et al., Annu. Rev. Cell Dev. Biol. 17:615 (2001)). Of the two Class I PI3Ks, Class IA PI3Ks are heterodimers composed of a catalytic p110 subunit (α, β, δ isoforms) constitutively associated with a regulatory subunit that can be p85α, p55α, p50α, p85β or p55γ. The Class IB sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class IA PI3Ks. Class IB PI3K is activated directly by G protein-coupled receptors that bind a diverse repertoire of peptide and non-peptide ligands (Stephens et al., Cell 89:105 (1997)); Katso et al., Annu. Rev. Cell Dev. Biol. 17:615-675 (2001)). Consequently, the resultant phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)). In many cases, PIP2 and PIP3 recruit Akt, the product of the human homologue of the viral oncogene v-Akt, to the plasma membrane where it acts as a nodal point for many intracellular signaling pathways important for growth and survival (Fant) et al., Cell 69:413-423 (1992); Bader et al., Nature Rev. Cancer 5:921 (2005); Vivanco and Sawyer, Nature Rev. Cancer 2:489 (2002)). Aberrant regulation of PI3K, which often increases survival through Akt activation, is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110a isoform, PIK3CA, and for Akt are amplified and increased protein expression of their gene products has been demonstrated in several human cancers. Furthermore, mutations and translocation of p85α that serve to up-regulate the p85-p110 complex have been described in human cancers. Also, somatic missense mutations in PIK3CA that activate downstream signaling pathways have been described at significant frequencies in a wide diversity of human cancers (Kang at al., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase and the upstream and downstream components of this signaling pathway is one of the most common deregulations associated with human cancers and proliferative diseases (Parsons et al., Nature 436:792 (2005); Hennessey et al., Nature Rev. Drug Disc. 4:988-1004 (2005)).

The mammalian target of rapamycin (mTOR) is a member of the class IV PI3K. mTOR assembles a signaling network that transduces nutrient signals and various other stimuli to regulate a wide range of cellular functions including cell growth, proliferation, survival, autophagy, various types of differentiation and metabolism. In mammalian cells, the mTOR protein is found complexed in two distinct entities called mTORC1 and mTORC2. The mTORC1 complex, that is to say mTOR associated with raptor, has been the matter of numerous studies. It is mTORC1 that integrates nutrient and growth factor input, and is in turn responsible for cell growth regulation, mainly through protein synthesis regulators such as 4EBP1 or RPS6. mTORC1 regulation requires PI3K and Akt activation for activation, meaning that mTORC1 is an effector of the PI3K pathway. mTOR when associated in the mTOR complex 2 (mTORC2) has been shown to be responsible for the activation of Akt by phosphorylation of S473 (Akt 1 numbering) (Sarbassov et al., Science 307:7098 (2005)). mTORC2 is hence here considered as an upstream activator of Akt. Interestingly mTOR can therefore be considered as being important both upstream and downstream of Akt. mTOR catalytic inhibition might therefore represent a unique way of addressing a very strong block in the PI3K-Akt pathway, by addressing both upstream and downstream effectors. Syndromes with an established or potential molecular link to dysregulation of mTOR kinase activity are, for instance, described in “K. Inoki et al.; Disregulation of the TSC-mTOR pathway in human disease, Nature Genetics, vol 37, 19-24”; “D. M. Sabatini; mTOR and cancer: insights into a complex relationship, Nature Reviews, vol. 6, 729-734”; and in “B. T. Hennessy et al.; Exploiting the PI3K/Akt pathway for cancer drug discovery, Nature Reviews, vol. 4, 988-1004”, and are as follows:
Organ or tissue transplant rejection, e.g. for the treatment of recipients of e.g. heart, lung, combined heart-lung, liver, kidney, pancreatic, skin or corneal transplants; graft-versus-host disease, such as following bone marrow transplantation;
Restenosis
Tuberous sclerosis
Lymphangioleiomyomatosis
Retinitis pigmentosis
Autoimmune diseases including encephalomyelitis, insulin-dependent diabetes mellitus, lupus, dermatomyositis, arthritis and rheumatic diseases
Steroid-resistant acute Lymphoblastic Leukaemia
Fibrotic diseases including scleroderma, pulmonary fibrosis, renal fibrosis, cystic fibrosis
Pulmonary hypertension
Immunomodulation
Multiple sclerosis
VHL syndrome
Carney complex
Familial adenonamtous polyposis
Juvenile polyposis syndrome
Birt-Hogg-Duke syndrome
Familial hypertrophic cardiomyopathy
Wolf-Parkinson-White syndrome
Neurodegenarative disorders such as Parkinson's, Huntington's, Alzheimer's and dementias caused by tau mutations, spinocerebellar ataxia type 3, motor neuron disease caused by SOD1 mutations, neuronal ceroid lipofucinoses/Batten disease (pediatric neurodegeneration)
wet and dry macular degeneration
muscle wasting (atrophy, cachexia) and myopathies such as Danon's disease.
bacterial and viral infections including M. tuberculosis, group A streptococcus, HSV type I, HIV infection
Neurofibromatosis including Neurofibromatosis type 1,
Peutz-Jeghers syndrome
   or further any combinations thereof.

Compounds with an inhibitory activity on mTORC1 have shown benefit in immunomodulation and in treating proliferative diseases such as advance renal cell carcinoma or Tubero-Sclerosis (TSC) germ line mutation associated disorders.

The catalytic inhibition of mTOR Ser/Thr kinase activity or class I PI3 kinases activity and in particular dual class I PI3-kinase(s) and mTOR kinase inhibition may be useful for the treatment of PI3K/Akt/mTOR pathway dependent diseases.

The efficacy of a dual PI3 kinase/mTOR inhibitor in malignant glioma has been recently described (Cancer Cell 9, 341-349).

In view of the above, inhibitors of class I and/or IV PI3Ks are considered to be of value in the treatment of proliferative disease and other disorders.

WO2003/097641, WO2005/054237, WO2005/054238 and WO2006/122806 describe imidazoquinolines for use in the treatment of protein kinase dependent diseases.

WO 2008/103636 describes imidazoquinolines as dual lipid kinase and mTor inhibitors.

It has now been found that the imidazoquinolinones derivatives of the formula (I) given below have advantageous pharmacological properties and inhibit, for example, lipid or protein kinases such as PI4K (phosphatidylinositol 4-kinase) and/or PI3 kinases (phosphatidylinositol 3-kinases), for example, inhibition of the PI3K superfamily which comprises PI3Kalpha, PI3Kbeta, PI3Kdelta, PI3Kgamma and mTOR, or one or more of the individual kinase members thereof. The class IV PI3K also called PI3-kinase-related protein kinase (PIKK) includes DNA-PK, ATM, ATR, hSMG-1 and mTOR. In particular, preferably imidazoquinolinones of the formula (I) given below show a high degree of selectivity in favour of one or more of the class I-IV PI3K against other protein kinases, such as the receptor tyrosine kinases and/or the Ser/Thr kinases outside of the PIKK family in the biochemical and/or in the cellular assay. In addition, the imidazoquinolinones of the formula (I) preferably display a favourable solubility and/or membrane permeability at physiological pH. Hence, the compounds of formula (I) are suitable, for example, to be used in the treatment of diseases dependent on PI3 kinase, especially proliferative diseases such as tumor diseases, leukaemias, and myeloproliferative disorders such as polycythemia vera, essential thrombocythemia, and myelofibrosis with myeloid metaplasia, and proliferative skin diseases including basal cell carcinoma, squamous cell carcinoma and actinic keratosis.

### DESCRIPTION OF THE INVENTION

In a first aspect, the present invention provides compounds of the formula (I)

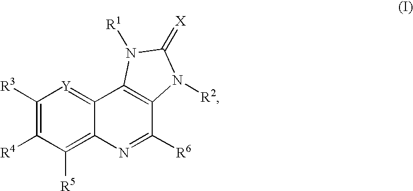

wherein
X is O or S;
Y is CH or N;
R¹ is substituted or unsubstituted pyridyl;
R² is hydrogen or lower alkyl;
R³ is a substituted or unsubstituted aryl or heterocyclyl; and
R⁴, R⁵ and R⁶ are hydrogen;
   or a pharmaceutically acceptable salt, solvate, hydrate or prodrug thereof.

The present invention is also directed to use of compounds of formula (I) in the treatment of protein and/or lipid kinase dependent diseases and in particular PI3K superfamily (especially class I PI3K and/or mTOR) dependent diseases; use of compounds of formula (I) for the manufacture of pharmaceutical preparations for the treatment of protein and/or lipid kinase dependent diseases and in particular PI3K superfamily (especially class I PI3K and/or mTOR) dependent diseases; methods of treating protein and/or lipid kinase dependant diseases and in particular PI3K superfamily (especially class I PI3K and/or mTOR) dependent diseases comprising administering imidazoquinolinone compounds of the formula (I) to a warm-blooded animal, especially a human; pharmaceutical preparations comprising an imidazoquinolinone compound of the formula (I), especially for the treatment of a protein and/or lipid kinase dependant disease and in particular a PI3K superfamilly (especially class I PI3K and/or mTOR) dependent disease; a process for the manufacture of the novel imidazoquinolinone compounds of the formula (I); the manufacture of a pharmaceutical preparation for the treatment of protein and/or lipid kinase dependant diseases and in particular PI3K superfamily (especially class I PI3K and/or mTOR) dependent diseases, and novel intermediates for their manufacture. Preferably, the PI3K superfamily dependent diseases are class I PI3K and/or mTOR dependent diseases.

The invention may be more fully appreciated by reference to the following description, including the following glossary of terms and the concluding examples. As used herein, the terms “including”, “containing” and “comprising” are used herein in their open, non-limiting sense.

Any formula given herein is intended to represent compounds having structures depicted by the structural formula as well as certain variations or forms. In particular, compounds of any formula given herein may have asymmetric centers and therefore exist in different stereoisomeric forms such as different enantiomeric forms. If at least one asymmetrical carbon atom is present in a compound of the formula I, such a compound may exist in optically active form or in the form of a mixture of optical isomers, e.g. in the form of a racemic mixture. Thus an asymmetric carbon atom may be present in the (R)-, (S)- or (R,S)-configuration, preferably in the (R)- or (S)-configuration. All optical isomers and their mixtures, including the racemic mixtures, are part of the present invention. Thus, any given formula given herein is intended to represent a racemate, one or more enantiomeric forms, one or more diastereomeric forms, one or more atropisomeric forms, and mixtures thereof. Furthermore, certain structures may exist as geometric isomers (e.g. cis and trans isomers), as tautomers, or as atropisomers. For example, substituents at a double bond or a ring may be present in cis-(=Z-) or trans (=E-) form. The compounds of the invention may thus be present as mixtures of isomers or preferably as pure isomers, preferably as enantiomer-pure diastereomers or pure enantiomers.

Any formula given herein is intended to represent hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, ²H, ³H, ¹¹C, ¹³C, ¹⁴C, ¹⁵N, ³¹F, ³²F, ¹⁸F ³⁵S, ³⁶Cl, phosphorous, fluorine, and chlorine, such as ¹²⁵I respectively. Various isotopically labeled compounds of the present invention, for example those into which radioactive isotopes such as ³H, ¹³C, and ¹⁴C are incorporated. Such isotopically labelled compounds are useful in metabolic studies (preferably with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula (where one or more up to all more general expressions in embodiments characterized as preferred above or below can be replaced with a more specific definition, thus leading to a more preferred embodiment of the invention, respectively).

Where the plural form (e.g. compounds, salts, pharmaceutical preparations, diseases and the like) is used, this includes the singular (e.g. a single compound, a single salt, a single pharmaceutical preparation, a single disease, and the like). “A compound” does not exclude that (e.g. in a pharmaceutical formulation) more than one compound of the formula (I) (or a salt thereof) is present.

Salts are preferably the pharmaceutically acceptable salts of compounds of formula (I) if they are carrying salt-forming groups.

The salts of compounds of formula (I) are preferably pharmaceutically acceptable salts; acids/bases required to form the salts are generally known in the field.

Salt-forming groups in a compound of formula (I) are groups or radicals having basic or acidic properties. Compounds having at least one basic group or at least one basic radical, e.g., amino; a secondary amino group not forming a peptide bond or a pyridyl radical, may form acid addition salts, e.g., with inorganic acids, such as hydrochloric acid, sulfuric acid or a phosphoric acid; or with suitable organic carboxylic or sulfonic acids, e.g., aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid; or amino acids, such as arginine or lysine; aromatic carboxylic acids, such as benzoic acid; 2-phenoxy-benzoic acid; 2-acetoxy-benzoic acid; salicylic acid; 4-aminosalicylic acid; aromatic-aliphatic carboxylic acids, such as mandelic acid or cinnamic acid; heteroaromatic carboxylic acids, such as nicotinic acid or isonicotinic acid; aliphatic sulfonic acids, such as methane-, ethane- or 2-hydroxyethanesulfonic acid; or aromatic sulfonic acids, e.g., benzene-, p-toluene- or naphthalene-2-sulfonic acid. When several basic groups are present mono- or poly-acid addition salts may be formed.

Compounds of formula (I) having acidic groups, a carboxy group or a phenolic hydroxy group, may form metal or ammonium salts, such as alkali metal or alkaline earth metal salts, e.g., sodium, potassium, magnesium or calcium salts; or ammonium salts with ammonia or suitable organic amines, such as tertiary monoamines, e.g., triethylamine or tri(2-hydroxyethyl)-amine, or heterocyclic bases, e.g., N-ethyl-piperidine or N,N′-dimethylpiperazine. Mixtures of salts are possible.

Compounds of formula (I) having both acidic and basic groups can form internal salts.

For the purposes of isolation or purification, as well as in the case of compounds that are used further as intermediates, it is also possible to use pharmaceutically-unacceptable salts, e.g., the picrates. Only pharmaceutically-acceptable, non-toxic salts may be used for therapeutic purposes, however, and those salts are therefore preferred.

Owing to the close relationship between the novel compounds in free form and in the form of their salts, including those salts that can be used as intermediates, e.g., in the purification of the novel compounds or for the identification thereof, any reference hereinbefore and hereinafter to the free compounds shall be understood as including the corresponding salts, where appropriate and expedient.

Compounds of the present invention may also form solvates and hydrates, and as such any reference to a compound of formula (I) is therefore to be understood as referring also to the corresponding solvate and/or hydrate of the compound of formula (I), as appropriate and expedient.

The present invention also relates to pro-drugs of a compound of formula (I) that convert in vivo to the compound of formula (I) as such. Any reference to a compound of formula (I) is therefore to be understood as referring also to the corresponding pro-drugs of the compound of formula (I), as appropriate and expedient.

The general terms used hereinbefore and hereinafter preferably have within the context of this disclosure the following meanings, unless otherwise indicated:

The prefix “lower” denotes a radical having 1 carbon atom up to and including a maximum of 7 carbon atoms, especially 1 carbon atom up to and including a maximum of 4 carbon atoms, the radicals in question being either linear or branched with single- or multiple-branching.

Halo or halogen is preferably fluoro, chloro, bromo or iodo, most preferably fluoro, chloro or bromo.

Alkyl preferably has 1 up 12 carbon atoms (C₁₋₁₂alkyl) and is linear or branched one or more times; in particular alkyl is lower alkyl, especially C₁-C₄alkyl. For example, alkyl includes methyl, ethyl, n-propyl, iso-propyl, n-butyl, isobutyl, sec-butyl, tert-butyl, n-pentyl, n-hexyl, n-heptyl, n-octyl, n-nonyl, n-decyl, n-undecyl, n-dodecyl, with particular preference given to methyl, ethyl, n-propyl, iso-propyl, n-butyl and iso-butyl. Lower alkyl, for example, is represented as C₁-C₇alkyl, e.g. methyl, ethyl, n-propyl, iso-propyl, n-butyl, isobutyl, sec-butyl, tert-butyl, n-pentyl, n-hexyl or n-heptyl, lower alkyl is especially represented as C₁-C₄alkyl, e.g. methyl, ethyl, n-propyl, iso-propyl, n-butyl, isobutyl, sec-butyl, tert-butyl, with preference given to methyl, ethyl or propyl.

Alkyl, in particular lower alkyl, is unsubstituted or substituted, preferably by one or more substituents independently selected from those mentioned below under “substituted”. Exemplary substituents include, but are not limited to hydroxy, alkoxy, aryl, heterocyclyl, cycloalkyl, halogen, amino and nitro. An example of a substituted alkyl is halo-alkyl, such as halomethyl, for example fluoromethyl, e.g. trifluoromethyl. Another example of a substituted alkyl is hydroxymethyl or hydroxyethyl. A further example of a substituted alkyl is alkoxyalkyl, such as methoxyethyl or methoxypropyl. The alkoxyalkyl may be further substituted, for example to give benzyloxyethyl or benzyloxypropyl.

Alkyl may also be cyclic as defined below under “cycloalkyl”. Cycloalkyl may also be a substituent to alkyl. Cycloalkyl-lower alkyl is preferably lower alkyl that is substituted (preferably terminally) by unsubstituted or substituted cycloalkyl as defined below. An example of cycloalkyl as a substituent to alkyl is alkandiyl-cycloalkyl, such as alkandiyl-cycloloweralkyl, e.g. alkandiyl-cyclopropyl, e.g. CH₂-cyclopropyl.

Aryl-lower alkyl is preferably lower alkyl that is substituted (preferably terminally or in 1-position) by unsubstituted or substituted aryl as defined below. Aryl-lower alkyl is especially phenyl-lower alkyl, such as benzyl (i.e. phenylmethyl) or phenylethyl, especially 1-phenylethyl.

Heterocyclyl-lower alkyl is preferably lower alkyl that is substituted (preferably terminally) by unsubstituted or substituted heterocyclyl as defined below.

Each alkyl part of other groups like “alkoxy”, “alkoxyalkyl”, “alkoxyalkoxy”, “alkoxycarbonyl”, “alkoxy-carbonylalkyl”, “alkylsulfonyl”, “alkylsulfonamide”, “alkylsulfinyl”, “alkylamino”, “halogenalkyl” shall have the same meaning as described in the above-mentioned definition of “alkyl”, including the prefix ‘lower’, including substitutions thereof.

For example, aryl-lower alkoxy is preferably lower alkoxy that is substituted (preferably terminally on the alkyl part) by unsubstituted or substituted aryl as defined below. Aryl-lower alkoxy is especially phenyl-lower alkoxy, such as phenyl methoxy (i.e. benzoxy) or phenyl ethoxy.

Alkoxyalkoxy is, for example, methoxyethoxy, or methoxypropoxy, and may include further substitutions e.g. by aryl such as phenyl, for example to give phenylmethoxyethoxy (alternatively stated benzoxy-ethoxy) or phenylmethoxypropoxy (alternatively stated benzoxy-propoxy).

Alkandiyl is a straight-chain or branched-chain divalent alkyl group. It preferably represents a straight-chain or branched-chain C₁₋₁₂ alkandiyl, particularly preferably represents a straight-chain or branched-chain C₁₋₆alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

Alkenyl is preferably a moiety with one or more double bonds and preferably has 2-12 carbon atoms; it is linear or branched one or more times (as far as possible in view of the number of carbon atoms). Preferred is C₂-C₇alkenyl, especially C₃-C₄alkenyl, such as allyl or crotyl. For example, —CH═CH—, —CH═C(CH₃)—, —CH═CH—CH₂—, —C(CH₃)═CH—CH₂—, —CH═C(CH₃)—CH₂—, —CH═CH—C(CH₃)H—, —CH═CH—CH═CH—, —C(CH₃)═CH—CH═CH—, —CH═C(CH₃)—CH═CH—. Alkenyl can be unsubstituted or substituted, especially by one or more, more especially up to three of the substituents mentioned below under “substituted”. Substituents, such as amino or hydroxy (with free dissociable hydrogen) preferably are not bound to carbon atoms that participate at a double bond, and also other substituents that are not sufficiently stable are preferably excluded. Unsubstituted alkenyl, in particular, C₂-C₇alkenyl is preferred.

Alkynyl is preferably a moiety with one or more triple bonds and preferably has 2-12 carbon atoms; it is linear of branched one or more times (as far as possible in view of the number of carbon atoms). Preferred is C₂-C₇alkynyl, especially C₃-C₄alkynyl, such as ethynyl or propyn-2-yl. Alkynyl can be unsubstituted or substituted, especially by one or more, more especially up to three of the substituents mentioned below under “substituted”. Substituents, such as amino or hydroxy (with free dissociable hydrogen) preferably are not bound to carbon atoms that participate at a triple bond, and also other substituents that are not sufficiently stable are preferably excluded. Unsubstituted alkynyl, in particular, C₂-C₇alkynyl is preferred.

Cycloalkyl is a saturated, monocyclic, fused polycyclic, or spiro polycyclic, carbocycle having from 3 to 12 ring atoms per carbocycle. Cycloalkyl is preferably C₃-C₁₀cycloalkyl, and includes cyclo lower alkyl, especially cyclopropyl, cyclobutyl, cyclopentyl, cyclohexyl or cycloheptyl; cycloalkyl being unsubstituted or substituted by one or more substituents, especially 1-3 substituents independently selected from the group consisting of the substituents defined below under “substituted”.

Cycloalkenyl is preferably C₅-C₁₀cycloalkenyl, especially cyclopentenyl, cyclohexenyl or cycloheptenyl; cycloalkenyl being unsubstituted or substituted by one or more substituents, especially 1-3 substituents, independently selected from the group consisting of the substituents defined below under “substituted”.

Aryl refers to an unsaturated carbocyclic aromatic ring system, preferably, having a ring system of not more than 16 carbon atoms, especially not more than 10 carbon atoms, e.g. having 6 to 16, preferably 6 to 10 ring carbon atoms, is preferably mono- or bi-cyclic, and is unsubstituted or substituted preferably as defined below under “substituted”. For example, aryl is selected from phenyl or naphthyl, preferably phenyl, and is preferably in each case unsubstituted or substituted with substituents described under “substituted”, in particular from the group consisting of halo, especially fluoro, chloro, bromo or iodo, in particular fluoro; halo-lower alkyl, especially fluoroalkyl, in particular trifluoromethyl; hydroxyl; amino, mono or disubstituted amino, especially alkyl-substituted amino, hydroxyalkyl-substituted amino or alkoxyalkyl-substituted amino, e.g. dimethyl amino, 2-hydroxyethyl amino or 2-methoxyethylamino; cyclic amino, such as aziridinyl, azetidinyl or pyrrolidinyl; amino-lower alkyl, e.g., aminomethyl, 2-aminoethyl or 3-aminopropyl; alkylamino-lower alkyl, e.g. methylaminomethyl, ethylaminomethyl, methylaminoethyl or ethylaminoethyl; dialkylamino-loweralkyl, e.g. dimethylaminomethyl, dimethylaminoethyl, methylethylaminomethyl, methylethylaminoethyl, diethylaminomethyl or diethylaminoethyl; cycloalkylaminoalkyl, e.g. cyclopropylaminomethyl, cyclopropylaminoethyl, cyclobutylaminomethyl, cyclobutylaminoethyl, cyclopentylaminomethyl or cyclopentylaminoethyl; dicycloalkylaminoalkyl, e.g. dicyclopropylaminomethyl, dicyclopropylaminoethyl, cyclopropylcyclobutylaminomethyl or cyclopropylcyclobutylaminoethyl; alkylcycloalkylaminoalkyl, e.g. cyclopropylmethylaminomethyl, cyclopropylmethylaminoethyl, cyclopropylethylaminomethyl or cyclopropylethylaminoethyl; lower alkoxy, e.g., methoxy, ethoxy or propyloxy; hydroxy-lower alkyl, e.g., hydroxymethyl or 2-hydroxyethyl; hydroxy lower alkoxy, e.g. hydroxyethoxy; alkoxy lower alkoxy, e.g. methoxyethoxy or ethoxyethoxy; lower alkyl, e.g., methyl, ethyl or iso-propyl; cyano; cyano-lower alkyl, e.g., 2-cyanoethyl, 2-cyanopropyl, 2-cyano-2-methylpropyl or 3-cyanopropyl; amidino; N-hydroxyamidino; amidino-lower alkyl, e.g., 2-amidino-ethyl; or N-hydroxyamidino-lower alkyl, e.g., 2-(N-hydroxyamidino)-ethyl; nitro; carboxylic acid; substituted sulfonyl, e.g. alkyl-substituted sulfonyl, such as methanesulfonyl; sulfonamide, e.g. N-methylsulfonamide or pyrrolidine-1-sulfonyl; lower alkyl sulfonyl amino, e.g. methylsulfonylamino; lower alkyl sulfonylalkandiylamino, e.g. methylsulfonylmethylamino; acylamino, e.g. acetylamino, acyl lower alkyl amino, e.g. acetyl methyl amino; [1,3]dioxolo; substituted [1,3]dioxolo, e.g. 2,2-difluoro-[1,3]dioxolo; alkoxy carbonyl, such as lower alkoxy carbonyl, e.g. methoxycarbonyl; carbamoyl; substituted carbamoyl, such as alkyl-substituted carbamoyl, e.g. methylcarbamoyl. The aryl group may also be substituted with a substituted or unsubstituted heterocycle, preferably a 4-7 membered ring, e.g. 1H-tetrazolyl (in particular 1H-tetrazol-5-yl), pyrazol, imidazole, triazole, azetidinyl, pyrrolidinyl, piperazinyl, methylpiperazinyl, ethylpiperazinyl, triazolonyl or methylimidazolyl. The aryl group may also be substituted with heterocyclyl lower alkyl, heteroaryl or heteroaryl lower alkyl as defined herein below.

Preferred unsubstituted or substituted aryl is selected from phenyl; hydroxyphenyl, e.g., 2-, 3- or 4-hydroxyphenyl; methoxyphenyl, e.g., 2-, 3- or 4-methoxyphenyl or 3,4-dimethoxyphenyl; ethoxyphenyl, e.g., 2-, 3- or 4-ethoxyphenyl or 3,4-diethoxyphenyl; methoxy ethoxy-phenyl, e.g. 3-methoxy-4-ethoxy phenyl or 4-methoxy-3-ethoxy phenyl, or other lower-alkoxy phenyl, e.g. 3-methoxy-4-(2-methoxy ethoxy)-phenyl; hydroxyalkoxy phenyl, e.g. 2-hydroxyethoxy-phenyl; hydroxy alkoxy phenyl, e.g. 3-methoxy-4-hydroxy phenyl; halo-hydroxy-phenyl, e.g. fluoro-hydroxy-phenyl such as 3-fluoro-5-hydroxy-phenyl; hydroxy-haloalkyl-phenyl, e.g. hydroxy-fluoroalkyl-phenyl such as 3-hydroxy-5-trifluoromethyl-phenyl; 2,2-difluoro-benzo[1,3]dioxolo, benzene sulfonamide, e.g. 3-N-methylbenzenesulfonamide, 3-(pyrrolidine-1-sulfonyl)-phenyl or N-(3-phenyl)-methanesulfonamide; alkyl-sulfonyl phenyl, e.g. 3-methanesulfonylphenyl; benzamide e.g. 3- or 4-benzamide, 3- or 4-N-methyl-benzamide or 2-, 3- or 4-N,N-dimethyl-benzamide; pyrazol-phenyl, e.g. 4-pyrazol-phenyl.

Heterocyclyl refers to a heterocyclic radical that is unsaturated (=carrying the highest possible number of conjugated double bonds in the ring(s) e.g. heteroaryl, for example pyrazolyl, pyridyl, pyrimidinyl), saturated or partially saturated in the bonding ring and is preferably a monocyclic or in a broader aspect of the invention bicyclic ring; has 3-16 ring atoms, more preferably 4-10 ring atoms, wherein at least in the ring bonding to the radical of the molecule of formula (I) one or more, preferably 1-4 ring atoms, especially one or two ring atoms are a heteroatom selected from the group consisting of nitrogen, oxygen and sulfur; the bonding ring preferably having 4-12 ring atoms, especially 4-7 ring atoms, for example 6-10 ring atoms, especially for heteroaryl, such as 6, 9 or 10 ring atoms. The heterocyclyl may be unsubstituted, or substituted by one or more, especially 1-4 substituents independently selected from the group consisting of the substituents defined below under “substituted”; especially being a heterocyclyl radical selected from the group consisting of oxiranyl, azirinyl, 1,2-oxathiolanyl, imidazolyl, thienyl, furyl, tetrahydrofuryl, tetrahydrothiophene, indolyl, 1-methyl-2,3-dihydro-1H-indolyl, 2-oxo-2,3-dihydro-1H-indolyl, azetidinyl, pyranyl, thiopyranyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, 3H-indolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, phthalazinyl, naphthyridinyl, 1H-pyrrolo[2,3-b]pyridinyl, quinoxalyl, quinazolinyl, quinazolinyl, cinnolinyl, pteridinyl, furazanyl, chromenyl, isochromanyl and chromanyl; each of these heterocycle radicals being unsubstituted or substituted by one to two radicals selected from the substituents described under “substituted”, in particular from the group consisting of halo, especially fluoro, chloro, bromo or iodo, more especially fluoro or chloro, in particular fluoro; halo-lower alkyl, especially fluoroalkyl, in particular trifluoromethyl; hydroxyl; amino, mono or disubstituted amino, especially alkyl-substituted amino, hydroxyalkyl-substituted amino or alkoxyalkyl-substituted amino, e.g. dimethyl amino, 2-hydroxyethyl amino or 2-methoxyethyl amino; cyclic amino, such as aziridinyl, azetidinyl or pyrrolidinyl; amino-lower alkyl, e.g., aminomethyl, 2-aminoethyl or 3-aminopropyl; alkylamino-lower alkyl, e.g. methylaminomethyl, ethylaminomethyl, methylaminoethyl or ethylaminoethyl; dialkylamino-loweralkyl, e.g. dimethylaminomethyl, dimethylaminoethyl, methylethylaminomethyl, methylethylaminoethyl, diethylaminomethyl or diethylaminoethyl; cycloalkylaminoalkyl, e.g. cyclopropylaminomethyl, cyclopropylaminoethyl, cyclobutylaminomethyl, cyclobutylaminoethyl, cyclopentylaminomethyl or cyclopentylaminoethyl; dicycloalkylaminoalkyl, e.g. dicyclopropylaminomethyl, dicyclopropylaminoethyl, cyclopropylcyclobutylaminomethyl or cyclopropylcyclobutylaminoethyl; alkylcycloalkylaminoalkyl, e.g. cyclopropylmethylaminomethyl, cyclopropylmethylaminoethyl, cyclopropylethylaminomethyl or cyclopropylethylaminoethyl; lower alkoxy, e.g., methoxy, ethoxy or propyloxy; hydroxy-lower alkyl, e.g., hydroxymethyl or 2-hydroxyethyl; hydroxy lower alkoxy, e.g. hydroxyethoxy; alkoxy lower alkoxy, e.g. methoxyethoxy or ethoxyethoxy; lower alkyl, e.g., methyl, ethyl or iso-propyl; cyano; cyano-lower alkyl, e.g., 2-cyanoethyl, 2-cyanopropyl, 2-cyano-2-methylpropyl or 3-cyanopropyl; amidino; N-hydroxyamidino; amidino-lower alkyl, e.g., 2-amidino-ethyl; or N-hydroxyamidino-lower alkyl, e.g., 2-(N-hydroxyamidino)-ethyl; nitro; carboxylic acid; substituted sulfonyl, e.g. alkyl-substituted sulfonyl, such as methanesulfonyl; sulfonamide, e.g. N-methylsulfonamide or pyrrolidine-1-sulfonyl; alkylsulfonylamino, e.g. methylsulfonylamino; alkylsulfonylalkylamino, e.g. methylsulfonylmethylamino; acylamino, e.g. acetylamino; acyl alkyl amino, e.g. acetyl methyl amino; [1,3]dioxolo; substituted [1,3]dioxolo, e.g. 2,2-difluoro-[1,3]dioxolo; alkoxy carbonyl, such as lower alkoxy carbonyl, e.g. methoxycarbonyl; carbamoyl; substituted carbamoyl, such as alkyl-substituted carbamoyl, e.g. methylcarbamoyl. The heterocycle group may also be substituted with another substituted or unsubstituted heterocycle, preferably a 4-7 membered ring, e.g. 1H-tetrazolyl (in particular 1H-tetrazol-5-yl), pyrazol, imidazole, triazole, azetidinyl, pyrrolidinyl, piperazinyl, methylpiperazinyl, ethylpiperazinyl, triazolonyl or methylimidazolyl. The heterocycle group may also be substituted with heterocyclyl lower alkyl, heteroaryl or heteroaryl lower alkyl as defined herein.

Very preferred heterocyclic groups, include pyridyl, pyrimidinyl, 1H-pyrrolo[2,3-b]pyridinyl, quinolyl, each of which may be unsubstituted or substituted.

Specifically preferred unsubstituted or substituted heterocyclyl is selected from pyridyl; alkylpyridyl, in particular lower-alkylpyridyl, e.g. methylpyridyl, e.g., 2-, 3- or 4-methylpyridyl, especially 2-methylpyridyl or 3-methylpyridyl; alkoxypyridyl, in particular lower-alkoxypyridyl, e.g. methoxypyridyl, e.g., 2-, 3- or 4-methoxypyridyl, especially 2-methoxypyridyl or 3-methoxypyridyl or di-methoxypyridyl, e.g. 2,3-dimethoxypyridyl, or ethoxypyridyl, e.g., 2-, 3- or 4-ethoxypyridyl especially 2-ethoxypyridyl or 3-ethoxypyridyl or di-ethoxypyridyl e.g. 2,3-diethoxypyridyl, or propyloxypyridyl, e.g., 2- or 3-propyloxypyridyl or isopropyloxypyridyl, e.g., 2- or 3-isopropyloxypyridyl; cycloalkyloxypyridyl, e.g. 2- or 3-cyclobutyloxypyridyl; cycloalkylalkoxypyridyl, e.g. cyclopropylmethoxy-pyridyl, e.g. 2 or 3-cyclopropylmethoxy-pyridyl; methoxymethylpyridyl, e.g. 2 or 3-methoxymethylpyridyl; alkoxyalkoxypyridyl, in particular methoxyethoxypyridyl, e.g. 2 or 3-methoxyethoxypyridyl; benzyloxyalkoxypyridyl, in particular benzyloxyethoxypyridyl or benzyloxypropoxypyridyl, e.g. 2-benzyloxyethoxypyridyl or 3-benzyloxypropoxypyridyl; hydroxyalkylpyridyl, e.g. 2 or 3-(2-hydroxyethyl)-pyridyl; alkyl-sulfonyl pyridyl, e.g. methanesulfonylpyridyl, especially 3-methanesulfonylpyridyl; hydroxyalkoxypyridyl, e.g. 2-(2-hydroxyethoxy)-pyridyl or 2-(3-hydroxypropoxy)-pyridyl; hydroxyalkylpyridyl, e.g. hydroxymethylpyridyl, especially 2-(hydroxymethyl)-pyridyl; alkoxycarbonylpyridyl, e.g. methoxycarbonylpyridyl, especially 2-methoxycarbonyl-pyridyl; aminopyridyl, e.g. 2- or 3-aminopyridyl; alkylaminopyridyl, in particular loweralkylaminopyridyl, e.g. 2-, 3- or 4-methylaminopyridyl, especially 2- or 3-methylaminopyridyl, 2-, 3- or 4-ethylaminopyridyl, especially 2- or 3-ethylaminopyridyl, e.g. 2- or 3-(1- or 2-propyl)aminopyridyl; dialkylaminopyridyl, in particular di-loweralkylaminopyridyl, e.g. 2-, 3- or 4-dimethylaminopyridyl, especially 2-dimethylaminopyridyl; cycloaminopyridyl, e.g. azetidinylpyridyl, especially 2-azetidin-1-yl-pyridyl or pyrrolidinylpyridyl, especially 2-pyrrolidin-2-ylpyridyl; hydroxyalkylaminopyridyl, e.g. 2-(2-hydroxyethylamino)-pyridyl; amino-haloloweralkyl-pyridyl, e.g. amino-trifluoromethyl-pyridyl, especially 2-amino-3-trifluoromethyl-pyridyl; haloalkylpyridinyl, in particular haloloweralkylpyridyl, especially, 2-, 3- or 4-trifluoromethylpyridyl, most especially 2-trifluoromethylpyridyl; halopyridyl, in particular fluoropyridyl, especially 2-fluoropyridyl; halo-alkoxy-pyridyl, e.g. fluoro-methoxy-pyridyl such as 3-fluoro-2-methoxy-pyridyl; carbamoylpyridyl, especially 2-(carbamoyl)pyridyl; alkyl-substituted carbamoyl, e.g. methylcarbamoyl, especially 2-(methylcarbamoyl)pyridyl; pyrimidinyl; loweralkylaminopyrimidinyl, e.g. 2- or 4-methylaminopyrimidinyl or 2- or 4-ethylaminopyrimidinyl; di-loweralkylaminopyrimidinyl, e.g. 2- or 4-methylethylaminopyrimidinyl, 2- or 4-dimethylaminopyrimidinyl, especially 2-dimethylaminopyrimidinyl; alkoxypyrimidinyl, in particular methoxypyrimidinyl or ethoxypyrimidinyl, e.g. 2-methoxypyrimidinyl or 2-ethoxypyrimidinyl; 1H-pyrrolo[2,3-b]pyridinyl; 1-methyl-1H-pyrrolo[2,3-b]pyridinyl; pyrazinyl; pyrazolyl; substituted pyrazolyl, e.g. hydroxyalkylpyrazolyl, especially 1-(2-hydroxy-ethyl)-1H-pyrazolyl or 1-[2-(tetrahydro-pyran-2-yloxy)-ethyl]-1H-pyrazolyl; quinolinyl; 2-oxo-2,3-dihydro-1H-indol-5-yl; 1-methyl-2,3-dihydro-1H-indol-5-yl.

“Substituted”, wherever used for a moiety, means that one or more hydrogen atoms in the respective moiety, especially up to 5 hydrogen atoms, more especially up to three of the hydrogen atoms are replaced independently of each other by a corresponding number of substituents, which preferably are independently selected from the group consisting of lower alkyl, e.g., methyl, ethyl, isopropyl or propyl; halo, e.g., F, Cl, Br or I; halo-lower alkyl, e.g., fluoroalkyl, such as trifluoromethyl; hydroxy; carboxy; lower alkoxy, e.g., methoxy, ethoxy, propyloxy or isopropyloxy; aryl-lower alkyl, e.g. phenyl-lower alkyl; aryl-lower alkoxy, e.g. phenyl-lower alkoxy; lower alkanoyloxy; lower alkanoyl; hydroxy-lower alkyl, e.g., hydroxymethyl or 2-hydroxyethyl; hydroxy lower alkoxy, e.g. hydroxyethoxy; amino; mono- or di-substituted amino; cyclic amino, e.g. aziridinyl, azetidinyl or pyrrolidinyl; amino-lower alkyl, e.g., aminomethyl, 2-aminoethyl or 3-aminopropyl; alkylamino-lower alkandiyl; dialkylamino-lower alkandiyl; N-lower alkylamino; N,N-di-lower alkylamino; amino lower alkoxy; lower alkanoylamino; lower alkanoyl-lower alkyl-amino; benzoylamino; carbamoyl-lower alkoxy; N-lower alkylcarbamoyl-lower alkoxy or N,N-di-lower alkylcarbamoyl-lower alkoxy; amidino; N-hydroxy-amidino; hydroxylamine; alkoxyamino; nitro; guanidino; amidino-lower alkyl, e.g., 2-amidinoethyl; N-hydroxyamidino-lower alkyl, e.g., N-hydroxy-amidino-methyl or -2-ethyl; carboxy; lower alkoxycarbonyl; phenyl-lower alkoxycarbonyl, e.g., benzyloxycarbonyl; lower alkanoyl; sulfo; lower alkanesulfonyl, e.g., methanesulfonyl (CH₃—S(O)₂—); sulfonamide (NH₂—S(O)₂—); dioxolo; phosphono (—P(═O)(OH)₂); hydroxy-lower alkoxy phosphoryl or di-lower alkoxyphosphoryl; carbamoyl; mono- or di-lower alkylcarbamoyl; sulfamoyl; sulfamide; mono- or di-lower alkylaminosulfonyl; lower alkanesulfonylamino; lower alkanesulfonyl-lower alkyl-amino; cyano; cyano-lower alkyl, e.g., cyanomethyl, 2-cyanoethyl or 3-cyanopropyl; aryl (e.g., phenyl), where aryl is unsubstituted or substituted with any of the substituents defined above, and especially is phenyl which is unsubstituted or substituted with up to 4 substituents, preferably up to 2 substituents, wherein the substituents are the same or different and are independently selected from halo, (e.g., Cl or F) cyano, cyano lower alkyl, (e.g., cyanomethyl, cyanoethyl and cyanopropyl) lower alkyl, lower alkoxy, amino-lower alkyl sulfanyl, thiol-lower alkyl, amino-lower alkyl, or amino-lower alkoxy, wherein the amino group in each case can be mono- or di-substituted, e.g., —(C₁-C₇)ₘNR₈R₉; or —O—(C₁-C₇)ₘNR₈R₉, wherein
m is 0 or 1; and
R₈ and R₉ can be the same or different and are independently H; lower alkyl, e.g., methyl, ethyl or propyl; lower cycloalkyl, e.g., cyclopropyl, or
R₈ and R₉, together with the N atom, form a 3- to 8-membered heterocyclic ring containing 1-4 nitrogen, oxygen or sulfur atoms, e.g., azetidinyl, pyrrolidinyl, piperidino, morpholinyl, imidazolinyl, piperazinyl or lower alkyl-piperazinyl.

“Substituted” also includes amino-carbonyl-lower alkyl, e.g., R₈R₉N—C(O)—CH₂—, wherein R₈ and R₉ are as defined above. “Substituted” also includes heterocyclyl, heterocyclyl-lower alkyl, heterocyclyl-lower alkoxy or heterocyclyl-lower alkanesulfanyl, wherein the heterocyclyl in each case is a substituted or unsubstituted 3- to 8-membered heterocyclic ring containing 1-4 nitrogen, oxygen or sulfur atoms, e.g., imidazolyl, imidazolinyl, pyrrolidinyl, morpholinyl, azetidinyl, pyridyl, pyrazolyl, piperidino, piperidyl, piperazinyl or lower alkyl-piperazinyl. “Substituted” also includes C₃-C₁₀cycloalkyl, e.g., cyclopropyl or cyclohexyl; hydroxyC₃-C₈cycloalkyl, e.g., hydroxy-cyclohexyl; heteroaryl with 4 or 6 ring atoms and 1-4 ring heteroatoms selected from O, N and S, especially furyl, 1,4 oxazinyl, or pyridyl. “Substituted” also includes —NR₈R₉, wherein R₈ and R₉ can be the same or different and are independently H; lower alkyl, e.g., methyl, ethyl or propyl; lower cycloalkyl, e.g., cyclopropyl; or the R₈ and R₉ can, with the N atom, form a 3- to 8-membered heterocyclic ring containing 1-4 nitrogen, oxygen or sulfur atoms, e.g., azetidinyl, pyrrolidinyl, piperidino, morpholinyl, imidazolinyl, piperazinyl or lower alkyl-piperazinyl.

For the avoidance of doubt, substituents are only bound at positions where chemically possible, the person skilled in the art being able to decide (either experimentally or theoretically) without inappropriate effort, which substitutions are possible and which are not. For example, amino or hydroxy groups with free hydrogen may be unstable if bound to carbon atoms with unsaturated, e.g., olefinic, bonds.

The terms “treatment” or “therapy” refer to the prophylactic or preferably therapeutic including, but not limited to, palliative, curing, symptom-alleviating, symptom-reducing, kinase-regulating and/or kinase-inhibiting, treatment of said diseases, especially of the diseases mentioned below.

Where subsequently or above the term “use” is mentioned (as verb or noun) (relating to the use of a compound of the formula (I) or a pharmaceutically acceptable salt thereof), this includes any one or more of the following embodiments of the invention, respectively: the use in the treatment of a protein or lipid kinase dependant disease and in particular PI3K dependent disease, the use for the manufacture of pharmaceutical compositions for use in the treatment of a protein or lipid kinase dependant disease and in particular PI3K dependent disease, methods of use of one or more compounds of the formula (I) in the treatment of a protein or lipid kinase dependant disease and in particular PI3K dependent disease, the use of pharmaceutical preparations comprising one or more compounds of the formula (I) for the treatment of a protein or lipid kinase dependent disease, and one or more compounds of the formula (I) for use in the treatment of a protein or lipid kinase dependent disease, as appropriate and expedient and if not stated otherwise. In particular, diseases to be treated and are thus preferred for “use” of a compound of formula (I) are selected from protein or lipid kinase dependent (“dependent” meaning also “supported”, not only “solely dependent”) diseases mentioned herein, especially proliferative diseases mentioned herein, more especially any one or more of these or other diseases that depend on one or more of protein or lipid kinases such as PI4K (phosphatidylinositol 4-kinase) and/or PI3 kinases (phosphatidylinositol 3-kinase), for example, inhibition of the PI3K superfamily which comprises PI3Kalpha, PI3Kbeta, PI3Kdelta, PI3Kgamma and mTOR, or one or more of the individual kinase members thereof, including Vps34 (class III PI3K), PI3-kinase-related protein kinase family (PIKK, class IV PI3K) which includes DNA-PK, ATM, ATR, hSMG-1 and mTOR, or any combinations of two or more of these, or a mutant of any one or more of these, and a compound of the formula (I) can therefore be used in the treatment of a lipid or protein kinase dependent disease, especially a disease depending on one or more of the kinases mentioned above and below, where (especially in the case of aberrantly highly-expressed, constitutively activated and/or mutated kinases or defective dephosphorylation of their substrate(s)) said kinase-dependent disease is dependent on the activity of one or more of the said kinases or the pathways in which they are involved.

The compounds of formula (I) have valuable pharmacological properties and are useful in the treatment of lipid and/or protein kinase dependent diseases, e.g., as drugs to treat proliferative diseases.

In the following detailed description and embodiments, which are to be taken independently, collectively or in any combination or sub-combination, the invention relates to a compound of the formula (I), in e.g. free base form or in acid addition salt form, wherein the substituents are as defined herein.

### DETAILED DESCRIPTION OF THE INVENTION

With respect to formula (I), the following detailed description is provided.

### R¹

As indicated hereinabove, R¹ is unsubstituted or substituted pyridyl, e.g. by the groups listed herein under “substituted”.

When the pyridyl is substituted, preferably by one, two or three substituents, said substituents may for example be independently selected from halo, unsubstituted or substituted alkyl, unsubstituted or substituted alkenyl, unsubstituted or substituted alkynyl, unsubstituted or substituted lower alkoxy, unsubstituted or substituted alkoxy lower alkyl, unsubstituted or substituted lower alkoxy lower alkoxy, unsubstituted or substituted cycloalkyl, unsubstituted or substituted cycloalkenyl, unsubstituted or substituted carbamoyl lower alkyl, lower mono- and di-alkyl carbamoyl lower alkyl, unsubstituted or substituted cycloaminocarbonyl lower alkyl, unsubstituted or substituted heterocyclylcarbonyl lower alkyl, unsubstituted or substituted hydroxyl lower alkyl, unsubstituted or substituted carboxy lower alkyl, halo-loweralkyl, hydroxycarbonyl-loweralkyl, unsubstituted or substituted pyrazinyl, hydroxyl, amino, mono- or di-loweralkyl substituted amino, unsubstituted or substituted piperazinyl, N-loweralkyl-N-lower alkoxy lower alkyl substituted amino, lower alkoxy lower alkyl carbonyl amino, oxo-piperazinyl, unsubstituted or substituted cycloamino, N-loweralkyl-N-hydroxy lower alkyl substituted amino.

In another embodiment, when the pyridyl is substituted, preferably by one, two or three substituents, said substituents may for example be independently selected from halo, unsubstituted or substituted alkyl, unsubstituted or substituted alkenyl, unsubstituted or substituted alkynyl, unsubstituted or substituted lower alkoxy, unsubstituted or substituted alkoxy lower alkyl, unsubstituted or substituted lower alkoxy lower alkoxy, unsubstituted or substituted cycloalkyl, unsubstituted or substituted cycloalkenyl, unsubstituted or substituted carbamoyl lower alkyl, lower mono- and di-alkyl carbamoyl lower alkyl, unsubstituted or substituted cycloaminocarbonyl lower alkyl, unsubstituted or substituted heterocyclylcarbonyl lower alkyl, unsubstituted or substituted hydroxyl lower alkyl, unsubstituted or substituted carboxy lower alkyl, halo-loweralkyl, hydroxycarbonyl-loweralkyl, unsubstituted or substituted pyrazinyl, hydroxyl, amino, mono- or di-loweralkyl substituted amino, unsubstituted or substituted piperazinyl.

Preferred substituents are independently selected from halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino.

In another embodiment, preferred pyridyl substituents are independently selected from halo, unsubstituted or substituted alkyl, unsubstituted or substituted lower alkoxy, unsubstituted or substituted lower alkoxy lower alkoxy, mono- or di-loweralkyl substituted amino, unsubstituted or substituted piperazinyl.

It is preferred that the substituent (if mono-substituted) or at least one of the substituents (if two or three substituents are present) is bonded to the pyridyl ring at the alpha position to the ring carbon atom which bonds the pyridyl ring to the rest of the molecule (that is, the alpha position is at a position on the pyridyl ring which is next to the pyridyl ring carbon atom which is bonded to the imidazoquinolinone part of the molecule). The pyridyl ring atom which is bonded to the rest of the molecule (i.e. the imidazoquinolinone part) is a carbon atom. Thus, preferred substituted pyridyl groups are alpha-substituted pyridyl, preferably alpha-substituted on a carbon ring-atom. For the avoidance of doubt the ring atom at the alpha position may be a carbon or nitrogen ring atom. Preferred pyridyl may be a pyridin-2-yl or a pyridin-3-yl.

An embodiment of the invention includes compounds of formula (I) wherein R¹ is a group selected from:

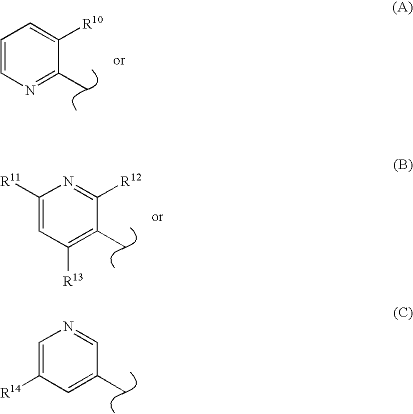

wherein, the curved line indicates the bonding position to the rest of the molecule, and

R¹⁰ is independently selected from hydrogen, halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino.

Preferably, R¹⁰ is independently selected from hydrogen, halo, unsubstituted or substituted alkyl, unsubstituted or substituted lower alkoxy.

More preferably, R¹⁰ is independently selected from hydrogen, halo, unsubstituted lower alkyl, unsubstituted lower alkoxy.

More preferably, R¹⁰ is independently selected from hydrogen, fluoro, lower alkyl such as methyl or ethyl, lower alkoxy such as methoxy or ethoxy.

Very preferably, R¹⁰ is independently selected from hydrogen, fluoro, methyl and methoxy.

Most preferred, R¹⁰ is methyl.

R¹¹ is independently selected from hydrogen, halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino.

Preferably, R¹¹ is independently selected from hydrogen, halo, unsubstituted lower alkyl, substituted lower alkyl such as hydroxy lower alkyl, unsubstituted lower alkoxy, unsubstituted lower alkoxy lower alkoxy, amino, mono- or di-loweralkyl substituted amino, unsubstituted or substituted piperazinyl, N-loweralkyl-N-lower alkoxy lower alkyl substituted amino, lower alkoxy lower alkyl carbonyl amino, substituted oxo-piperazinyl, cycloamino, halo-substituted cycloamino, hydroxy-substituted cycloamino, alkoxy-substituted cycloamino, N-loweralkyl-N-hydroxy lower alkyl substituted amino.

More preferably, R¹¹ is independently selected from hydrogen, fluoro, lower alkyl such as methyl or ethyl, hydroxy lower alkyl such as hydroxy propyl, lower alkoxy such as methoxy or ethoxy, lower alkoxy lower alkoxy such as methoxyethoxy or ethoxymethoxy or ethoxyethoxy, amino, di-loweralkyl substituted amino such as di-methyl amino, alkyl substituted piperazinyl such as methyl or ethyl substituted piperazinyl, alkyl substituted oxo-piperazinyl such as methyl or ethyl substituted oxo-piperazinyl, N-loweralkyl-N-lower alkoxy lower alkyl substituted amino such as N-methyl-N-methoxyethyl amino or N-methyl-N-methoxymethyl amino, lower alkoxy lower alkyl carbonyl amino such as methoxymethyl carbonyl amino or methoxyethyl carbonyl amino, cycloamino such as azetidinyl or pyrrolidinyl, halo-substituted cycloamino such as fluoro-substituted azetidinyl or fluoro-substituted pyrrolidinyl, hydroxy-substituted cycloamino such as hydroxy azetidinyl or hydroxy pyrrolidinyl, alkoxy-substituted cycloamino such as methoxy azetidinyl or methoxy pyrrolidinyl, N-loweralkyl-N-hydroxy lower alkyl substituted amino such as N-hydroxyethyl-N-methoxy amino.

Very preferably, R¹¹ is independently selected from hydrogen, fluoro, methyl, methoxy, methoxyethoxy, piperazinyl, 1-methylpiperazinyl, amino, N-ethyl-N-methyl amino, N-(methyl)-N-(methoxyethyl)amino, dimethyl amino, 4-methyl-3-oxo-piperazin-1-yl, azetidin-1-yl, 3,3-difluoro azetidin-1-yl, 3-hydroxy azetidin-1-yl, pyrrolidinyl, 3-hydroxy pyrrolidinyl, 3-methoxy pyrrolidinyl, 3,3,4,4-tetrafluoro pyrrolidinyl, trifluoromethyl, 2-hydroxy propan-2-yl, N-hydroxyethyl-N-methoxy amino.

R¹² is independently selected from hydrogen, halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino.

Preferably, R¹² is independently selected from hydrogen, halo, unsubstituted or substituted alkyl, unsubstituted or substituted lower alkoxy.

More preferably, R¹² is independently selected from hydrogen, halo, unsubstituted lower alkyl, unsubstituted lower alkoxy.

More preferably, R¹² is independently selected from hydrogen, fluoro, chloro, lower alkyl such as methyl or ethyl, lower alkoxy such as methoxy or ethoxy.

Very preferably, R¹² is independently selected from hydrogen, fluoro, chloro, methyl and methoxy.

R¹³ is independently selected from hydrogen, halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino.

Preferably, R¹³ is independently selected from hydrogen, halo, unsubstituted or substituted alkyl, unsubstituted or substituted lower alkoxy.

More preferably, R¹³ is independently selected from hydrogen, halo, unsubstituted lower alkyl, unsubstituted lower alkoxy.

More preferably, R¹³ is independently selected from hydrogen, fluoro, lower alkyl such as methyl or ethyl, lower alkoxy such as methoxy or ethoxy.

Very preferably, R¹³ is independently selected from hydrogen, fluoro, methyl and methoxy.

Most preferably, R¹³ is hydrogen or methyl.

R¹⁴ is independently selected from hydrogen, halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino.

Preferably, R¹⁴ is independently selected from hydrogen, halo, unsubstituted or substituted alkyl, unsubstituted or substituted lower alkoxy.

More preferably, R¹⁴ is independently selected from hydrogen, halo, unsubstituted lower alkyl, unsubstituted lower alkoxy.

More preferably, R¹⁴ is independently selected from hydrogen, fluoro, lower alkyl such as methyl or ethyl, lower alkoxy such as methoxy or ethoxy.

Very preferably, R¹⁴ is independently selected from hydrogen, fluoro, methyl and methoxy.

Most preferably, R¹⁴ is hydrogen or methyl.

In an embodiment of the present invention, with respect to formula (B) shown above, it is preferred that when R¹¹ and R¹² are hydrogen, R¹³ is not hydrogen, preferably then, R¹³ is a group listed above, most preferably, lower alkyl, especially methyl or lower alkyoxy, especially methoxy.

In another embodiment, the invention includes compounds of formula (I) wherein R¹ is a group selected from:

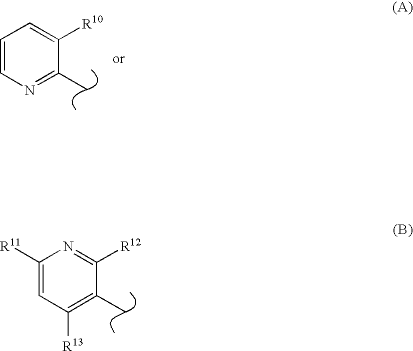

wherein, the curved line indicates the bonding position to the rest of the molecule, and

R¹⁰ is independently selected from hydrogen, halo, unsubstituted or substituted alkyl, unsubstituted or substituted lower alkoxy, unsubstituted or substituted lower alkoxy lower alkoxy, mono- or di-loweralkyl substituted amino, unsubstituted or substituted piperazinyl.

Preferably, R¹⁰ is independently selected from hydrogen, halo, unsubstituted or substituted alkyl, unsubstituted or substituted lower alkoxy.

More preferably, R¹⁰ is independently selected from hydrogen, halo, unsubstituted lower alkyl, unsubstituted lower alkoxy.

More preferably, R¹⁰ is independently selected from hydrogen, fluoro, lower alkyl such as methyl or ethyl, lower alkoxy such as methoxy or ethoxy.

Very preferably, R¹⁰ is independently selected from hydrogen, fluoro, methyl and methoxy.

Most preferred, R¹⁰ is methyl.

R¹¹ is independently selected from hydrogen, halo, unsubstituted or substituted alkyl, unsubstituted or substituted lower alkoxy, unsubstituted or substituted lower alkoxy lower alkoxy, mono- or di-loweralkyl substituted amino, unsubstituted or substituted piperazinyl.

Preferably, R¹¹ is independently selected from hydrogen, halo, unsubstituted lower alkyl, unsubstituted lower alkoxy, unsubstituted lower alkoxy lower alkoxy, mono- or di-loweralkyl substituted amino, unsubstituted or substituted piperazinyl.

More preferably, R¹¹ is independently selected from hydrogen, fluoro, lower alkyl such as methyl or ethyl, lower alkoxy such as methoxy or ethoxy, lower alkoxy lower alkoxy such as methoxyethoxy or ethoxymethoxy or ethoxyethoxy, alkyl substituted piperazinyl such as methyl or ethyl substituted piperazinyl.

Very preferably, R¹¹ is independently selected from hydrogen, fluoro, methyl, methoxy, methoxyethoxy, 1-methylpiperazinyl.

R¹² is independently selected from hydrogen, halo, unsubstituted or substituted alkyl, unsubstituted or substituted lower alkoxy, unsubstituted or substituted lower alkoxy lower alkoxy, mono- or di-loweralkyl substituted amino, unsubstituted or substituted piperazinyl.

Preferably, R¹² is independently selected from hydrogen, halo, unsubstituted or substituted alkyl, unsubstituted or substituted lower alkoxy.

More preferably, R¹² is independently selected from hydrogen, halo, unsubstituted lower alkyl, unsubstituted lower alkoxy.

More preferably, R¹² is independently selected from hydrogen, fluoro, lower alkyl such as methyl or ethyl, lower alkoxy such as methoxy or ethoxy.

Very preferably, R¹² is independently selected from hydrogen, fluoro, methyl and methoxy.

R¹³ is independently selected from hydrogen, halo, unsubstituted or substituted alkyl, unsubstituted or substituted lower alkoxy, unsubstituted or substituted lower alkoxy lower alkoxy, mono- or di-loweralkyl substituted amino, unsubstituted or substituted piperazinyl.

Preferably, R¹³ is independently selected from hydrogen, halo, unsubstituted or substituted alkyl, unsubstituted or substituted lower alkoxy.

More preferably, R¹³ is independently selected from hydrogen, halo, unsubstituted lower alkyl, unsubstituted lower alkoxy.

More preferably, R¹³ is independently selected from hydrogen, fluoro, lower alkyl such as methyl or ethyl, lower alkoxy such as methoxy or ethoxy.

Very preferably, R¹³ is independently selected from hydrogen, fluoro, methyl and methoxy.

Most preferably, R¹³ is methyl.

In an embodiment of the present invention, with respect to formula (B) shown above, it is preferred that when R¹¹ and R¹² are hydrogen, R¹³ is not hydrogen, preferably then, R¹³ is a group listed above, most preferably, lower alkyl, especially methyl or lower alkyoxy, especially methoxy.

In another embodiment, the invention includes compounds of formula (I) wherein R¹ is the group:

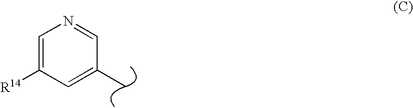

wherein, the curved line indicates the bonding position to the rest of the molecule, and

R¹⁴ is independently selected from hydrogen, halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino.

Preferably, R¹⁴ is independently selected from hydrogen, halo, unsubstituted or substituted alkyl, unsubstituted or substituted lower alkoxy.

More preferably, R¹⁴ is independently selected from hydrogen, halo, unsubstituted lower alkyl, unsubstituted lower alkoxy.

More preferably, R¹⁴ is independently selected from hydrogen, fluoro, lower alkyl such as methyl or ethyl, lower alkoxy such as methoxy or ethoxy.

Very preferably, R¹⁴ is independently selected from hydrogen, fluoro, methyl and methoxy.

Most preferably, R¹⁴ is hydrogen or methyl.

Very preferably, R¹ is a selected from the group consisting of pyridin-3-yl, 2-methyl-pyridin-3-yl, 3-methyl-pyridin-2-yl, 4-methyl-pyridin-3-yl, 2,6-dimethyl-pyridin-3-yl, 2-fluoro-pyridin-3-yl, 6-fluoro-2-methyl-pyridin-3-yl, 2,6-dimethoxy-pyridin-3-yl, 6-methoxy-2-methyl-pyridin-3-yl, 2-methoxy-6-piperazin-1-yl-pyridin-3-yl, 2-methoxy-6-(4-methyl-piperazin-1-yl)-pyridin-3-yl, 6-(N-methyl-N-ethyl amino)-2-methyl-pyridin-3-yl, 6-(methoxyethoxy)-2-methyl-pyridin-3-yl, 6-(N-methyl-N-methoxyethyl amino)-2-methyl-pyridin-3-yl, 6-fluoro-4-methyl-pyridin-3-yl, 5-methyl-pyridin-3-yl, 2-chloro-6-methoxy-pyridin-3-yl, 2-methyl-6-dimethylamino-pyridin-3-yl, 2-methyl-6-[N-methoxymethyl carbonyl]-amino-pyridin-3-yl, 2-methyl-6-(4-methyl-3-oxo-piperazin-1-yl)-pyridin-3-yl, 6-azetidinyl-2-methyl-pyridin-3-yl, 6-pyrrolidinyl-2-methyl-pyridin-3-yl, 6-(3,3-difluoro-azetidinyl)-2-methyl-pyridin-3-yl, 6-[N-(2-hydroxyethyl)-N-methylamino]-2-methyl-pyridin-3-yl, 6-(3-hydroxypyrrolidinyl)-2-methyl-pyridin-3-yl, 6-((S)-3-hydroxypyrrolidinyl)-2-methyl-pyridin-3-yl, 6-((R)-3-hydroxypyrrolidinyl)-2-methyl-pyridin-3-yl, 6-(3-methoxypyrrolidinyl)-2-methyl-pyridin-3-yl, 6-(3-hydroxy-azetidinyl)-2-methyl-pyridin-3-yl, 6-amino-2-methyl-pyridin-3-yl, 6-(2-hydroxyprop-2-yl)-2-methyl-pyridin-3-yl, 6-trifluoromethyl-2-methyl-pyridin-3-yl, 6-(3,3,4,4-tetrafluoropyrrolidinyl)-2-methyl-pyridin-3-yl.

In another embodiment, R¹ is a selected from the group consisting of 6-(N-methyl-N-ethyl amino)-2-methyl-pyridin-3-yl, 6-(methoxyethoxy)-2-methyl-pyridin-3-yl, 6-(N-methyl-N-methoxyethyl amino)-2-methyl-pyridin-3-yl, 6-fluoro-4-methyl-pyridin-3-yl, 5-methyl-pyridin-3-yl, 2-chloro-6-methoxy-pyridin-3-yl, 2-methyl-6-dimethylamino-pyridin-3-yl, 2-methyl-6-[N-methoxymethyl carbonyl]amino-pyridin-3-yl, 2-methyl-6-(4-methyl-3-oxo-piperazin-1-yl)-pyridin-3-yl, 6-azetidinyl-2-methyl-pyridin-3-yl, 6-pyrrolidinyl-2-methyl-pyridin-3-yl, 6-(3,3-difluoro-azetidinyl)-2-methyl-pyridin-3-yl, 6-[N-(2-hydroxyethyl)-N-methylamino]-2-methyl-pyridin-3-yl, 6-(3-hydroxypyrrolidinyl)-2-methyl-pyridin-3-yl, 6-((S)-3-hydroxypyrrolidinyl)-2-methyl-pyridin-3-yl, 6-((R)-3-hydroxypyrrolidinyl)-2-methyl-pyridin-3-yl, 6-(3-methoxypyrrolidinyl)-2-methyl-pyridin-3-yl, 6-(3-hydroxy-azetidinyl)-2-methyl-pyridin-3-yl, 6-amino-2-methyl-pyridin-3-yl, 6-(2-hydroxyprop-2-yl)-2-methyl-pyridin-3-yl, 6-trifluoromethyl-2-methyl-pyridin-3-yl, 6-(3,3,4,4-tetrafluoropyrrolidinyl)-2-methyl-pyridin-3-yl.

In another embodiment, R¹ is a selected from the group consisting of pyridin-3-yl, 2-methyl-pyridin-3-yl, 3-methyl-pyridin-2-yl, 4-methyl-pyridin-3-yl, 2,6-dimethyl-pyridin-3-yl, 2-fluoro-pyridin-3-yl, 6-fluoro-2-methyl-pyridin-3-yl, 2,6-dimethoxy-pyridin-3-yl, 6-methoxy-2-methyl-pyridin-3-yl, 2-methoxy-6-piperazin-1-yl-pyridin-3-yl, 2-methoxy-6-piperazin-1-yl-pyridin-3-yl, 2-methoxy-6-(4-methyl-piperazin-1-yl)-pyridin-3-yl. R²

R² is preferably selected from hydrogen or C₁-C₄alkyl. Most preferably R² is selected from hydrogen, methyl and ethyl.

R² is very preferably methyl.

### R³

R³ is unsubstituted or substituted aryl or unsubstituted or substituted heterocycyl as defined herein above, each of which, when substituted, is substituted by one or more, especially 1-3, substituents independently selected from the group consisting of the substituents defined under “substituted”.

When R³ is unsubstituted or substituted aryl, it is preferably C₆-C₁₀aryl which is unsubstituted, or substituted by one or more, especially 1-3, substituents independently selected from the group consisting of the substituents defined under “substituted”.

When R³ is an aryl group, (especially C₆-C₁₀aryl, more especially phenyl) it is unsubstituted, or substituted by one or more, especially 1-3, substituents independently selected from the group consisting of the substituents defined under “substituted”.

When the R₃ group is an aryl group, (especially C₆-C₁₀aryl, more especially phenyl) when substituted, the substituents may be selected from the group consisting of halo, especially fluoro, chloro, bromo or iodo, in particular fluoro; halo-lower alkyl, especially fluoroalkyl, in particular trifluoromethyl; hydroxyl; amino; mono or disubstituted amino, especially alkyl-substituted amino or hydroxyalkyl-substituted amino, e.g. dimethyl amino or 2-hydroxyethyl amino; cyclic amino, such as aziridinyl or azetidinyl; amino-lower alkyl, e.g., aminomethyl, 2-aminoethyl or 3-aminopropyl; lower alkoxy, e.g., methoxy or ethoxy; hydroxy-lower alkyl, e.g., hydroxymethyl or 2-hydroxyethyl; hydroxy lower alkoxy, e.g. hydroxyethoxy; lower alkyl, e.g., methyl, ethyl or iso-propyl; cyano; cyano-lower alkyl, e.g., 2-cyanoethyl or 3-cyanopropyl; amidino; N-hydroxyamidino; hydroxyamino; alkoxyamino; nitro; amidino-lower alkyl, e.g., 2-amidino-ethyl; or N-hydroxyamidino-lower alkyl, e.g., 2-(N-hydroxyamidino)-ethyl; substituted phenyl or (especially 1- or 2-)naphthyl; sulfonyl; substituted sulfonyl, e.g. alkyl-substituted sulfonyl, such as methanesulfonyl; sulfonamide, e.g. N-methylsulfonamide or pyrrolidine-1-sulfonyl; [1,3]dioxolo; substituted [1,3]dioxolo, e.g. 2,2-difluoro-[1,3]dioxolo; alkoxy carbonyl, such as lower alkoxy carbonyl, e.g. methoxycarbonyl; carbamoyl; substituted carbamoyl, such as alkyl-substituted carbamoyl, e.g. methylcarbamoyl; heterocycle, e.g. pyrazol; heterocyclyl lower alkyl; heteroaryl or heteroaryl lower alkyl.

Preferred unsubstituted or substituted aryl for R₃ is selected from phenyl; hydroxyphenyl, e.g., 2-, 3- or 4-hydroxyphenyl; methoxyphenyl, e.g., 2-, 3- or 4-methoxyphenyl or 3,4-dimethoxyphenyl; ethoxyphenyl, e.g., 2-, 3- or 4-ethoxyphenyl or 3,4-diethoxyphenyl; methoxy ethoxy-phenyl, e.g. 3-methoxy-4-ethoxy phenyl or 4-methoxy-3-ethoxy phenyl, other lower-alkoxy phenyl, e.g. 3-methoxy-4-(2-methoxy ethoxy)-phenyl, hydroxy alkoxy phenyl, e.g. 3-methoxy-4-hydroxy phenyl; halo-hydroxy-phenyl, e.g. fluoro-hydroxy-phenyl such as 3-fluoro-5-hydroxy-phenyl; hydroxy-haloalkyl-phenyl, e.g. hydroxy-fluoroalkyl-phenyl such as 3-hydroxy-5-trifluoromethyl-phenyl; 2,2-difluoro-benzo[1,3]dioxolo; benzene sulfonamide, e.g. N-methylbenzenesulfonamide; 3-(pyrrolidine-1-sulfonyl)-phenyl, N-(phen-3-yl)-methanesulfonamide or N-methyl-N-phen-3-yl-methanesulfonamide; alkyl-sulfonyl phenyl, e.g. 3-methanesulfonylphenyl; benzamide e.g. 2-, 3- or 4-benzamide, 2-, 3- or 4-N-methyl-benzamide or 2-, 3- or 4-N,N-dimethyl-benzamide; pyrazol-phenyl, e.g. 4-(pyrazol)-phenyl or alternatively (1H-pyrazol-1-yl)-phenyl, in particular 4-(1H-pyrazol-1-yl)-phenyl; imidazol-phenyl, especially (1H-imidazol-2-yl)phenyl, in particular 4-(1H-imidazol-2-yl)phenyl.

When the R₃ group is a heterocyclyl or preferably heteroaryl, said heterocyclyl or heteroaryl may be selected from the group consisting of indolyl, 2,3-dihydro-1H-indol-5-yl, 1-methyl-2,3-dihydro-1H-indol-5-yl, 2-oxo-2,3-dihydro-1H-indol-5-yl, pyridyl (e.g. pyridin-2-yl, pyridin-3-yl or pyridin-4-yl), pyrimidinyl (especially pyrimidin-5-yl), 1H-pyrrolo[2,3-b]pyridin-5-yl, 1-methyl-1H-pyrrolo[2,3-b]pyridin-5-yl, pyrazolyl, pyrazol-4-yl, pyrazinyl, quinolyl, quinol-3-yl, 1H-imidazo[4,5-b]pyridin-2(3H)-one-6-yl, 3H-imidazo[4,5-b]pyridin-6-yl, 3H-[1,2,3]triazolo[4,5-b]pyridin-6-yl, imidazo[1,2-a]pyridin-6-yl each of these heterocycle radicals being unsubstituted or substituted by one or two radicals selected from the substituents described under “substituted”, in particular from the group consisting of halo, especially fluoro, chloro, bromo or iodo, more especially fluoro or chloro; halo-lower alkyl, especially fluoroalkyl, in particular trifluoromethyl; hydroxyl; amino, mono or disubstituted amino, especially alkyl-substituted amino or hydroxyalkyl-substituted amino, e.g. methyl amino, ethyl amino, dimethyl amino, iso-propyl amino or 2-hydroxyethyl amino; cyclic amino, such as aziridinyl or azetidinyl; amino-lower alkyl, e.g., aminomethyl, 2-aminoethyl or 3-aminopropyl; lower alkoxy, e.g., methoxy or ethoxy or iso-propoxy; lower alkoxy lower alkyl, including di-lower alkoxy lower alkyl, e.g. 2-methoxyethyl, dimethoxy-methyl, methoxymethyl, ethoxymethyl; hydroxy-lower alkyl, e.g., hydroxymethyl or 2-hydroxyethyl or 2-hydroxy prop-2-yl; hydroxy lower alkoxy, e.g. hydroxyethoxy; lower alkoxy lower alkoxy, e.g. 2-methoxyethoxy; lower alkyl, e.g., methyl, ethyl or iso-propyl; cycloalkyl; substituted cycloalkyl, e.g. cyano-substituted cycloalkyl e.g. 1-cyanocyclobut-1-yl; cyano; cyano-lower alkyl, e.g., 2-cyanoethyl, 3-cyanopropyl, and 2-cyanoprop-2-yl; amidino; N-hydroxyamidino; amidino-lower alkyl, e.g., 2-amidino-ethyl; or N-hydroxyamidino-lower alkyl, e.g., 2-(N-hydroxyamidino)-ethyl; substituted phenyl or (especially 1- or 2-) naphthyl; sulfonyl; substituted sulfonyl, e.g. alkyl-substituted sulfonyl, such as methanesulfonyl; sulfonamide, e.g. N-methylsulfonamide or pyrrolidine-1-sulfonyl; [1,3]dioxolo; substituted [1,3]dioxolo, e.g. 2,2-difluoro-[1,3]dioxolo; alkoxy carbonyl, such as lower alkoxy carbonyl, e.g. methoxycarbonyl; carbamoyl; substituted carbamoyl, such as alkyl-substituted carbamoyl, e.g. methylcarbamoyl; aminocarbonylalkyl, such as aminocarbonyl loweralkyl, e.g. aminocarbonylmethyl; N-mono-substituted aminocarbonylalkyl, such as N-loweralkyl aminocarbonyl loweralkyl, e.g. methylaminocarbonylmethyl; N-di-substituted aminocarbonylalkyl, such as N-di-loweralkyl aminocarbonyl loweralkyl, e.g. dimethylaminocarbonylmethyl, or other N-di-substituted aminocarbonylalkyl, such as 4-morpholinecarbonylmethyl; aminoloweralkoxy; N-loweralkyl aminoloweralkoxy; N,N-di-loweralkyl aminoloweralkoxy, such as N,N-di-methylaminoloweralkoxy, e.g. N,N-dimethylaminopropoxy, N,N-dimethylaminoethoxy, N,N-dimethylaminomethoxy, especially 3-N,N-dimethylaminopropoxy. The heterocycle group may also be substituted with another heterocycle, e.g. 3H-tetrazolyl (in particular 3H-tetrazol-5-yl), pyrazol, heterocyclyl lower alkyl, heteroaryl or heteroaryl lower alkyl as defined herein.

In an embodiment preferred heterocyclic groups for R³, include pyridyl, pyrimidinyl, 1H-pyrrolo[2,3-b]pyridinyl, 1-methyl-1H-pyrrolo[2,3-b]pyridinyl, imidazo[1,2-a]pyridinyl, quinolyl, each of which may be unsubstituted or substituted as indicated above.

In another embodiment, preferred heterocyclic groups for R³, include pyridyl, pyrimidinyl, 1H-pyrrolo[2,3-b]pyridinyl, quinolyl, each of which may be unsubstituted or substituted as indicated above.

In an embodiment, R³ is unsubstituted pyrid-3-yl or pyrid-3-yl substituted by one or two substituents independently selected from those listed under “substituted” or, in another embodiment, one or two substituents independently selected from those recited in relation to substituted heterocyclyl or heteroaryl.

In an embodiment, R³ is said substituted pyrid-3-yl.

When the R₃ group is a heterocyclyl or preferably heteroaryl, said heterocyclyl or heteroaryl may be selected from the group consisting of indolyl, 2,3-dihydro-1H-indol-5-yl, 1-methyl-2,3-dihydro-1H-indol-5-yl, 2-oxo-2,3-dihydro-1H-indol-5-yl, pyridyl (e.g. pyridin-2-yl, pyridin-3-yl or pyridin-4-yl), pyrimidinyl (especially pyrimidin-5-yl), 1H-pyrrolo[2,3-b]pyridin-5-yl, 1-methyl-1H-pyrrolo[2,3-b]pyridin-5-yl, pyrazolyl, pyrazol-4-yl, pyrazinyl, quinolyl, quinol-3-yl, 1H-imidazo[4,5-b]pyridin-2(3H)-one-6-yl, 3H-imidazo[4,5-b]pyridin-6-yl, 3H-[1,2,3]triazolo[4,5-b]pyridin-6-yl each of these heterocycle radicals being unsubstituted or substituted by one or two radicals selected from the substituents described under “substituted”, in particular from the group consisting of halo, especially fluoro, chloro, bromo or iodo, more especially fluoro or chloro; halo-lower alkyl, especially fluoroalkyl, in particular trifluoromethyl; hydroxyl; amino, mono or disubstituted amino, especially alkyl-substituted amino or hydroxyalkyl-substituted amino, e.g. methyl amino, dimethyl amino or 2-hydroxyethyl amino; cyclic amino, such as aziridinyl or azetidinyl; amino-lower alkyl, e.g., aminomethyl, 2-aminoethyl or 3-aminopropyl; lower alkoxy, e.g., methoxy or ethoxy; lower alkoxy lower alkyl, e.g. 2-methoxyethyl; hydroxy-lower alkyl, e.g., hydroxymethyl or 2-hydroxyethyl; hydroxy lower alkoxy, e.g. hydroxyethoxy; lower alkyl, e.g., methyl, ethyl or iso-propyl; cyano; cyano-lower alkyl, e.g., 2-cyanoethyl and 3-cyanopropyl; amidino; N-hydroxyamidino; amidino-lower alkyl, e.g., 2-amidino-ethyl; or N-hydroxyamidino-lower alkyl, e.g., 2-(N-hydroxyamidino)-ethyl; substituted phenyl or (especially 1- or 2-) naphthyl; sulfonyl; substituted sulfonyl, e.g. alkyl-substituted sulfonyl, such as methanesulfonyl; sulfonamide, e.g. N-methylsulfonamide or pyrrolidine-1-sulfonyl; [1,3]dioxolo; substituted [1,3]dioxolo, e.g. 2,2-difluoro-[1,3]dioxolo; alkoxy carbonyl, such as lower alkoxy carbonyl, e.g. methoxycarbonyl; carbamoyl; substituted carbamoyl, such as alkyl-substituted carbamoyl, e.g. methylcarbamoyl; aminocarbonylalkyl, such as aminocarbonyl loweralkyl, e.g. aminocarbonylmethyl; N-mono-substituted aminocarbonylalkyl, such as N-loweralkyl aminocarbonyl loweralkyl, e.g. methylaminocarbonylmethyl; N-di-substituted aminocarbonylalkyl, such as N-di-loweralkyl aminocarbonyl loweralkyl, e.g. dimethylaminocarbonylmethyl, or other N-di-substituted aminocarbonylalkyl, such as 4-morpholinecarbonylmethyl; aminoloweralkoxy; N-loweralkyl aminoloweralkoxy; N,N-di-loweralkyl aminoloweralkoxy, such as N,N-di-methylaminoloweralkoxy, e.g. N,N-dimethylaminopropoxy, N,N-dimethylaminoethoxy, N,N-dimethylaminomethoxy, especially 3-N,N-dimethylaminopropoxy. The heterocycle group may also be substituted with another heterocycle, e.g. 3H-tetrazolyl (in particular 3H-tetrazol-5-yl), pyrazol, heterocyclyl lower alkyl, heteroaryl or heteroaryl lower alkyl as defined herein. Very preferred heterocyclic groups for R³, include pyridyl, pyrimidinyl, 1H-pyrrolo[2,3-b]pyridinyl, quinolyl, each of which may be unsubstituted or substituted as indicated above.

Specifically preferred unsubstituted or substituted heterocyclyl or heteroaryl for R₃ is selected from pyridyl, especially pyridin-3-yl;

alkylpyridyl, in particular loweralkylpyridyl, e.g. methylpyridyl, e.g., 2-, 3- or 4-methylpyridyl, especially 2-methylpyridyl or 3-methylpyridyl, in particular 2-methylpyrid-5-yl or 3-methylpyrid-5-yl;
alkoxypyridyl, in particular lower-alkoxypyridyl, e.g. methoxypyridyl, e.g., 2-, 3- or 4-methoxypyridyl, especially 2-methoxypyridyl or 3-methoxypyridyl (in particular 2-methoxpyridin-5-yl, 3-methoxpyridin-5-yl); ethoxypyridyl, e.g., 2-, 3- or 4-ethoxypyridyl especially 2-ethoxypyridyl or 3-ethoxypyridyl (in particular 3-ethoxypyridin-5-yl, 2-ethoxypyridin-5-yl, 2-ethoxypyridin-4-yl); propoxypyridyl, e.g. n-propoxypyridyl or iso-propoxypyridyl (in particular 6-(n-propoxy)pyrid-3-yl or 3-(iso-propoxy)pyridin-5-yl);
cycloalkylalkoxypyridyl, e.g. cyclopropylmethoxy-pyridyl, especially 2-cyclopropylmethoxy-pyridyl (in particular 2-cyclopropylmethoxy-pyrid-5-yl);
alkoxyalkylpyridyl e.g. ethoxymethylpyridyl, 2-methoxymethylpyridyl (in particular 2-methoxymethylpyrid-5-yl);
alkoxyalkoxypyridyl, in particular methoxyethoxypyridyl, e.g. 3-(2-methoxyethoxy)pyridyl or 2-(2-methoxyethoxy)pyridyl (in particular 3-(2-methoxyethoxy)pyrid-5-yl or 2-(2-methoxyethoxy)pyrid-5-yl);
benzyloxyalkoxypyridyl, in particular benzyloxyethoxypyridyl or benzyloxypropoxypyridyl, e.g. 2-benzyloxyethoxypyridyl or 3-benzyloxypropoxypyridyl (in particular 2-benzyloxyethoxypyrid-5-yl or 3-benzyloxypropoxypyrid-5-yl);
hydroxyalkylpyridyl, e.g. hydroxymethylpyridyl or (hydroxyprop-2-yl)pyridyl, especially 2-(hydroxymethyl)-pyridyl or 3-(2-hydroxyprop-2-yl)pyridyl (in particular 2-hydroxymethylpyrid-5-yl or 3-(2-hydroxyprop-2-yl)pyrid-5-yl);
alkyl-sulfonyl pyridyl, e.g. methanesulfonylpyridyl, especially 3-methanesulfonylpyridyl (in particular 3-methanesulfonylpyrid-5-yl);
hydroxyalkoxypyridyl, e.g. 2-(2-hydroxyethoxy)-pyridyl or 2-(3-hydroxypropoxy)-pyridyl (in particular 2-(2-hydroxyethoxy)-pyrid-5-yl or 2-(3-hydroxypropoxy)-pyrid-5-yl); alkoxycarbonylpyridyl, e.g. methoxycarbonylpyridyl, especially 2-methoxycarbonyl-pyridyl (in particular 2-methoxycarbonyl-pyrid-5-yl);
aminopyridyl, e.g. 2- or 3-aminopyridyl (in particular 2-aminopyridin-5-yl or 3-aminopyrid-5-yl);
alkylaminopyridyl, e.g. loweralkylaminopyridyl, in particular methylaminopyridyl, more particularly 2-methylaminopyridyl or 3-methylaminopyridyl (especially 2-methylaminopyrid-5-yl or 3-methylaminopyrid-5-yl) or (iso-propyl amino)pyridyl, more particularly 3-(iso-propyl amino)pyridyl (especially 3-(iso-propyl amino)pyrid-5-yl);
dialkylaminopyridyl, in particular di-loweralkylaminopyridyl, e.g. 2-, 3- or 4-dimethylaminopyridyl, especially 2-dimethylaminopyridyl (in particular 2-dimethylaminopyrid-5-yl);
cycloaminopyridyl, e.g. azetidinylpyridyl, especially 2-azetidin-1-yl-pyridinyl or 3-azetidin-1-yl-pyridinyl (in particular 2-azetidin-1-yl-pyridin-5-yl or 3-azetidin-1-yl-pyridin-5-yl);
(cyanoloweralkyl)-pyridinyl such as (2-cyanoprop-2-yl)-pyridinyl, especially 3-(2-cyanoprop-2-yl)-pyridinyl (in particular 3-(2-cyanoprop-2-yl)-pyridin-5-yl);
(cyanocycloloweralkyl)-pyridinyl such as (1-cyanocyclobut-1-yl)-pyridinyl, especially 3-(1-cyanocyclobut-1-yl)-pyridinyl (in particular 3-(1-cyanocyclobut-1-yl)-pyridin-5-yl);
hydroxyalkylaminopyridyl, e.g. 2-(2-hydroxyethylamino)-pyridyl (in particular 2-(2-hydroxyethylamino)-pyrid-5-yl);
amino-haloloweralkyl-pyridyl, e.g. amino-trifluoromethyl-pyridyl, especially 2-amino-3-trifluoromethyl-pyridyl (in particular 2-amino-3-trifluoromethyl-pyrid-5-yl);
haloalkylpyridinyl, in particular haloloweralkylpyridyl, especially, 2-, 3- or 4-trifluoromethylpyridyl, most especially 2-trifluoromethylpyridyl (in particular 2-trifluoromethylpyrid-5-yl);
halopyridyl, in particular fluoropyridyl, especially 2-fluoropyridyl (in particular 2-fluoropyrid-3-yl or 2-fluoropyrid-4-yl or 2-fluoropyrid-5-yl);
halo-alkoxy-pyridyl, e.g. fluoro-methoxy-pyridyl such as 3-fluoro-2-methoxy-pyridyl (in particular 3-fluoro-2-methoxy-pyrid-5-yl);
carbamoylpyridyl, especially 2-(carbamoyl)pyridyl (in particular 2-(carbamoyl)pyrid-5-yl);
alkyl-substituted carbamoyl, e.g. methylcarbamoyl, especially 2-(methylcarbamoyl)pyridyl (in particular 2-(methylcarbamoyl)pyrid-5-yl);
piperazinylpyridyl, e.g. 1-piperazinylpyridyl, especially 2-(1-piperazinyl)pyridyl (in particular 2-(1-piperazinyl)pyrid-5-yl);
N-alkylpiperazinylpyridyl, such as N-loweralkylpiperazinylpyridyl, e.g. N-methylpiperazinylpyridyl, especially 2-(4-methylpiperazin-1-yl)-pyridyl (in particular 2-(4-methylpiperazin-1-yl)-pyrid-5-yl);
alkylsulfonamidopyridyl, such as loweralkylsulfonamidopyridyl, especially methylsulfonamidopyridyl, e.g. 3-(methylsulfonamido) pyridyl (in particular 3-(methylsulfonamido)-pyridin-5-yl);
dialkylsulfonamidopyridyl, such as diloweralkylsulfonamidopyridyl, especially dimethylsulfonamidopyridyl, e.g. 3-(dimethylsulfonamido)pyridyl (in particular 3-(dimethylsulfonamido)-pyridin-5-yl);
(alkylsulfonamido)(alkyl)pyridyl such as (loweralkylsulfonamido)(loweralkyl)pyridyl, especially (methylsulfonamido)(methyl)pyridyl, e.g. 3-(methylsulfonamido)(methyl)pyridyl (in particular 3-(methylsulfonamido)-2-methylpyridin-5-yl);
dialkylsulfonamido(alkyl)pyridyl, such as diloweralkylsulfonamido(loweralkyl)pyridyl, especially dimethylsulfonamido(methyl)pyridyl, e.g. 3-(dimethylsulfonamido)(2-methyl)pyridyl (in particular 3-(dimethylsulfonamido)-(2-methyl)-pyridin-5-yl);
3H-tetrazol-5-yl pyridyl, e.g. 2-(3H-tetrazol-5-yl)pyridyl (in particular 2-(3H-tetrazol-5-yl)pyrid-5-yl);
(alkoxy)(alkylcarbonylamino)pyridyl, such as (loweralkoxy)(loweralkylcarbonylamino)pyridyl, e.g. (methoxy)(methylcarbonylamino)pyridyl or (ethoxy)(methylcarbonylamino)pyridyl, especially 2-(methoxy)-3-(methylcarbonylamino)pyridyl or 2-(ethoxy)-3-(methylcarbonylamino)pyridyl (in particular 2-(methoxy)-3-(methylcarbonylamino)pyrid-5-yl or 2-(ethoxy)-3-(methylcarbonylamino)pyrid-5-yl);
(alkoxy)(alkylcarbonyl-N-alkylamino)pyridyl, such as (loweralkoxy)(loweralkylcarbonyl-N-loweralkylamino)pyridyl, e.g. (methoxy)(methylcarbonyl-N-methylamino)pyridyl or (ethoxy)(methylcarbonyl-N-methylamino)pyridyl, especially 2-(methoxy)-3-(methylcarbonyl-N-methylamino)pyridyl or 2-(ethoxy)-3-(methylcarbonyl-N-methylamino)pyridyl (in particular 2-(methoxy)-3-(methylcarbonyl-N-methylamino)pyrid-5-yl or 2-(ethoxy)-3-(methylcarbonyl-N-methylamino)pyrid-5-yl);
(alkoxy)(nitro)pyridyl, such as (loweralkoxy)(nitro)pyridyl, e.g. (methoxy)(nitro)pyridyl, especially 2-(methoxy)-3-(nitro)-pyridyl (in particular 2-(methoxy)-3-(nitro)-pyrid-5-yl);
(alkoxy)(amino)pyridyl, such as (loweralkoxy)(amino)pyridyl, e.g. (methoxy)(amino)pyridyl, especially 2-(methoxy)-3-(amino)-pyridyl (in particular 2-(methoxy)-3-(amino)-pyrid-5-yl);
(alkoxy)(alkylaminocarbonyl)pyridyl, such as (loweralkoxy)(loweralkylaminocarbonyl)pyridyl, e.g. (methoxy)(methylaminocarbonyl)pyridyl, especially 2-(methoxy)-3-(methylaminocarbonyl)pyridyl (in particular 2-(methoxy)-3-(methylaminocarbonyl)pyrid-5-yl;
(alkoxy)(hydroxycarbonyl)pyridyl, such as (loweralkoxy)(hydroxycarbonyl)pyridyl, e.g. (methoxy)(hydroxycarbonyl)pyridyl, especially 2-(methoxy)-3-(hydroxycarbonyl)pyridyl (in particular 2-(methoxy)-3-(hydroxycarbonyl)pyrid-5-yl;
N,N-di-loweralkyl aminoloweralkoxy, such as (N,N-dimethylaminopropoxy)pyridyl, e.g. 2-(N,N-dimethylaminopropoxy)pyridyl) (especially 2-(3-N,N-dimethylaminopropoxy)pyridin-5-yl);
(alkyl)(alkoxy)pyridyl, such as (loweralkyl)(loweralkoxy)pyridyl, e.g. (methyl)(methoxy)pyridyl or (methyl)(ethoxy)pyridyl or (methyl)(isopropoxy)pyridyl, especially 2-(methyl)-3-(methoxy)pyridyl or 2-(methyl)-3-(ethoxy)pyridyl or 2-(methyl)-3-(isopropoxy)pyridyl (in particular 2-(methyl)-3-(methoxy)pyrid-5-yl or 2-(methyl)-3-(ethoxy)pyrid-5-yl or 2-(methyl)-3-(isopropoxy)pyrid-5-yl);
(dialkoxy-alkyl)(alkoxy)pyridyl, such as (di-loweralkoxy-loweralkyl)(loweralkoxy)pyridyl e.g. (dimethoxy-methyl)(methoxy)pyridyl, especially 2-(dimethoxy-methyl)-3-(methoxy)pyridyl (in particular 2-(dimethoxy-methyl)-3-(methoxy)pyrid-5-yl);
(alkoxyalkyl)(alkoxy)pyridyl, such as (loweralkoxy loweralkyl)(loweralkoxy)pyridyl, e.g. (methoxy-methyl)(methoxy)pyridyl or (methoxy-methyl)(ethoxy)pyridyl or (methoxy-methyl)(iso-propoxy)pyridyl, especially 2-(methoxy-methyl)-3-(methoxy)pyridyl or 2-(methoxy-methyl)-3-(ethoxy)pyridyl or 2-(methoxy-methyl)-3-(iso-propoxy)pyridyl (in particular 2-(methoxy-methyl)-3-(methoxy)pyrid-5-yl or 2-(methoxy-methyl)-3-(ethoxy)pyrid-5-yl or 2-(methoxy-methyl)-3-(iso-propoxy)pyrid-5-yl);
(hydroxyalkyl)(alkylamino)pyridyl, such as (hydroxyloweralkyl)(loweralkylamino)pyridyl, e.g. (hydroxymethyl)(ethylamino)pyridyl or (hydroxymethyl)(methylamino)pyridyl, especially 2-(hydroxymethyl)-3-(ethylamino)pyridyl or 2-(hydroxymethyl)-3-(methylamino)pyridyl (in particular 2-(hydroxymethyl)-3-(ethylamino)pyrid-5-yl or 2-(hydroxymethyl)-3-(methylamino)pyrid-5-yl);
(alkyl)(alkylamino)pyridyl, such as (loweralkyl)(loweralkylamino)pyridyl, e.g. (methyl)(methylamino)pyridyl or (methyl)(ethylamino)pyridyl, especially 2-(methyl)-3-(methylamino)pyridyl or 2-(methyl)-3-(ethylamino)pyridyl (in particular 2-(methyl)-3-(methylamino)pyrid-5-yl or 2-(methyl)-3-(ethylamino)pyrid-5-yl);
(halo)(alkylamino)pyridyl, such as (fluoro)(loweralkylamino)pyridyl, e.g. (fluoro)(methylamino)pyridyl or (chloro)(methylamino)pyridyl, especially 3-(fluoro)-2-(methylamino)pyridyl or 3-(chloro)-2-(methylamino)pyridyl (in particular 3-(fluoro)-2-(methylamino)pyrid-5-yl or 3-(chloro)-2-(methylamino)pyrid-5-yl);
(haloalkyl)(alkylamino)pyridyl, such as (fluoro-loweralkyl)(loweralkylamino)pyridyl, e.g. (trifluoromethyl)(methylamino)pyridyl or (trifluoromethyl)(ethylamino)pyridyl, especially 3-(trifluoromethyl)-2-(methylamino)pyridyl or 3-(trifluoromethyl)-2-(ethylamino)pyridyl (in particular 3-(trifluoromethyl)-2-(methylamino)pyrid-5-yl or 3-(trifluoromethyl)-2-(ethylamino)pyrid-5-yl);
(haloalkyl)(amino)pyridyl, such as (fluoro-loweralkyl)(amino)pyridyl, e.g. (trifluoromethyl)(amino)pyridyl, especially 3-(trifluoromethyl)-2-(amino)pyridyl (in particular 3-(trifluromethyl)-2-(amino)pyrid-5-yl);
(hydroxyalkyl)(alkoxy)pyridyl, such as (hydroxyloweralkyl)(loweralkoxy)pyridyl, e.g. (hydroxymethyl)(methoxy)pyridyl, especially 2-(hydroxymethyl)-3-(methoxy)pyridyl (in particular 2-(hydroxymethyl)-3-(methoxy)pyrid-5-yl);
(hydroxyalkyl)(amino)pyridyl, such as (hydroxyloweralkyl)(amino)pyridyl, e.g. (hydroxymethyl)(amino)pyridyl, especially 3-(hydroxymethyl)-2-(amino)pyridyl (in particular 3-(hydroxymethyl)-2-(amino)pyrid-5-yl);
(alkoxyalkyl)(amino)pyridyl, such as (loweralkoxyloweralkyl)(amino)pyridyl, e.g. (methoxymethyl)(amino)pyridyl or (ethoxymethyl)(amino)pyridyl, especially 3-(methoxymethyl)-2-(amino)pyridyl or 3-(ethoxymethyl)-2-(amino)pyridyl (in particular 3-(methoxymethyl)-2-(amino)pyrid-5-yl or 3-(ethoxymethyl)-2-(amino)pyrid-5-yl);
(alkyl)(alkoxyalkoxy)pyridyl, such as (loweralkyl)(loweralkoxyloweralkoxy)pyridyl, e.g. (methyl)(methoxyethoxy)pyridyl, especially 2-(methyl)-3-(2-methoxyethoxy)pyridyl (in particular 2-(methyl)-3-(2-methoxyethoxy)pyrid-5-yl);
(alkoxyalkyl)(alkylamino)pyridyl, such as (loweralkoxyloweralkyl)(loweralkylamino)pyridyl, e.g. (ethoxymethyl)(ethylamino)pyridyl or (methoxymethyl)(methylamino)pyridyl, especially 3-(ethoxymethyl)-2-(ethylamino)pyridyl or 3-(methoxymethyl)-2-(methylamino)pyridyl (in particular 3-(ethoxymethyl)-2-(ethylamino)pyrid-5-yl or 3-(methoxymethyl)-2-(methylamino)pyrid-5-yl);
(amino)(alkylaminocarbonyl)pyridyl, such as (amino)(loweralkylaminocarbonyl)pyridyl, e.g. (amino)(methylaminocarbonyl)pyridyl, especially 2-(amino)-3-(methylaminocarbonyl)pyridyl (in particular 2-(amino)-3-(methylaminocarbonyl)pyrid-5-yl);
pyrimidinyl, in particular pyrimidin-5-yl;
loweralkylaminopyrimidinyl, e.g. 2- or 4-methylaminopyrimidinyl or 2- or 4-ethylaminopyrimidinyl, especially 2-methylaminopyrimidinyl or 2-ethylaminopyrimidinyl (in particular 2-methylaminopyrimidin-5-yl or 2-ethylaminopyrimidin-5-yl);
di-loweralkylaminopyrimidinyl, e.g. 2- or 4-dimethylaminopyrimidinyl, especially 2-dimethylaminopyrimidinyl (in particular 2-dimethylaminopyrimidin-5-yl);
alkoxypyrimidinyl, in particular methoxypyrimidinyl or ethoxypyrimidinyl, e.g. 2-methoxypyrimidinyl or 2-ethoxypyrimidinyl (in particular 2-methoxypyrimidin-5-yl or 2-ethoxypyrimidin-5-yl);
di-loweralkoxypyrimidinyl, e.g. 2,4-dimethoxypyrimidinyl or 2,4-diethoxypyrimidinyl, especially 2,4-dimethoxypyrimidinyl (in particular 2,4-dimethoxypyrimidin-5-yl);
(alkylamino)(alkoxy)pyrimidinyl such as (loweralkylamino)(loweralkoxy)pyrimidinyl, e.g. (ethylamino)(methoxy)pyrimidinyl or (methylamino)(methoxy)pyrimidinyl, especially 2-(ethylamino)-4-(methoxy)pyrimidinyl (in particular 2-(ethylamino)-4-(methoxy)pyrimidin-5-yl);
1H-pyrrolo[2,3-b]pyridinyl (in particular 1H-pyrrolo[2,3-b]pyridin-5-yl); 1-methyl-1H-pyrrolo[2,3-b]pyridinyl (in particular 1-methyl-1H-pyrrolo[2,3-b]pyridin-5-yl); pyrazinyl; pyrazolyl, e.g. pyrazol-4-yl; substituted pyrazolyl, e.g. hydroxyalkylpyrazolyl, especially 1-(2-hydroxy-ethyl)-1H-pyrazolyl (in particular 1-(2-hydroxy-ethyl)-1H-pyrazol-4-yl) or 1-[2-(tetrahydro-pyran-2-yloxy)-ethyl]-1H-pyrazolyl (in particular 1-[2-(tetrahydro-pyran-2-yloxy)-ethyl]-1H-pyrazol-4-yl); quinolinyl (in particular quinolin-3-yl); 2-oxo-2,3-dihydro-1H-indol-5-yl;
1-methyl-2,3-dihydro-1H-indol-5-yl; 1H-imidazo[4,5-b]pyridin-2(3H)-one-6-yl (in particular 1,3-dimethyl-1H-imidazo[4,5-b]pyridin-2(3H)-one-6-yl, 1-ethyl-3-methyl-1H-imidazo[4,5-b]pyridin-2(3H)-one-6-yl or 1-(2-methoxyethyl)-3-methyl-1H-imidazo[4,5-b]pyridin-2(3H)-one-6-yl; 3H-imidazo[4,5-b]pyridin-6-yl, e.g. (3-methyl)-3H-imidazo[4,5-b]pyridin-6-yl, (2-methyl)-(3-methyl)-3H-imidazo[4,5-b]pyridin-6-yl, (2-methoxy)-(3-methyl)-3H-imidazo[4,5-b]pyridin-6-yl, (2-dimethylamino)-(3-methyl)-3H-imidazo[4,5-b]pyridin-6-yl; 3H-[1,2,3]triazolo[4,5-b]pyridin-6-yl, e.g. (3-methyl)-3H-[1,2,3]triazolo[4,5-b]pyridin-6-yl, imidazo[1,2-a]pyridinyl (in particular imidazo[1,2-a]pyridin-6-yl.

In another embodiment, specifically preferred unsubstituted or substituted heterocyclyl or heteroaryl for R₃ is selected from pyridyl, especially pyridin-3-yl; alkylpyridyl, in particular loweralkylpyridyl, e.g. methylpyridyl, e.g., 2-, 3- or 4-methylpyridyl, especially 2-methylpyridyl or 3-methylpyridyl, in particular 2-methylpyrid-5-yl or 3-methylpyrid-5-yl; alkoxypyridyl, in particular lower-alkoxypyridyl, e.g. methoxypyridyl, e.g., 2-, 3- or 4-methoxypyridyl, especially 2-methoxypyridyl or 3-methoxypyridyl (in particular 2-methoxpyridin-5-yl, 3-methoxpyridin-5-yl); ethoxypyridyl, e.g., 2-, 3- or 4-ethoxypyridyl especially 2-ethoxypyridyl or 3-ethoxypyridyl (in particular 3-ethoxypyridin-5-yl, 2-ethoxypyridin-5-yl, 2-ethoxypyridin-4-yl); propoxypyridyl, e.g. n-propoxypyridyl or iso-propoxypyridyl (in particular 6-(n-propoxy)pyrid-3-yl or 3-(iso-propoxy)pyridin-5-yl); cycloalkylalkoxypyridyl, e.g. cyclopropylmethoxy-pyridyl, especially 2-cyclopropylmethoxy-pyridyl (in particular 2-cyclopropylmethoxy-pyrid-5-yl); alkoxyalkylpyridyl e.g. ethoxymethylpyridyl, 2-methoxymethylpyridyl (in particular 2-methoxymethylpyrid-5-yl); alkoxyalkoxypyridyl, in particular methoxyethoxypyridyl, e.g. 3-(2-methoxyethoxy)pyridyl or 2-(2-methoxyethoxy)pyridyl (in particular 3-(2-methoxyethoxy)pyrid-5-yl or 2-(2-methoxyethoxy)pyrid-5-yl); benzyloxyalkoxypyridyl, in particular benzyloxyethoxypyridyl or benzyloxypropoxypyridyl, e.g. 2-benzyloxyethoxypyridyl or 3-benzyloxypropoxypyridyl (in particular 2-benzyloxyethoxypyrid-5-yl or 3-benzyloxypropoxypyrid-5-yl); hydroxyalkylpyridyl, e.g. hydroxymethylpyridyl, especially 2-(hydroxymethyl)-pyridyl (in particular 2-hydroxymethylpyrid-5-yl); alkyl-sulfonyl pyridyl, e.g. methanesulfonylpyridyl, especially 3-methanesulfonylpyridyl (in particular 3-methanesulfonylpyrid-5-yl); hydroxyalkoxypyridyl, e.g. 2-(2-hydroxyethoxy)-pyridyl or 2-(3-hydroxypropoxy)-pyridyl (in particular 2-(2-hydroxyethoxy)-pyrid-5-yl or 2-(3-hydroxypropoxy)-pyrid-5-yl); alkoxycarbonylpyridyl, e.g. methoxycarbonylpyridyl, especially 2-methoxycarbonyl-pyridyl (in particular 2-methoxycarbonyl-pyrid-5-yl); aminopyridyl, e.g. 2- or 3-aminopyridyl (in particular 2-aminopyridin-5-yl or 3-aminopyrid-5-yl); alkylaminopyridyl, e.g. loweralkylaminopyridyl, in particular methylaminopyridyl, more particularly 2-methylaminopyridyl (especially 2-methylaminopyrid-5-yl); dialkylaminopyridyl, in particular di-loweralkylaminopyridyl, e.g. 2-, 3- or 4-dimethylaminopyridyl, especially 2-dimethylaminopyridyl (in particular 2-dimethylaminopyrid-5-yl); cycloaminopyridyl, e.g. azetidinylpyridyl, especially 2-azetidin-1-yl-pyridinyl (in particular 2-azetidin-1-yl-pyridin-5-yl); hydroxyalkylaminopyridyl, e.g. 2-(2-hydroxyethylamino)-pyridyl (in particular 2-(2-hydroxyethylamino)-pyrid-5-yl); amino-haloloweralkyl-pyridyl, e.g. amino-trifluoromethyl-pyridyl, especially 2-amino-3-trifluoromethyl-pyridyl (in particular 2-amino-3-trifluoromethyl-pyrid-5-yl); haloalkylpyridinyl, in particular haloloweralkylpyridyl, especially, 2-, 3- or 4-trifluoromethylpyridyl, most especially 2-trifluoromethylpyridyl (in particular 2-trifluoromethylpyrid-5-yl); halopyridyl, in particular fluoropyridyl, especially 2-fluoropyridyl (in particular 2-fluoropyrid-3-yl or 2-fluoropyrid-4-yl); halo-alkoxy-pyridyl, e.g. fluoro-methoxy-pyridyl such as 3-fluoro-2-methoxy-pyridyl (in particular 3-fluoro-2-methoxy-pyrid-5-yl); carbamoylpyridyl, especially 2-(carbamoyl)pyridyl (in particular 2-(carbamoyl)pyrid-5-yl); alkyl-substituted carbamoyl, e.g. methylcarbamoyl, especially 2-(methylcarbamoyl)pyridyl (in particular 2-(methylcarbamoyl)pyrid-5-yl); piperazinylpyridyl, e.g. 1-piperazinylpyridyl, especially 2-(1-piperazinyl)pyridyl (in particular 2-(1-piperazinyl)pyrid-5-yl); N-alkylpiperazinylpyridyl, such as N-loweralkylpiperazinylpyridyl, e.g. N-methylpiperazinylpyridyl, especially 2-(4-methylpiperazin-1-yl)-pyridyl (in particular 2-(4-methylpiperazin-1-yl)-pyrid-5-yl); alkylsulfonamidopyridyl, such as loweralkylsulfonamidopyridyl, especially methylsulfonamidopyridyl, e.g. 3-(methylsulfonamido) pyridyl (in particular 3-(methylsulfonamido)-pyridin-5-yl); dialkylsulfonamidopyridyl, such as diloweralkylsulfonamidopyridyl, especially dimethylsulfonamidopyridyl, e.g. 3-(dimethylsulfonamido)pyridyl (in particular 3-(dimethylsulfonamido)-pyridin-5-yl); (alkylsulfonamido)(alkyl)pyridyl such as (loweralkylsulfonamido)(loweralkyl)pyridyl, especially (methylsulfonamido)(methyl)pyridyl, e.g. 3-(methylsulfonamido)(methyl)pyridyl (in particular 3-(methylsulfonamido)-2-methyl pyridin-5-yl); dialkylsulfonamido(alkyl)pyridyl, such as diloweralkylsulfonamido(loweralkyl)pyridyl, especially dimethylsulfonamido(methyl)pyridyl, e.g. 3-(dimethylsulfonamido)(2-methyl)pyridyl (in particular 3-(dimethylsulfonamido)-(2-methyl)-pyridin-5-yl); 3H-tetrazol-5-yl pyridyl, e.g. 2-(3H-tetrazol-5-yl)pyridyl (in particular 2-(3H-tetrazol-5-yl)pyrid-5-yl); (alkoxy)(alkylcarbonylamino)pyridyl, such as (loweralkoxy)(loweralkylcarbonylamino)pyridyl, e.g. (methoxy)(methylcarbonylamino)pyridyl or (ethoxy)(methylcarbonylamino)pyridyl, especially 2-(methoxy)-3-(methylcarbonylamino)pyridyl or 2-(ethoxy)-3-(methylcarbonylamino)pyridyl (in particular 2-(methoxy)-3-(methylcarbonylamino)pyrid-5-yl or 2-(ethoxy)-3-(methylcarbonylamino)pyrid-5-yl); (alkoxy)(alkylcarbonyl-N-alkylamino)pyridyl, such as (loweralkoxy)(loweralkylcarbonyl-N-loweralkylamino)pyridyl, e.g. (methoxy)(methylcarbonyl-N-methylamino)pyridyl or (ethoxy)(methylcarbonyl-N-methylamino)pyridyl, especially 2-(methoxy)-3-(methylcarbonyl-N-methylamino)pyridyl or 2-(ethoxy)-3-(methylcarbonyl-N-methylamino)pyridyl (in particular 2-(methoxy)-3-(methylcarbonyl-N-methylamino)pyrid-5-yl or 2-(ethoxy)-3-(methylcarbonyl-N-methylamino)pyrid-5-yl); (alkoxy)(nitro)pyridyl, such as (loweralkoxy)(nitro)pyridyl, e.g. (methoxy)(nitro)pyridyl, especially 2-(methoxy)-3-(nitro)-pyridyl (in particular 2-(methoxy)-3-(nitro)-pyrid-5-yl); (alkoxy)(amino)pyridyl, such as (loweralkoxy)(amino)pyridyl, e.g. (methoxy)(amino)pyridyl, especially 2-(methoxy)-3-(amino)-pyridyl (in particular 2-(methoxy)-3-(amino)-pyrid-5-yl); (alkoxy)(alkylaminocarbonyl)pyridyl, such as (loweralkoxy)(loweralkylaminocarbonyl)pyridyl, e.g. (methoxy)(methylaminocarbonyl)pyridyl, especially 2-(methoxy)-3-(methylaminocarbonyl)pyridyl (in particular 2-(methoxy)-3-(methylaminocarbonyl)pyrid-5-yl; (alkoxy)(hydroxycarbonyl)pyridyl, such as (loweralkoxy)(hydroxycarbonyl)pyridyl, e.g. (methoxy)(hydroxycarbonyl)pyridyl, especially 2-(methoxy)-3-(hydroxycarbonyl)pyridyl (in particular 2-(methoxy)-3-(hydroxycarbonyl)pyrid-5-yl; (N,N-dimethylaminopropoxy)pyridyl, e.g. 2-(N,N-dimethylaminopropoxy)pyridyl) (especially 2-(3-N,N-dimethylaminopropoxy)pyridin-5-yl), pyrimidinyl, in particular pyrimidin-5-yl; loweralkylaminopyrimidinyl, e.g. 2- or 4-methylaminopyrimidinyl, especially 2-methylaminopyrimidinyl (in particular 2-methylaminopyrimidin-5-yl); di-loweralkylaminopyrimidinyl, e.g. 2- or 4-dimethylaminopyrimidinyl, especially 2-dimethylaminopyrimidinyl (in particular 2-dimethylaminopyrimidin-5-yl); alkoxypyrimidinyl, in particular methoxypyrimidinyl or ethoxypyrimidinyl, e.g. 2-methoxypyrimidinyl or 2-ethoxypyrimidinyl (in particular 2-methoxypyrimidin-5-yl or 2-ethoxypyrimidin-5-yl); 1H-pyrrolo[2,3-b]pyridinyl (in particular 1H-pyrrolo[2,3-b]pyridin-5-yl); 1-methyl-1H-pyrrolo[2,3-b]pyridinyl (in particular 1-methyl-1H-pyrrolo[2,3-b]pyridin-5-yl); pyrazinyl; pyrazolyl, e.g. pyrazol-4-yl; substituted pyrazolyl, e.g. hydroxyalkylpyrazolyl, especially 1-(2-hydroxy-ethyl)-1H-pyrazolyl (in particular 1-(2-hydroxy-ethyl)-1H-pyrazol-4-yl) or 1-[2-(tetrahydro-pyran-2-yloxy)-ethyl]-1H-pyrazolyl (in particular 1-[2-(tetrahydro-pyran-2-yloxy)-ethyl]-1H-pyrazol-4-yl); quinolinyl (in particular quinolin-3-yl); 2-oxo-2,3-dihydro-1H-indol-5-yl; 1-methyl-2,3-dihydro-1H-indol-5-yl; 1H-imidazo[4,5-b]pyridin-2(3H)-one-6-yl (in particular 1,3-dimethyl-1H-imidazo[4,5-b]pyridin-2(3H)-one-6-yl, 1-ethyl-3-methyl-1H-imidazo[4,5-b]pyridin-2(3H)-one-6-yl or 1-(2-methoxyethyl)-3-methyl-1H-imidazo[4,5-b]pyridin-2(3H)-one-6-yl; 3H-imidazo[4,5-b]pyridin-6-yl, e.g. (3-methyl)-3H-imidazo[4,5-b]pyridin-6-yl, (2-methyl)-(3-methyl)-3H-imidazo[4,5-b]pyridin-6-yl, (2-methoxy)-(3-methyl)-3H-imidazo[4,5-b]pyridin-6-yl, (2-dimethylamino)-(3-methyl)-3H-imidazo[4,5-b]pyridin-6-yl; 3H-[1,2,3]triazolo[4,5-b]pyridin-6-yl, e.g. (3-methyl)-3H-[1,2,3]triazolo[4,5-b]pyridin-6-yl.

In an embodiment, R³ is selected from pyridinyl, and/or selected from the following substituted pyridyl groups: 2-methylpyridyl, 3-methylpyridyl, 2-methoxpyridinyl, 3-methoxpyridinyl, 3-ethoxypyridinyl, 2-ethoxypyridinyl, 2-ethoxypyridinyl, 6-(n-propoxy)pyridyl, 3-(iso-propoxy)pyridinyl), 2-cyclopropylmethoxy-pyridyl, 2-methoxymethylpyridyl, 3-(2-methoxyethoxy)pyridyl, 2-(2-methoxyethoxy)pyridyl, 2-benzyloxyethoxypyridyl, 3-benzyloxypropoxypyridyl, 2-hydroxymethylpyridyl, 3-(2-hydroxyprop-2-yl)pyridyl, 3-methanesulfonylpyridyl, 2-(2-hydroxyethoxy)-pyridyl, 2-(3-hydroxypropoxy)-pyridyl, 2-methoxycarbonyl-pyridyl, 2-aminopyridinyl, 3-aminopyridyl, 2-methylaminopyridyl, 3-methylaminopyridyl, 3-(iso-propyl amino)pyridyl, 2-dimethylaminopyridyl, 2-azetidin-1-yl-pyridinyl, 3-azetidin-1-yl-pyridinyl, 3-(2-cyanoprop-2-yl)-pyridinyl, 3-(1-cyanocyclobut-1-yl)-pyridinyl, 2-(2-hydroxyethylamino)-pyridyl, 2-amino-3-trifluoromethyl-pyridyl, 2-trifluoromethylpyridyl, 2-fluoropyridyl, 3-fluoro-2-methoxy-pyridyl, 2-(carbamoyl)pyridyl, 2-(methylcarbamoyl)pyridyl, 2-(1-piperazinyl)pyridyl, 2-(4-methylpiperazin-1-yl)-pyridyl, 3-(methylsulfonamido)-pyridinyl, 3-(dimethylsulfonamido)-pyridinyl, 3-(methylsulfonamido)-2-methylpyridinyl, 3-(dimethylsulfonamido)-(2-methyl)-pyridinyl, 2-(3H-tetrazol-5-yl)pyridyl, 2-(methoxy)-3-(methylcarbonylamino)pyridyl, 2-(ethoxy)-3-(methylcarbonylamino)pyridyl, 2-(methoxy)-3-(methylcarbonyl-N-methylamino)pyridyl, 2-(ethoxy)-3-(methylcarbonyl-N-methylamino)pyridyl, 2-(methoxy)-3-(nitro)-pyridyl, 2-(methoxy)-3-(amino)-pyridyl, 2-(methoxy)-3-(methylaminocarbonyl)pyridyl, 2-(methoxy)-3-(hydroxycarbonyl)pyridyl, 2-(3-N,N-dimethylaminopropoxy)pyridinyl, 2-(methyl)-3-(methoxy)pyridyl, 2-(dimethoxy-methyl)-3-(methoxy)pyridyl, 2-(methoxy-methyl)-3-(methoxy)pyridyl, 2-(methoxy-methyl)-3-(ethoxy)pyridyl, 2-(methoxy-methyl)-3-(iso-propoxy)pyridyl, 2-(hydroxymethyl)-3-(ethylamino)pyridyl, 2-(hydroxymethyl)-3-(methylamino)pyridyl, 2-(methyl)-3-(methylamino)pyridyl, 2-(methyl)-3-(ethylamino)pyridyl, 3-(fluoro)-2-(methylamino)pyridyl, 3-(chloro)-2-(methylamino)pyridyl, 3-(trifluoromethyl)-2-(methylamino)pyridyl, 3-(trifluoromethyl)-2-(ethylamino)pyridyl, 3-(trifluoromethyl)-2-(amino)pyridyl, 2-(hydroxymethyl)-3-(methoxy)pyridyl, 3-(hydroxymethyl)-2-(amino)pyridyl, 3-(methoxymethyl)-2-(amino)pyridyl, 3-(ethoxymethyl)-2-(amino)pyridyl, 2-(methyl)-3-(2-methoxyethoxy)pyridyl, 3-(ethoxymethyl)-2-(ethylamino)pyridyl, 3-(methoxymethyl)-2-(methylamino)pyridyl, 2-(amino)-3-(methylaminocarbonyl)pyridyl.

In another embodiment, R³ is selected from the above said pyridyl and/or substituted pyridyl groups, and/or the following pyrimidinyl groups:

pyrimidinyl and/or 2-methylaminopyrimidinyl, 2-ethylaminopyrimidinyl, 2-dimethylaminopyrimidinyl, 2-methoxypyrimidinyl, 2-ethoxypyrimidinyl, 2,4-dimethoxypyrimidinyl, 2-(ethylamino)-4-(methoxy)pyrimidinyl.

In another embodiment, R³ is selected from the above said pyridyl and/or substituted pyridyl groups, and/or the above pyrimidinyl and/or substituted pyrimidinyl groups, and/or selected from the following groups:

1H-pyrrolo[2,3-b]pyridinyl, 1-methyl-1H-pyrrolo[2,3-b]pyridinyl, pyrazinyl, pyrazolyl, 1-(2-hydroxy-ethyl)-1H-pyrazolyl, 1-[2-(tetrahydro-pyran-2-yloxy)-ethyl]-1H-pyrazolyl, quinolinyl, 2-oxo-2,3-dihydro-1H-indolyl, 1-methyl-2,3-dihydro-1H-indolyl, 1,3-dimethyl-1H-imidazo[4,5-b]pyridin-2(3H)-oneyl, 1-ethyl-3-methyl-1H-imidazo[4,5-b]pyridin-2(3H)-oneyl, 1-(2-methoxyethyl)-3-methyl-1H-imidazo[4,5-b]pyridin-2(3H)-oneyl, (3-methyl)-3H-imidazo[4,5-b]pyridinyl, (2-methyl)-(3-methyl)-3H-imidazo[4,5-b]pyridinyl, (2-methoxy)-(3-methyl)-3H-imidazo[4,5-b]pyridinyl, (2-dimethylamino)-(3-methyl)-3H-imidazo[4,5-b]pyridinyl, (3-methyl)-3H-[1,2,3]triazolo[4,5-b]pyridinyl and imidazo[1,2-a]pyridinyl.

In a further embodiment, R³ is selected from pyridin-3-yl, and/or selected from the following substituted pyridyl groups: 2-methylpyrid-5-yl, 3-methylpyrid-5-yl, 2-methoxpyridin-5-yl, 3-methoxpyridin-5-yl, 3-ethoxypyridin-5-yl, 2-ethoxypyridin-5-yl, 2-ethoxypyridin-4-yl, 6-(n-propoxy)pyrid-3-yl, 3-(iso-propoxy)pyridin-5-yl), 2-cyclopropylmethoxy-pyrid-5-yl, 2-methoxymethylpyrid-5-yl, 3-(2-methoxyethoxy)pyrid-5-yl, 2-(2-methoxyethoxy)pyrid-5-yl, 2-benzyloxyethoxypyrid-5-yl, 3-benzyloxypropoxypyrid-5-yl, 2-hydroxymethylpyrid-5-yl, 3-(2-hydroxyprop-2-yl)pyrid-5-yl, 3-methanesulfonylpyrid-5-yl, 2-(2-hydroxyethoxy)-pyrid-5-yl, 2-(3-hydroxypropoxy)-pyrid-5-yl, 2-methoxycarbonyl-pyrid-5-yl, 2-aminopyridin-5-yl, 3-aminopyrid-5-yl, 2-methylaminopyrid-5-yl, 3-methylaminopyrid-5-yl, 3-(iso-propyl amino)pyrid-5-yl, 2-dimethylaminopyrid-5-yl, 2-azetidin-1-yl-pyridin-5-yl, 3-azetidin-1-yl-pyridin-5-yl, 3-(2-cyanoprop-2-yl)-pyridin-5-yl, 3-(1-cyanocyclobut-1-yl)-pyridin-5-yl, 2-(2-hydroxyethylamino)-pyrid-5-yl, 2-amino-3-trifluoromethyl-pyrid-5-yl, 2-trifluoromethylpyrid-5-yl, 2-fluoropyrid-3-yl, 2-fluoropyrid-4-yl, 3-fluoro-2-methoxy-pyrid-5-yl, 2-(carbamoyl)pyrid-5-yl, 2-(methylcarbamoyl)pyrid-5-yl, 2-(1-piperazinyl)pyrid-5-yl, 2-(4-methylpiperazin-1-yl)-pyrid-5-yl, 3-(methylsulfonamido)-pyridin-5-yl, 3-(dimethylsulfonamido)-pyridin-5-yl, 3-(methylsulfonamido)-2-methylpyridin-5-yl, 3-(dimethylsulfonamido)-(2-methyl)-pyridin-5-yl, 2-(3H-tetrazol-5-yl)pyrid-5-yl, 2-(methoxy)-3-(methylcarbonylamino)pyrid-5-yl, 2-(ethoxy)-3-(methylcarbonylamino)pyrid-5-yl, 2-(methoxy)-3-(methylcarbonyl-N-methylamino)pyrid-5-yl, 2-(ethoxy)-3-(methylcarbonyl-N-methylamino)pyrid-5-yl, 2-(methoxy)-3-(nitro)-pyrid-5-yl, 2-(methoxy)-3-(amino)-pyrid-5-yl, 2-(methoxy)-3-(methylaminocarbonyl)pyrid-5-yl, 2-(methoxy)-3-(hydroxycarbonyl)pyrid-5-yl, 2-(3-N,N-dimethylaminopropoxy)pyridin-5-yl, 2-(methyl)-3-(methoxy)pyrid-5-yl, 2-(dimethoxy-methyl)-3-(methoxy)pyrid-5-yl, 2-(methoxy-methyl)-3-(methoxy)pyrid-5-yl, 2-(methoxy-methyl)-3-(ethoxy)pyrid-5-yl, 2-(methoxy-methyl)-3-(iso-propoxy)pyrid-5-yl, 2-(hydroxymethyl)-3-(ethylamino)pyrid-5-yl, 2-(hydroxymethyl)-3-(methylamino)pyrid-5-yl, 2-(methyl)-3-(methylamino)pyrid-5-yl, 2-(methyl)-3-(ethylamino)pyrid-5-yl, 3-(fluoro)-2-(methylamino)pyrid-5-yl, 3-(chloro)-2-(methylamino)pyrid-5-yl, 3-(trifluoromethyl)-2-(methylamino)pyrid-5-yl, 3-(trifluoromethyl)-2-(ethylamino)pyrid-5-yl, 3-(trifluoromethyl)-2-(amino)pyrid-5-yl, 2-(hydroxymethyl)-3-(methoxy)pyrid-5-yl, 3-(hydroxymethyl)-2-(amino)pyrid-5-yl, 3-(methoxymethyl)-2-(amino)pyrid-5-yl, 3-(ethoxymethyl)-2-(amino)pyrid-5-yl, 2-(methyl)-3-(2-methoxyethoxy)pyrid-5-yl, 3-(ethoxymethyl)-2-(ethylamino)pyrid-5-yl, 3-(methoxymethyl)-2-(methylamino)pyrid-5-yl, 2-(amino)-3-(methylaminocarbonyl)pyrid-5-yl.

In another further embodiment, R³ is selected from the above said pyrid-3-yl and/or substituted pyridyl groups, and/or the following pyrimidinyl groups: pyrimidin-5-yl and/or 2-methylaminopyrimidin-5-yl, 2-ethylaminopyrimidin-5-yl, 2-dimethylaminopyrimidin-5-yl, 2-methoxypyrimidin-5-yl, 2-ethoxypyrimidin-5-yl, 2,4-dimethoxypyrimidin-5-yl, 2-(ethylamino)-4-(methoxy)pyrimidin-5-yl.

In another further embodiment, R³ is selected from the above said pyrid-3-yl and/or substituted pyridyl groups, and/or the above pyrimidin-5-yl and/or substituted pyrimidinyl groups, and/or selected from the following groups:

1H-pyrrolo[2,3-b]pyridin-5-yl, 1-methyl-1H-pyrrolo[2,3-b]pyridin-5-yl, pyrazinyl, pyrazol-4-yl, 1-(2-hydroxy-ethyl)-1H-pyrazol-4-yl, 1-[2-(tetrahydro-pyran-2-yloxy)-ethyl]-1H-pyrazol-4-yl, quinolin-3-yl, 2-oxo-2,3-dihydro-1H-indol-5-yl, 1-methyl-2,3-dihydro-1H-indol-5-yl, 1,3-dimethyl-1H-imidazo[4,5-b]pyridin-2(3H)-one-6-yl, 1-ethyl-3-methyl-1H-imidazo[4,5-b]pyridin-2(3H)-one-6-yl, 1-(2-methoxyethyl)-3-methyl-1H-imidazo[4,5-b]pyridin-2(3H)-one-6-yl, (3-methyl)-3H-imidazo[4,5-b]pyridin-6-yl, (2-methyl)-(3-methyl)-3H-imidazo[4,5-b]pyridin-6-yl, (2-methoxy)-(3-methyl)-3H-imidazo[4,5-b]pyridin-6-yl, (2-dimethylamino)-(3-methyl)-3H-imidazo[4,5-b]pyridin-6-yl, (3-methyl)-3H-[1,2,3]triazolo[4,5-b]pyridin-6-yl and imidazo[1,2-a]pyridin-6-yl.

With the groups of preferred compounds of formula (I) mentioned herein, definitions of substituents from the general definitions mentioned hereinbefore may reasonably be used, e.g., to replace more general definitions with more specific definitions or especially with definitions characterized as being preferred.

A preferred group of compounds of the present invention of formula (I) are those wherein X is O.

Another preferred group of compounds of the present invention of formula (I) are those wherein Y is CH.

Another preferred group of compounds of the present invention of formula (I) are those wherein X is O or S and Y is CH.

Another preferred group of compounds of the present invention of formula (I) are those wherein X is O and Y is CH.

An alternative group of compounds of the present invention of formula (I) are those wherein Y is N.

An alternative group of compounds of the present invention of formula (I) are those wherein X is O or S and Y is N.

Another alternative group of compounds of the present invention of formula (I) are those wherein X is O and Y is N.

An embodiment of the present invention includes compounds of formula (I) or pharmaceutically acceptable salts, solvates or hydrates thereof, wherein:
X is O or S;
Y is CH or N;
R¹ is unsubstituted pyridyl or pyridyl substituted with one, two or three substituents (preferably one or two substituents), at least one of which is in the alpha-position, said substituents being independently selected from halo; lower alkyl; lower alkoxy; lower alkoxy lower alkoxy; amino; mono- or di-loweralkyl substituted amino; N-loweralkyl-N-lower alkoxy lower alkyl substituted amino, lower alkoxy lower alkyl carbonyl amino, unsubstituted piperazinyl, lower alkyl-substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino;
R² is hydrogen or lower alkyl;
R³ is unsubstituted phenyl, or phenyl substituted with one or two groups independently selected from the group consisting of halo; halo-lower alkyl; hydroxyl; amino; mono or disubstituted amino; cyclic amino; amino-lower alkyl; lower alkoxy; hydroxy-lower alkyl; hydroxy lower alkoxy; lower alkyl; cyano; cyano-lower alkyl; amidino; N-hydroxyamidino; amidino-lower alkyl; or N-hydroxyamidino-lower alkyl; sulfonyl; alkyl-substituted sulfonyl; sulfonamide; pyrrolidinesulfonyl; [1,3]dioxolo; halo-substituted [1,3]dioxolo; alkoxy carbonyl; carbamoyl; substituted carbamoyl; heterocycle; heterocyclyl lower alkyl; heteroaryl or heteroaryl lower alkyl; or
R³ is indolyl, 2,3-dihydro-1H-indol-5-yl, 1-methyl-2,3-dihydro-1H-indol-5-yl, 2-oxo-2,3-dihydro-1H-indol-5-yl, pyridyl, pyrimidinyl, 1H-pyrrolo[2,3-b]pyridin-5-yl, 1-methyl-1H-pyrrolo[2,3-b]pyridin-5-yl, pyrazolyl, pyrazol-4-yl, pyrazinyl, quinolyl, quinol-3-yl, 1H-imidazo[4,5-b]pyridin-2(3H)-one-6-yl, 3H-imidazo[4,5-b]pyridin-6-yl, 3H-[1,2,3]triazolo[4,5-b]pyridin-6-yl, imidazo[1,2-a]pyridin-6-yl each independently being unsubstituted or substituted by one or two radicals independently selected from the group consisting of halo; halo-lower alkyl; hydroxyl; amino; mono or disubstituted amino, wherein the substituents are independently selected from alkyl and hydroxyalkyl; cyclic amino; amino-lower alkyl; lower alkoxy; lower alkoxy lower alkyl; di-lower alkoxy lower alkyl; hydroxy-lower alkyl; hydroxy lower alkoxy; lower alkoxy lower alkoxy; lower alkyl; cycloalkyl; cyano-cycloalkyl; cyano; cyano-lower alkyl; amidino; N-hydroxyamidino; amidino-lower alkyl; or N-hydroxyamidino-lower alkyl; sulfonyl; alkyl-substituted sulfonyl; sulfonamide; pyrrolidine-1-sulfonyl; [1,3]dioxolo; halo substituted [1,3]dioxolo; alkoxy carbonyl; carbamoyl; aminocarbonylalkyl; N-mono-substituted aminocarbonylalkyl; N-di-substituted aminocarbonylalkyl; 3H-tetrazolyl, pyrazol, heterocyclyl lower alkyl, heteroaryl or heteroaryl lower alkyl.

A further embodiment of the present invention includes compounds of formula (I) or pharmaceutically acceptable salts, solvates or hydrates thereof, wherein:
X is O or S (preferably O);
Y is CH or N (preferably CH);
R¹ is unsubstituted pyridinyl (e.g. pyridin-3-yl) or 2-methyl-pyridin-3-yl, 3-methyl-pyridin-2-yl, 4-methyl-pyridin-3-yl, 2,6-dimethyl-pyridin-3-yl, 2-fluoro-pyridin-3-yl, 6-fluoro-2-methyl-pyridin-3-yl, 2,6-dimethoxy-pyridin-3-yl, 6-methoxy-2-methyl-pyridin-3-yl, 2-methoxy-6-piperazin-1-yl-pyridin-3-yl, 2-methoxy-6-(4-methyl-piperazin-1-yl)-pyridin-3-yl, 6-(N-methyl-N-ethyl amino)-2-methyl-pyridin-3-yl, 6-(methoxyethoxy)-2-methyl-pyridin-3-yl, 6-(N-methyl-N-methoxyethyl amino)-2-methyl-pyridin-3-yl, 6-fluoro-4-methyl-pyridin-3-yl, 5-methyl-pyridin-3-yl, 2-chloro-6-methoxy-pyridin-3-yl, 2-methyl-6-dimethylamino-pyridin-3-yl, 2-methyl-6-[N-methoxymethyl carbonyl]-amino-pyridin-3-yl, 2-methyl-6-(4-methyl-3-oxo-piperazin-1-yl)-pyridin-3-yl, 6-azetidinyl-2-methyl-pyridin-3-yl, 6-pyrrolidinyl-2-methyl-pyridin-3-yl, 6-(3,3-difluoro-azetidinyl)-2-methyl-pyridin-3-yl, 6-[N-(2-hydroxyethyl)-N-methylamino]-2-methyl-pyridin-3-yl, 6-(3-hydroxypyrrolidinyl)-2-methyl-pyridin-3-yl, 6-((S)-3-hydroxypyrrolidinyl)-2-methyl-pyridin-3-yl, 6-((R)-3-hydroxypyrrolidinyl)-2-methyl-pyridin-3-yl, 6-(3-methoxypyrrolidinyl)-2-methyl-pyridin-3-yl, 6-(3-hydroxy-azetidinyl)-2-methyl-pyridin-3-yl, 6-amino-2-methyl-pyridin-3-yl, 6-(2-hydroxyprop-2-yl)-2-methyl-pyridin-3-yl, 6-trifluoromethyl-2-methyl-pyridin-3-yl, 6-(3,3,4,4-tetrafluoropyrrolidinyl)-2-methyl-pyridin-3-yl;
R² is hydrogen or lower alkyl;
R³ is selected from phenyl; hydroxyphenyl; methoxyphenyl; 3,4-dimethoxyphenyl;
   ethoxyphenyl; 3,4-diethoxyphenyl; methoxy ethoxy-phenyl; 3-methoxy-4-(2-methoxy ethoxy)-phenyl; 3-methoxy-4-hydroxy phenyl; fluoro-hydroxy-phenyl; hydroxy-fluoroalkyl-phenyl; 2,2-difluoro-benzo[1,3]dioxolo; benzene sulfonamide; N-(phen-3-yl)-methanesulfonamide; N-methyl-N-phen-3-yl-methanesulfonamide; 3-methanesulfonylphenyl; 3- or 4-benzamide; 3- or 4-N-methyl-benzamide; 3- or 4-N,N-dimethyl-benzamide; pyrazol-phenyl; imidazol-phenyl; pyridyl; alkylpyridyl; alkoxypyridyl; cycloalkylalkoxypyridyl; alkoxyalkylpyridyl; alkoxyalkoxypyridyl, benzyloxyalkoxypyridyl, hydroxyalkylpyridyl; alkyl-sulfonyl pyridyl; hydroxyalkoxypyridyl; alkoxycarbonylpyridyl; aminopyridyl; alkylaminopyridyl; dialkylaminopyridyl; cycloaminopyridyl; (cyanoloweralkyl)-pyridinyl; (cyanocycloloweralkyl)-pyridinyl; hydroxyalkylaminopyridyl; amino-haloloweralkyl-pyridyl; haloalkylpyridinyl; halopyridyl; halo-alkoxy-pyridyl; carbamoylpyridyl; alkyl-substituted carbamoyl; piperazinylpyridyl; N-alkylpiperazinylpyridyl; alkylsulfonamidopyridyl; dialkylsulfonamido(alkyl)pyridyl; (alkylsulfonamido)(alkyl)pyridyl; dialkylsulfonamido(alkyl)pyridyl; 3H-tetrazol-5-yl pyridyl; (alkoxy)(alkylcarbonylamino)pyridyl; (alkoxy)(alkylcarbonyl-N-alkylamino)pyridyl; (alkoxy)(nitro)pyridyl; (alkoxy)(amino)pyridyl; (alkoxy)(alkylaminocarbonyl)pyridyl; (alkoxy)(hydroxycarbonyl)pyridyl; (N,N-dimethylaminopropoxy)pyridyl; (alkyl)(alkoxy)pyridyl; (dialkoxy-alkyl)(alkoxy)pyridyl; (alkoxyalkyl)(alkoxy)pyridyl; (hydroxyalkyl)(alkylamino)pyridyl; (alkyl)(alkylamino)pyridyl; (halo)(alkylamino)pyridyl; (haloalkyl)(alkylamino)pyridyl; (haloalkyl)(amino)pyridyl; (hydroxyalkyl)(alkoxy)pyridyl; (hydroxyalkyl)(amino)pyridyl; (alkoxyalkyl)(amino)pyridyl; (alkyl)(alkoxyalkoxy)pyridyl; (alkoxyalkyl)(alkylamino)pyridyl; (amino)(alkylaminocarbonyl)pyridyl; pyrimidinyl; loweralkylaminopyrimidinyl; di-loweralkylaminopyrimidinyl; alkoxypyrimidinyl; di-loweralkoxypyrimidinyl; (alkylamino)(alkoxy)pyrimidinyl; 1H-pyrrolo[2,3-b]pyridinyl; 1-methyl-1H-pyrrolo[2,3-b]pyridinyl; quinolinyl; 2-oxo-2,3-dihydro-1H-indol-5-yl; 1-methyl-2,3-dihydro-1H-indol-5-yl; imidazo[1,2-a]pyridinyl;

A further embodiment of the present invention includes compounds of formula (I) or pharmaceutically acceptable salts, solvates or hydrates thereof, wherein:
X is O or S;
Y is CH or N;
R¹ is unsubstituted pyridyl or pyridyl substituted with one, two or three substituents (preferably one or two substituents), at least one of which is in the alpha-position, said substituents being independently selected from halo; lower alkyl; lower alkoxy; lower alkoxy lower alkoxy; amino; mono- or di-loweralkyl substituted amino; N-loweralkyl-N-lower alkoxy lower alkyl substituted amino, lower alkoxy lower alkyl carbonyl amino, unsubstituted piperazinyl, lower alkyl-substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino;
R² is hydrogen or lower alkyl;
R³ is unsubstituted phenyl, or phenyl substituted with one or two groups independently selected from the group consisting of lower alkoxy; carbamoyl; mono- or di-lower alkyl-substituted carbamoyl; or
R³ is quinolinyl, 1H-pyrrolo[2,3-b]pyridin-5-yl, 1-methyl-1H-pyrrolo[2,3-b]pyridin-5-yl, imidazo[1,2-a]pyridin-6-yl or
R³ is pyridyl or pyrimidinyl, each independently being unsubstituted or substituted by one or two radicals independently selected from the group consisting of halo; loweralkyl; halo-lower alkyl; amino; cyclo amino; mono- or di-loweralkyl substituted amino; lower alkoxy; hydroxy-lower alkyl; N,N-di-loweralkyl aminoloweralkoxy, loweralkoxy-loweralkoxy; lower alkoxy lower alkyl; di-loweralkoxy-loweralkyl; cyano lower alkyl; cyanocycloloweralkyl; loweralkylaminocarbonyl.

A further embodiment of the present invention includes compounds of formula (I) or pharmaceutically acceptable salts, solvates or hydrates thereof, wherein:
X is O or S (preferably O);
Y is CH or N (preferably CH);
R¹ is unsubstituted pyridinyl (e.g. pyridin-3-yl) or 2-methyl-pyridin-3-yl, 3-methyl-pyridin-2-yl, 4-methyl-pyridin-3-yl, 2,6-dimethyl-pyridin-3-yl, 2-fluoro-pyridin-3-yl, 6-fluoro-2-methyl-pyridin-3-yl, 2,6-dimethoxy-pyridin-3-yl, 6-methoxy-2-methyl-pyridin-3-yl, 2-methoxy-6-piperazin-1-yl-pyridin-3-yl, 2-methoxy-6-(4-methyl-piperazin-1-yl)-pyridin-3-yl, 6-(N-methyl-N-ethyl amino)-2-methyl-pyridin-3-yl, 6-(methoxyethoxy)-2-methyl-pyridin-3-yl, 6-(N-methyl-N-methoxyethyl amino)-2-methyl-pyridin-3-yl, 6-fluoro-4-methyl-pyridin-3-yl, 5-methyl-pyridin-3-yl, 2-chloro-6-methoxy-pyridin-3-yl, 2-methyl-6-dimethylamino-pyridin-3-yl, 2-methyl-6-[N-methoxymethyl carbonyl]-amino-pyridin-3-yl, 2-methyl-6-(4-methyl-3-oxo-piperazin-1-yl)-pyridin-3-yl, 6-azetidinyl-2-methyl-pyridin-3-yl, 6-pyrrolidinyl-2-methyl-pyridin-3-yl, 6-(3,3-difluoro-azetidinyl)-2-methyl-pyridin-3-yl, 6-[N-(2-hydroxyethyl)-N-methylamino]-2-methyl-pyridin-3-yl, 6-(3-hydroxypyrrolidinyl)-2-methyl-pyridin-3-yl, 6-((S)-3-hydroxypyrrolidinyl)-2-methyl-pyridin-3-yl, 6-((R)-3-hydroxypyrrolidinyl)-2-methyl-pyridin-3-yl, 6-(3-methoxypyrrolidinyl)-2-methyl-pyridin-3-yl, 6-(3-hydroxy-azetidinyl)-2-methyl-pyridin-3-yl, 6-amino-2-methyl-pyridin-3-yl, 6-(2-hydroxyprop-2-yl)-2-methyl-pyridin-3-yl, 6-trifluoromethyl-2-methyl-pyridin-3-yl, 6-(3,3,4,4-tetrafluoropyrrolidinyl)-2-methyl-pyridin-3-yl;
R² is hydrogen or lower alkyl;
R³ is selected from 3,4-dimethoxyphenyl, 4-benzamide, 4-N-methyl-benzamide, 2-methoxpyridin-5-yl, 2-ethoxypyridin-5-yl, 3-ethoxypyridin-5-yl, 2-methoxpyridin-4-yl, 3-(iso-propoxy)pyridin-5-yl, 2-hydroxymethylpyrid-5-yl, 3-(2-hydroxyprop-2-yl)pyrid-5-yl, 2-aminopyridin-5-yl, 3-aminopyridin-5-yl, 2-dimethylaminopyrid-5-yl, 2-amino-3-trifluoromethyl-pyrid-5-yl, 2-(3-N,N-dimethylaminopropoxy)pyridin-5-yl, 3-(2-methoxyethoxy)pyrid-5-yl, 2-methylaminopyrid-5-yl, 3-methylaminopyrid-5-yl, 3-(iso-propyl amino)pyrid-5-yl, 3-azetidin-1-yl-pyridin-5-yl, 3-(2-cyanoprop-2-yl)-pyridin-5-yl, 3-(1-cyanocyclobut-1-yl)-pyridin-5-yl, 2-fluoropyrid-5-yl, 2-(methyl)-3-(methoxy)pyrid-5-yl, 2-(methyl)-3-(ethoxy)pyrid-5-yl, 2-(methyl)-3-(isopropoxy)pyrid-5-yl, 2-(dimethoxy-methyl)-3-(methoxy)pyrid-5-yl, 2-(methoxy-methyl)-3-(methoxy)pyrid-5-yl, 2-(methoxy-methyl)-3-(ethoxy)pyrid-5-yl, 2-(methoxy-methyl)-3-(iso-propoxy)pyrid-5-yl, 2-(hydroxymethyl)-3-(ethylamino)pyrid-5-yl, 2-(hydroxymethyl)-3-(methylamino)pyrid-5-yl, 2-(methyl)-3-(methylamino)pyrid-5-yl, 2-(methyl)-3-(ethylamino)pyrid-5-yl, 3-(fluoro)-2-(methylamino)pyrid-5-yl, 3-(chloro)-2-(methylamino)pyrid-5-yl, 3-(trifluoromethyl)-2-(methylamino)pyrid-5-yl, 3-(trifluoromethyl)-2-(ethylamino)pyrid-5-yl, 3-(trifluoromethyl)-2-(amino)pyrid-5-yl, 2-(hydroxymethyl)-3-(methoxy)pyrid-5-yl, 3-(hydroxymethyl)-2-(amino)pyrid-5-yl, 3-(methoxymethyl)-2-(amino)pyrid-5-yl, 3-(ethoxymethyl)-2-(amino)pyrid-5-yl, 2-(methyl)-3-(2-methoxyethoxy)pyrid-5-yl, 3-(ethoxymethyl)-2-(ethylamino)pyrid-5-yl, 3-(methoxymethyl)-2-(methylamino)pyrid-5-yl, 2-(amino)-3-(methylaminocarbonyl)pyrid-5-yl, 2-methylaminopyrimidin-5-yl, 2-ethylaminopyrimidin-5-yl, 2-dimethylaminopyrimidin-5-yl, 2-ethoxypyrimidin-5-yl, 2,4-dimethoxypyrimidin-5-yl, 2-(ethylamino)-4-(methoxy)pyrimidin-5-yl, 1H-pyrrolo[2,3-b]pyridin-5-yl, 1-methyl-1H-pyrrolo[2,3-b]pyridin-5-yl, quinolin-3-yl, imidazo[1,2-a]pyridin-6-yl.

A further embodiment of the present invention includes compounds of formula (I) or pharmaceutically acceptable salts, solvates or hydrates thereof, wherein:
X is O or S;
Y is CH or N;
R¹ is unsubstituted pyridyl or pyridyl substituted with one, two or three substituents (preferably one or two substituents), at least one of which is in the alpha-position, said substituents being independently selected from halo; lower alkyl; lower alkoxy; lower alkoxy lower alkoxy; mono- or di-loweralkyl substituted amino; unsubstituted piperazinyl or lower alkyl-substituted piperazinyl;
R² is hydrogen or lower alkyl;
R³ is unsubstituted phenyl, or phenyl substituted with one or two groups independently selected from the group consisting of halo; halo-lower alkyl; hydroxyl; amino; mono or disubstituted amino; cyclic amino; amino-lower alkyl; lower alkoxy; hydroxy-lower alkyl; hydroxy lower alkoxy; lower alkyl; cyano; cyano-lower alkyl; amidino; N-hydroxyamidino; amidino-lower alkyl; or N-hydroxyamidino-lower alkyl; sulfonyl; alkyl-substituted sulfonyl; sulfonamide; pyrrolidinesulfonyl; [1,3]dioxolo; halo-substituted [1,3]dioxolo; alkoxy carbonyl; carbamoyl; substituted carbamoyl; heterocycle; heterocyclyl lower alkyl; heteroaryl or heteroaryl lower alkyl; or
R³ is indolyl, 2,3-dihydro-1H-indol-5-yl, 1-methyl-2,3-dihydro-1H-indol-5-yl, 2-oxo-2,3-dihydro-1H-indol-5-yl, pyridyl, pyrimidinyl, 1H-pyrrolo[2,3-b]pyridin-5-yl, 1-methyl-1H-pyrrolo[2,3-b]pyridin-5-yl, pyrazolyl, pyrazol-4-yl, pyrazinyl, quinolyl, quinol-3-yl, 1H-imidazo[4,5-b]pyridin-2(3H)-one-6-yl, 3H-imidazo[4,5-b]pyridin-6-yl, 3H-[1,2,3]triazolo[4,5-b]pyridin-6-yl each independently being unsubstituted or substituted by one or two radicals independently selected from the group consisting of halo; halo-lower alkyl; hydroxyl; amino; mono or disubstituted amino; cyclic amino; amino-lower alkyl; lower alkoxy; lower alkoxy lower alkyl; hydroxy-lower alkyl; hydroxy lower alkoxy; lower alkyl; cyano; cyano-lower alkyl; amidino; N-hydroxyamidino; amidino-lower alkyl; or N-hydroxyamidino-lower alkyl; sulfonyl; alkyl-substituted sulfonyl; sulfonamide; pyrrolidine-1-sulfonyl; [1,3]dioxolo; halo substituted [1,3]dioxolo; alkoxy carbonyl; carbamoyl; aminocarbonylalkyl; N-mono-substituted aminocarbonylalkyl; N-di-substituted aminocarbonylalkyl; 3H-tetrazolyl, pyrazol, heterocyclyl lower alkyl, heteroaryl or heteroaryl lower alkyl.

A further embodiment of the present invention includes compounds of formula (I) or pharmaceutically acceptable salts, solvates or hydrates thereof, wherein:
X is O or S (preferably O);
Y is CH or N (preferably CH);
R¹ is unsubstituted pyridinyl or 2-methyl-pyridin-3-yl, 3-methyl-pyridin-2-yl, 4-methyl-pyridin-3-yl, 2,6-dimethyl-pyridin-3-yl, 2-fluoro-pyridin-3-yl, 6-fluoro-2-methyl-pyridin-3-yl, 2,6-dimethoxy-pyridin-3-yl, 6-methoxy-2-methyl-pyridin-3-yl, 2-methoxy-6-piperazin-1-yl-pyridin-3-yl, 2-methoxy-6-piperazin-1-yl-pyridin-3-yl, 2-methoxy-6-(4-methyl-piperazin-1-yl)-pyridin-3-yl;
R² is hydrogen or lower alkyl;
R³ is selected from phenyl; hydroxyphenyl; methoxyphenyl; 3,4-dimethoxyphenyl; ethoxyphenyl; 3,4-diethoxyphenyl; methoxy ethoxy-phenyl; 3-methoxy-4-(2-methoxy ethoxy)-phenyl; 3-methoxy-4-hydroxy phenyl; fluoro-hydroxy-phenyl; hydroxy-fluoroalkyl-phenyl; 2,2-difluoro-benzo[1,3]dioxolo; benzene sulfonamide; N-(phen-3-yl)-methanesulfonamide; N-methyl-N-phen-3-yl-methanesulfonamide; 3-methanesulfonylphenyl; 3- or 4-benzamide; 3- or 4-N-methyl-benzamide; 3- or 4-N,N-dimethyl-benzamide; pyrazol-phenyl; imidazol-phenyl; pyridyl; alkylpyridyl; alkoxypyridyl; cycloalkylalkoxypyridyl; alkoxyalkylpyridyl; alkoxyalkoxypyridyl, benzyloxyalkoxypyridyl, hydroxyalkylpyridyl; alkyl-sulfonyl pyridyl; hydroxyalkoxypyridyl; alkoxycarbonylpyridyl; aminopyridyl; alkylaminopyridyl; dialkylaminopyridyl; cycloaminopyridyl; hydroxyalkylaminopyridyl; amino-haloloweralkyl-pyridyl; haloalkylpyridinyl; halopyridyl; halo-alkoxy-pyridyl; carbamoylpyridyl; alkyl-substituted carbamoyl; piperazinylpyridyl; N-alkylpiperazinylpyridyl; alkylsulfonamidopyridyl; dialkylsulfonamido(alkyl)pyridyl; (alkoxy)(alkylcarbonylamino)pyridyl; (alkoxy)(alkylcarbonyl-N-alkylamino)pyridyl; (alkoxy)(amino)pyridyl; (alkoxy)(alkylaminocarbonyl)pyridyl; (alkoxy)(hydroxycarbonyl)pyridyl; pyrimidinyl; di-loweralkylaminopyrimidinyl; alkoxypyrimidinyl; 1H-pyrrolo[2,3-b]pyridinyl; 1-methyl-1H-pyrrolo[2,3-b]pyridinyl; quinolinyl; 2-oxo-2,3-dihydro-1H-indol-5-yl; 1-methyl-2,3-dihydro-1H-indol-5-yl;

A further embodiment of the present invention includes compounds of formula (I) or pharmaceutically acceptable salts, solvates or hydrates thereof, wherein:
X is O or S;
Y is CH or N;
R¹ is unsubstituted pyridyl or pyridyl substituted with one, two or three substituents (preferably one or two substituents), at least one of which is in the alpha-position, said substituents being independently selected from halo; lower alkyl; lower alkoxy; lower alkoxy lower alkoxy; mono- or di-loweralkyl substituted amino; unsubstituted piperazinyl or lower alkyl-substituted piperazinyl;
R² is hydrogen or lower alkyl;
R³ is unsubstituted phenyl, or phenyl substituted with one or two groups independently selected from the group consisting of lower alkoxy; carbamoyl; mono- or di-lower alkyl-substituted carbamoyl; or
R³ is pyridyl, pyrimidinyl, 1H-pyrrolo[2,3-b]pyridin-5-yl, each independently being unsubstituted or substituted by one or two radicals independently selected from the group consisting of halo-lower alkyl; amino; mono- or di-loweralkyl substituted amino; lower alkoxy; hydroxy-lower alkyl; N,N-di-loweralkyl aminoloweralkoxy.

A further embodiment of the present invention includes compounds of formula (I) or pharmaceutically acceptable salts, solvates or hydrates thereof, wherein:
X is O or S (preferably O);
Y is CH or N (preferably CH);
R¹ is unsubstituted pyridinyl or 2-methyl-pyridin-3-yl, 3-methyl-pyridin-2-yl, 4-methyl-pyridin-3-yl, 2,6-dimethyl-pyridin-3-yl, 2-fluoro-pyridin-3-yl, 6-fluoro-2-methyl-pyridin-3-yl, 2,6-dimethoxy-pyridin-3-yl, 6-methoxy-2-methyl-pyridin-3-yl, 2-methoxy-6-piperazin-1-yl-pyridin-3-yl, 2-methoxy-6-piperazin-1-yl-pyridin-3-yl, 2-methoxy-6-(4-methyl-piperazin-1-yl)-pyridin-3-yl;
R² is hydrogen or lower alkyl;
   R³ is selected from 3,4-dimethoxyphenyl, 4-benzamide, 4-N-methyl-benzamide, 2-methoxpyridin-5-yl, 2-ethoxypyridin-5-yl, 2-hydroxymethylpyrid-5-yl, 2-aminopyridin-5-yl, 2-dimethylaminopyrid-5-yl, 2-amino-3-trifluoromethyl-pyrid-5-yl, 2-(3-N,N-dimethylaminopropoxy)pyridin-5-yl, 2-methylaminopyrimidin-5-yl, 2-ethoxypyrimidin-5-yl, 1H-pyrrolo[2,3-b]pyridin-5-yl, quinolin-3-yl.

Most preferred is a compound of the formula (I), or a pharmaceutically acceptable salt, solvate or hydrate thereof, as exemplified hereinbelow under “Examples”.

The present invention is further directed to a method of treating a protein kinase dependent disease comprising administering a compound of formula (I), where the disease to be treated is a proliferative disease, preferably a benign or especially malignant tumor, more preferably carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, lymphomas, a mammary carcinoma or a leukemia, and including proliferative diseases such as tumor diseases, leukaemias and myeloproliferative disorders such as polycythemia vera, essential thrombocythemia, and myelofibrosis with myeloid metaplasia, basal cell carcinoma, squamous cell carcinoma and actinic keratosis.

Other diseases include Cowden syndrome, Lhermitte-Dudos disease and Bannayan-Zonana syndrome or diseases in which the PI3K/PKB pathway is aberrantly activated.

Most preferred is the use in accordance with the present invention of a compound of the formula (I), or a pharmaceutically acceptable salt, solvate or hydrate thereof, as exemplified hereinbelow under “Examples”.

The compounds of formula (I) that inhibit the protein or lipid kinase activities mentioned, especially mTOR Ser/Thr kinase activity and/or class I PI3 kinases activity, may therefore be useful in the treatment of protein or lipid kinase dependant diseases, especially diseases depending on class I and/or class IV PI3 kinases, for example, PI3Kalpha, PI3Kbeta, PI3Kdelta, PI3Kgamma and/or mTOR, or one or more of the individual kinase members thereof, including other PI3-kinases such as DNA-PK, ATM, ATR, hSMG-1 and Vps34 or any combination of two or more of the mentioned kinases.

Protein or lipid kinase dependent diseases are especially proliferative diseases, a benign or especially malignant tumor, a carcinoma of the brain, kidney, liver, adrenal gland, bladder, breast, stomach (especially gastric tumors), ovaries, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head, other diseases such as Cowden syndrome, Lhermitte-Duclos disease and Bannayan-Zonana syndrome, or diseases in which the PI3K/PKB pathway is aberrantly activated, an epidermal hyperproliferation, especially psoriasis, prostate hyperplasia, a neoplasia, especially of epithelial character, preferably mammary carcinoma or squamous cell carcinoma, or a leukemia. The compounds desirably are able to bring about the regression of tumors and to prevent the formation of tumor metastases and the growth of (also micro) metastases. In addition, they may be used in epidermal hyperproliferation, e.g., psoriasis; in prostate hyperplasia; in the treatment of neoplasias, especially of epithelial character, e.g., mammary carcinoma; and in leukemias; and in basal cell carcinoma, squamous cell carcinoma and actinic keratosis. It may also be possible to use the compounds of formula (I) in the treatment of diseases of the immune system insofar as several or, especially, individual lipid kinases and/or (further) serine/threonine protein kinases are involved.

The compounds of the formula (I) can be prepared according to the following methods:

A compound of formula (I) is converted into a compound of formula (I) by known in the art chemical reactions such as protecting group deprotection, e.g. tert-butyloxycarbonyl (boc) group deprotection with TFA, neat or in presence of a solvent such as a polyhalogeneated alkane, e.g. dichloromethane, at a temperature between 0° C. to 40° C.; functional group substitution e.g. alkylation of a hydroxyl group to form an alkoxy group by treatment with a strong based such as metal hydride, e.g. sodium hydride, in an aprotic solvent, e.g. THF or dimethylformamide, followed by addition of an alkylhalide, e.g. methyliodide, at a temperature between −20° C. and 40° C.; or functional group modification e.g. conversion of a carbonyl to a thiocarbonyl by treatment with Lawesson's reagent in a cycylether solvent, e.g. dioxane at a temperature between 60° C. and 120° C. or at reflux.

A compound of formula (I) wherein Y═CH is prepared by reacting a compound of the formula (II)

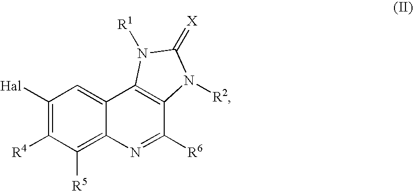

wherein
Hal refers to halogen preferably bromine; and
X, R¹, R², R⁴, R⁵ and R⁶ are as defined herein above;
   A: with a boronic acid of the formula III

R₃—B(OH)₂ (III)
or a boronate ester such as of formula IIIa

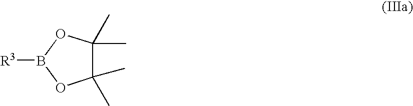

wherein R³ is as defined for a compound of the formula (I), in the presence of a base and a catalyst in a suitable solvent; to provide a compound of formula (I);
where the above starting compounds II, IIa, III, IIIa and IIIb may also be present with functional groups in protected form if necessary and/or in the form of salts, provided a salt-forming group is present and the reaction in salt form is possible;
any protecting groups in a protected derivative of a compound of the formula I are optionally removed; and
if desired, transforming an obtainable compound of formula (I) into a different compound of formula (I), or a N-oxide thereof, transforming a salt of an obtainable compound of formula (I) into the free compound or a different salt, or an obtainable free compound of formula (I) into a salt; and/or separating an obtainable mixture of isomers of compounds of formula (I) into the individual isomers.

In the following, more detailed description of the preferred process conditions, X, R¹, R², R³, R⁴, R⁵ and R⁶ have the meanings given herein for compounds of the formula (I), if not indicated otherwise.

Conversion of R² from H to a substituent different from H as defined above for R² can be achieved by treating compound of formula (I) or (II) in presence of a strong base in a suitable solvent and subsequent adjunction of a halogenated reagent Hal-R² wherein Hal refers to halogen preferably iodine or bromine, e.g. methyliodide.

A compound of formula (II) is prepared by reacting a compound of formula (IV)

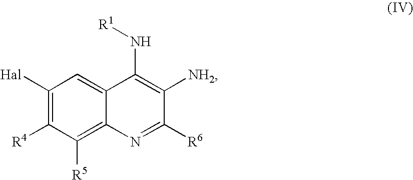

with phosgene or trichloromethyl chloroformate in a suitable solvent in presence of a base wherein
R¹, R⁴, R⁵ and R⁶ are as mentioned for a compound of the formula (I) and Hal refers to halogen preferably bromine.

A compound of the formula (IV) is prepared by reduction of a compound of the formula (V)

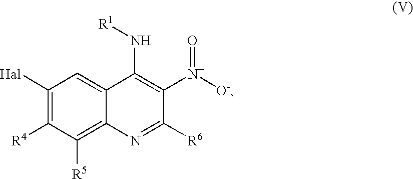

wherein the substituents and symbols are defined as for compounds of the formula (I) and Hal refers to halogen preferably bromine, in the presence of an appropriate catalyst, e.g., a skeleton based catalyst, such as Raney-Ni with hydrogen in an appropriate solvent, e.g., an alcohol and or a cycloalkylether, such as methanol and/or tetrahydrofurane; at preferred temperatures e.g. between 0° C. and 50° C., e.g., at RT.

A compound of formula (V) is preferably prepared by reacting a compound of the formula (VI)

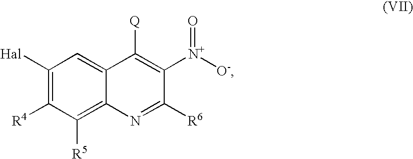

wherein
Q is halo, especially chloro;
Hal refers to halogen preferably bromine; and
the other moieties and symbols have the meanings indicated for compounds of the formula (I) with a compound of the formula (VII)

R₁—NH₂ (VII),
wherein R₁ is as defined for a compound of the formula (I), in the presence of a base such as a tertiaryamine, e.g. 1,2,2,6,6-pentamethylpiperidine, in an appropriate solvent; preferably a polar aprotic solvent such as dimethylacetamide, at preferred temperatures between 20° C. and 120° C. temperature of the reaction mixture, e.g., between 20° C. and 70° C.

A compound of the formula (VI) can be prepared by reacting a compound of the formula (VIII)

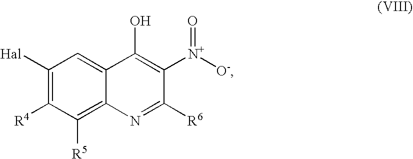

wherein the moieties and symbols have the meanings indicated for a compound of the formula (I), and Hal refers to halogen preferably bromine, with an inorganic acid halogenide, especially POCl₃ (preferably without solvent) at elevated temperatures, e.g., between 100° C. and 150° C. or under reflux.

A compound of the formula (VIII) is known in the art, can be synthesized according to methods known in the art and/or is commercially-available. For example, it can be synthesized by reacting a compound of the formula (IX)

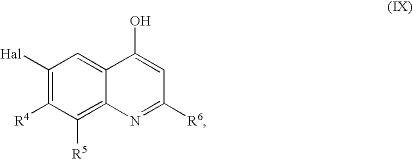

wherein the moieties and symbols have the meanings indicated for a compound of the formula (I) (x is preferably zero), and Hal refers to halogen preferably bromine, with nitric acid (aqueous) at a preferred temperature between 50° C. and 100° C., e.g., at 85° C.

A compound of the formula (VIII), can alternatively be synthesized by reacting a compound of the formula (X)

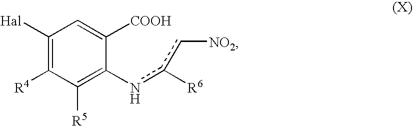

wherein the moieties and symbols have the meanings indicated for a compound of the formula (I), and Hal refers to halogen preferably bromine, with an anhydride of a carbonic acid, especially acetic anhydride, preferably in the presence of an alkali metal salt of a carboxylic acid, e.g., potassium acetate, at a preferred temperature between 50° C. and 150° C., e.g., at ca. 100-140° C.

A compound of the formula (X) can be obtained, e.g., by converting a compound of the formula (XI)

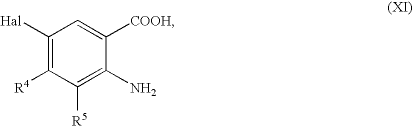

wherein Hal refers to halogen preferably bromine, to the corresponding compound of the formula (X) by reacting nitromethane in the presence of an alkali metal hydroxide, especially sodium hydroxide, at preferred temperatures between approximately 0° C. and 60° C., e.g., between 0° C. and RT; then pouring the product under cooling to approximately 0° C. into concentrated HCl and adding the compound of the formula (XI) and further concentrated HCl, subsequently allowing for further reaction at preferred temperatures between 0° C. and RT to result in the corresponding compound of formula (X).

A compound of formula (I) wherein Y═N is prepared in two steps by saponification of the ester group by treatment with a base such as an alkali metal hydroxide, e.g. lithium hydroxide in a solvent such as wet cycloaklylether, e.g. dioxane/water, at a temperature between 20° C. and 100° C., preferably between 30° C. and 60° C., a compound of the formula (XII)

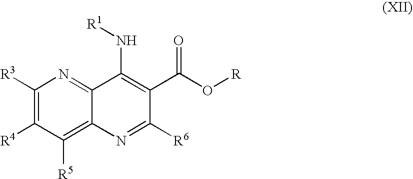

wherein
R¹, R³, R⁴, R⁵ and R⁶ are as defined herein above and R is unsubstituted or substituted lower alkyl, e.g. ethyl;

The freed intermediate obtained after neutralization of the reaction mixture with an acid, such as a mineral acid, e.g. hydrochloric acid, and evaporation to dryness is converted by a Cursius rearrangement to compound of formula (I) via in situ formation of the acylazide intermediate by treatment with diphenylphosphorylazide in an aprotic solvent, such as polar aprotic and/or polar protic, e.g. toluene/N-methylpyrrolidinone, in presence of a base, such as a tertiaryamine, e.g. triethylamine, at temperature between 60° C. and 120° C., e.g. between 80° C. and 110° C.; the isocyanato intermediate spontaneously cyclized to form compound of formula (I) in the reaction conditions.

A compound of formula (XII) is preferably prepared by reacting a compound of the formula (XIII)

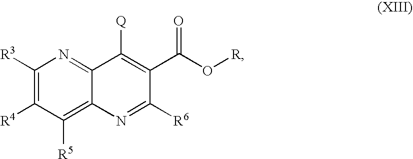

wherein
Q is halo, especially chloro; and
the other moieties have the meanings indicated for compounds of the formula (I), and R is unsubstituted or substituted lower alkyl, e.g. ethyl, with a compound of the formula (VII)

R₁—NH₂ (VII),

wherein R₁ is as defined for a compound of the formula (I), in the presence of a base such as a tertiaryamine, e.g. 1,2,2,6,6-pentamethylpiperidine, in an appropriate solvent; preferably a polar aprotic solvent such as dimethylacetamide, at preferred temperatures between 20° C. and 120° C. temperature of the reaction mixture, e.g., between 20° C. and 70° C.

A compound of formula (XIII) is preferably prepared by reacting a compound of the formula (XIV)

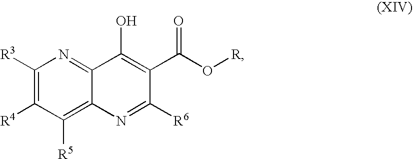

wherein the moieties have the meanings indicated for a compound of the formula (I) and R is unsubstituted or substituted lower alkyl, e.g. ethyl, with an inorganic acid halogenide, especially POCl₃ (preferably without solvent) at elevated temperatures, e.g., between 100° C. and 150° C. or under reflux.

A compound of formula (XIV) is preferably prepared by reacting a compound of the formula (XV)

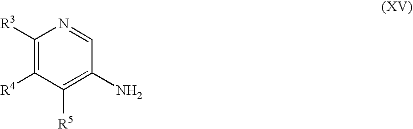

wherein the moieties have the meanings indicated for a compound of the formula (I) with a compound of formula (XVI)

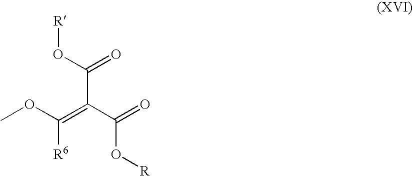

R and R′ being selected independently from unsubstituted or substituted alkyl, e.g. being both ethyl; in a solvent and pressure condition, e.g. xylene in a seal tube, allowing reaction at a temperature between 150° C. and 300° C., e.g. between 220° C. and 250° C.

A compound of formula (XV) is known in the art and can be prepared by reduction of a compound of the formula (XVII)

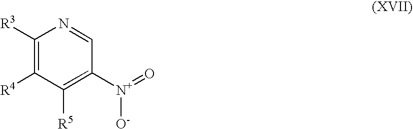

wherein the moieties have the meanings indicated for a compound of the formula (I) with a compound of formula (XVI) in the presence of an appropriate catalyst, e.g., a skeleton based catalyst, such as Raney-Ni with hydrogen in an appropriate solvent, e.g., an alcohol and or a cycloalkylether, such as methanol and/or tetrahydrofurane; at preferred temperatures e.g. between 0° C. and 50° C., e.g., at RT.

A compound of formula (XVII) is known in the art and can be prepared by reacting a compound of formula (XVIII)

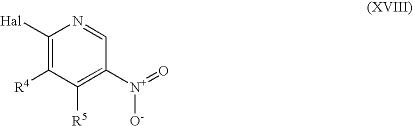

with a boronic acid of the formula III

R₃—B(OH)₂ (III)
or a boronate ester such as of formula IIIa

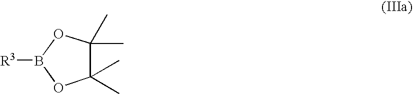

wherein R₃ is as defined for a compound of the formula (I), in the presence of a base and a catalyst in a suitable solvent.

Other starting materials are either known in the art, can be prepared according to methods that are known in the art, e.g., in analogy to the methods described hereinabove or in the examples, and/or are commercially-available.

The present invention relates also to novel starting materials and/or intermediates and to processes for their preparation. The starting materials used and the reaction conditions selected are preferably those that result in the compounds described as being preferred.

Other starting materials are either known in the art, can be prepared according to methods that are known in the art, e.g., in analogy to the methods described hereinabove or in the examples, and/or are commercially-available.

The present invention relates also to novel starting materials and/or intermediates and to processes for their preparation. The starting materials used and the reaction conditions selected are preferably those that result in the compounds described as being preferred.

Salts of compounds of formula (I) having at least one salt-forming group may be prepared in a manner known per se. For example, salts of compounds of formula (I) having acid groups may be formed, e.g., by treating the compounds with metal compounds, such as alkali metal salts of suitable organic carboxylic acids, e.g., the sodium salt of 2-ethylhexanoic acid; with organic alkali metal or alkaline earth metal compounds, such as the corresponding hydroxides, carbonates or hydrogen carbonates, such as sodium or potassium hydroxide, carbonate or hydrogen carbonate; with corresponding calcium compounds or with ammonia or a suitable organic amine, stoichiometric amounts or only a small excess of the salt-forming agent preferably being used. Acid addition salts of compounds of formula (I) are obtained in customary manner, e.g., by treating the compounds with an acid or a suitable anion exchange reagent. Internal salts of compounds of formula (I) containing acid and basic salt-forming groups, e.g., a free carboxy group and a free amino group, may be formed, e.g., by the neutralization of salts, such as acid addition salts, to the isoelectric point, e.g., with weak bases, or by treatment with ion exchangers.

Salts can be converted in customary manner into the free compounds; metal and ammonium salts can be converted, e.g., by treatment with suitable acids; and acid addition salts, e.g., by treatment with a suitable basic agent.

Mixtures of isomers obtainable according to the invention can be separated in a manner known per se into the individual isomers; diastereoisomers can be separated, e.g., by partitioning between polyphasic solvent mixtures, recrystallization and/or chromatographic separation, e.g., over silica gel or by, e.g., medium pressure liquid chromatography over a reversed phase column; and racemates can be separated, e.g., by the formation of salts with optically pure salt-forming reagents and separation of the mixture of diastereoisomers so obtainable, e.g., by means of fractional crystallization, or by chromatography over optically active column materials.

Intermediates and final products can be worked up and/or purified according to standard methods, e.g., using chromatographic methods, distribution methods, re-crystallization and the like.

### Additional Process Steps

In the additional process steps, carried out as desired, functional groups of the starting compounds which should not take part in the reaction may be present in unprotected form or may be protected, e.g., by one or more protecting groups. The protecting groups are then wholly or partly removed according to one of the known methods.

Protecting groups, and the manner in which they are introduced and removed are described, e.g., Protective Groups in Organic Chemistry, Plenum Press, London, N.Y. (1973); Methoden der organischen Chemie, Houben-Weyl, 4^{th} Edition, Vol. 15/1, Georg-Thieme-Verlag, Stuttgart (1974); and Theodora W. Greene, Protective Groups in Organic Synthesis, John Wiley & Sons, NY (1981). A characteristic of protecting groups is that they can be removed readily, i.e., without the occurrence of undesired secondary reactions, e.g., by solvolysis, reduction, photolysis or alternatively under physiological conditions.

The end products of formula (I) may however also contain substituents that can also be used as protecting groups in starting materials for the preparation of other end products of formula (I). Thus, within the scope of this text, only a readily removable group that is not a constituent of the particular desired end product of formula (I) is designated a “protecting group”, unless the context indicates otherwise.

### General Process Conditions

The following applies in general to all processes mentioned hereinbefore and hereinafter, while reaction conditions specifically mentioned above or below are preferred:

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, e.g., ion exchangers, such as cation exchangers, e.g., in the H⁺ form; depending on the nature of the reaction and/or of the reactants at reduced, normal or elevated temperature, e.g., in a temperature range of from about −100° C. to about 190° C.; preferably from approximately −80° C. to approximately 150° C., e.g., at from −80° C. to −60° C. at RT, at from −20° C. to 40° C. or at reflux temperature; under atmospheric pressure or in a closed vessel, where appropriate under pressure and/or in an inert atmosphere, e.g., under an argon or nitrogen atmosphere.

At all stages of the reactions, mixtures of isomers that are formed can be separated into the individual isomers, e.g., diastereoisomers or enantiomers; or into any desired mixtures of isomers, e.g., racemates or mixtures of diastereoisomers, e.g., analogously to the methods described under “additional process steps”.

The solvents from which those solvents that are suitable for any particular reaction may be selected include those mentioned specifically or, e.g., water; esters, such as lower alkyl-lower alkanoates, e.g., ethyl acetate; ethers, such as aliphatic ethers, e.g., diethyl ether; or cyclic ethers, e.g., tetrahydrofuran or dioxane; liquid aromatic hydrocarbons, such as benzene or toluene; alcohols, such as methanol, ethanol or 1- or 2-propanol; nitriles, such as acetonitrile; halogenated hydrocarbons, such as methylene chloride or chloroform; acid amides, such as dimethylformamide or dimethyl acetamide; bases, such as heterocyclic nitrogen bases, e.g., pyridine or N-methylpyrrolidin-2-one; carboxylic acid anhydrides, such as lower alkanoic acid anhydrides, e.g., acetic anhydride; cyclic, linear or branched hydrocarbons, such as cyclohexane, hexane or isopentane; or mixtures of those solvents, e.g., aqueous solutions, unless otherwise indicated in the description of the processes. Such solvent mixtures may also be used in working up, e.g., by chromatography or partitioning.

The compounds, including their salts, may also be obtained in the form of hydrates, or their crystals may, e.g., include the solvent used for crystallization. Different crystalline forms may be present.

The invention relates also to those forms of the process in which a compound obtainable as intermediate at any stage of the process is used as starting material and the remaining process steps are carried out, or in which a starting material is formed under the reaction conditions or is used in the form of a derivative, e.g., in protected form or in the form of a salt, or a compound obtainable by the process according to the invention is produced under the process conditions and processed further in situ. In the process of the present invention those starting materials are preferably used which result in new compounds of formula (I) described at the beginning as being especially valuable. Special preference is given to reaction conditions that are analogous to those mentioned in the examples.

### Pharmaceutical Compositions

The invention relates also to pharmaceutical compositions comprising a compound of formula (I), to their use in the therapeutic (in a broader aspect of the invention also prophylactic) treatment or a method of treatment of a lipid or protein kinase dependent disease, especially the preferred diseases mentioned above, to the compounds for said use and to the preparation of pharmaceutical preparations, especially for said uses.

The present invention also relates to pro-drugs of a compound of formula (I) that convert in vivo to the compound of formula (I) as such. Any reference to a compound of formula (I) is therefore to be understood as referring also to the corresponding pro-drugs of the compound of formula (I), as appropriate and expedient. The pharmacologically acceptable compounds of the present invention may be used, e.g., for the preparation of pharmaceutical compositions that comprise an effective amount of a compound of the formula (I), or a pharmaceutically acceptable salt thereof, as active ingredient together or in admixture with a significant amount of one or more inorganic or organic, solid or liquid, pharmaceutically acceptable carriers.

The invention relates also to a pharmaceutical composition that is suitable for administration to a warm-blooded animal, especially a human (or to cells or cell lines derived from a warm-blooded animal, especially a human, e.g., lymphocytes), for the treatment or, in a broader aspect of the invention, prevention of (=prophylaxis against) a disease that responds to inhibition of protein kinase activity, comprising an amount of a compound of formula (I) or a pharmaceutically acceptable salt thereof, which is effective for said inhibition, especially the in, together with at least one pharmaceutically acceptable carrier.

The pharmaceutical compositions according to the invention are those for enteral, such as nasal; rectal or oral; or parenteral, such as intramuscular or intravenous; or topical, such as dermal administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to a method of treatment for a disease that responds to inhibition of a lipid or protein kinase, which comprises administering an (against the mentioned disease) prophylactically or especially therapeutically effective amount of a compound of formula (I) according to the invention, especially to a warm-blooded animal, e.g., a human, that, on account of one of the mentioned diseases, requires such treatment.

The dose of a compound of the formula (I) or a pharmaceutically acceptable salt thereof to be administered to warm-blooded animals, e.g., humans of approximately 70 kg body weight, is preferably from approximately 3 mg to approximately 10 g, more preferably from approximately 10 mg to approximately 1.5 g, most preferably from about 100 mg to about 1000 mg/person/day, divided preferably into 1-3 single doses which may, e.g., be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, e.g., in unit dose form, such as in the form of ampoules, vials, suppositories, dragées, tablets or capsules.

The pharmaceutical compositions of the present invention are prepared in a manner known per se, e.g., by means of conventional dissolving, lyophilizing, mixing, granulating or confectioning processes.

Solutions of the active ingredient, and also suspensions, and especially isotonic aqueous solutions or suspensions, are preferably used, it being possible, e.g., in the case of lyophilized compositions that comprise the active ingredient alone or together with a carrier, e.g., mannitol, for such solutions or suspensions to be produced prior to use. The pharmaceutical compositions may be sterilized and/or may comprise excipients, e.g., preservatives, stabilizers, wetting and/or emulsifying agents, solubilizers, salts for regulating the osmotic pressure and/or buffers; and are prepared in a manner known per se, e.g., by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22 carbon atoms, especially from 12-22 carbon atoms, e.g., lauric acid, tridecylic acid, myristic acid, pentadecylic acid, palmitic acid, margaric acid, stearic acid, arachidic acid, behenic acid or corresponding unsaturated acids, e.g., oleic acid, elaidic acid, erucic acid, brasidic acid or linoleic acid, if desired with the addition of antioxidants, e.g., vitamin E, β-carotene or 3,5-di-tert-butyl-4-hydroxytoluene. The alcohol component of those fatty acid esters has a maximum of 6 carbon atoms and is a mono- or poly-hydroxy, e.g., a mono-, di- or tri-hydroxy; alcohol, e.g., methanol, ethanol, propanol, butanol or pentanol; or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C₈-C₁₂, Hüls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, e.g., lactose, saccharose, mannitol or sorbitol; cellulose preparations and/or calcium phosphates, e.g., tricalcium phosphate or calcium hydrogen phosphate; and binders, such as starch pastes using, e.g., corn, wheat, rice or potato starch, gelatin, tragacanth, methylcellulose, hydroxypropylmethylcellulose, sodium carboxymethylcellulose and/or polyvinylpyrrolidone; and/or, if desired, disintegrators, such as the above-mentioned starches; and/or carboxymethyl starch, crosslinked polyvinylpyrrolidone, agar, alginic acid or a salt thereof, such as sodium alginate. Excipients are especially flow conditioners and lubricants, e.g., silicic acid, talc, stearic acid or salts thereof, such as magnesium or calcium stearate; and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide; or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, e.g., with fillers, such as lactose; binders, such as starches; and/or glidants, such as talc or magnesium stearate; and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, e.g., for identification purposes or to indicate different doses of active ingredient.

Pharmaceutical compositions for topical administration can be obtained by combining the active ingredient with a liquid carrier (e.g. an aqueous liquid carrier) to dissolve or disperse the active, together with further optional formulating ingredients such as solvents/solubilisers, gelling agents, oils, stabilisers, buffers and preservatives to provide for example a solution, lotion, cream, gel or ointment.

### Combinations

A compound of the formula (I) may also be used to advantage in combination with each other or in combination with other therapeutic agents, especially other antiproliferative agents. Such antiproliferative agents include, but are not limited to, aromatase inhibitors; antiestrogens; topoisomerase I inhibitors; topoisomerase II inhibitors; microtubule active agents; alkylating agents; histone deacetylase inhibitors; compounds, which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; temozolomide (TEMODAL®); and leucovorin.

The term “aromatase inhibitor”, as used herein, relates to a compound which inhibits the estrogen production, i.e., the conversion of the substrates androstenedione and testosterone to estrone and estradiol, respectively. The term includes, but is not limited to, steroids, especially atamestane, exemestane and formestane; and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g., under the trademark AROMASIN. Formestane can be administered, e.g., in the form as it is marketed, e.g., under the trademark LENTARON. Fadrozole can be administered, e.g., in the form as it is marketed, e.g., under the trademark AFEMA. Anastrozole can be administered, e.g., in the form as it is marketed, e.g., under the trademark ARIMIDEX. Letrozole can be administered, e.g., in the form as it is marketed, e.g., under the trademark FEMARA or FEMAR. Aminoglutethimide can be administered, e.g., in the form as it is marketed, e.g., under the trademark ORIMETEN. A combination of the invention comprising a chemotherapeutic agent which is an aromatase inhibitor is particularly useful for the treatment of hormone receptor positive tumors, e.g., breast tumors.

The term “anti-estrogen”, as used herein, relates to a compound which antagonizes the effect of estrogens at the estrogen receptor level. The term includes, but is not limited to, tamoxifen, fulvestrant, raloxifene and raloxifene hydrochloride. Tamoxifen can be administered, e.g., in the form as it is marketed, e.g., under the trademark NOLVADEX. Raloxifene hydrochloride can be administered, e.g., in the form as it is marketed, e.g., under the trademark EVISTA. Fulvestrant can be formulated as disclosed in U.S. Pat. No. 4,659,516 or it can be administered, e.g., in the form as it is marketed, e.g., under the trademark FASLODEX. A combination of the invention comprising a chemotherapeutic agent which is an antiestrogen is particularly useful for the treatment of estrogen receptor positive tumors, e.g., breast tumors.

The term “anti-androgen”, as used herein, relates to any substance which is capable of inhibiting the biological effects of androgenic hormones and includes, but is not limited to, bicalutamide (CASODEX), which can be formulated, e.g., as disclosed in U.S. Pat. No. 4,636,505.

The term “gonadorelin agonist”, as used herein, includes, but is not limited to, abarelix, goserelin and goserelin acetate. Goserelin is disclosed in U.S. Pat. No. 4,100,274 and can be administered, e.g., in the form as it is marketed, e.g., under the trademark ZOLADEX. Abarelix can be formulated, e.g., as disclosed in U.S. Pat. No. 5,843,901.

The term “topoisomerase I inhibitor”, as used herein, includes, but is not limited to, topotecan, gimatecan, irinotecan, camptothecian and its analogues, 9-nitrocamptothecin and the macromolecular camptothecin conjugate PNU-166148 (compound A1 in WO 99/17804). Irinotecan can be administered, e.g., in the form as it is marketed, e.g., under the trademark CAMPTOSAR. Topotecan can be administered, e.g., in the form as it is marketed, e.g., under the trademark HYCAMTIN.

The term “topoisomerase II inhibitor”, as used herein, includes, but is not limited to, the anthracyclines, such as doxorubicin, including liposomal formulation, e.g., CAELYX; daunorubicin; epirubicin; idarubicin; nemorubicin; the anthraquinones mitoxantrone and losoxantrone; and the podophillotoxines etoposide and teniposide. Etoposide can be administered, e.g., in the form as it is marketed, e.g., under the trademark ETOPOPHOS. Teniposide can be administered, e.g., in the form as it is marketed, e.g., under the trademark VM 26-BRISTOL. Doxorubicin can be administered, e.g., in the form as it is marketed, e.g., under the trademark ADRIBLASTIN or ADRIAMYCIN. Epirubicin can be administered, e.g., in the form as it is marketed, e.g., under the trademark FARMORUBICIN. Idarubicin can be administered, e.g., in the form as it is marketed, e.g., under the trademark ZAVEDOS. Mitoxantrone can be administered, e.g., in the form as it is marketed, e.g., under the trademark NOVANTRON.

The term “microtubule active agent” relates to microtubule stabilizing, microtubule destabilizing agents and microtublin polymerization inhibitors including, but not limited to, taxanes, e.g., paclitaxel and docetaxel; vinca alkaloids, e.g., vinblastine, especially vinblastine sulfate; vincristine, especially vincristine sulfate and vinorelbine; discodermolides; cochicine; and epothilones and derivatives thereof, e.g., epothilone B or D or derivatives thereof. Paclitaxel may be administered, e.g., in the form as it is marketed, e.g., TAXOL. Docetaxel can be administered, e.g., in the form as it is marketed, e.g., under the trademark TAXOTERE. Vinblastine sulfate can be administered, e.g., in the form as it is marketed, e.g., under the trademark VINBLASTIN R.P. Vincristine sulfate can be administered, e.g., in the form as it is marketed, e.g., under the trademark FARMISTIN. Discodermolide can be obtained, e.g., as disclosed in U.S. Pat. No. 5,010,099. Also included are epothilone derivatives which are disclosed in WO 98/10121, U.S. Pat. No. 6,194,181, WO 98/25929, WO 98/08849, WO 99/43653, WO 98/22461 and WO 00/31247. Especially preferred are epothilone A and/or B.

The term “alkylating agent”, as used herein, includes, but is not limited to, cyclophosphamide, ifosfamide, melphalan or nitrosourea (BCNU or Gliadel). Cyclophosphamide can be administered, e.g., in the form as it is marketed, e.g., under the trademark CYCLOSTIN. Ifosfamide can be administered, e.g., in the form as it is marketed, e.g., under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes suberoylanilide hydroxamic acid (SAHA).

The term “antineoplastic antimetabolite” includes, but is not limited to, 5-fluorouracil or 5-FU; capecitabine; gemcitabine; DNA demethylating agents, such as 5-azacytidine and decitabine; methotrexate and edatrexate; and folic acid antagonists, such as pemetrexed. Capecitabine can be administered, e.g., in the form as it is marketed, e.g., under the trademark XELODA. Gemcitabine can be administered, e.g., in the form as it is marketed, e.g., under the trademark GEMZAR. Also included is the monoclonal antibody trastuzumab which can be administered, e.g., in the form as it is marketed, e.g., under the trademark HERCEPTIN.

The term “platin compound”, as used herein, includes, but is not limited to, carboplatin, cis-platin, cisplatinum and oxaliplatin. Carboplatin can be administered, e.g., in the form as it is marketed, e.g., under the trademark CARBOPLAT. Oxaliplatin can be administered, e.g., in the form as it is marketed, e.g., under the trademark ELOXATIN.

The term “compounds targeting/decreasing a protein or lipid kinase activity; or a protein or lipid phosphatase activity; or further anti-angiogenic compounds”, as used herein, includes, but is not limited to, protein tyrosine kinase and/or serine and/or threonine kinase inhibitors or lipid kinase inhibitors, e.g.,
a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g., a N-phenyl-2-pyrimidine-amine derivative, e.g., imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I (IGF-IR), such as compounds which target, decrease or inhibit the activity of IGF-IR, especially compounds which inhibit the IGF-IR receptor, such as those compounds disclosed in WO 02/092599;
d) compounds targeting, decreasing or inhibiting the activity of the Trk receptor tyrosine kinase family;
e) compounds targeting, decreasing or inhibiting the activity of the Axl receptor tyrosine kinase family;
f) compounds targeting, decreasing or inhibiting the activity of the c-Met receptor;
g) compounds targeting, decreasing or inhibiting the activity of the Kit/SCFR receptor tyrosine kinase;
h) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g., imatinib;
i) compounds targeting, decreasing or inhibiting the activity of members of the c-Abl family and their gene-fusion products, e.g., BCR-Abl kinase, such as compounds which target decrease or inhibit the activity of c-Abl family members and their gene fusion products, e.g., a N-phenyl-2-pyrimidine-amine derivative, e.g., imatinib, PD180970, AG957, NSC 680410 or PD173955 from ParkeDavis;
j) compounds targeting, decreasing or inhibiting the activity of members of the protein kinase C (PKC) and Raf family of serine/threonine kinases, members of the MEK, SRC, JAK, FAK, PDK and Ras/MAPK family members, or PI(3) kinase family, or of the PI(3)-kinase-related kinase family, and/or members of the cyclin-dependent kinase family (CDK) and are especially those staurosporine derivatives disclosed in U.S. Pat. No. 5,093,330, e.g., midostaurin; examples of further compounds include, e.g., UCN-01; safingol; BAY 43-9006; Bryostatin 1; Perifosine; Ilmofosine; RO 318220 and RO 320432; GO 6976; Isis 3521; LY333531/LY379196; isochinoline compounds, such as those disclosed in WO 00/09495; FTIs; PD184352; or QAN697 (a PI3K inhibitor);
k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810, AG 99, Tyrphostin AG 213, Tyrphostin AG 1748, Tyrphostin AG 490, Tyrphostin B44, Tyrphostin B44 (+) enantiomer, Tyrphostin AG 555, AG 494, Tyrphostin AG 556, AG957 and adaphostin (4-{[(2,5-dihydroxyphenyl)methyl]amino}-benzoic acid adamantyl ester, NSC 680410, adaphostin; and
l) compounds targeting, decreasing or inhibiting the activity of the epidermal growth factor family of receptor tyrosine kinases (EGFR, ErbB2, ErbB3, ErbB4 as homo- or hetero-dimers), such as compounds which target, decrease or inhibit the activity of the epidermal growth factor receptor family are especially compounds, proteins or antibodies which inhibit members of the EGF receptor tyrosine kinase family, e.g., EGF receptor, ErbB2, ErbB3 and ErbB4 or bind to EGF or EGF related ligands, and are in particular those compounds, proteins or monoclonal antibodies generically and specifically disclosed in WO 97/02266, e.g., the compound of Example 39, or in EP 0 564 409; WO 99/03854; EP 0520722; EP 0 566 226; EP 0 787 722; EP 0 837 063; U.S. Pat. No. 5,747,498; WO 98/10767; WO 97/30034; WO 97/49688; WO 97/38983 and, especially, WO 96/30347, e.g., compound known as CP 358774; WO 96/33980, e.g., compound ZD 1839; and WO 95/03283, e.g., compound ZM105180, e.g., trastuzumab (HERCEPTIN), cetuximab, Iressa, Tarceva, OSI-774, CI-1033, EKB-569, GW-2016, E1.1, E2.4, E2.5, E6.2, E6.4, E2.11, E6.3 or E7.6.3; and 7H-pyrrolo-[2,3-d]pyrimidine derivatives which are disclosed in WO 03/013541.

Further anti-angiogenic compounds include compounds having another mechanism for their activity, e.g., unrelated to protein or lipid kinase inhibition, e.g., thalidomide (THALOMID) and TNP-470.

Compounds which target, decrease or inhibit the activity of a protein or lipid phosphatase are, e.g., inhibitors of phosphatase 1, phosphatase 2A, PTEN or CDC25, e.g., okadaic acid or a derivative thereof.

Compounds which induce cell differentiation processes are e.g. retinoic acid, α- γ- or δ-tocopherol or α- γ- or δ-tocotrienol.

The term cyclooxygenase inhibitor, as used herein, includes, but is not limited to, e.g., Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g., 5-methyl-2-(2′-chloro-6′-fluoroanilino)phenyl acetic acid or lumiracoxib.

The term “bisphosphonates”, as used herein, includes, but is not limited to, etridonic, clodronic, tiludronic, pamidronic, alendronic, ibandronic, risedronic and zoledronic acid. “Etridonic acid” can be administered, e.g., in the form as it is marketed, e.g., under the trademark DIDRONEL. “Clodronic acid” can be administered, e.g., in the form as it is marketed, e.g., under the trademark BONEFOS. “Tiludronic acid” can be administered, e.g., in the form as it is marketed, e.g., under the trademark SKELID. “Pamidronic acid” can be administered, e.g., in the form as it is marketed, e.g., under the trademark AREDIA™. “Alendronic acid” can be administered, e.g., in the form as it is marketed, e.g., under the trademark FOSAMAX. “Ibandronic acid” can be administered, e.g., in the form as it is marketed, e.g., under the trademark BONDRANAT. “Risedronic acid” can be administered, e.g., in the form as it is marketed, e.g., under the trademark ACTONEL. “Zoledronic acid” can be administered, e.g., in the form as it is marketed, e.g., under the trademark ZOMETA.

The term “mTOR inhibitors” relates to compounds which inhibit the mammalian target of rapamycin (mTOR) and which possess antiproliferative activity, such as sirolimus (Rapamune®), everolimus (Certican™), CCI-779 and ABT578.

The term “heparanase inhibitor”, as used herein, refers to compounds which target, decrease or inhibit heparin sulphate degradation. The term includes, but is not limited to, PI-88.

The term “biological response modifier”, as used herein, refers to a lymphokine or interferons, e.g., interferon γ.

The term “inhibitor of Ras oncogenic isoforms”, e.g., H-Ras, K-Ras or N-Ras, as used herein, refers to compounds which target, decrease or inhibit the oncogenic activity of Ras, e.g., a “farnesyl transferase inhibitor”, e.g., L-744832, DK8G557 or R115777 (Zarnestra).

The term “telomerase inhibitor”, as used herein, refers to compounds which target, decrease or inhibit the activity of telomerase. Compounds which target, decrease or inhibit the activity of telomerase are especially compounds which inhibit the telomerase receptor, e.g., telomestatin.

The term “methionine aminopeptidase inhibitor”, as used herein, refers to compounds which target, decrease or inhibit the activity of methionine aminopeptidase. Compounds which target, decrease or inhibit the activity of methionine aminopeptidase are, e.g., bengamide or a derivative thereof.

The term “proteasome inhibitor”, as used herein, refers to compounds which target, decrease or inhibit the activity of the proteasome. Compounds which target, decrease or inhibit the activity of the proteasome include, e.g., PS-341 and MLN 341.

The term “matrix metalloproteinase inhibitor” or “MMP inhibitor”, as used herein, includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “agents used in the treatment of hematologic malignancies”, as used herein, includes, but is not limited to, FMS-like tyrosine kinase inhibitors, e.g., compounds targeting, decreasing or inhibiting the activity of FMS-like tyrosine kinase receptors (Flt-3R); interferon, 1-b-D-arabinofuransylcytosine (ara-c) and bisulfan; and ALK inhibitors, e.g., compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, e.g., PKC412, midostaurin, a staurosporine derivative, SU11248 and MLN518.

The term “HSP90 inhibitors”, as used herein, includes, but is not limited to, compounds targeting, decreasing or inhibiting the intrinsic ATPase activity of HSP90; degrading, targeting, decreasing or inhibiting the HSP90 client proteins via the ubiquitin proteasome pathway. Compounds targeting, decreasing or inhibiting the intrinsic ATPase activity of HSP90 are especially compounds, proteins or antibodies which inhibit the ATPase activity of HSP90, e.g., 17-allylamino, 17-demethoxygeldanamycin (17AAG), a geldanamycin derivative, other geldanamycin related compounds, radicicol and HDAC inhibitors.

The term “antiproliferative antibodies”, as used herein, includes, but is not limited to, trastuzumab (Herceptin™), Trastuzumab-DM1, erlotinib (Tarceva™), bevacizumab (Avastin™), rituximab (Rituxan®), PRO64553 (anti-CD40) and 2C4 antibody. By antibodies is meant, e.g., intact monoclonal antibodies, polyclonal antibodies, multispecific antibodies formed from at least two intact antibodies, and antibodies fragments so long as they exhibit the desired biological activity.

For the treatment of acute myeloid leukemia (AML), compounds of formula (I) can be used in combination with standard leukemia therapies, especially in combination with therapies used for the treatment of AML. In particular, compounds of formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The structure of the active agents identified by code nos., generic or trade names may be taken from the actual edition of the standard compendium “The Merck Index” or from databases, e.g., Patents International, e.g., IMS World Publications.

The above-mentioned compounds, which can be used in combination with a compound of the formula (I), can be prepared and administered as described in the art, such as in the documents cited above.

A compound of the formula (I) may also be used to advantage in combination with known therapeutic processes, e.g., the administration of hormones or especially radiation.

A compound of formula (I) may in particular be used as a radiosensitizer, especially for the treatment of tumors which exhibit poor sensitivity to radiotherapy.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g., synergistic, effect or any combination thereof. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula I and a combination partner, are both administered to a patient simultaneously in the form of a single entity or dosage. The term “non-fixed combination” means that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The following examples are merely illustrative and not meant to limit the scope of the present claims in any manner.

### EXAMPLES

The following examples serve to illustrate the invention without limiting the scope thereof:

Temperatures are measured in degrees Celsius. Unless otherwise indicated, the reactions take place at rt. The following HPLC/MS and MS methods are used in the preparation of the Intermediates and Examples:

### HPLC Method:

### Method A (Always Used Unless Otherwise Noted)

### Equipment: Shimadzu SIL-10A

Method: Linear gradient 2-100% CH₃CN (0.1% TFA) and H₂O (0.1% TFA) in 4 min+2 min 100% CH₃CN (0.1% TFA); back to −100% CH₃CN (0.1% TFA) in 3 min.; detection at 215 nm, flow rate 2 mL/min at RT. Column: Nucleosil OD-5-100 C18 (150×4.6 mm)

### HPLC Method:

### Method B

System: Agilent 1100 Series with Waters Micromass ZQ
HPLC linear gradient between A=H₂O/TFA 1000:1 and B=acetonitrile/TFA 1000:1
Grad 1: 2-100% B in 7 min then 100% B for 2 min and finally 100-2% B in 1 min; column: CC125/4 Nucleosil 100-3 C18HD;
flow rate 1.0 ml/min. Detection at 215 nM.

### HPLC Method:

### Method C

HPLC linear gradient between A=H₂O/TFA 1000:1 and B=acetonitrile/TFA 1000:1
Grad 1: 2-100% Bin 4.5 min and 1 min at 100% B; column: Chromolith Performance 100 mm×4.5 mm (Merck, Darmstadt, Germany); flow rate 2 ml/min. Detection at 215 nM.

### LC-MS Method:

System: Agilent 1100 Series with Waters Micromass ZQ
Column: XBridge C18, 3×30 mm, 2.5 micron
Flow Rate: 1.4-2.4 mL/min
Eluent A: H₂O, containing 5% acetonitrile and 0.8% HCOOH
Eluent B: acetonitrile, containing 0.6% HCOOH

### Gradient: 0-2.4 min: 10% to 95% of B

In the following examples, the abbreviations given below are used:
DCM dichloromethane
DIPEA N,N-diisopropylethylamine
DMA N,N-dimethylacetamide
DME 1,2-dimethoxyethane
DMF N,N-dimethylformamide
DMSO dimethyl sulfoxide
EtOAc ethyl acetate
EtOH ethanol
h hour(s)
HPLC High Performance Liquid Chromatography
HV high vacuum
iPrMgCl isopropylmagnesium chloride
LC-MS liquid chromatography coupled with mass spectrometry
MeOH methanol
mL milliliter(s)
min minute(s)
MS−ES electrospray mass spectrometry
NBS N-bromosuccinimide
NMP N-methyl-2-pyrrolidone
PdCl₂(dppf) [1,1′-bis(diphenylphosphino)ferrocene]dichloropalladium (II)
PdCl₂(PPh₃)₂ bis(triphenylphosphine)palladium (II)dichloride
Pd₂(dba)₃ tris(dibenzylideneacetone)dipalladium (0)
PE pinacol ester
Prep.HPLC preparative high performance liquid chromatography
R_{f} ratio of fronts in TLC
RM reaction mixture
RT room temperature
TBME tert. Butyl methyl ether
TEA triethylamine
TFA trifluoroacetic acid
THF tedrahydrofurane
TLC thin layer chromatography
t_{R} retention time
SPhos 2-dicyclohexylphosphino-2′,6′-dimethoxybephenyl
TPTU O-(2-Oxo-1(2H)Pyridyl)-N,N,N′,N′-tetramethyluronium tetrafluoroborate
UV Ultraviolet

All starting materials are from commercial source such as but not limited to: ABCR, Acros, Aldrich, Alfa Aesar, Akos, Avocado, ChemBridge, Combi-Blocks, Fluka, Frontier Scientific, Lancaster, Matrix, Maybridge, unless otherwise noted.

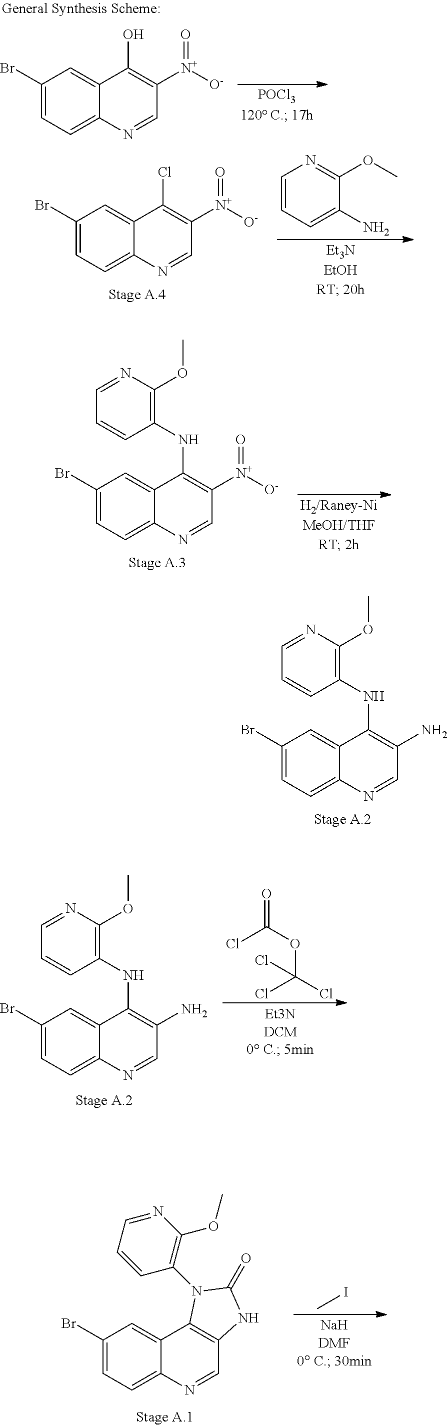

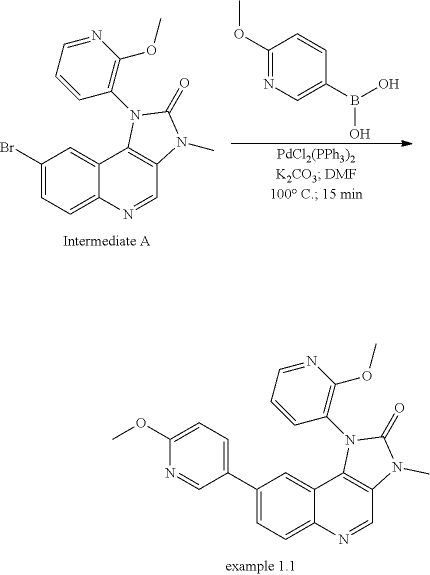

### Intermediate A 8-Bromo-1-(2-methoxy-pyridin-3-yl)-3-methyl-1,3-dihydro-imidazo[4,5-c]quinolin-2-one

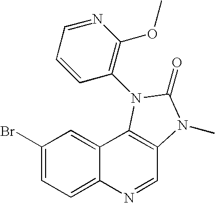

To a mixture of 8-bromo-1-(2-methoxy-pyridin-3-yl)-1,3-dihydro-imidazo[4,5-c]quinolin-2-one (Stage A.1, 606 mg, 1.633 mmol) in DMF anhydrous (10 ml) cooled to 0° C. was added NaH 55% (142 mg, 3.27 mmol) and stirred at 0° C. for 30 min. Then iodomethane (0.153 ml, 2.449 mmol) was added, the ice bath was removed and the RM was stirred at rt for 30 min. After that, the RM was extracted with EtOAc/H₂O. The organic layers were dried over Na₂SO₄ and evaporated. The residue was liophylised out of dioxane to give the title compound as a light brown solid. (HPLC: t_{R} 4.24 min (Method A); M+H=387.0, 385.0 Br-Pattern MS−ES)

### Stage A.1: 8-Bromo-1-(2-methoxy-pyridin-3-yl)-1,3-dihydro-imidazo[4,5-c]quinolin-2-one

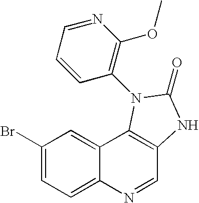

To a mixture of 6-bromo-N*4*-(2-methoxy-pyridin-3-yl)-quinoline-3,4-diamine (Stage A.2, 845 mg, 2.448 mmol) and TEA (1.71 ml, 12.24 mmol) in DCM (20 ml) cooled to 0° C. was added trichloromethyl carbonochloridate (0.310 ml, 2.57 mmol) and stirred at 0° C. for 30 min. After 5 min the product precipitated. The RM was filtered and the solid cake was washed with DCM and dried under high vacuum to give the title compound as a beige solid. (HPLC: t_{R} 4.07 min (Method A); M+H=372.9, 371.0 Br-Pattern MS−ES; ¹H-NMR (d₆-DMSO, 600 MHz) 11.89 (s, br, 1H), 8.80 (s, 1H), 8.61-8.36 (m, 1H), 8.25-8.02 (m, 1H), 8.02-7.86 (m, 1H), 7.78-7.55 (m, 1H), 7.46-7.22 (m, 1H), 7.18-6.92 (m, 1H), 3.77 (s, 3H))

### Stage A.2 6-Bromo-N*4*-(2-methoxy-pyridin-3-yl)-quinoline-3,4-diamine

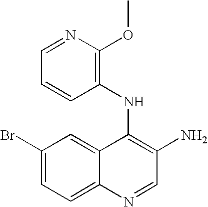

To a solution of (6-bromo-3-nitro-quinolin-4-yl)-(2-methoxy-pyridin-3-yl)-amine (Stage A.3, 934 mg, 2.489 mmol) in MeOH/THF=1:1 (20 ml) was added Ra-Ni (500 mg, 2.489 mmol) and the RM was stirred under H₂ atmosphere at rt for 2 h. The RM was then filtered over Celite and washed several times with MeOH. The filtrate was evaporated to dryness to give the title compound as a yellow solid. (HPLC: t_{R} 3.80 min (Method A); M+H=347.0, 345.0 Br-Pattern MS−ES; ¹H-NMR (d₆-DMSO, 600 MHz) 8.61 (s, 1H), 7.89-7.71 (m, 1H), 7.71-7.63 (m, 1H), 7.50-7.48 (m, 1H), 7.47-7.46 (m, 1H), 7.39-7.27 (m, 1H), 6.75-6.58 (m, 1H), 6.08-6.00 (m, 1H), 5.59 (s, 2H), 4.01 (s, 3H).

### Stage A.3 (6-Bromo-3-nitro-quinolin-4-yl)-(2-methoxy-pyridin-3-yl)-amine

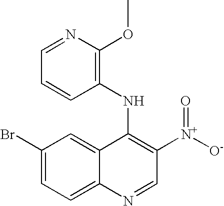

To a mixture of 6-bromo-4-chloro-3-nitro-quinoline (Stage A.4, 958 mg, 3.00 mmol) and TEA (0.836 ml, 6.00 mmol) in EtOH (20 ml) was added 2-methoxypyridin-3-amine (ABCR, Karlsruhe, Germany, 745 mg, 6.00 mmol) and stirred at rt overnight. Then the RM was filtered and the solid cake was washed with EtOH and dried under high vacuum to give the title compound as an orange solid. (HPLC: t_{R} 5.03 min (Method A); M+H=376.9, 374.9 Br-Pattern MS−ES)

### Stage A.4 6-Bromo-4-chloro-3-nitro-quinoline

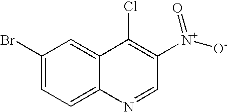

6-Bromo-3-nitro-quinolin-4-ol (Fluorochem Ltd., Derbyshire, United Kingdom, 10 g, 37.2 mmol) was added to POCl₃ (70 ml). The RM was stirred at 120° C. for 17 h. Then the RM was cooled with an ice-bath, before being slowly dropped onto ice-water. The precipitate was filtered and washed with cold water. The residue was dissolved in DCM, washed with brine, dried over Na₂SO₄, filtered and evaporated to give the title compound as a beige solid (HPLC t_{R} 3.64 min (Method A))

The following intermediates were synthesized in a similar manner as described for intermediate A using as replacement for the 2-methoxypyridin-3-amine a different aminopyridine starting material:
2-Methyl-pyridin-3-ylamine (B, Aldrich, Buchs, Switzerland)
3-Methyl-pyridin-2-ylamine (C, Aldrich, Buchs, Switzerland)
4-Methyl-pyridin-3-ylamine (D, Aldrich, Buchs, Switzerland)
Pyridin-3-ylamine (E, Aldrich, Buchs, Switzerland)
2-Fluoro-pyridin-3-ylamine (F, 3B Scientific, Libertyville, USA)
6-Fluoro-2-methyl-pyridin-3-ylamine (G, Fluorochem, Derbyshire, UK)
2,6-Dimethoxy-pyridin-3-ylamine (H, AB Chemicals, Quebec, Canada)
4-(5-Amino-6-methoxy-pyridin-2-yl)-piperazine-1-carboxylic acid tert-butyl ester (I); synthesis see example 9.1; stage 9.1.1
4-[5-(8-Bromo-3-methyl-2-oxo-2,3-dihydro-imidazo[4,5-c]quinolin-1-yl)-6-methoxy-pyridin-2-yl]-piperazine-1-carboxylic acid tert-butyl ester (J); synthesis see example 10.1; stage 10.1.1
6-Methoxy-2-methyl-pyridin-3-ylamine (K, Ark Pharm Inc., Libertyville, Ill. 60048 USA)
N*2*-Ethyl-6,N*2*-dimethyl-pyridine-2,5-diamine (L), synthesis see example 12.1; stage 10.1.1 and 10.1.2.
6-(2-Methoxy-ethoxy)-2-methyl-pyridin-3-ylamine (M), synthesis see example 13.1; stage 13.1.1
2,6-Dimethyl-pyridin-3-ylamine (N, Lancaster, Frankfurt am Main, Germany)
N*2*-(2-Methoxy-ethyl)-6,N*2*-dimethyl-pyridine-2,5-diamine (0, synthesis see example 15.1; stage 15.1.1)
6-Fluoro-4-methyl-pyridin-3-ylamine (P, Apollo Scientific Ltd., Bredbury, UK)
5-Methyl-pyridin-3-ylamine (Q, Aldrich, Buchs, Switzerland)
2-Chloro-6-methoxy-pyridin-3-ylamine (R, Aldrich, Buchs, Switzerland)
6,N*2*,N*2*-Trimethyl-pyridine-2,5-diamine (S, synthesis see example 19.1; stage 19.1.1 and 19.1.2)
N-(5-Amino-6-methyl-pyridin-2-yl)-2-methoxy-acetamide (T, synthesis see example 20.1.1 and 20.1.2)
4-(5-Amino-6-methyl-pyridin-2-yl)-1-methyl-piperazin-2-one (U, synthesis see example 21.1; stage 21.1.1 and 21.1.2)
6-(2-Methoxy-ethoxy)-2-methyl-pyridin-3-ylamine (V, synthesis see example 22.1; stage 22.1.1 and 22.1.2)
6-Azetidin-1-yl-2-methyl-pyridin-3-ylamine (W, synthesis see example 23.1; stage 23.1.1 and 23.1.2)
6-(3,3-Difluoro-azetidin-1-yl)-2-methyl-pyridin-3-ylamine (X, synthesis see example 24.1; stage 24.1.1 and 24.1.2)
2-Methyl-6-pyrrolidin-1-yl-pyridin-3-ylamine (Y, synthesis see example 25.1; stage 25.1.1 and 25.1.2)
N*2*-[2-(tert-Butyl-diphenyl-silanyloxy)-ethyl]-6,N*2*-dimethyl-pyridine-2,5-diamine (Z1, synthesis see example 26.1; stage 26.1.1-25.1.3)
1-(5-Amino-6-methyl-pyridin-2-yl)-pyrrolidin-3-ol (Z2; synthesis see example 27.1; stage 27.1.1)
6-[3-(tert-Butyl-diphenyl-silanyloxy)-azetidin-1-yl]-2-methyl-pyridin-3-ylamine (Z4, synthesis see example 29.1; stage 29.1.1)
2-(5-Amino-6-methyl-pyridin-2-yl)-propan-2-ol (Z5, synthesis see example 31.1; stage 31.1.1-31.1.3)
(S)-1-(5-Amino-6-methyl-pyridin-2-yl)-pyrrolidin-3-ol (Z6, synthesis see example 32.1; stage 32.1.1)
2-Methyl-6-(trifluoromethyl)pyridine-3-amine (Z7, AKL Research LLP)
(R)-1-(5-Amino-6-methyl-pyridin-2-yl)-pyrrolidin-3-ol (Z8, synthesis see example 34.1; stage 34.1.1)
Stage 35.1.2 2-Methyl-6-(3,3,4,4-tetrafluoro-pyrrolidin-1-yl)-pyridin-3-ylamine (Z9, synthesis see example 35.1; stage 35.1.1)

| | | | | HPLC |
|---|---|---|---|---|
| | | | | (method |
| | | | MS-ES | A) |
| Intermediate | structure | Name of the intermediate | (M + H) | t_{R} (min) |
| B | 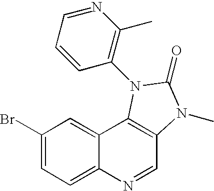 | 8-Bromo-3-methyl-1-(2-methyl-pyridin- 3-yl)-1,3-dihydro-imidazo[4,5-c]quinolin- 2-one | 370.9 | 3.86 |
| C | 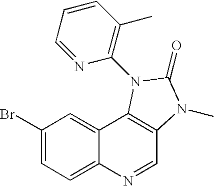 | 8-Bromo-3-methyl-1-(3-methyl-pyridin- 2-yl)-1,3-dihydro-imidazo[4,5-c]quinolin- 2-one | 370.9 | 4.22 |
| D | 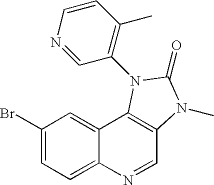 | 8-Bromo-3-methyl-1-(4-methyl-pyridin- 3-yl)-1,3-dihydro-imidazo[4,5-c]quinolin- 2-one | 370.9 | 3.91 |
| E | 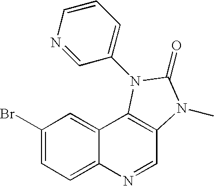 | 8-Bromo-3-methyl-1-pyridin-3-yl-1,3- dihydro-imidazo[4,5-c]quinolin-2-one | 357.0 | 3.88 |
| F | 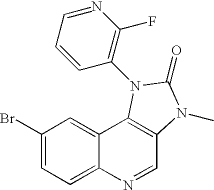 | 8-Bromo-1-(2-fluoro-pyridin-3-yl)-3- methyl-1,3-dihydro-imidazo[4,5-c] quinolin-2-one | 374.8 | 4.28 |
| G | 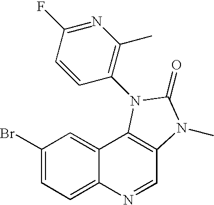 | 8-Bromo-1-(6-fluoro-2-methyl-pyridin- 3-yl)-3-methyl-1,3-dihydro-imidazo[4, 5-c]quinolin-2-one | 389.0 | 4.32 |
| H | 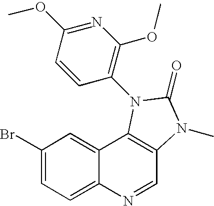 | 8-Bromo-1-(2,6-dimethoxy-pyridin-3- yl)-3-methyl-1,3-dihydro-imidazo[4, 5-c]quinolin-2-one | 416.9 | 4.59 |
| I | 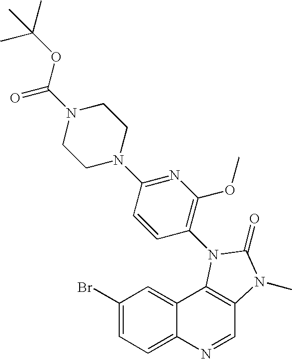 | 4-[5-(8-Bromo-3-methyl-2-oxo-2,3- dihydro-imidazo[4,5-c]quinolin-1-yl)- 6-methoxy-pyridin-2-yl]-piperazine- 1-carboxylic acid tert-butyl ester | 570.9 | 5.06 |
| J | 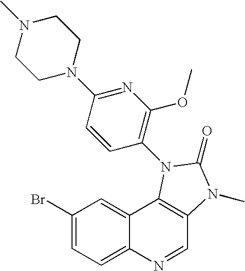 | 8-Bromo-1-[2-methoxy-6-(4-methyl- piperazin-1-yl)-pyridin-3-yl]-3-methyl- 1,3-dihydro-imidazo[4,5-c]quinolin-2- one | 484.8 | 3.68 |
| K | 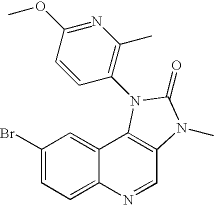 | 8-Bromo-1-(6-methoxy-2-methyl-pyridin- 3-yl)-3-methyl-1,3-dihydro-imidazo[4, 5-c]quinolin-2-one | 400.9 | 4.50 |
| L | 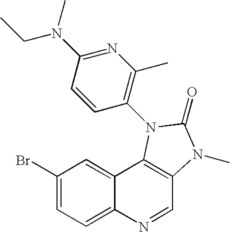 | 8-Bromo-1-[6-(ethyl-methyl-amino)-2- methyl-pyridin-3-yl]-3-methyl-1,3- dihydro-imidazo[4,5-c]quinolin-2-one | 426/428 | 4.34 (system B) |
| M | 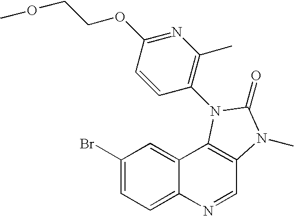 | 8-Bromo-1-[6-(2-methoxy-ethoxy)-2- methyl-pyridin-3-yl]-3-methyl-1,3- dihydro-imidazo[4,5-c]quinolin-2-one | 443/445 | 4.34 (system B) |
| N | 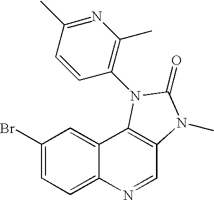 | 8-Bromo-1-(2,6-dimethyl-pyridin-3-yl)- 3-methyl-1,3-dihydro-imidazo[4,5-c] quinolin-2-one | 384.9 | 3.77 |
| O | 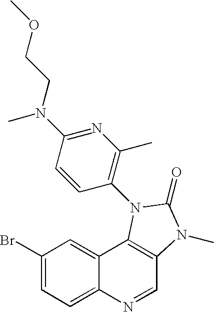 | 8-Bromo-1-{6-[(2-methoxy-ethyl)- methyl-amino]-2-methyl-pyridin-3-yl}- 3-methyl-1,3-dihydro-imidazo[4,5-c] quinolin-2-one | 458.0 | 3.97 |
| P | 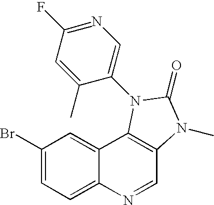 | 8-Bromo-1-(6-fluoro-4-methyl-pyridin- 3-yl)-3-methyl-1,3-dihydro-imidazo[4,5-c] quinolin-2-one | 389.0 | 4.33 |
| Q | 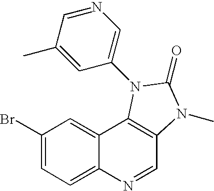 | 8-Bromo-3-methyl-1-(5-methyl-pyridin- 3-yl)-1,3-dihydro-imidazo[4,5-c]quinolin- 2-one | 371.0 | 4.03 |
| R | 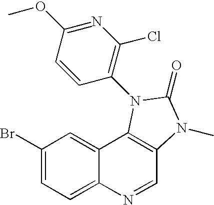 | 8-Bromo-1-(2-chloro-6-methoxy- pyridin-3-yl)-3-methyl-1,3-dihydro- imidazo[4,5-c]quinolin-2-one | 420.9 | 4.68 |
| S | 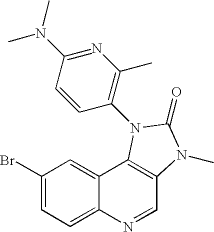 | 8-Bromo-1-(6-dimethylamino-2- methyl-pyridin-3-yl)-3-methyl-1,3- dihydro-imidazo[4,5-c]quinolin-2-one | 414.0 | 3.767 |
| T | 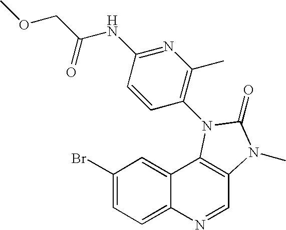 | N-[5-(8-Bromo-3-methyl-2-oxo-2,3- dihydro-imidazo[4,5-c]quinolin-1-yl)-6- methyl-pyridin-2-yl]-2-methoxy- acetamide | 456.0 | 4.217 |
| U | 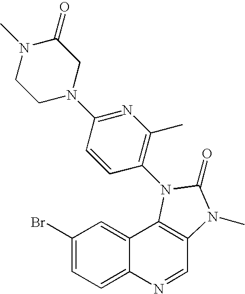 | 8-Bromo-3-methyl-1-[2-methyl-6-(4- methyl-3-oxo-piperazin-1-yl)-pyridin-3- yl]-1,3-dihydro-imidazo[4,5-c]quinolin- 2-one | 483.1 | 4.604 (method B) |
| V | 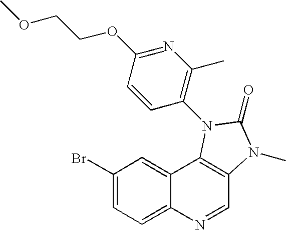 | 8-Bromo-1-[6-(2-methoxy-ethoxy)-2- methyl-pyridin-3-yl]-3-methyl-1,3- dihydro-imidazo[4,5-c]quinolin-2-one | 445.1 | 5.280 (method B) |
| W | 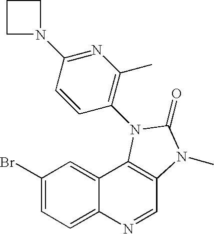 | 1-(6-Azetidin-1-yl-2-methyl-pyridin-3- yl)-8-bromo-3-methyl-1,3-dihydro- imidazo[4,5-c]quinolin-2-one | 424.0 | 3.875 |
| X | 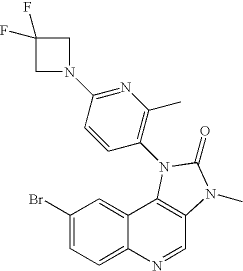 | 8-Bromo-1-[6-(3,3-difluoro-azetidin-1- yl)-2-methyl-pyridin-3-yl]-3-methyl-1,3- dihydro-imidazo[4,5-c]quinolin-2-one | 462.1 | 5.312 (method B) |
| Y | 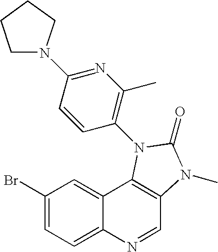 | 8-Bromo-3-methyl-1-(2-methyl-6- pyrrolidin-1-yl-pyridin-3-yl)-1,3- dihydro-imidazo[4,5-c]quinolin-2-one | 440.1 | 3.800 |
| Z1 | 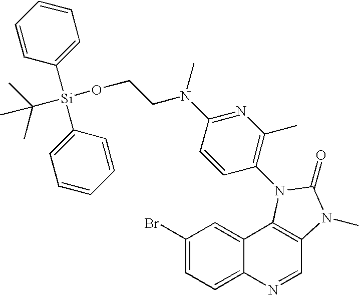 | 8-Bromo-1-(6-{[2-(tert-butyl-diphenyl- silanyloxy)-ethyl]-methyl-amino}-2- methyl-pyridin-3-yl)-3-methyl-1,3- dihydro-imidazo[4,5-c]quinolin-2-one | 682 | 7.675 (method B) |
| Z2 | 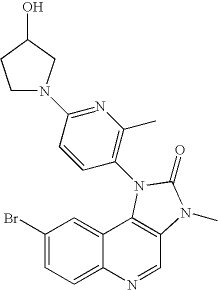 | 8-Bromo-1-[6-(3-hydroxy-pyrrolidin-1- yl)-2-methyl-pyridin-3-yl]-3-methyl-1,3- dihydro-imidazo[4,5-c]quinolin-2-one | 456.1 | 3.583 |
| Z3 | 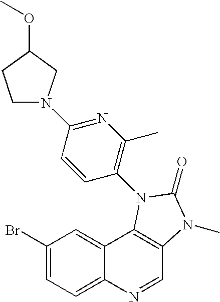 | 8-Bromo-1-[6-(3-methoxy-pyrrolidin-1- yl)-2-methyl-pyridin-3-yl]-3-methyl-1,3- dihydro-imidazo[4,5-c]quinolin-2-one (same synthesis as Intermediate Z2, second product in alkylation of imidazo-N) | 468.1 | 3.833 |
| Z4 | 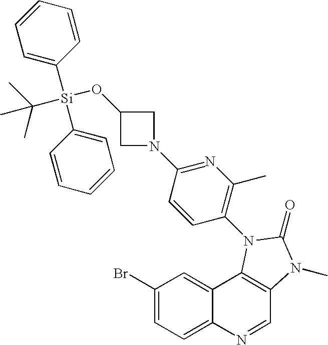 | 8-Bromo-1-{6-[3-(tert-butyl-diphenyl- silanyloxy)-azetidin-1-yl]-2-methyl- pyridin-3-yl}-3-methyl-1,3-dihydro- imidazo[4,5-c]quinolin-2-one | 680.2 | 7.300 (method B) |
| Z5 | 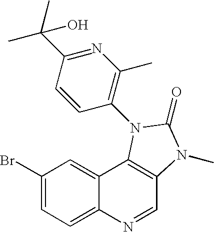 | 8-Bromo-1-[6-(1-hydroxy-1-methyl- ethyl)-2-methyl-pyridin-3-yl]-3-methyl- 1,3-dihydro-imidazo[4,5-c]quinolin-2-one | 429.2 | 4.802 (method B) |
| Z6 | 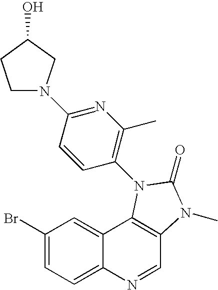 | 8-Bromo-1-[6-((S)-3-hydroxy- pyrrolidin-1-yl)-2-methyl-pyridin-3-yl]- 3-methyl-1,3-dihydro-imidazo[4,5-c] quinolin-2-one | 456.0 | 3.583 |
| Z7 | 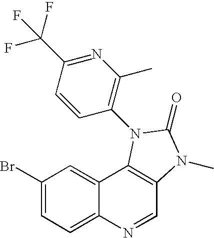 | 8-Bromo-3-methyl-1-(2-methyl-6- trifluoromethyl-pyridin-3-yl)-1,3- dihydro-imidazo[4,5-c]quinolin-2-one | 439.1 | 5.799 (method B) |
| Z8 | 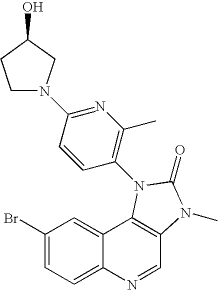 | 8-Bromo-1-[6-((R)-3-hydroxy- pyrrolidin-1-yl)-2-methyl-pyridin-3-yl]- 3-methyl-1,3-dihydro-imidazo[4,5-c] quinolin-2-one | 456.0 | 3.575 |
| Z9 | 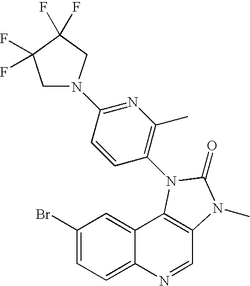 | 8-Bromo-3-methyl-1-[2-methyl-6- (3,3,4,4-tetrafluoro-pyrrolidin-1-yl)- pyridin-3-yl]-1,3-dihydro-imidazo[4,5-c] quinolin-2-one | 512.0 | 4.95 |

### Boronic Acid and Esters

2-Methoxy-5-pyridine boronic acid (Aldrich, Buchs, Switzerland)

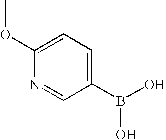

5-(4,4,5,5-Tetramethyl-1,3,2-dioxoborolan-2-yl)-1H-pyrrolo[2,3-b]pyridine (ABCR, Karlsruhe, Germany)

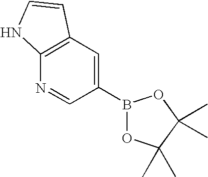

5-(4,4,5,5-Tetramethyl-[1,3,2]dioxaborolan-2-yl)-3-trifluoro-methyl-pyridin-2-ylamine (see example 1.1; stage 1.3.1 and 1.3.2)

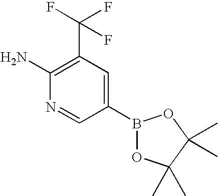

Methyl-[5-(4,4,5,5-tetramethyl-[1,3,2]dioxaborolan-2-yl)-pyrimidin-2-yl]-amine

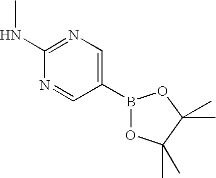

Methyl-[5-(4,4,5,5-tetramethyl-[1,3,2]dioxaborolan-2-yl)-pyrimidin-2-yl]-amine is prepared from 2-methylamino-5-bromopyrimidine and 4,4,5,5,-tetramethyl-2-(4,4,5,5,-tetramethyl-1,3,2-dioxaborolan-2-yl)-1,3,2-dioxaborolane as described in WO2006/79791 (ASTRA ZENECA) and WO2007/84786 (Novartis AG).
4-Aminocarbonylphenylboronic acid (Aldrich, Buchs, Switzerland)

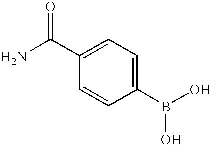

4-(N-Methylaminocarbonyl)phenylboronic acid (ABCR, Karlsruhe, Germany)

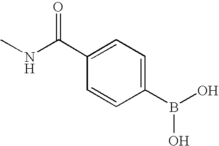

6-Ethoxypyridine-3-boronic acid (ABCR, Karlsruhe, Germany)

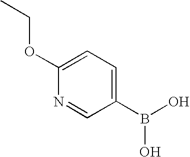

6-(Hydroxymethyl)pyridine-3-boronic acid (Aalen Chemical, Nanjing, China)

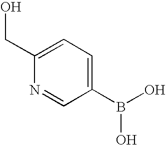

2-Amino-5-(4,4,5,5-tetramethyl-1,3,2-dioxoborolan-2-yl)pyridine (Aldrich, Buchs, Switzerland)

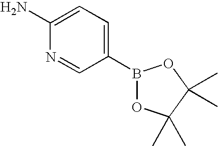

3,4-Dimethoxyphenylboronic acid (Aldrich, Buchs, Switzerland)

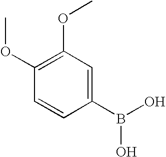

2-(3-N,N-Dimethylaminopropoxy)pyridin-5-boronic acid pinacol ester (ABCR, Karlsruhe, Germany)

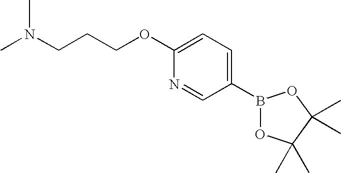

3-Quinolineboronic acid (Aldrich, Buchs, Switzerland)

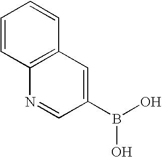

6-(Dimethylamino)pyridine-3-boronic acid (Fluorochem Ltd, Derbyshire, United Kingdom)

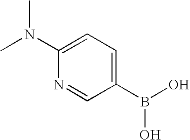

2-Ethoxy-pyrimidine-5-boronic acid (Apollo Scientific Ltd., Stockport, UK)

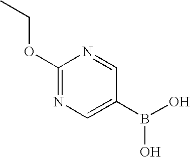

6-(Boc-methylamino)pyridine-3-boronic acid pinacol ester (Alfa Aesar, Ward Hill, USA)

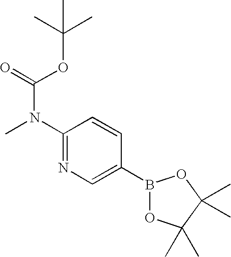

1-Methyl-5-(4,4,5,5-tetramethyl-[1,3,2]dioxaborolan-2-yl)-1H-pyrrolo[2,3-b]pyridine (see example 1.7; stage 1.7.1)

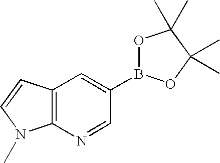

Dimethyl-[5-(4,4,5,5-tetramethyl-[1,3,2]dioxaborolan-2-yl)-pyrimidin-2-yl]-amine (Aldrich, Buchs, Switzerland)

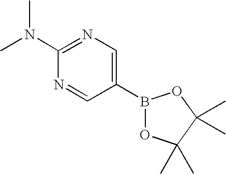

3-Ethoxy-5-(4,4,5,5-tetramethyl-[1,3,2]dioxaborolan-2-yl)-pyridine (synthesis see example 2.16; stage 2.16.1)

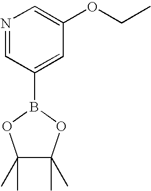

3-(2-Methoxy-ethoxy)-5-(4,4,5,5-tetramethyl-[1,3,2]dioxaborolan-2-yl)-pyridine (synthesis see example 2.17; stage 2.17.1)

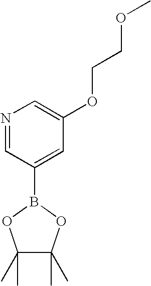

2-Ethoxy-pyridine-4-boronic acid (Combi Blocks, San Diego, Calif., USA)

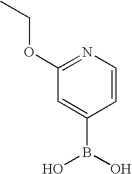

3-Isopropoxy-2-methyl-5-(4,4,5,5-tetramethyl-[1,3,2]dioxaborolan-2-yl)-pyridine (synthesis see example 2.19.1)

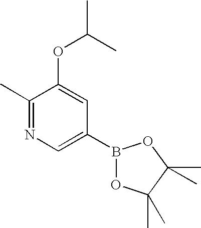

3-Ethoxy-2-methyl-5-(4,4,5,5-tetramethyl-[1,3,2]dioxaborolan-2-yl)-pyridine (synthesis see example 2.20.1)

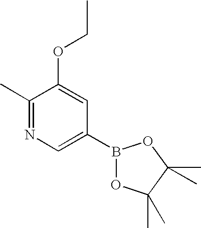

Ethyl-[2-methyl-5-(4,4,5,5-tetramethyl-[1,3,2]dioxaborolan-2-yl)-pyridin-3-yl]-amine (synthesis see example 2.21.1)

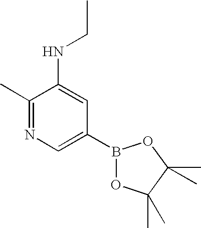

3-(2-Methoxy-ethoxy)-5-(4,4,5,5-tetramethyl-[1,3,2]dioxaborolan-2-yl)-pyridine (synthesis see example 8.8.1)

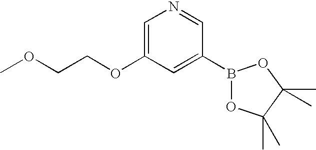

3-(2-Methoxy-ethoxy)-2-methyl-5-(4,4,5,5-tetramethyl-[1,3,2]dioxaborolan-2-yl)-pyridine (synthesis see example 8.9.1)

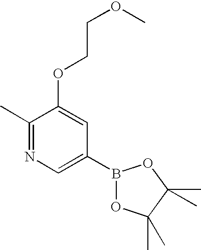

Imidazo[1,2-A]pyridine-6-boronic acid (Combi Blocks, San Diego, Calif., USA)

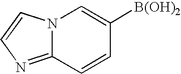

Isopropyl-[5-(4,4,5,5-tetramethyl-[1,3,2]dioxaborolan-2-yl)-pyridin-3-yl]-amine (synthesis see example 2.24)

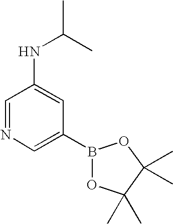

5-(4,4,5,5-Tetramethyl-[1,3,2]dioxaborolan-2-yl)-pyridin-3-ylamine (Apollo Scientific Ltd., Bredbury, UK)

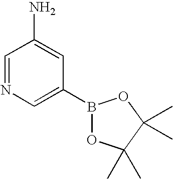

Methyl-[5-(4,4,5,5-tetramethyl-[1,3,2]dioxaborolan-2-yl)-pyridin-3-yl]-amine (synthesis see example 2.26)

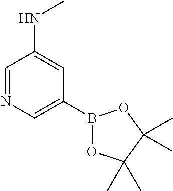

3-Ethoxy-2-methoxymethyl-5-(4,4,5,5-tetramethyl-[1,3,2]dioxaborolan-2-yl)-pyridine (synthesis see example 2.28)

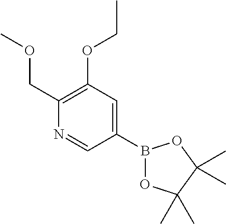

3-Azetidin-1-yl-5-(4,4,5,5-tetramethyl-[1,3,2]dioxaborolan-2-yl)-pyridine (synthesis see example 2.29)

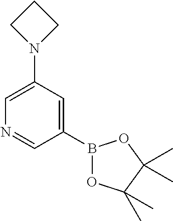

3-Methoxy-2-methoxymethyl-5-(4,4,5,5-tetramethyl-[1,3,2]dioxaborolan-2-yl)-pyridine (synthesis see example 2.30)

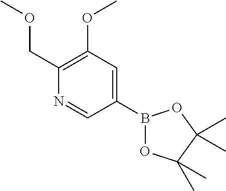

Acetic acid 3-(tert-butoxycarbonyl-ethyl-amino)-5-(4,4,5,5-tetramethyl-[1,3,2]dioxaborolan-2-yl)-pyridin-2-ylmethyl ester (synthesis see example 2.31)

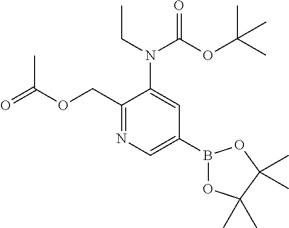

Methyl-[2-methyl-5-(4,4,5,5-tetramethyl-[1,3,2]dioxaborolan-2-yl)-pyridin-3-yl]-amine (synthesis see example 2.32)

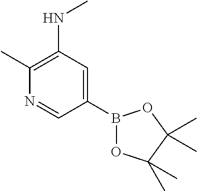

Stage 7.8.1. FS-2384=FS-2330 2-[5-(4,4,5,5-Tetramethyl-[1,3,2]dioxaborolan-2-yl)-pyridin-3-yl]-propan-2-ol (synthesis see example 7.8)

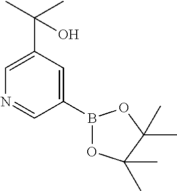

Acetic acid 3-methoxy-5-(4,4,5,5-tetramethyl-[1,3,2]dioxaborolan-2-yl)-pyridin-2-ylmethyl ester (synthesis see example 7.10.1)

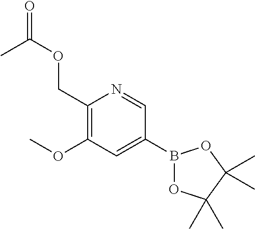

[3-Fluoro-5-(4,4,5,5-tetramethyl-[1,3,2]dioxaborolan-2-yl)-pyridin-2-yl]-methyl-amine (synthesis see example 7.12.1)

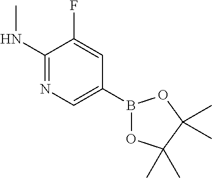

Methyl-[5-(4,4,5,5-tetramethyl-[1,3,2]dioxaborolan-2-yl)-3-trifluoromethyl-pyridin-2-yl]-amine (synthesis see example 7.13.1)

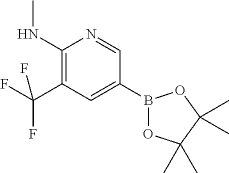

2-Amino-N-methyl-5-(4,4,5,5-tetramethyl-[1,3,2]dioxaborolan-2-yl)-nicotinamide (synthesis see example 7.14.1)

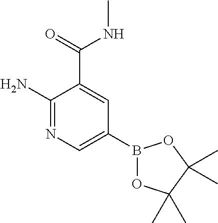

3-Ethoxymethyl-5-(4,4,5,5-tetramethyl-[1,3,2]dioxaborolan-2-yl)-pyridin-2-ylamine (synthesis see example 7.15.1)

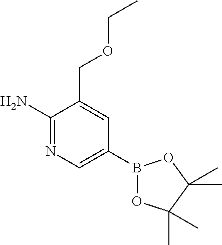

Acetic acid 2-diacetylamino-5-(4,4,5,5-tetramethyl-[1,3,2]dioxaborolan-2-yl)-pyridin-3-ylmethyl ester (synthesis see example 7.17.1)

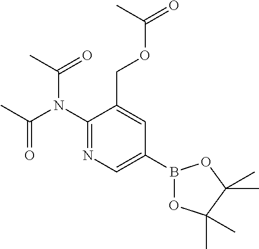

Ethyl-[5-(4,4,5,5-tetramethyl-[1,3,2]dioxaborolan-2-yl)-pyrimidin-2-yl]-amine (synthesis see example 7.18.1)

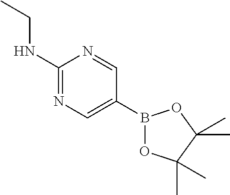

[4-Methoxy-5-(4,4,5,5-tetramethyl-[1,3,2]dioxaborolan-2-yl)-pyrimidin-2-yl]-methyl-amine (synthesis see example 7.19.1)

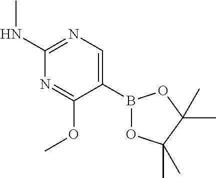

3-Isopropoxy-5-(4,4,5,5-tetramethyl-[1,3,2]dioxaborolan-2-yl)-pyridine (synthesis see example 20.5)

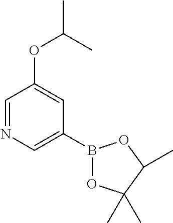

3-Isopropoxy-2-methoxymethyl-5-(4,4,5,5-tetramethyl-[1,3,2]dioxaborolan-2-yl)-pyridine (synthesis see example 23.2)

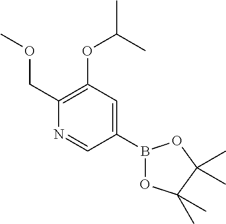

1-[5-(4,4,5,5-Tetramethyl-[1,3,2]dioxaborolan-2-yl)-pyridin-3-yl]-cyclo-butanecarbonitrile (synthesis see example 31.8)

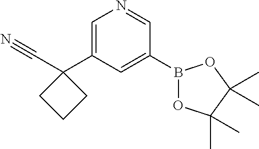

[3-Chloro-5-(4,4,5,5-tetramethyl-[1,3,2]dioxaborolan-2-yl)-pyridin-2-yl]-methyl-amine (synthesis see example 32.4)

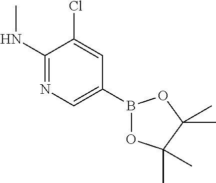

Ethyl-[4-methoxy-5-(4,4,5,5-tetramethyl-[1,3,2]dioxaborolan-2-yl)-pyrimidin-2-yl]-amine (synthesis see example 32.9)

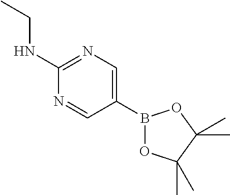

Acetic acid 3-(tert-butoxycarbonyl-methyl-amino)-5-(4,4,5,5-tetramethyl-[1,3,2]dioxaborolan-2-yl)-pyridin-2-ylmethyl ester (synthesis see example 33.2)

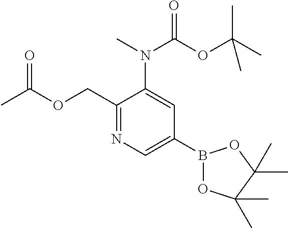

Ethyl-[5-(4,4,5,5-tetramethyl-[1,3,2]dioxaborolan-2-yl)-3-trifluoromethyl-pyridin-2-yl]-amine (synthesis see example 34.2)

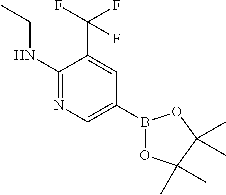

2-Methyl-2-[5-(4,4,5,5-tetramethyl-[1,3,2]dioxaborolan-2-yl)-pyridin-3-yl]-propionitrile (synthesis see example 34.5)

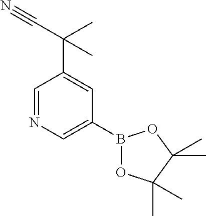

### Example 1.1

### 1-(2-Methoxy-pyridin-3-yl)-8-(6-methoxy-pyridin-3-yl)-3-methyl-1,3-dihydro-imidazo[4,5-c]quinolin-2-one

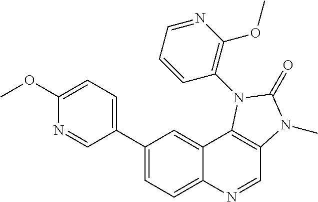

A mixture of 8-bromo-1-(2-methoxy-pyridin-3-yl)-3-methyl-1,3-dihydro-imidazo[4,5-c]quinolin-2-one (Intermediate A, 53.9 mg, 0.140 mmol) in DMF anhydrous (5 ml), 2-methoxy-5-pyridine boronic acid (Aldrich, Buchs, Switzerland, 32.1 mg, 0.210 mmol), PdCl₂(PPh₃)₂ (4.91 mg, 0.007 mmol) and K₂CO₃ 1M (0.350 ml, 0.350 mmol) were stirred at 100° C. for 15 min. Then the RM was cooled to rt and extracted with EtOAc/H₂O. The organic layers were dried over Na₂SO₄ and evaporated. The residue was purified with silica gel (DCM/MeOH 4%). Fractions containing product were evaporated together to liophylised under high vacuum to give the title compound as a white solid. (HPLC: t_{R} 4.21 min (Method A); M+H=414.1 MS−ES; ¹H-NMR (d₆-DMSO, 600 MHz) 9.00 (s, 1H), 8.55-8.45 (m, 1H), 8.23-8.01 (m, 3H), 7.95-7.85 (m, 1H), 7.75-7.65 (m, 1H), 7.40-7.30 (m, 1H), 7.15-7.05 (m, 1H), 6.95-6.85 (m, 1H), 3.87 (s, 3H), 3.76 (s, 3H), 3.61 (s, 3H)).

### Example 1.2

### 1-(2-Methoxy-pyridin-3-yl)-3-methyl-8-(1H-pyrrolo[2,3-b]pyridin-5-yl)-1,3-dihydro-imidazo[4,5-c]quinolin-2-one

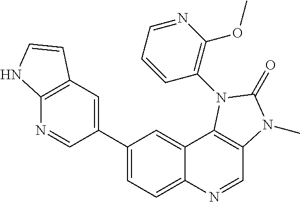

The title compound was synthesized in a similar manner as described for Example 1.1 using 5-(4,4,5,5-tetramethyl-1,3,2-dioxoborolan-2-yl)-1H-pyrrolo[2,3-b]pyridine and intermediate A.

(HPLC: t_{R} 3.85 min (Method A); M+H=423.1 MS−ES; ¹H-NMR (d₆-DMSO, 600 MHz) 11.75 (s, 1H), 9.00 (s, 1H), 8.55-8.45 (m, 1H), 8.22-8.05 (m, 3H), 8.05-7.89 (m, 2H), 7.60-7.45 (m, 1H), 7.44-7.30 (m, 1H), 7.25-7.15 (m, 1H), 6.55-6.45 (m, 1H), 3.78 (s, 3H), 3.62 (s, 3H)).

### Example 1.3

### 8-(6-Amino-5-trifluoromethyl-pyridin-3-yl)-1-(2-methoxy-pyridin-3-yl)-3-methyl-1,3-dihydro-imidazo[4,5-c]quinolin-2-one

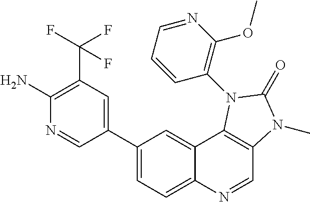

The title compound was synthesized in a similar manner as described for Example 1.1 using Stage 1.3.1 and intermediate A.

(HPLC: t_{R} 4.04 min (Method A); M+H=467.1 MS−ES; ¹H-NMR (d₆-DMSO, 600 MHz) 8.98 (s, 1H), 8.55-8.40 (m, 1H), 8.35-8.25 (m, 1H), 8.10-7.90 (m, 3H), 7.70-7.55 (m, 1H), 7.40-7.28 (m, 1H), 7.10-7.00 (m, 1H), 6.75 (s, br, 2H), 3.78 (s, 3H), 3.62 (s, 3H)).

### Stage 1.3.1 5-(4,4,5,5-tetramethyl-[1,3,2]dioxaborolan-2-yl)-3-trifluoro-methyl-pyridin-2-ylamine

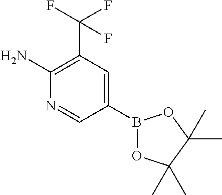

8.04 g (31.7 mmol) of 5-bromo-3-trifluoromethyl-pyridin-2-ylamine (preparation see Stage 1.3.2), 10.5 g (41.2 mmol) of 4,4,5,5,4′,4′,5′,5′-octamethyl-[2,2′]bi[[1,3,2]dioxaborolanyl] (Aldrich), 9.62 g (95.1 mmol) of KOAc in 100 ml dioxane are degassed with argon for 15 min. Then 776 mg (0.951 mmol) of bis(diphenylphosphino)ferrocene dichloropalla-dium(II)di-chloromethane (ABCR) are added and the mixture is degassed for 15 more minutes. The reaction mixture is heated at 115° C. for 8 h. After that time, the reaction mixture is filtered and the solvent evaporated. The residue is purified by simple filtration on silicagel (solvent system: t-butyl-methyl ether-EtOAc-NEt3=50:50:0.1) to yield the title compound as almost colorless solid. ES-MS: (M+1)=289; Tlc: R_{f}=0.77 in t-butyl-methyl ether-EtOAc 1:1.

### Stage 1.3.2 5-Bromo-3-trifluoromethyl-pyridin-2-ylamine.

To a solution of 5.37 g (32.8 mmol) of 3-trifluoromethyl-pyridin-2-ylamine (Fluorochem Ltd., Derbyshire, United Kingdom) in 100 ml of dry CH₃CN, 6.45 g of NBS are added in 4 equal portions over a period of 1 h at 0-5° C. under argon. The cooling bath is removed and stirring is continued for 3 h. The solvent is evaporated under vacuum, the residue is dissolved in EtOAc and washed with water and brine. The organic phase is dried over Na₂SO₄ and evaporated. The title compound is a red-dish-yellow oil which is used after drying in the dark for 5 h at RT and under high vacuum in the next step without further purification. ES-MS (M+1): =239; 241; HPLC: t_{R}=5.501 min.

### Example 1.4

### 1-(2-Methoxy-pyridin-3-yl)-3-methyl-8-(6-methylamino-pyridin-3-yl)-1,3-dihydro-imidazo[4,5-c]quinolin-2-one

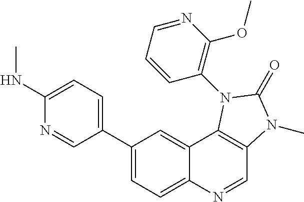

The title compound was synthesized in a similar manner as described for Example 1.1 using 6-(boc-methylamino)pyridine-3-boronic acid pinacol ester and intermediate A. Boc was removed in situ (0.5 mL for 5 min at RT) before purification by dissolving the crude product in TFA (0.5 ml) during 5 min.

(HPLC: t_{R} 3.54 min (Method A); M+H=413.1 MS−ES; ¹H-NMR (d₆-DMSO, 600 MHz) 8.94 (s, 1H), 8.57-8.44 (m, 1H), 8.18-8.10 (m, 1H), 8.08-8.00 (m, 1H), 8.00-7.92 (m, 1H), 7.86-7.78 (m, 1H), 7.44-7.36 (m, 1H), 7.36-7.32 (m, 1H), 7.06-6.98 (m, 1H), 6.9-6.8 (m, br, 1H), 6.54-6.43 (m, 1H), 3.75 (s, 3H), 3.59 (s, 3H), 2.78 (d, 3H).

### Example 1.5

### 8-(6-Ethoxy-pyridin-3-yl)-1-(2-methoxy-pyridin-3-yl)-3-methyl-1,3-dihydro-imidazo[4,5-c]quinolin-2-one

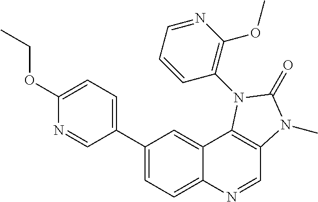

The title compound was synthesized in a similar manner as described for Example 1.1 using 6-ethoxypyridine-3-boronic acid and intermediate A.

(HPLC: t_{R} 4.45 min (Method A); M+H=427.8 MS−ES; ¹H-NMR (d₆-DMSO, 600 MHz) 8.99 (s, 1H), 8.55-8.45 (m, 1H), 8.18-8.05 (m, 3H), 7.91-7.82 (m, 1H), 7.70-7.62 (m, 1H), 7.37-7.30 (m, 1H), 7.12-7.04 (m, 1H), 6.90-6.79 (m, 1H), 4.31 (q, 2H), 3.75 (s, 3H), 3.60 (s, 3H), 1.31 (t, 3H)).

### Example 1.6

### 4-[1-(2-Methoxy-pyridin-3-yl)-3-methyl-2-oxo-2,3-dihydro-1H-imidazo[4,5-c]quinolin-8-yl]-benzamide

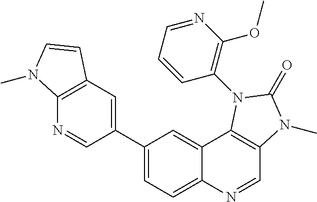

The title compound was synthesized in a similar manner as described for Example 1.1 using 4-aminocarbonylphenylboronic acid and intermediate A.

(HPLC: t_{R} 3.84 min (Method A); M+H=426.0 MS−ES; ¹H-NMR (d₆-DMSO, 600 MHz) 9.01 (s, 1H), 8.55-8.45 (m, 1H), 8.18-8.14 (m, 1H), 8.14-8.09 (m, 1H), 8.04-8.00 (m, 1H), 7.96-7.92 (m, 1H), 7.92-7.88 (m, 2H), 7.45-7.39 (m, 3H), 7.38-7.32 (m, 1H), 7.24-7.19 (m, 1H), 3.75 (s, 3H), 3.60 (s, 3H)).

### Example 1.7

### 1-(2-Methoxy-pyridin-3-yl)-3-methyl-8-(1-methyl-1H-pyrrolo[2,3-b]pyridin-5-yl)-1,3-dihydro-imidazo[4,5-c]quinolin-2-one

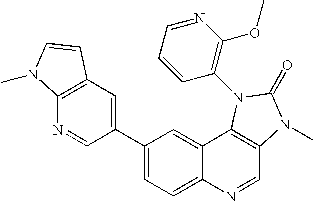

The title compound was synthesized in a similar manner as described for Example 1.1 using 1-methyl-5-(4,4,5,5-tetramethyl-[1,3,2]dioxaborolan-2-yl)-1H-pyrrolo[2,3-b]pyridine and intermediate A.

(HPLC: t_{R} 4.18 min (Method A); M+H=437.1 MS−ES; ¹H-NMR (d₆-DMSO, 600 MHz) 9.02 (s, 1H), 8.56-8.50 (m, 1H), 8.22-8.17 (m, 2H), 8.16-8.11 (m, 1H), 8.03-7.99 (m, 1H), 7.99-7.95 (m, 1H), 7.61-7.56 (m, 1H), 7.40-7.34 (m, 1H), 7.24-7.18 (m, 1H), 6.54-6.48 (m, 1H), 3.85 (s, 3H), 3.78 (s, 3H), 3.60 (s, 3H)).

### Stage 1.7.1 1-Methyl-5-(4,4,5,5-tetramethyl-[1,3,2]dioxaborolan-2-yl)-1H-pyrrolo[2,3-b]pyridine

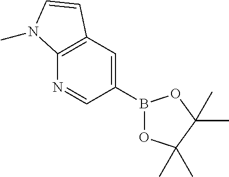

To a mixture of 5-(4,4,5,5-tetramethyl-[1,3,2]dioxaborolan-2-yl)-1H-pyrrolo[2,3-b]pyridine (ABCR, Karlsruhe, Germany, 366 mg, 1.50 mmol) in DMF (5 ml) cooled to 0° C. was added NaH 55% and stirred for 30 min. Then icebath was removed and iodomethane (0.140 ml, 2.25 mmol) was added and the RM was stirred at rt for 1 h. After that, EtOAc and H₂O were added and the mixture was extracted. The organic layer was dried over Na₂SO₄ and evaporated to give the title compound as a beige solid.

(M+H=259.1 MS−ES; ¹H-NMR (d₆-DMSO, 600 MHz) 8.46 (s, 1H), 8.20 (s, 1H), 7.51 (m, 1H), 6.48 (m, 1H), 3.80 (s, 3H), 1.29 (s, 12H)).

### Example 1.8

### 8-(6-Fluoro-pyridin-3-yl)-1-(2-methoxy-pyridin-3-yl)-3-methyl-1,3-dihydro-imidazo[4,5-c]quinolin-2-one

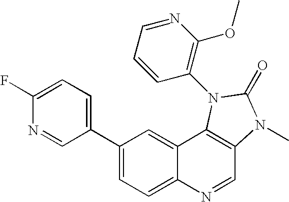

The title compound was synthesized in a similar manner as described for Example 1.1 using 6-fluoropyridine-3-boronic acid and intermediate A.

(HPLC: t_{R} 4.16 min (Method A); M+H=402.1 MS−ES; ¹H-NMR (d₆-DMSO, 600 MHz) 9.05 (s, 1H), 8.53-8.48 (m, 1H), 8.22-8.13 (m, 3H), 8.03-7.97 (m, 1H), 7.97-7.92 (m, 1H), 7.40-7.34 (m, 1H), 7.34-7.27 (m, 1H), 7.19-7.15 (m, 1H), 3.77 (s, 3H), 3.63 (s, 3H)).

### Example 1.9

### 8-(2-Dimethylamino-pyrimidin-5-yl)-1-(2-methoxy-pyridin-3-yl)-3-methyl-1,3-dihydro-imidazo[4,5-c]quinolin-2-one

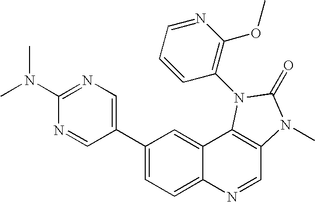

The title compound was synthesized in a similar manner as described for Example 1.1 using dimethyl-[5-(4,4,5,5-tetramethyl-[1,3,2]dioxaborolan-2-yl)-pyrimidin-2-yl]-amine and intermediate A.

(HPLC: t_{R} 4.06 min (Method A); M+H=428.1 MS−ES; ¹H-NMR (d₆-DMSO, 600 MHz) 8.99 (s, 1H), 8.53-8.52 (m, 1H), 8.36 (s, 2H), 8.16-8.15 (m, 1H), 8.10-8.08 (m, 1H), 7.89-7.87 (m, 1H), 7.38-7.36 (m, 1H), 7.05 (s, 1H), 3.77 (s, 3H), 3.61 (s, 3H), 3.15 (s, 6H).

### Example 2.1

### 8-(6-Ethoxy-pyridin-3-yl)-3-methyl-1-(2-methyl-pyridin-3-yl)-1,3-dihydro-imidazo[4,5-c]quinolin-2-one

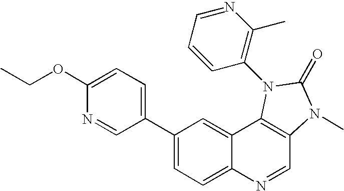

The title compound was synthesized in a similar manner as described for Example 1.1 using 6-ethoxypyridin-3-yl boronic acid and intermediate B.

(HPLC: t_{R} 4.08 min (Method A); M+H=412.1 MS−ES; ¹H-NMR (d₆-DMSO, 600 MHz) 9.04 (s, 1H), 8.80-8.70 (m, 1H), 8.21-8.00 (m, 3H), 7.95-7.85 (m, 1H), 7.75-7.55 (m, 2H), 7.00-6.87 (m, 1H), 6.90-6.80 (m, 1H), 4.31 (q, 2H), 3.63 (s, 3H), 2.30 (s, 3H), 1.31 (t, 3H)).

### Example 2.2

### 3-Methyl-8-(2-methylamino-pyrimidin-5-yl)-1-(2-methyl-pyridin-3-yl)-1,3-dihydro-imidazo[4,5-c]quinolin-2-one

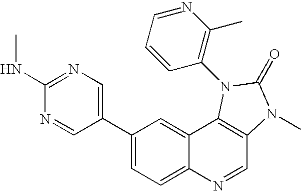

The title compound was synthesized in a similar manner as described for Example 1.1 using 2-methylaminopyrimidin-5-boronic acid PE and intermediate B.

(HPLC: t_{R} 3.58 min (Method A); M+H=398.2 MS−ES; ¹H-NMR (d₆-DMSO, 600 MHz) 9.00 (s, 1H), 8.85-8.70 (m, 1H), 8.35-8.00 (m, 4H), 7.90-7.75 (m, 1H), 7.65-7.50 (m, 1H), 7.40-7.30 (m, 1H), 6.90-6.80 (m, 1H), 3.61 (s, 3H), 2.80 (s, 3H), 2.29 (s, 3H)).

### Example 2.3

### 4-[3-Methyl-1-(2-methyl-pyridin-3-yl)-2-oxo-2,3-dihydro-1H-imidazo[4,5-c]quinolin-8-yl]-benzamide

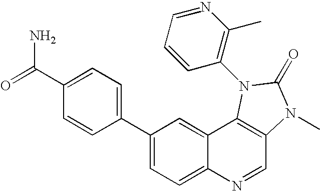

The title compound was synthesized in a similar manner as described for Example 1.1 using 4-aminocarbonylphenylboronic acid and intermediate B.

(HPLC: t_{R} 3.63 min (Method A); M+H=410.1 MS−ES; ¹H-NMR (d₆-DMSO, 600 MHz) 9.05 (s, 1H), 8.81-8.74 (m, 1H), 8.17-8.11 (m, 1H), 8.11-8.06 (m, 1H), 8.04-7.99 (m, 1H), 7.97-7.92 (m, 1H), 7.91-7.86 (m, 2H), 7.63-7.56 (m, 1H), 7.44-7.39 (m, 1H), 7.39-7.35 (m, 2H), 7.07-6.98 (m, 1H), 3.63 (s, 3H), 2.29 (s, 3H)).

### Example 2.4

### N-Methyl-4-[3-methyl-1-(2-methyl-pyridin-3-yl)-2-oxo-2,3-dihydro-1H-imidazo[4,5-c]quinolin-8-yl]-benzamide

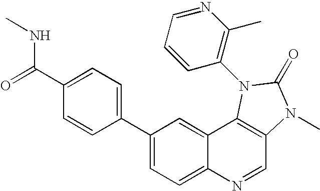

The title compound was synthesized in a similar manner as described for Example 1.1 using 4-(N-methylaminocarbonyl)phenylboronic acid and intermediate B.

(HPLC: t_{R} 3.73 min (Method A); M+H=424.1 MS−ES; ¹H-NMR (d₆-DMSO, 600 MHz) 9.05 (s, 1H), 8.82-8.71 (m, 1H), 8.53-8.46 (m, 1H), 8.16-8.11 (m, 1H), 8.11-8.06 (m, 1H), 7.96-7.91 (m, 1H), 7.86-7.81 (m, 2H), 7.62-7.57 (m, 1H), 7.41-7.35 (m, 2H), 7.05-7.00 (m, 1H), 3.62 (s, 3H), 2.77 (d, 3H), 2.29 (s, 3H)).

### Example 2.5

### 8-(2-Ethoxy-pyrimidin-5-yl)-3-methyl-1-(2-methyl-pyridin-3-yl)-1,3-dihydro-imidazo[4,5-c]quinolin-2-one

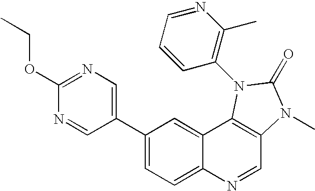

The title compound was synthesized in a similar manner as described for Example 1.1 using 6-ethoxypyrimidine-3-boronic acid and intermediate B.

(HPLC: t_{R} 3.91 min (Method A); M+H=413.1 MS−ES; ¹H-NMR (d₆-DMSO, 600 MHz) 9.06 (s, 1H), 8.80-8.70 (m, 1H), 8.60-8.45 (m, 2H), 8.25-7.90 (m, 3H), 7.65-7.52 (m, 1H), 7.00-6.85 (m, 1H), 4.36 (q, 2H), 3.62 (s, 3H), 2.29 (s, 3H), 1.32 (t, 3H)).

### Example 2.6

### 8-(6-Hydroxymethyl-pyridin-3-yl)-3-methyl-1-(2-methyl-pyridin-3-yl)-1,3-dihydro-imidazo[4,5-c]quinolin-2-one

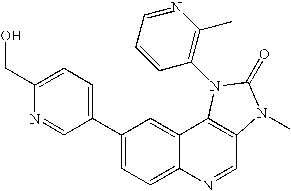

The title compound was synthesized in a similar manner as described for Example 1.1 using 6-(hydroxymethyl)pyridine-3-boronic acid and intermediate B.

(HPLC: t_{R} 3.30 min (Method A); M+H=398.1 MS−ES; ¹H-NMR (d₆-DMSO, 600 MHz): 9.07 (s, 1H), 8.84-8.70 (m, 1H), 8.45-8.30 (m, 1H), 8.25-7.90 (m, 3H), 7.85-7.75 (m, 1H), 7.70-7.44 (m, 2H), 7.05-6.95 (m, 1H), 5.55-5.40 (m, 1H), 4.60-4.50 (m, 2H), 3.64 (s, 3H), 2.30 (s, 3H).

### Example 2.7

### 8-(6-Amino-pyridin-3-yl)-3-methyl-1-(2-methyl-pyridin-3-yl)-1,3-dihydro-imidazo[4,5-c]quinolin-2-one

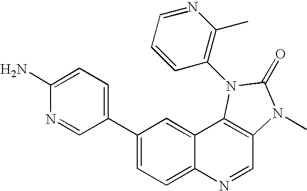

The title compound was synthesized in a similar manner as described for Example 1.1 using 2-amino-5-(4,4,5,5-tetramethyl-1,3,2-dioxoborolan-2-yl)pyridine and intermediate B.

(HPLC: t_{R} 3.35 min (Method A); M+H=383.1 MS−ES; ¹H-NMR (d₆-DMSO, 600 MHz) 8.98 (s, 1H), 8.83-8.72 (m, 1H), 8.15-7.99 (m, 2H), 7.88-7.76 (m, 2H), 7.65-7.56 (m, 1H), 7.42-7.32 (m, 1H), 6.93-6.80 (m, 1H), 6.53-6.38 (m, 1H), 6.19 (s, 2H), 3.62 (s, 3H), 2.29 (s, 3H)).

### Example 2.8

### 3-Methyl-1-(2-methyl-pyridin-3-yl)-8-(1H-pyrrolo[2,3-b]pyridin-5-yl)-1,3-dihydro-imidazo[4,5-c]quinolin-2-one

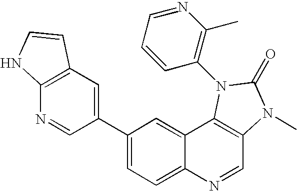

The title compound was synthesized in a similar manner as described for Example 1.1 using 5-(4,4,5,5-tetramethyl-1,3,2-dioxoborolan-2-yl)-1H-pyrrolo[2,3-b]pyridine and intermediate B.

(HPLC: t_{R} 3.69 min (Method A); M+H=407.1 MS−ES; ¹H-NMR (d₆-DMSO, 600 MHz) 11.70 (s, br, 1H), 9.03 (s, 1H), 8.82-8.72 (m, 1H), 8.56-8.45 (m, 1H), 8.25-8.18 (m, 1H), 8.18-8.06 (m, 2H), 8.00-7.93 (m, 1H), 7.93-7.89 (m, 1H), 7.56-7.43 (m, 1H), 7.04-6.96 (m, 1H), 6.53-6.40 (m, 1H), 3.63 (s, 3H), 2.30 (s, 3H).

### Example 2.9

### 8-(3,4-Dimethoxy-phenyl)-3-methyl-1-(2-methyl-pyridin-3-yl)-1,3-dihydro-imidazo[4,5-c]quinolin-2-one

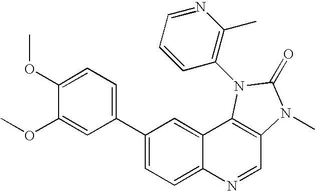

The title compound was synthesized in a similar manner as described for Example 1.1 using 3,4-dimethoxyphenylboronic acid and intermediate B.

(HPLC: t_{R} 3.99 min (Method A); M+H=427.1 MS−ES; ¹H-NMR (d₆-DMSO, 600 MHz) 9.01 (s, 1H), 8.82-8.71 (m, 1H), 8.14-8.02 (m, 2H), 7.96-7.88 (m, 1H), 7.63-7.54 (m, 1H), 7.04-6.91 (m, 3H), 6.79-6.71 (m, 1H), 3.77 (s, 6H), 3.63 (s, 3H), 2.31 (s, 3H)).

### Example 2.10

### 8-[6-(3-Dimethylamino-propoxy)-pyridin-3-yl]-3-methyl-1-(2-methyl-pyri din-3-yl)-1,3-dihydro-imidazo[4,5-c]quinolin-2-one

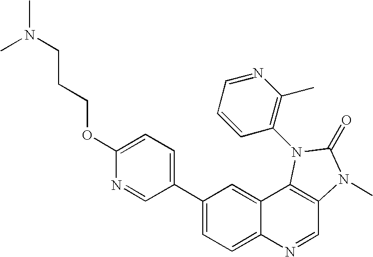

The title compound was synthesized in a similar manner as described for Example 1.1 using 2-(3-N,N-dimethylaminopropoxy)pyridin-5-boronic acid pinacol ester and intermediate B.

(HPLC: t_{R} 3.51 min (Method A); M+H=469.6 MS−ES; ¹H-NMR (d₆-DMSO, 600 MHz) 9.05 (s, 1H), 8.81-8.71 (m, 1H), 8.19-8.10 (m, 1H), 8.10-8.04 (m, 2H), 7.92-7.84 (m, 1H), 7.73-7.65 (m, 1H), 7.62-7.57 (m, 1H), 6.96-6.91 (m, 1H), 6.91-6.83 (m, 1H), 4.33 (t, 2H), 3.63 (s, 3H), 3.23-3.13 (m, 2H), 2.79 (s, 6H), 2.30 (s, 3H), 2.15-2.03 (m, 2H)).

### Example 2.11

### 8-(6-Amino-5-trifluoromethyl-pyridin-3-yl)-3-methyl-1-(2-methyl-pyridin-3-yl)-1,3-dihydro-imidazo[4,5-c]quinolin-2-one

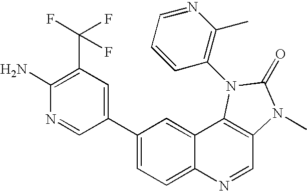

The title compound was synthesized in a similar manner as described for Example 1.1 using Stage 1.3.1 and intermediate B.

(HPLC: t_{R} 3.80 min (Method A); M+H=451.0 MS−ES; ¹H-NMR (d₆-DMSO, 600 MHz) 9.02 (s, 1H), 8.78-8.70 (m, 1H), 8.28-8.20 (m, 1H), 8.13-8.03 (m, 2H), 7.98-7.91 (m, 1H), 7.63-7.52 (m, 2H), 6.92-6.83 (m, 1H), 6.80-6.69 (m, 2H), 3.63 (s, 3H), 2.29 (s, 3H)).

### Example 2.12

### 8-(6-Methoxy-pyridin-3-yl)-3-methyl-1-(2-methyl-pyridin-3-yl)-1,3-dihydro-imidazo[4,5-c]quinolin-2-one

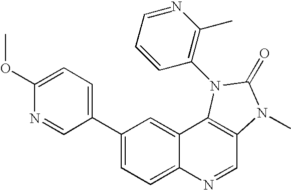

The title compound was synthesized in a similar manner as described for Example 1.1 using 2-methoxy-5-pyridine boronic acid and intermediate B.

(HPLC: t_{R} 3.90 min (Method A); M+H=398.2 MS−ES; ¹H-NMR (d₆-DMSO, 600 MHz) 9.04 (s, 1H), 8.84-8.72 (m, 1H), 8.19-8.10 (m, 2H), 8.10-8.04 (m, 1H), 7.93-7.85 (m, 1H), 7.70-7.63 (m, 1H), 7.63-7.56 (m, 1H), 7.01-6.91 (m, 1H), 6.91-6.82 (m, 1H), 3.87 (s, 3H), 3.63 (s, 3H), 2.30 (s, 3H)).

### Example 2.13

### 3-Methyl-8-(6-methylamino-pyridin-3-yl)-1-(2-methyl-pyridin-3-yl)-1,3-dihydro-imidazo[4,5-c]quinolin-2-one

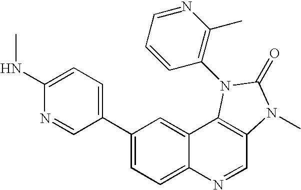

The title compound was synthesized in a similar manner as described for Example 1.1 using 6-(boc-methylamino)pyridine-3-boronic acid pinacol ester and intermediate B. Boc was removed in situ (0.5 mL for 5 min at RT) before purification by dissolving the crude product in TFA (0.5 ml) during 5 min.

(HPLC: t_{R} 3.46 min (Method A); M+H=396.9 MS−ES; ¹H-NMR (d₆-DMSO, 600 MHz) 8.97 (s, 1H), 8.82-8.74 (m, 1H), 8.09-8.00 (m, 2H), 7.98-7.91 (m, 1H), 7.84-7.76 (m, 1H), 7.63-7.56 (m, 1H), 7.36-7.29 (m, 1H), 6.88-6.81 (m, 1H), 6.77-6.70 (m, 1H), 6.48-6.39 (m, 1H), 3.60 (s, 3H), 2.77 (d, 3H), 2.28 (s, 3H)).

### Example 2.14

### 3-Methyl-1-(2-methyl-pyridin-3-yl)-8-(1-methyl-1H-pyrrolo[2,3-b]pyridin-5-yl)-1,3-dihydro-imidazo[4,5-c]quinolin-2-one

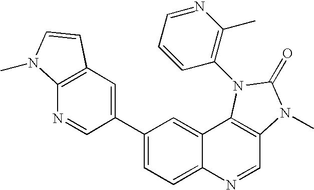

The title compound was synthesized in a similar manner as described for Example 1.1 using 1-methyl-5-(4,4,5,5-tetramethyl-[1,3,2]dioxaborolan-2-yl)-1H-pyrrolo[2,3-b]pyridine and intermediate B.

(HPLC: t_{R} 3.93 min (Method A); M+H=421.1 MS−ES; ¹H-NMR (d₆-DMSO, 600 MHz) 9.05 (s, 1H), 8.84-8.75 (m, 1H), 8.20-8.13 (m, 2H), 8.13-8.06 (m, 1H), 8.00-7.95 (m, 2H), 7.67-7.60 (m, 1H), 7.60-7.56 (m, 1H), 7.08-6.99 (m, 1H), 6.54-6.46 (m, 1H), 3.84 (s, 3H), 3.65 (s, 3H), 2.32 (s, 3H)).

### Example 2.15

### 8-(2-Dimethylamino-pyrimidin-5-yl)-3-methyl-1-(2-methyl-pyridin-3-yl)-1,3-dihydro-imidazo[4,5-c]quinolin-2-one

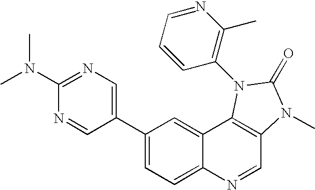

The title compound was synthesized in a similar manner as described for Example 1.1 using dimethyl-[5-(4,4,5,5-tetramethyl-[1,3,2]dioxaborolan-2-yl)-pyrimidin-2-yl]-amine and intermediate B.

(HPLC: t_{R} 3.78 min (Method A); M+H=412.1 MS−ES; ¹H-NMR (d₆-DMSO, 600 MHz) 9.03 (s, 1H), 8.83-8.78 (m, 1H), 8.36-8.30 (m, 2H), 8.14-8.08 (m, 1H), 8.08-8.03 (m, 1H), 7.91-7.84 (m, 1H), 7.65-7.59 (m, 1H), 6.91-6.86 (m, 1H), 3.63 (s, 3H), 3.15 (s, 6H), 2.31 (s, 3H)).

### Example 2.16

### 8-(5-Methoxymethyl-pyridin-3-yl)-3-methyl-1-(2-methyl-pyridin-3-yl)-1,3-dihydro-imidazo[4,5-c]quinolin-2-one

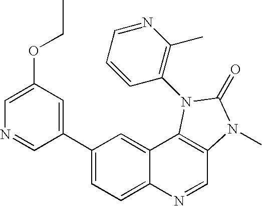

The title compound was synthesized in a similar manner as described for Example 1.1 using 3-ethoxy-5-(4,4,5,5-tetramethyl-[1,3,2]dioxaborolan-2-yl)-pyridine and intermediate B.

(HPLC: t_{R} 4.06 min (Method B); M+H=412.1 MS−ES.)

### Stage 2.16.1 3-Ethoxy-5-(4,4,5,5-tetramethyl-[1,3,2]dioxaborolan-2-yl)-pyridine

The title compound was synthesized in a similar manner as described for Stage 1.3.1 using 3-bromo-5-ethoxypyridine (SynChem, Des Plaines, USA) to give the title compound as a brown oil. (HPLC: t_{R} 2.45 min (Method C); M+H=250 MS−ES).

### Example 2.17

### 8-[5-(2-Methoxy-ethoxy)-pyridin-3-yl]-3-methyl-1-(2-methyl-pyridin-3-yl)-1,3-dihydro-imidazo[4,5-c]quinolin-2-one

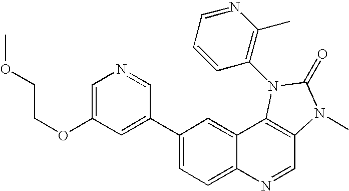

The title compound was synthesized in a similar manner as described for Example 1.1 using 3-(2-Methoxy-ethoxy)-5-(4,4,5,5-tetramethyl-[1,3,2]dioxaborolan-2-yl)-pyridine and intermediate B.

(HPLC: t_{R} 3.95 min (Method C); M+H=442.1 MS−ES. ¹H-NMR (d₆-DMSO, 600 MHz) 9.09 (s, 1H), 8.76 (m, 1H), 8.27 (m, 1H), 8.17-8.09 (m, 3H), 8.01-7.99 (m, 1H), 7.62-7.59 (m, 1H), 7.30 (s, 1H), 7.05 (s, 1H), 4.21-4.19 (m, 2H), 3.74-3.72 (m, 2H), 3.65 (m, 3H), 3.35 (s, 3H), 2.32 (s, 3H)).

### Stage 2.17.1 3-(2-Methoxy-ethoxy)-5-(4,4,5,5-tetramethyl-[1,3,2]dioxaborolan-2-yl)-pyridine

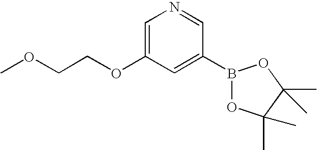

The title compound was synthesized in a similar manner as described for Stage 1.3.1 using 3-bromo-5-(2-methoxy-ethoxy)-pyridine (Stage 2.17.2) to give the title compound as a brown oil. (HPLC: t_{R} 2.10 min (Method C)).

### Stage 2.17.2 3-Bromo-5-(2-methoxy-ethoxy)-pyridine

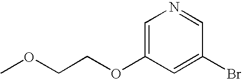

A mixture of 3-bromo-5-hydroxypyridine (Aldrich, Buchs, Switzerland, 611 mg, 3.51 mmol), potassium carbonate (971 mg, 7.02 mmol) and 2-bromoethyl methyl ether (537 mg, 3.86 mmol) in 30 ml DMF was stirred for 14 h at rt and for 2 h at 80° C. The reaction mixture was quenched with water and extracted with EtOAc (2×). The organic layers were washed with brine (3×), dried over Na₂SO₄, filtered and evaporated. The residue was purified by flash chromatography (dichloromethane/MeOH 0% to 3%) to give the title compound as an oil. (HPLC: t_{R} 2.38 min (Method C); M+H=232, 234 MS−ES).

### Example 2.18

### 8-(2-Ethoxy-pyridin-4-yl)-3-methyl-1-(2-methyl-pyridin-3-yl)-1,3-dihydro-imidazo[4,5-c]quinolin-2-one

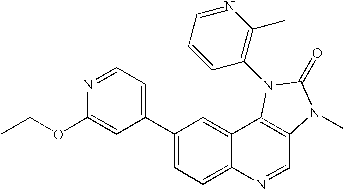

The title compound was synthesized in a similar manner as described for Example 1.1 using 2-ethoxy-pyridine-4-boronic acid and intermediate B.

(HPLC: t_{R} 4.58 min (Method B); M+H=412.1 MS−ES.)

### Example 2.19

### 8-(5-Isopropoxy-6-methyl-pyridin-3-yl)-3-methyl-1-(2-methyl-pyridin-3-yl)-1,3-dihydro-imidazo[4,5-c]quinolin-2-one

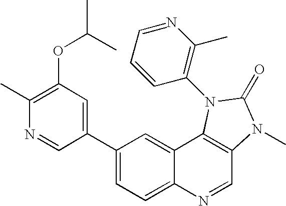

The title compound was synthesized in a similar manner as described for Example 1.1 using 5-bromo-3-isopropoxy-2-methyl-pyridine and intermediate B.

(HPLC: t_{R} 4.27 min (Method B); M+H=440.1 MS−ES, ¹H-NMR (d₆-DMSO, 600 MHz) 9.07 (s, 1H), 8.76-8.75 (m, 1H), 8.16-8.14 (m, 1H), 8.10-8.08 (m, 1H), 8.00 (s, 1H), 7.98-7.96 (m, 1H), 7.61-7.59 (m, 1H), 7.16 (s, 1H), 7.04 (s, 1H), 4.63-4.61 (m, 1H), 3.64 (s, 3H), 2.34 (s, 3H), 2.31 (s, 3H), 1.33 (s, 3H), 1.32 (s, 3H)).

### Stage 2.19.1. 3-Isopropoxy-2-methyl-5-(4,4,5,5-tetramethyl-[1,3,2]dioxaborolan-2-yl)-pyridine

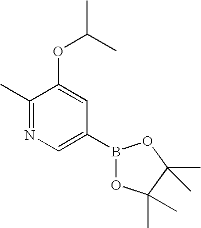

The title compound was synthesized in a similar manner as described for stage 1.3.1 using 5-bromo-3-isopropoxy-2-methyl-pyridine (Stage 2.19.2) to give the title compound as a brown oil. (HPLC: t_{R} 2.21 min (Method C); M+H=278 MS−ES).

### Stage 2.19.2. 5-Bromo-3-isopropoxy-2-methyl-pyridine

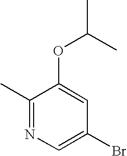

To a solution of 5-bromo-2-methyl-pyridin-3-ylamine (Stage 2.19.3, 400 mg, 2.13 mmol) in isopropanol (33 ml) were added 4 M HCl in dioxane (0.535 ml, 2.13 mmol) and isoamyl nitrite (1.25 g, 10.7 mmol). The reaction mixture was heated at 80° C. for 2.5 h, before being evaporated to dryness. The residue was dissolved in EtOAc and washed with saturated aqueous NaHCO₃. The aqueous layer was extracted with EtOAc and the combined organic layers were dried over Na₂SO₄, filtered and evaporated. The residue was dry loaded on silica gel and purified by MPLC (heptane/EtOAc 0% to 30%) to give the title compound as an orange oil (HPLC: t_{R} 2.47 min (Method C); M+H=230, 232 MS−ES).

### Stage 2.19.3 5-Bromo-2-methyl-pyridin-3-ylamine

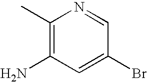

To a solution of 5-bromo-2-methyl-3-nitro-pyridine (stage 2.19.4, 765 mg, 3.53 mmol) in acetic acid (7 ml) and water (1.75 ml) was added in three portions iron powder (591 mg, 10.6 mmol). The reaction mixture was stirred for 2.5 h at rt then quenched with 20 ml of 10 M aqueous NaOH, 20 g ice and 20 ml EtOAc before being filtered over Celite. The solid was washed with EtOAc and the filtrate was extracted with EtOAc. The combined organic layers were washed with brine, dried over Na₂SO₄, filtered and evaporated to give the title compound as a gray solid. (HPLC: t_{R} 1.52 min (Method C); M+H=187, 189 MS−ES).

Stage 2.19.4 5-Bromo-2-methyl-3-nitro-pyridine

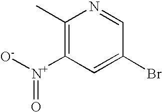

To a suspension of NaH 55% in oil (300 mg, 6.87 mmol) in 6 ml DMF was added dropwise diethyl malonate (Aldrich, Buchs, Switzerland, 1.0 g, 6.24 mmol). The reaction mixture was stirred for 20 min at rt before adding 5-bromo-2-chloro-3-nitropyridine (Matrix, Columbia, USA, 1.19 g, 5.0 mmol). The reaction mixture was stirred for 30 min at rt and for 1 h at 40° C. The reaction mixture was quenched with 10% aqueous NH₄Cl and extracted with EtOAc (2×). The combined organic layers were washed with brine (4×), dried over Na₂SO₄, filtered and evaporated. The residue was stirred in concentrated HCl (20 ml) for 13 h at 100° C. After cooling, the reaction mixture was basified with 10 M aqueous NaOH and extracted with EtOAc (2×). The organic layers were washed with brine, dried over Na₂SO₄, filtered and evaporated. The residue was dry loaded on silica gel and purified by MPLC (heptane/EtOAc 0% to 15%) to give the title compound as a yellow solid. (HPLC: t_{R} 2.84 (Method C).

### Example 2.20

### 8-(5-Ethoxy-6-methyl-pyridin-3-yl)-3-methyl-1-(2-methyl-pyridin-3-yl)-1,3-dihydro-imidazo[4,5-c]quinolin-2-one

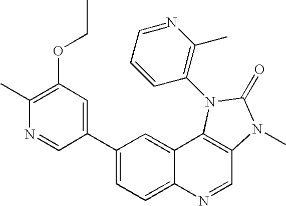

The title compound was synthesized in a similar manner as described for Example 1.1 using 3-ethoxy-2-methyl-5-(4,4,5,5-tetramethyl-[1,3,2]dioxaborolan-2-yl)-pyridine (Stage 2.20) and intermediate B.

(HPLC: t_{R} 4.08 min (Method B); M+H=426.1 MS−ES)

### Stage 2.20.1. 3-Ethoxy-2-methyl-5-(4,4,5,5-tetramethyl-[1,3,2]dioxaborolan-2-yl)-pyridine

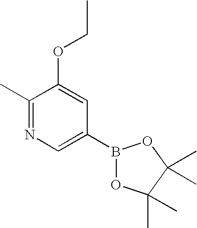

The title compound was synthesized in a similar manner are described for stage 2.19.2 and 2.19.1 using ethanol instead of isopropanol. (HPLC: tR 2.03 min (Method A); M+H=264 MS−ES)

### Example 2.21

### 8-(5-Ethylamino-6-methyl-pyridin-3-yl)-3-methyl-1-(2-methyl-pyridin-3-yl)-1,3-dihydro-imidazo[4,5-c]quinolin-2-one

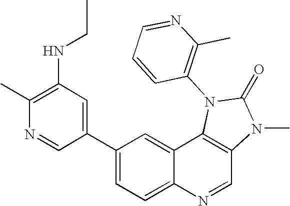

The title compound was synthesized in a similar manner as described for Example 1.1 using ethyl-[2-methyl-5-(4,4,5,5-tetramethyl-[1,3,2]dioxaborolan-2-yl)-pyridin-3-yl]-amine (Stage 2.21.1) and intermediate B.

(HPLC: t_{R} 3.95 min (Method B); M+H=425.1 MS−ES, ¹H-NMR (d₆-DMSO, 600 MHz) 9.05 (s, 1H), 8.76-8.75 (m, 1H), 8.16-8.14 (m, 1H), 8.13-8.11 (m, 1H), 8.09-8.08 (m, 1H), 7.94-7.92 (m, 1H), 7.74 (s, 1H), 7.60-7.58 (m, 1H), 7.05 (s, 1H), 6.58 (s, 1H), 5.25-5.23 (m, 1H), 3.64 (s, 3H), 3.09-3.06 (m, 2H), 2.31 (s, 6H), 1.24-1.23 (m, 3H)).

### Stage 2.21.1 Ethyl-[2-methyl-5-(4,4,5,5-tetramethyl-[1,3,2]dioxaborolan-2-yl)-pyridin-3-yl]-amine

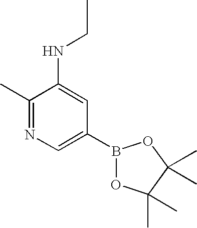

The title compound was synthesized in a similar manner as described for stage 1.3.1 using (5-bromo-2-methyl-pyridin-3-yl)-ethyl-amine (Stage 2.21.2) to give the title compound which is used without further purification in the next step. (HPLC: t_{R} 1.85 min (Method C); M+H=263.2 MS−ES).

### Stage 2.21.2. (5-Bromo-2-methyl-pyridin-3-yl)-ethyl-amine

300 mg (1.604 mMol) of 5-bromo-2-methyl-pyridin-3-ylamine (Stage 2.19.3) is dissolved in CH₂Cl₂ (30 mL), followed by addition of acetic acid (0.3 mL; 5.24 mMol) and acetaldehyde (70.7 mg, 1.604 mMol) and the mixture is kept stirring for 1.5 h at RT. To this NaBH(OAc)₃ (850 mg; 4.01 mMol) is added and the reaction mixture kept at RT for 4 h.

The reaction mixture is quenched with NaHCO₃ (20 mL satd. soln.) and extracted with CH₂Cl₂ (2×50 mL). The combined organic layers are dried over Na₂SO₄, filtered and evaporated. Purification of the crude product is done on silica gel (eluting with CH₂Cl₂/iProp 0% to 3%) to obtain the title compound as an oil.

(M+H=217.0 MS−ES).

### Example 2.22

### 8-(5-Ethoxy-6-methyl-pyridin-3-yl)-3-methyl-1-(2-methyl-pyridin-3-yl)-1,3-dihydro-imidazo[4,5-c]quinolin-2-one

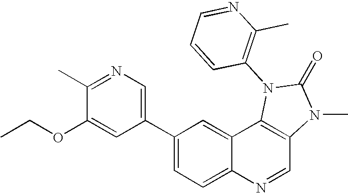

The title compound was synthesized in a similar manner as described for Example 1.1 using 3-ethoxy-2-methyl-5-(4,4,5,5-tetramethyl-[1,3,2]dioxaborolan-2-yl)-pyridine and intermediate B.

(HPLC: t_{R} 4.08 min (Method B); M+H=426.1 MS−ES).

### Example 2.23

### 8-Imidazo[1,2-a]pyridin-6-yl-3-methyl-1-(2-methyl-pyridin-3-yl)-1,3-dihydro-imidazo[4,5-c]quinolin-2-one

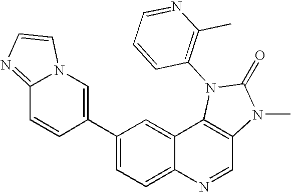

The title compound was synthesized in a similar manner as described for Example 1.1 using imidazo[1,2-A]pyridine-6-boronic acid and intermediate B.

(HPLC: t_{R} 3.41 min (Method A); M+H=407.1 MS−ES); ¹H NMR (600 MHz, DMSO-d₆) δ ppm 9.07 (s, 1H), 8.80 (d, 1H), 8.76 (s, 1H), 8.17 (d, 1H), 8.11 (d, 1H), 7.89-8.00 (m, 2H), 7.53-7.67 (m, 3H), 6.90-7.02 (m, 2H), 3.65 (s, 3H), 2.32 (s, 3H)).

### Example 2.24

### 8-(5-Isopropylamino-pyridin-3-yl)-3-methyl-1-(2-methyl-pyridin-3-yl)-1,3-dihydro-imidazo[4,5-c]quinolin-2-one

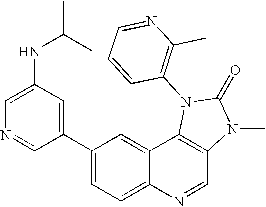

The title compound was synthesized in a similar manner as described for Example 1.1 using isopropyl-[5-(4,4,5,5-tetramethyl-[1,3,2]dioxaborolan-2-yl)-pyridin-3-yl]-amine (CM-1383) and intermediate B.

(HPLC: t_{R} 3.60 min (Method A); M+H=425.2 MS−ES, ¹H-NMR (d₆-DMSO, 600 MHz) 9.06 (s, 1H), 8.75-8.74 (m, 1H), 8.14-8.13 (m, 1H), 8.08 (m, 1H), 7.92-7.91 (m, 1H), 7.70 (s, 1H), 7.60-7.58 (m, 1H), 7.04 (s, 1H), 6.69 (s, 1H), 5.90-5.88 (m, 1H), 3.64 (s, 3H), 3.52 (m, 2H), 2.31 (s, 3H), 1.17-1.16 (m, 6H)).

### Stage 2.24.1. Isopropyl-[5-(4,4,5,5-tetramethyl-[1,3,2]dioxaborolan-2-yl)-pyridin-3-yl]-amine

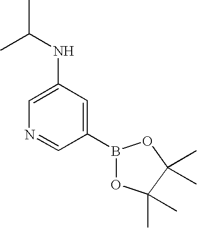

The title compound was synthesized in a similar manner as described in example 2.27, stage 2.27.1 using (5-bromo-pyridin-3-yl)-isopropyl-amine (Stage 2.24.2) to give the title compound as a dark foam. (HPLC: t_{R} 1.91 min (Method C); M+H=263.2 MS−ES).

### Stage 2.24.2 (5-Bromo-pyridin-3-yl)isopropyl-amine

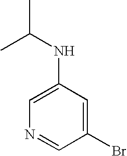

3-Bromo-5-fluoropyridine (507 mg, 2.85 mmol) was dissolved in 5 mL of NMP under Ar. Isopropylamine (0.545 mL, 6.27 mmol) was added and the mixture was heated at 190° C. for 10.5 h. NaHCO₃ was added to the reaction mixture and extracted with EtOAc. The organic phase was washed with water (3×) and brine (3×). It was dried over Na₂SO₄, filtered and evaporated. After drying under vacuum, the title compound was obtained as a brown solid (556 mg). (HPLC: t_{R} 2.14 min (Method C); M+H=215.0 MS−ES).

### Example 2.25

### 8-(5-Amino-pyridin-3-yl)-3-methyl-1-(2-methyl-pyridin-3-yl)-1,3-dihydro-imidazo[4,5-c]quinolin-2-one

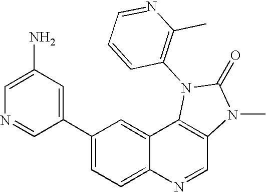

The title compound was synthesized in a similar manner as described for Example 1.1 using 3-Aminopyridin-5-boronic acid pinacolester and intermediate B.

(HPLC: t_{R} 3.358 min (Method A); M+H=383.1 MS−ES; ¹H NMR (400 MHz, DMSO-d₆) δ ppm 9.05 (s, 1H), 8.76 (dd, 1H), 8.13 (d, 1H), 8.08 (dd, 1H), 7.88 (d, 1H), 7.78 (dd, 1H), 7.53-7.65 (m, 2H), 6.97 (d, 1H), 6.89 (t, 1H), 5.44 (s, 2H), 3.63 (s, 3H), 2.29 (s, 3H)).

### Example 2.26

### 3-Methyl-8-(5-methylamino-pyridin-3-yl)-1-(2-methyl-pyridin-3-yl)-1,3-dihydro-imidazo[4,5-c]quinolin-2-one

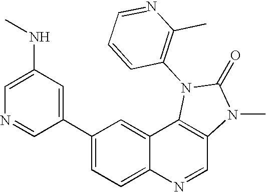

The title compound was synthesized in a similar manner as described for Example 1.1 using methyl-[4-(4,4,5,5-tetramethyl-[1,3,2]dioxaborolan-2-yl)-pyridin-3-yl]-amine and intermediate B.

(HPLC: t_{R} 3.492 min (Method A); M+H=397.1 MS−ES, ¹H NMR (600 MHz, DMSO-d₆) δ ppm 9.06 (s, 1H), 8.76 (d, 1H), 8.13 (d, 1H), 8.09 (d, 1H), 7.85-7.98 (m, 2H), 7.77 (d, 1H), 7.60 (dd, 1H), 7.04 (d, 1H), 6.63 (s, 1H), 6.11 (d, 1H), 3.64 (s, 3H), 2.70 (d, 3H), 2.31 (s, 3H).

### Stage 2.26.1. Methyl-[5-(4,4,5,5-tetramethyl-[1,3,2]dioxaborolan-2-yl)-pyridin-3-yl]-amine CM-1486

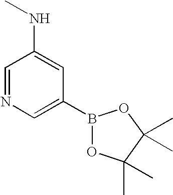

The title compound was synthesized in a similar manner as described in example 2.27, stage 2.27.1 using (5-bromo-pyridin-3-yl)-methyl-amine (Stage 2.26.2) to give the title compound as a dark foam. (HPLC: t_{R} 1.58 min (Method C); M+H=235.2 MS−ES).

### Stage 2.26.2 (5-Bromo-pyridin-3-yl)-methyl-amine

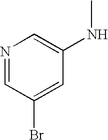

3-Bromo-5-fluoropyridine (534 mg, 2.94 mmol) was dissolved in 5 mL of NMP under Ar. Methylamine (0.809 mL, 6.48 mmol) was added and the mixture was heated at 200° C. for 1 h. NaHCO₃ was added to the reaction mixture and extracted with EtOAc. The organic phase was washed with water (3×) and brine (3×). It was dried over Na₂SO₄, filtered and evaporated. After drying under vacuum, the title compound was obtained as a white solid (505 mg). (HPLC: t_{R} 1.73 min (Method C); M+H=187.0 MS−ES).

### Example 2.27

### 8-(6-Dimethoxymethyl-5-methoxy-pyridin-3-yl)-3-methyl-1-(2-methyl-pyridin-3-yl)-1,3-dihydro-imidazo[4,5-c]quinolin-2-one

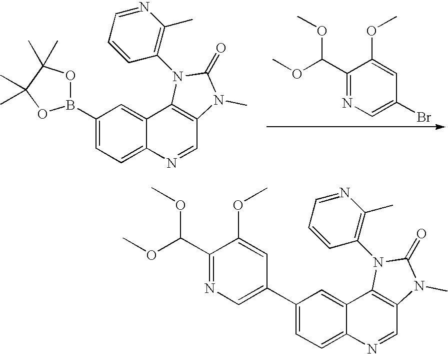

The title compound was synthesized in a similar manner as described for Example 1.1 but using 3-Methyl-1-(2-methyl-pyridin-3-yl)-8-(4,4,5,5-tetramethyl-[1,3,2]dioxaborolan-2-yl)-1,3-dihydro-imidazo[4,5-c]quinolin-2-one and 5-bromo-2-dimethoxymethyl-3-methoxy-pyridine (Adesis Inc., New Castle, Del., USA).

(HPLC: t_{R} 3.775 min (Method A); M+H=472.2 MS−ES, ¹H NMR (600 MHz, DMSO-d₆) δ ppm 9.09 (s, 1H), 8.72-8.82 (m, 1H), 8.18 (d, 1H), 8.08-8.15 (m, 2H), 8.03 (dd, 1H), 7.61 (dd, 1H), 7.30 (d, 1H), 7.08 (d, 1H), 5.57 (s, 1H,) 3.87 (s, 3H), 3.65 (s, 3H), 3.20-3.40 (m, 6H), 2.32 (s, 3H)).

### Stage 2.27.1 3-Methyl-1-(2-methyl-pyridin-3-yl)-8-(4,4,5,5tetramethyl-[1,3,2]dioxaborolan-2-yl)-1,3-dihydro-imidazo[4,5-c]quinolin-2-one

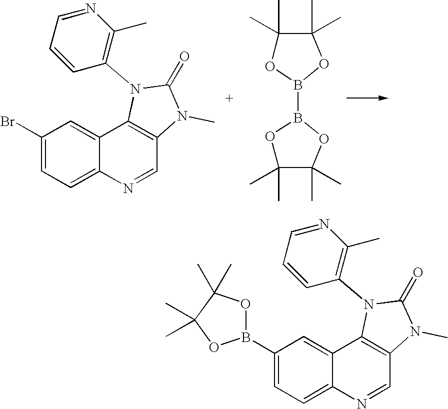

8-Bromo-3-methyl-1-(2-methyl-pyridin-3-yl)-1,3-dihydro-imidazo[4,5-c]quinolin-2-one (Intermediate B) (180 mg; 0.488 mmol) was dissolved in dioxane (10 mL), followed by addition of bis-(pinacolato)-diboron (149 mg; 0.585 mmol), PdCl₂(dppf) (15.7 mg; 0.021 mmol) and potassium acetate (144 mg; 1.472 mmol). The mixture was kept under an atmosphere of argon and stirred for 17 h at 100° C. After cooling to RT, EtOAc (100 mL) is added and the mixture extracted with water (2×). After removal of the solvent under reduced pressure the title compound is obtained as a brown oil which is used in the next step without further purification. (HPLC: t_{R} 3.350 min (Method A); M+H=417.2 MS−ES).

### Example 2.28

### 8-(5-Ethoxy-6-methoxymethyl-pyridin-3-yl)-3-methyl-1-(2-methyl-pyridin-3-yl)-1,3-dihydro-imidazo[4,5-c]quinolin-2-one

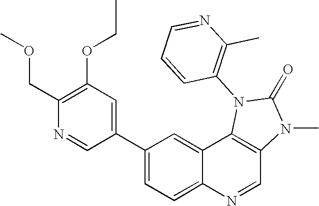

The title compound was synthesized in a similar manner as described for Example 1.1 using 3-ethoxy-2-methoxymethyl-5-(4,4,5,5-tetramethyl-[1,3,2]dioxaborolan-2-yl)-pyridine and intermediate B.

(HPLC: t_{R} 3.883 min (Method A); M+H=456.2 MS−ES).

### Stage 2.28.1. 3-Ethoxy-2-methoxymethyl-5-(4,4,5,5-tetramethyl-[1,3,2]dioxaborolan-2-yl)-pyridine

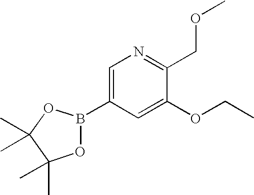

The title compound was prepared as described in example 2.27, stage 2.27.1 using 5-bromo-3-ethoxy-2-methoxymethyl-pyridine instead and was obtained as a black oil. The title compound was degrading under the HPLC condition: t_{R} 2.27 min (Method C); M+H=2.94 MS−ES).

### Stage 2.28.2. 5-Bromo-3-ethoxy-2-methoxymethyl-pyridine

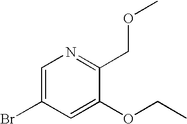

The title compound was prepared as described in example 2.30, stage 2.30.2 using (5-bromo-3-ethoxy-pyridin-2-yl)-methanol (stage 2.28.3) instead. (HPLC: t_{R} 2.62 min (Method C); M+H=248.0 MS−ES).

### Stage 2.28.3. (5-Bromo-3-ethoxy-pyridin-2-yl)-methanol

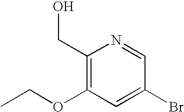

To a solution of 5-bromo-3-ethoxy-2-methyl-pyridine-1-oxide (1.12 g; 4.63 mmol) in anhydrous THF (25 mL) was added, under argon, 3.29 ml trifluoroacetic anhydride (3.29 mL; 23.16 mmol) and the solution wasn stirred at RT for 3 h. After that, the mixture was concentrated, followed by addition of sat. aqueous NaHCO₃ (35 mL) and stirring at rt for 15 h. Then, the suspension was diluted with water and extracted with EtOAc (2×100 ml). The combined organic layers are washed with 50 ml sat. aqueous NaHCO₃ and 50 ml brine, dried over Na₂SO₄, filtered and evaporated to dryness to obtain the title compound as 1.06 g of an orange solid is obtained (1.06 g). (HPLC: t_{R} 2.03 min (Method C); M+H=232, 234 MS−ES).

### Stage 2.28.4. 5-Bromo-3-ethoxy-2-methyl-pyridine-1-oxide

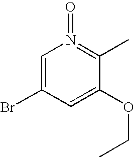

To a solution of 5-bromo-3-ethoxy-2-methyl-pyridine (1.05 g; 4.86 mmol) in CH₂Cl₂ (35 mL) was added m-CPBA (1.45 g; 5.88 mmol) and the mixture was stirred at RT under argon for 14 h. Then, The reaction mixture was diluted with 200 ml CH₂Cl₂ (200 mL), washed with sat. aqueous Na₂CO₃ (1×100 mL), sat. aqueous NaHCO₃ (3×100 mL) and 100 ml brine, before being dried over Na₂SO₄, filtered and evaporated to dryness. The title compound (1.13 g) was obtained as an off-white solid. (HPLC: t_{R} 2.51 min (Method C); M+H=232.0 MS+ES).

### Stage 2.28.5. 5-Bromo-3-ethoxy-2-methyl-pyridine

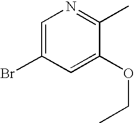

To a solution of 5-bromo-2-methyl-pyridin-3-ylamine (1.5 g; 7.62 mmol) in EtOH (110 mL) under argon were added 4N HCl in dioxane (1.89 mL; 7.62 mmol) and isoamyl nitrite (5.13 mL; 38.1 mmol) and the mixture was heated at 80° C. for 4 h. Then the mixture was evaporated to dryness. The residue was taken into 200 ml EtOAc (200 mL) and washed with sat. aqueous NaHCO₃ (50 mL) and 50 brine, dried over Na₂SO₄, filtered and evaporated. Purification was done on preabsorbed silica gel and (Combi flash chromatography (40 g column, Heptane/EtOAc 0%-30% in 25 min) to obtain the title compound (1.06 g) as a lightly yellow oil. (HPLC: t_{R} 2.24 min (Method C); M+H=218.0 MS+ES).

### Stage 2.28.6. 5-Bromo-2-methyl-pyridin-3-ylamine

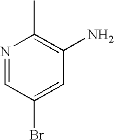

5-Bromo-2-methyl-3-nitro-pyridine (54.g; 24.14. mmol) was dissolved in AcOH (48 mL) and water (12 mL) under Ar. At 0° C., iron (4.04 g; 72.4 mmol) was added in 2 portions and the mixture was stirred at RT. Ice and 85 ml of 10 M aqueous NaOH were added, followed by. EtOAc and filtration over Celite. The residue was washed with EtOAc. The cake was taken in EtOAc, sonicated and filtered again. The layers were separated and the aqueous phase was extracted with EtOAc (1×). The combined organic layers was washed with brine, dried over Na₂SO₄, filtered and evaporated. After drying under vacuum the title compound was obtained as a black solid, (4.47 g). (HPLC: t_{R} 1.49 min (Method C); M+H=186.9 MS+ES).

### Stage 2.28.7. 5-Bromo-2-methyl-3-nitro-pyridine

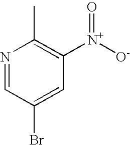

NaH (1.8 g; 41.3 mmol) was suspended in 35 mL of DMF under Ar. Diethyl malonate (5.66 mL, 36.1 mmol) was added dropwise and the mixture was stirred for 30 min at RT. Finally, 5-Bromo-2-chloro-3-nitro-pyridine (7 g, 28.9 mmol) was added and the mixture was stirred 30 min at RT and 1 h at 40° C. After 1 h, 10% aqueous NH₄Cl was added followed by extraction with EtOAc (2×). The combined organic layers were washed with brine (4×), dried over Na₂SO₄, filtered and evaporated. The residue was taken in 70 mL of HCl conc. and the mixture was heated at 100° C. After 16 h, the mixture was basified with 10 M NaOH (85 mL) and then extracted with EtOAc (2×). The combined organic layers were washed with water, brine, dried over Na₂SO₄, filtered and evaporated. After drying under vacuum the title compound was obtained as a brown oil, (5.43 g). (HPLC: t_{R} 2.92 min (Method C); M+H=217.0 MS+ES).

### Example 2.29

### 8-(5-Azetidin-1-yl-pyridin-3-yl)-3-methyl-1-(2-methyl-pyridin-3-yl)-1,3-dihydro-imidazo[4,5-c]quinolin-2-one

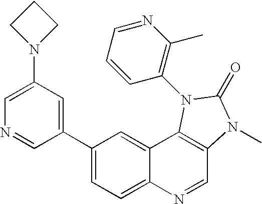

The title compound was synthesized in a similar manner as described for Example 1.1 using 3-azetidin-1-yl-5-(4,4,5,5-tetramethyl-[1,3,2]dioxaborolan-2-yl)-pyridine and intermediate B.

(HPLC: t_{R} 3.650 min (Method A); M+H=423.2 MS−ES).

### Stage 2.29.1. 3-Azetidin-1-yl-5-(4,4,5,5-tetramethyl-[1,3,2]dioxaborolan-2-yl)-pyridine

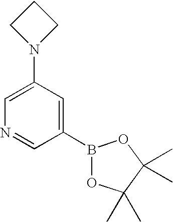

The title compound was synthesized in a similar manner as described in example 2.27, stage 2.27.1 using 3-azetidin-1-yl-5-bromo-pyridine (Stage 2.29.2) to give the title compound as a dark foam. (HPLC: t_{R} 1.93 min (Method C); M+H=261.1 MS−ES).

### Stage 2.29.2 3-Azetidin-1-yl-5-bromo-pyridine

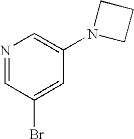

The title compound was synthesized in a similar manner as described in example 2.26, stage 2.26.2 using azetidine instead (to give the title compound as a yellow solid. (HPLC: t_{R} 2.10 min (Method C); M+H=213.0 MS−ES).

### Example 2.30

### 8-(5-Methoxy-6-methoxymethyl-pyridin-3-yl)-3-methyl-1-(2-methyl-pyridin-3-yl)-1,3-dihydro-imidazo[4,5-c]quinolin-2-one

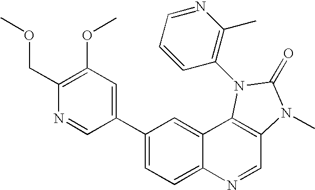

The title compound was synthesized in a similar manner as described for Example 1.1 using 3-methoxy-2-methoxymethyl-5-(4,4,5,5-tetramethyl-[1,3,2]dioxaborolan-2-yl)-pyridine and intermediate B.

(HPLC: t_{R} 3.625 min (Method A); M+H=442.2 MS−ES).

### Stage 2.30.1. 3-Methoxy-2-methoxymethyl-5-(4,4,5,5-tetramethyl-[1,3,2]dioxaborolan-2-yl)-pyridine

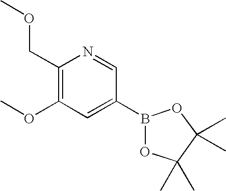

The title compound was prepared as described in example 2.27, stage 2.27.1 using 5-bromo-3-methoxy-2-methoxymethyl-pyridine (HPLC: t_{R} 3.467 min (Method A); M+H=280.1 MS−ES).

### Stage 2.30.2. 5-Bromo-3-methoxy-2-methoxymethyl-pyridine

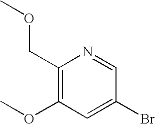

(5-Bromo-3-methoxypyridin-2-yl)methanol (615 mg; 2.82 mmol) was dissolved in dry DMF (10 mL), and at 0° C. treated with NaH (55% in mineral oil; 185 mg; 4.23 mmol) for 30 min., followed by addition of CH₃I (480 mg; 3.38 mmol). The reaction was allowed to warm up to RT and kept stirring for 3 h. EtOAc (100 mL) was added and the mixture extracted with water (2×). After removal of the solvent under reduced pressure the crude was purified by chromatography over silica gel (eluting with CH₂Cl₂/MeOH 98/2) to obtain the title compound (607 mg). (HPLC: t_{R} 4.433 min (Method A); M+H=233.0 MS−ES).

### Example 2.31

### 8-(5-Ethylamino-6-hydroxymethyl-pyridin-3-yl)-3-methyl-1-(2-methyl-pyridin-3-yl)-1,3-dihydro-imidazo[4,5-c]quinolin-2-one

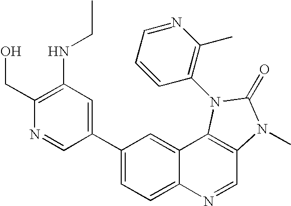

The title compound was synthesized in a similar manner as described for Example 1.1 using acetic acid 3-(tert-butoxycarbonyl-ethyl-amino)-5-(4,4,5,5-tetramethyl-[1,3,2]dioxaborolan-2-yl)-pyridin-2-ylmethyl ester and intermediate B. The acetyl protecting group as well as the BOC protecting group were removed together in situ by dissolving the crude product in an aqueous solution of LiOH (1 M, 4 equivalents), stirring for 5 min at RT and the removing the solvent under reduced pressure.

(HPLC: t_{R} 3.517 min (Method A); M+H=441.2 MS−ES).

### Stage 2.31.1. Acetic acid 3-(tert-butoxycarbonyl-ethyl-amino)-5-(4,4,5,5-tetramethyl-[1,3,2]dioxaborolan-2-yl)-pyridin-2-ylmethyl ester

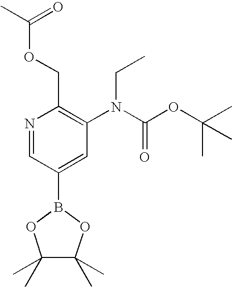

The title compound was synthesized in a similar manner as described in example 2.27, stage 2.27.1 using acetic acid 5-bromo-3-(tert-butoxycarbonyl-ethyl-amino)-pyridin-2-yl-methyl ester (Stage 2.31.2) to give the title compound as a crude black oil. (degrading under the HPLC condition: t_{R} 3.09 min (Method C); M+H=421 MS−ES).

### Stage 2.31.2. Acetic acid 5-bromo-3-(tert-butoxycarbonyl-ethyl-amino)-pyridin-2-yl-methyl ester

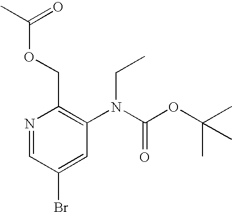

(5-Bromo-2-methyl-1-oxy-pyridin-3-yl)-ethyl-carbamic acid tert-butyl ester (Stage 2.31.3, 1.467 mmol) in acetic anhydride (1.5 ml) was stirred under Ar for 35 min at 120° C. The RM was quenched with EtOH and stirred then diluted with water, neutralized with saturated aqueous NaHCO₃ and extracted with EtOAc. The organic layer was washed with brine, dried over Na₂SO₄, filtered and evaporated. The residue was purified by flash chromatography (heptane/EtOAc 0% to 30%) to give after evaporation of the fractions containing the title compound an oil (HPLC: t_{R} 3.51 min (Method C); M+H=373, 375 MS−ES).

### Stage 2.31.3. (5-Bromo-2-methyl-1-oxy-pyridin-3-yl)-ethyl-carbamic acid tert-butyl ester

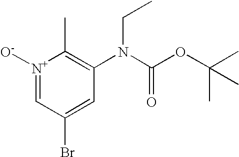

A mixture of (5-bromo-2-methyl-pyridin-3-yl)-ethyl-carbamic acid tert-butyl ester (stage 2.31.4, 1.508 mmol) in dichloromethane (13 ml) and 65% m-chloroperbenzoic acid (3.02 mmol) was stirred for 4 h at rt. The RM was diluted with dichloromethane, washed with saturated aqueous Na₂CO₃, saturated aqueous NaHCO₃ (4×) and brine, dried over Na₂SO₄, filtered, evaporated and dried under vacuum to give the title compound as an oil (HPLC: t_{R} 3.02 min (Method C); M+H=331, 333 MS−ES).

### Stage 2.31.4. (5-Bromo-2-methyl-pyridin-3-yl)-ethyl-carbamic acid tert-butyl ester

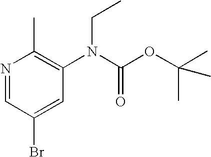

A mixture of (5-bromo-2-methyl-pyridin-3-yl)-ethyl-amine (Stage 2.21.2., 1.634 mmol) and di-tertbutyl dicarbonate (Fluka, Buchs, Switzerland, 1.961 mmol) in THF (1.6 ml) was stirred for 1 h at rt and 3 h at 65° C. Was added di-tertbutyl dicarbonate (Fluka, Buchs, Switzerland, 1.961 mmol) and an extra stirring for 22 h at 65° C. to bring reaction to completion. The RM was diluted with EtOAc, washed with saturated aqueous NaHCO₃, with brine, dried over Na₂SO₄, filtered and evaporated. The residue was absorbed on silica gel and purified by flash chromatography (heptane/EtOAc 0% to 30%) to give after evaporation of the fractions containing the title compound an oil (HPLC: t_{R} 3.41 min (Method C); M+H=315, 317 MS−ES).

### Example 2.32

### 3-Methyl-8-(6-methyl-5-methylamino-pyridin-3-yl)-1-(2-methyl-pyridin-3-yl)-1,3-dihydro-imidazo[4,5-c]quinolin-2-one

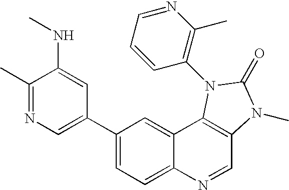

The title compound was synthesized in a similar manner as described for Example 1.1 using methyl-[2-methyl-5-(4,4,5,5-tetramethyl-[1,3,2]dioxaborolan-2-yl)-pyridin-3-yl]-amine and intermediate B.

(HPLC: t_{R} 3.567 min (Method A); M+H=411.1 MS−ES, ¹H NMR (600 MHz, DMSO-d₆) δ ppm 9.05 (s, 1H), 8.76 (d, 1H), 8.10 (dd, 2H), 7.93 (dd, 1H), 7.74 (d, 1H), 7.59 (dd, 1H), 7.05 (d, 1H), 6.51 (s, 1H) 5.52 (d, 1H), 3.64 (s, 3H), 2.73 (d, 3H), 2.30 (d, 6H)).

### Stage 2.32.1. Methyl-[2-methyl-5-(4,4,5,5-tetramethyl-[1,3,2]dioxaborolan-2-yl)pyridin-3-yl]amine

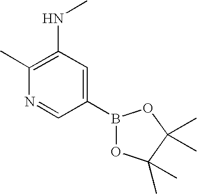

The title compound was prepared as described in example 2.27, stage 2.27.1 using (5-bromo-2-methyl-pyridin-3-yl)-methyl-amine (HPLC: t_{R} 1.64 min (Method C); M+H=249.2 MS+ES).

### Stage 2.32.2. (5-Bromo-2-methyl-pyridin-3-yl)-methyl-amine

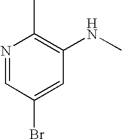

The title compound was prepared as described in example 2.27, stage 2.27.1 using 5-bromo-2-methyl-pyridin-3-ylamine (Stage 2.19.3) and methyl iodide. (HPLC: t_{R} 1.88 min (Method C); M+H=203.1 MS+ES).

### Example 3.1

### 8-(2-Ethoxy-pyrimidin-5-yl)-3-methyl-1-(3-methyl-pyridin-2-yl)-1,3-dihydro-imidazo[4,5-c]quinolin-2-one

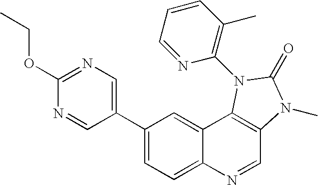

The title compound was synthesized in a similar manner as described for Example 1.1 using 6-ethoxypyridine-3-boronic acid and intermediate C.

(HPLC: t_{R} 4.08 min (Method A); M+H=413.1 MS−ES; ¹H-NMR (d₆-DMSO, 600 MHz) 9.06 (s, 1H), 8.65-8.55 (m, 1H), 8.55-8.50 (m, 2H), 8.20-8.08 (m, 2H), 8.00-7.90 (m, 1H), 7.75-7.65 (m, 1H), 6.75-6.60 (m, 1H), 4.36 (q, 2H), 3.63 (s, 3H), 2.25 (s, 3H), 1.32 (t, 3H)).

### Example 3.2

### 3-Methyl-1-(3-methyl-pyridin-2-yl)-8-(1H-pyrrolo[2,3-b]pyridin-5-yl)-1,3-dihydro-imidazo[4,5-c]quinolin-2-one

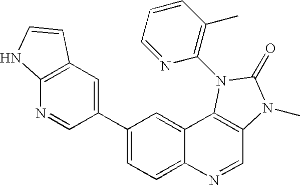

The title compound was synthesized in a similar manner as described for Example 1.1 using 5-(4,4,5,5-tetramethyl-1,3,2-dioxoborolan-2-yl)-1H-pyrrolo[2,3-b]pyridine and intermediate C.

(HPLC: t_{R} 3.82 min (Method A); M+H=407.1 MS−ES; ¹H-NMR (d₆-DMSO, 600 MHz) 11.73 (s, 1H), 9.03 (s, 1H), 8.68-8.59 (m, 1H), 8.19-8.06 (m, 3H), 8.01-7.94 (m, 1H), 7.94-7.86 (m, 1H), 7.77-7.67 (m, 1H), 7.59-7.49 (m, 1H), 6.80-6.68 (m, 1H), 6.54-6.43 (m, 1H), 3.64 (s, 3H), 2.24 (s, 3H)).

### Example 3.3

### 4-[3-Methyl-1-(3-methyl-pyridin-2-yl)-2-oxo-2,3-dihydro-1H-imidazo[4,5-c]quinolin-8-yl]-benzamide

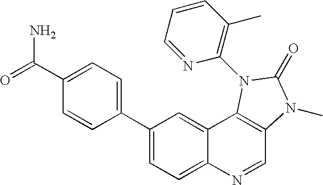

The title compound was synthesized in a similar manner as described for Example 1.1 using 4-aminocarbonylphenylboronic acid and intermediate C.

(HPLC: t_{R} 3.74 min (Method A); M+H=410.1 MS−ES; ¹H-NMR (d₆-DMSO, 600 MHz) 9.06 (s, 1H), 8.67-8.58 (m, 1H), 8.16-8.09 (m, 2H), 8.04-8.00 (m, 1H), 7.96-7.92 (m, 1H), 7.92-7.86 (m, 2H), 7.69-7.75 (m, 1H), 7.43-7.36 (m, 3H), 6.82-6.76 (m, 1H), 3.64 (s, 3H), 2.24 (s, 3H)).

### Example 3.4

### 3-Methyl-8-(2-methylamino-pyrimidin-5-yl)-1-(3-methyl-pyridin-2-yl)-1,3-dihydro-imidazo[4,5-c]quinolin-2-one

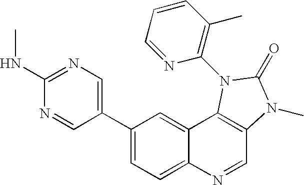

The title compound was synthesized in a similar manner as described for Example 1.1 using 2-methylaminopyrimidin-5-boronic acid PE and intermediate C.

(HPLC: t_{R} 3.72 min (Method A); M+H=398.1 MS−ES; ¹H-NMR (d₆-DMSO, 600 MHz) 9.01 (s, 1H), 8.66-8.56 (m, 1H), 8.35-8.15 (m, 2H), 8.15-8.10 (m, 1H), 8.10-8.06 (m, 1H), 7.88-7.84 (m, 1H), 7.74-7.68 (m, 1H), 7.37-7.33 (m, 1H), 6.62-6.58 (m, 1H), 3.62 (s, 3H), 2.81 (d, 3H), 2.24 (s, 3H)).

### Example 3.5

### N-Methyl-4-[3-methyl-1-(3-methyl-pyridin-2-yl)-2-oxo-2,3-dihydro-1H-imidazo[4,5-c]quinolin-8-yl]-benzamide

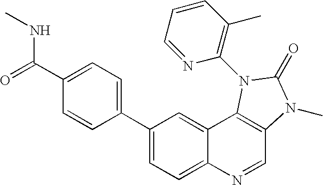

The title compound was synthesized in a similar manner as described for Example 1.1 using 4-(N-methylaminocarbonyl)phenylboronic acid and intermediate C.

(HPLC: t_{R} 3.85 min (Method A); M+H=424.1 MS−ES; ¹H-NMR (d₆-DMSO, 600 MHz) 9.06 (s, 1H), 8.66-8.61 (m, 1H), 8.53-8.45 (m, 1H), 8.17-8.07 (m, 2H), 7.97-7.91 (m, 1H), 7.90-7.82 (m, 2H), 7.769-7.67 (m, 1H), 7.43-7.36 (m, 2H), 6.83-6.75 (m, 1H), 3.63 (s, 3H), 2.78 (d, 3H), 2.24 (s, 3H)).

### Example 3.6

### 8-(6-Ethoxy-pyridin-3-yl)-3-methyl-1-(3-methyl-pyridin-2-yl)-1,3-dihydro-imidazo[4,5-c]quinolin-2-one

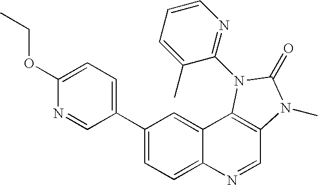

The title compound was synthesized in a similar manner as described for Example 1.1 using 6-ethoxypyridine-3-boronic acid and intermediate C.

(HPLC: t_{R} 4.33 min (Method A); M+H=412.1 MS−ES; ¹H-NMR (d₆-DMSO, 600 MHz) 9.03 (s, 1H), 8.64-8.57 (m, 1H), 8.15-8.06 (m, 3H), 7.91-7.84 (m, 1H), 7.75-7.67 (m, 1H), 7.67-7.61 (m, 1H), 6.87-6.81 (m, 1H), 6.70-6.65 (m, 1H), 4.31 (q, 2H), 3.63 (s, 3H), 2.24 (s, 3H), 1.31 (t, 3H)).

### Example 3.7

### 8-[6-(3-Dimethylamino-propoxy)-pyridin-3-yl]-3-methyl-1-(3-methyl-pyridin-2-yl)-1,3-dihydro-imidazo[4,5-c]quinolin-2-one

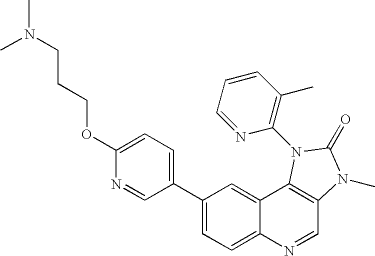

The title compound was synthesized in a similar manner as described for Example 1.1 using 2-(3-N,N-dimethylaminopropoxy)pyridin-5-boronic acid pinacol ester and intermediate C.

(HPLC: t_{R} 3.61 min (Method A); M+H=469.1 MS−ES; ¹H-NMR (d₆-DMSO, 600 MHz) 9.04 (s, 1H), 8.64-8.57 (m, 1H), 8.15-8.06 (m, 3H), 7.92-7.81 (m, 1H), 7.74-7.68 (m, 1H), 7.68-7.60 (m, 1H), 6.87-6.81 (m, 1H), 6.70-6.64 (m, 1H), 4.28 (t, 2H), 3.63 (s, 3H), 2.45-2.35 (m, 2H), 2.24 (s, 3H), 2.22-2.14 (m, 6H), 1.91-1.80 (m, 2H)).

### Example 3.8

### 3-Methyl-1-(3-methyl-pyridin-2-yl)-8-quinolin-3-yl-1,3-dihydro-imidazo[4,5-c]quinolin-2-one

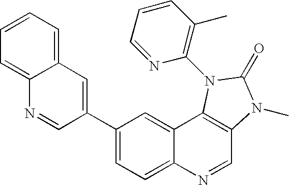

The title compound was synthesized in a similar manner as described for Example 1.1 using 3-quinolineboronic acid and intermediate C.

(HPLC: t_{R} 3.90 min (Method A); M+H=418.1 MS−ES; ¹H-NMR (d₆-DMSO, 600 MHz) 9.09 (s, 1H), 8.84-8.74 (m, 1H), 8.71-8.63 (m, 1H), 8.42-8.33 (m, 1H), 8.23-8.17 (m, 1H), 8.17-8.09 (m, 2H), 8.06-7.99 (m, 2H), 7.82-7.72 (m, 2H), 7.69-7.63 (m, 1H), 6.93-6.89 (m, 1H), 3.65 (s, 3H), 2.26 (s, 3H)).

### Example 3.9

### 8-(6-Hydroxymethyl-pyridin-3-yl)-3-methyl-1-(3-methyl-pyridin-2-yl)-1,3-dihydro-imidazo[4,5-c]quinolin-2-one

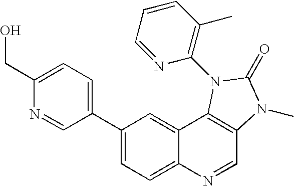

The title compound was synthesized in a similar manner as described for Example 1.1 using 6-(hydroxymethyl)pyridine-3-boronic acid and intermediate C.

(HPLC: t_{R} 3.41 min (Method A); M+H=398.1 MS−ES; ¹H-NMR (d₆-DMSO, 600 MHz) 9.06 (s, 1H), 8.65-8.58 (m, 1H), 8.41-8.35 (m, 1H), 8.18-8.08 (m, 2H), 7.96-7.89 (m, 1H), 7.81-7.75 (m, 1H), 7.75-7.67 (m, 1H), 7.54-7.44 (m, 1H), 6.81-6.72 (m, 1H), 5.47 (t, 1H), 4.57 (d, 2H), 3.63 (s, 3H), 2.24 (s, 3H)).

### Example 3.10

### 8-(3,4-Dimethoxy-phenyl)-3-methyl-1-(3-methyl-pyridin-2-yl)-1,3-dihydro-imidazo[4,5-c]quinolin-2-one

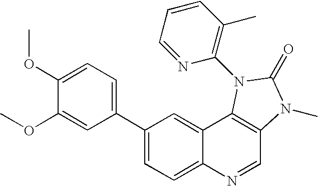

The title compound was synthesized in a similar manner as described for Example 1.1 using 3,4-dimethoxyphenylboronic acid and intermediate C.

(HPLC: t_{R} 4.18 min (Method A); M+H=427.1 MS−ES; ¹H-NMR (d₆-DMSO, 600 MHz) 9.01 (s, 1H), 8.68-8.59 (m, 1H), 8.17-8.03 (m, 2H), 7.93-7.86 (m, 1H), 7.74-7.66 (m, 1H), 7.01-6.92 (m, 2H), 6.78-6.70 (m, 2H), 3.76 (s, 6H), 3.62 (s, 3H), 2.23 (s, 3H)).

### Example 3.11

### 8-(6-Dimethylamino-pyridin-3-yl)-3-methyl-1-(3-methyl-pyridin-2-yl)-1,3-dihydro-imidazo[4,5-c]quinolin-2-one

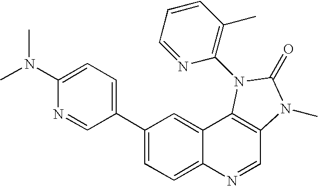

The title compound was synthesized in a similar manner as described for Example 1.1 using 6-(dimethylamino)pyridine-3-boronic acid and intermediate C.

(HPLC: t_{R} 3.55 min (Method A); M+H=411.1 MS−ES; ¹H-NMR (d₆-DMSO, 600 MHz) 8.98 (s, 1H), 8.68-8.53 (m, 1H), 8.17-8.09 (m, 1H), 8.09-7.99 (m, 2H), 7.90-7.78 (m, 1H), 7.75-7.66 (m, 1H), 7.47-7.38 (m, 1H), 6.69-6.59 (m, 2H), 3.62 (s, 3H), 3.04 (s, 6H), 2.24 (s, 3H)).

### Example 3.12

### 8-(6-Amino-5-trifluoromethyl-pyridin-3-yl)-3-methyl-1-(3-methyl-pyridin-2-yl)-1,3-dihydro-imidazo[4,5-c]quinolin-2-one

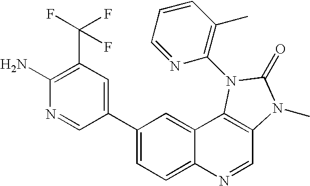

The title compound was synthesized in a similar manner as described for Example 1.1 using Stage 1.3.1 and intermediate C.

(HPLC: t_{R} 4.00 min (Method A); M+H=451.0 MS−ES; ¹H-NMR (d₆-DMSO, 600 MHz) 9.01 (s, 1H), 8.64-8.56 (m, 1H), 8.27-8.20 (m, 1H), 8.15-8.03 (m, 2H), 7.96-7.90 (m, 1H), 7.71-7.62 (m, 1H), 7.59-7.52 (m, 1H), 6.73 (s, 2H), 6.64-6.58 (m, 1H), 3.62 (s, 3H), 2.23 (s, 3H)).

### Example 3.13

### 8-(6-Methoxy-pyridin-3-yl)-3-methyl-1-(3-methyl-pyridin-2-yl)-1,3-dihydro-imidazo[4,5-c]quinolin-2-one

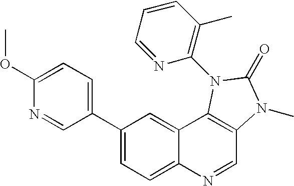

The title compound was synthesized in a similar manner as described for Example 1.1 using 2-methoxy-5-pyridine boronic acid and intermediate C.

(HPLC: t_{R} 4.13 min (Method A); M+H=398.1 MS−ES; ¹H-NMR (d₆-DMSO, 600 MHz) 9.01 (s, 1H), 8.65-8.56 (m, 1H), 8.15-8.07 (m, 3H), 7.91-7.82 (m, 1H), 7.75-7.68 (m, 1H), 7.68-7.62 (m, 1H), 6.90-6.82 (m, 1H), 6.72-6.64 (m, 1H), 3.86 (s, 3H), 3.62 (s, 3H), 2.23 (s, 3H)).

### Example 4.1

### 8-(6-Hydroxymethyl-pyridin-3-yl)-3-methyl-1-(4-methyl-pyridin-3-yl)-1,3-dihydro-imidazo[4,5-c]quinolin-2-one

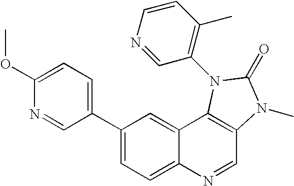

The title compound was synthesized in a similar manner as described for Example 1.1 using 6-(hydroxymethyl)pyridine-3-boronic acid and intermediate D.

(HPLC: t_{R} 3.33 min (Method A); M+H=398.1 MS−ES; ¹H-NMR (d₆-DMSO, 600 MHz) 9.07 (s, 1H), 8.80-8.70 (m, 2H), 8.42-8.33 (m, 1H), 8.20-8.10 (m, 1H), 8.00-7.90 (m, 1H), 7.83-7.72 (m, 1H), 7.71-7.62 (m, 1H), 7.55-7.44 (m, 1H), 7.05-6.95 (m, 1H), 5.50-5.40 (m, 1H), 4.69-4.52 (m, 2H), 3.64 (s, 3H), 2.17 (s, 3H)).

### Example 4.2

### 8-(6-Ethoxy-pyridin-3-yl)-3-methyl-1-(4-methyl-pyridin-3-yl)-1,3-dihydro-imidazo[4,5-c]quinolin-2-one

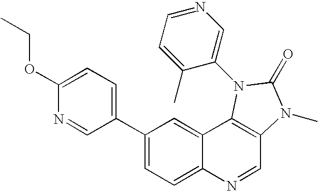

The title compound was synthesized in a similar manner as described for Example 1.1 using 6-ethoxypyridine-3-boronic acid and intermediate D.

(HPLC: t_{R} 4.09 min (Method A); M+H=412.1 MS−ES; ¹H-NMR (d₆-DMSO, 600 MHz) 9.04 (s, 1H), 8.80-8.69 (m, 2H), 8.18-8.05 (m, 2H), 7.92-7.84 (m, 1H), 7.72-7.66 (m, 1H), 7.66-7.57 (m, 1H), 6.96-6.89 (m, 1H), 6.86-6.81 (m, 1H), 4.32 (q, 2H), 3.63 (s, 3H), 2.16 (s, 3H), 1.31 (t, 3H)).

### Example 4.3

### 8-(6-Methoxy-pyridin-3-yl)-3-methyl-1-(4-methyl-pyridin-3-yl)-1,3-dihydro-imidazo[4,5-c]quinolin-2-one

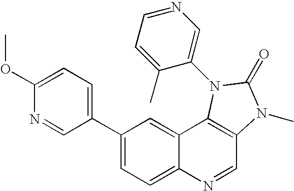

The title compound was synthesized in a similar manner as described for Example 1.1 using 2-methoxy-5-pyridine boronic acid and intermediate D.

(HPLC: t_{R} 3.93 min (Method A); M+H=398.2 MS−ES; ¹H-NMR (d₆-DMSO, 600 MHz) 9.06 (s, 1H), 8.80-8.71 (m, 2H), 8.16-8.06 (m, 2H), 7.95-7.87 (m, 1H), 7.71-7.60 (m, 2H), 6.96-6.91 (m, 1H), 6.91-6.81 (m, 1H), 3.87 (s, 3H), 3.63 (s, 3H), 2.16 (s, 3H)).

### Example 4.4

### 8-(6-Dimethylamino-pyridin-3-yl)-3-methyl-1-(4-methyl-pyridin-3-yl)-1,3-dihydro-imidazo[4,5-c]quinolin-2-one

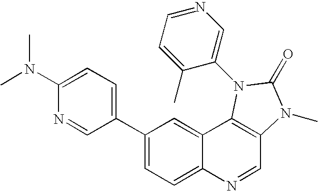

The title compound was synthesized in a similar manner as described for Example 1.1 using 6-(dimethylamino)pyridine-3-boronic acid and intermediate D.

(HPLC: t_{R} 3.47 min (Method A); M+H=411.2 MS−ES; ¹H-NMR (d₆-DMSO, 600 MHz) 8.99 (s, 1H), 8.79-8.70 (m, 2H), 8.10-8.01 (m, 2H), 7.89-7.80 (m, 1H), 7.72-7.63 (m, 1H), 7.46-7.36 (m, 1H), 6.92-6.85 (m, 1H), 6.71-6.60 (m, 1H), 3.63 (s, 3H), 3.04 (s, 6H), 2.16 (s, 3H)).

### Example 5.1

### 8-(6-Hydroxymethyl-pyridin-3-yl)-3-methyl-1-pyridin-3-yl-1,3-dihydro-imidazo[4,5-c]quinolin-2-one

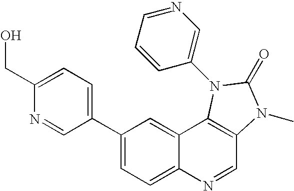

The title compound was synthesized in a similar manner as described for Example 1.1 using 6-(hydroxymethyl)pyridine-3-boronic acid and intermediate E.

(HPLC: t_{R} 3.29 min (Method A); M+H=384.1 MS−ES; ¹H-NMR (d₆-DMSO, 600 MHz) 9.06 (s, 1H), 8.99-8.80 (m, 2H), 8.50-8.40 (m, 1H), 8.28-8.10 (m, 2H), 8.00-7.90 (m, 1H), 7.85-7.71 (m, 2H), 7.55-7.45 (m, 1H), 7.22-7.15 (m, 1H), 5.55-5.43 (m, 1H), 4.66-4.47 (m, 2H), 3.62 (s, 3H)).

### Example 5.2

### 8-(3,4-Dimethoxy-phenyl)-3-methyl-1-pyridin-3-yl-1,3-dihydro-imidazo[4,5-c]quinolin-2-one

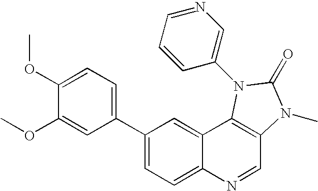

The title compound was synthesized in a similar manner as described for Example 1.1 using 3,4-dimethoxyphenylboronic acid and intermediate E.

(HPLC: t_{R} 4.05 min (Method A); M+H=413.1 MS−ES; ¹H-NMR (d₆-DMSO, 600 MHz) 9.00 (s, 1H), 8.96-8.90 (m, 1H), 8.89-8.80 (m, 1H), 8.25-8.15 (m, 1H), 8.12-8.05 (m, 1H), 7.95-7.87 (m, 1H), 7.81-7.74 (m, 1H), 7.17-7.10 (m, 1H), 7.04-6.94 (m, 2H), 6.82-6.75 (m, 1H), 3.77 (s, 6H), 3.61 (s, 3H)).

### Example 5.3

### 8-(6-Dimethylamino-pyridin-3-yl)-3-methyl-1-pyridin-3-yl-1,3-dihydro-imidazo[4,5-c]quinolin-2-one

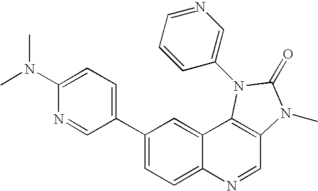

The title compound was synthesized in a similar manner as described for Example 1.1 using 6-(dimethylamino)pyridine-3-boronic acid and intermediate E.

(HPLC: t_{R} 3.43 min (Method A); M+H=397.1 MS−ES; ¹H-NMR (d₆-DMSO, 600 MHz) 8.98 (s, 1H), 8.92-8.86 (m, 2H), 8.20-8.15 (m, 1H), 8.13-8.10 (m, 1H), 8.10-8.04 (m, 1H), 7.89-7.83 (m, 1H), 7.80-7.76 (m, 1H), 7.49-7.43 (m, 1H), 7.08-7.02 (m, 1H), 6.69-6.63 (m, 1H), 3.60 (s, 3H), 3.04 (s, 6H)).

### Example 5.4

### 8-(6-Methoxy-pyridin-3-yl)-3-methyl-1-pyridin-3-yl-1,3-dihydro-imidazo[4,5-c]quinolin-2-one

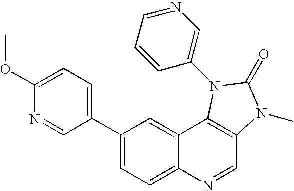

The title compound was synthesized in a similar manner as described for Example 1.1 using 2-methoxy-5-pyridine boronic acid and intermediate E.

(HPLC: t_{R} 3.93 min (Method A); M+H=384.1 MS−ES; ¹H-NMR (d₆-DMSO, 600 MHz) 9.03 (s, 1H), 8.92-8.89 (m, 1H), 8.88-8.85 (m, 1H), 8.21-8.16 (m, 1H), 8.16-8.14 (m, 1H), 8.14-8.10 (m, 1H), 7.91-7.86 (m, 1H), 7.80-7.75 (m, 1H), 7.72-7.66 (m, 1H), 7.13-7.08 (m, 1H), 6.90-6.85 (m, 1H), 3.87 (s, 3H), 3.61 (s, 3H)).

### Example 6.1

### 8-(6-Ethoxy-pyridin-3-yl)-1-(2-fluoro-pyridin-3-yl)-3-methyl-1,3-dihydro-imidazo[4,5-c]quinolin-2-one

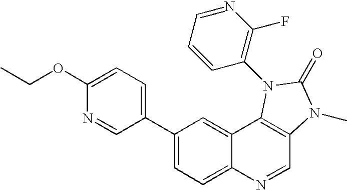

The title compound was synthesized in a similar manner as described for Example 1.1 using 6-ethoxypyridine-3-boronic acid and intermediate F.

(HPLC: t_{R} 4.39 min (Method A); M+H=416.0 MS−ES; ¹H-NMR (d₆-DMSO, 600 MHz) 9.04 (s, 1H), 8.62-8.53 (m, 1H), 8.52-8.43 (m, 1H), 8.21-8.07 (m, 2H), 7.95-7.86 (m, 1H), 7.80-7.70 (m, 2H), 7.19-7.12 (m, 1H), 6.90-6.80 (m, 1H), 4.31 (q, 2H), 3.61 (s, 3H), 1.30 (t, 3H)).

### Example 6.2

### 8-(6-Amino-5-trifluoromethyl-pyridin-3-yl)-1-(2-fluoro-pyridin-3-yl)-3-methyl-1,3-dihydro-imidazo[4,5-c]quinolin-2-one

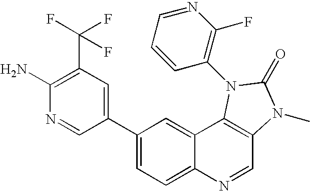

The title compound was synthesized in a similar manner as described for Example 1.1 using Stage 1.3.1 and intermediate F.

(HPLC: t_{R} 4.09 min (Method A); M+H=455.3 MS−ES; ¹H-NMR (d₆-DMSO, 600 MHz) 9.01 (s, 1H), 8.60-8.52 (m, 1H), 8.52-8.45 (m, 1H), 8.34-8.26 (m, 1H), 8.13-8.05 (m, 1H), 8.00-7.93 (m, 1H), 7.77-7.71 (m, 1H), 7.68-7.63 (m, 1H), 7.12-7.06 (m, 1H), 6.74 (s, 2H), 3.61 (s, 3H)).

### Example 6.3

### 1-(2-Fluoro-pyridin-3-yl)-3-methyl-8-(1H-pyrrolo[2,3-b]pyridin-5-yl)-1,3-dihydro-imidazo[4,5-c]quinolin-2-one

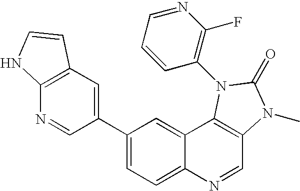

The title compound was synthesized in a similar manner as described for Example 1.1 using 5-(4,4,5,5-tetramethyl-1,3,2-dioxoborolan-2-yl)-1H-pyrrolo[2,3-b]pyridine and intermediate F.

(HPLC: t_{R} 3.88 min (Method A); M+H=411.0 MS−ES; ¹H-NMR (d₆-DMSO, 600 MHz) 11.74 (s, 1H), 9.04 (s, 1H), 8.65-8.55 (m, 1H), 8.55-8.45 (m, 1H), 8.24-8.11 (m, 2H), 8.02-7.95 (m, 1H), 7.83-7.72 (m, 2H), 7.57-7.48 (m, 1H), 7.26-7.15 (m, 1H), 6.54-6.41 (m, 1H), 3.62 (s, 3H)).

### Example 7.1

### 8-(6-Amino-5-trifluoromethyl-pyridin-3-yl)-1-(6-fluoro-2-methyl-pyridin-3-yl)-3-methyl-1,3-dihydro-imidazo[4,5-c]quinolin-2-one

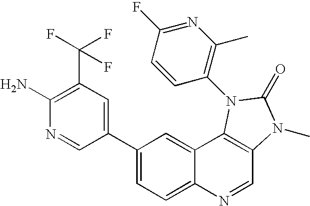

The title compound was synthesized in a similar manner as described for Example 1.1 using 5-(4,4,5,5-tetramethyl-1,3,2-dioxoborolan-2-yl)-1H-pyrrolo[2,3-b]pyridine and intermediate G.

(HPLC: t_{R} 4.09 min (Method A); M+H=469.1 MS−ES; ¹H-NMR (d₆-DMSO, 600 MHz) 9.03 (s, 1H), 8.39-8.32 (m, 1H), 8.32-8.25 (m, 1H), 8.14-8.07 (m, 1H), 8.01-7.91 (m, 1H), 7.62-7.57 (m, 1H), 7.43-7.36 (m, 1H), 6.98-6.94 (m, 1H), 6.77 (s, br, 2H), 3.63 (s, 3H), 2.26 (s, 3H)).

### Example 7.2

### 1-(6-Fluoro-2-methyl-pyridin-3-yl)-3-methyl-8-(1-methyl-1H-pyrrolo[2,3-b]pyridin-5-yl)-1,3-dihydro-imidazo[4,5-c]quinolin-2-one

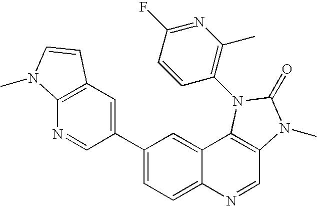

The title compound was synthesized in a similar manner as described for Example 1.1 using 1-methyl-5-(4,4,5,5-tetramethyl-[1,3,2]dioxaborolan-2-yl)-1H-pyrrolo[2,3-b]pyridine and intermediate G.

(HPLC: t_{R} 4.20 min (Method A); M+H=439.1 MS−ES; ¹H-NMR (d₆-DMSO, 600 MHz) 9.06 (s, 1H), 8.36-8.28 (m, 1H), 8.25-8.21 (m, 1H), 8.18-8.13 (m, 1H), 8.04-7.95 (m, 2H), 7.63-7.55 (m, 1H), 7.47-7.40 (m, 1H), 7.11-7.07 (m, 1H), 6.54-6.44 (m, 1H), 3.85 (s, 3H), 3.64 (s, 3H), 2.29 (s, 3H)).

### Example 7.3

### 8-(6-Ethoxy-pyridin-3-yl)-1-(6-fluoro-2-methyl-pyridin-3-yl)-3-methyl-1,3-dihydro-imidazo[4,5-c]quinolin-2-one

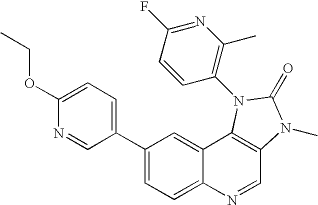

The title compound was synthesized in a similar manner as described for Example 1.1 using 6-ethoxypyridine-3-boronic acid and intermediate G.

(HPLC: t_{R} 4.48 min (Method A); M+H=430.1 MS−ES; ¹H-NMR (d₆-DMSO, 600 MHz) 9.04 (s, 1H), 8.34-8.25 (m, 1H), 8.19-8.11 (m, 2H), 7.93-7.87 (m, 1H), 7.76-7.69 (m, 1H), 7.44-7.37 (m, 1H), 7.04-6.99 (m, 1H), 6.90-6.84 (m, 1H), 4.33 (q, 2H), 3.63 (s, 3H), 2.28 (s, 3H), 1.33 (t, 3H)).

### Example 7.4

### 1-(6-Fluoro-2-methyl-pyridin-3-yl)-3-methyl-8-(6-methylamino-pyridin-3-yl)-1,3-dihydro-imidazo[4,5-c]quinolin-2-one

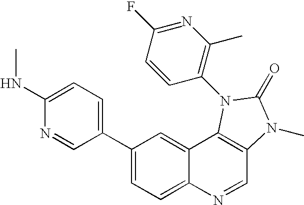

The title compound was synthesized in a similar manner as described for Example 1.1 using 6-(boc-methylamino)pyridine-3-boronic acid pinacol ester and intermediate G. Boc was removed in situ (0.5 mL for 5 min at RT) before purification by dissolving the crude product in TFA (0.5 ml) during 5 min.

(HPLC: t_{R} 3.57 min (Method A); M+H=415.1 MS−ES; ¹H-NMR (d₆-DMSO, 600 MHz) 9.02 (s, 1H), 8.34-8.25 (m, 1H), 8.13-8.06 (m, 1H), 8.03-7.97 (m, 1H), 7.90-7.81 (m, 1H), 7.53-7.44 (m, 1H), 7.44-7.38 (m, 1H), 6.98-6.92 (m, 1H), 6.64-6.51 (m, 1H), 3.63 (s, 3H), 2.82 (d, 3H), 2.28 (s, 3H)).

### Example 7.5

### 8-(5-Ethoxy-6-methoxymethyl-pyridin-3-yl)-1-(6-fluoro-2-methyl-pyridin-3-yl)-3-methyl-1,3-dihydro-imidazo[4,5-c]quinolin-2-one

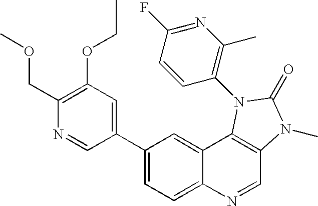

The title compound was synthesized in a similar manner as described for Example 1.1 using 3-ethoxy-2-methoxymethyl-5-(4,4,5,5-tetramethyl-[1,3,2]dioxaborolan-2-yl)-pyridine and intermediate G.

(HPLC: t_{R} 4.00 min (Method A); M+H=474.2 MS−ES; ¹H NMR (600 MHz, DMSO-d₆) δ ppm 9.09 (s, 1H), 8.31 (t, 1H), 8.22 (d, 1H), 8.17 (d, 1H), 8.04 (dd, 1H), 7.42 (dd, 1H), 7.26 (d, 1H), 7.15 (d, 1H), 4.49 (s, 2H), 4.10 (dd, 2H), 3.59-3.69 (m, 3H), 3.26-3.32 (m, 3H), 2.28 (s, 3H), 1.37-1.47 (m, 3H)).

### Example 7.6

### 8-(5-Azetidin-1-yl-pyridin-3-yl)-1-(6-fluoro-2-methyl-pyridin-3-yl)-3-methyl-1,3-dihydro-imidazo[4,5-c]quinolin-2-one

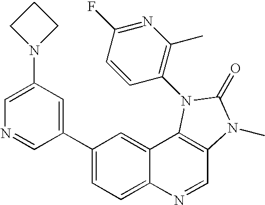

The title compound was synthesized in a similar manner as described for Example 1.1 using 3-Azetidin-1-yl-5-(4,4,5,5-tetramethyl-[1,3,2]dioxaborolan-2-yl)-pyridine and intermediate G.

(HPLC: t_{R} 3.775 min (Method A); M+H=441.2 MS−ES; ¹H NMR (600 MHz, DMSO-d₆) δ ppm 9.08 (s, 1H), 8.31 (t, 1H), 8.15 (d, 1H), 8.04 (m, 1H), 7.97 (dd, 1H) 7.80 (br. s., 1H), 7.43 (dd, 1H), 7.11 (d, 1H), 6.61 (s, 1H) 3.82-4.01 (m, 4H), 3.59-3.70 (m, 3H), 2.36-2.46 (m, 2H), 2.25-2.32 (m, 3H)).

### Example 7.7

### 1-(6-Fluoro-2-methyl-pyridin-3-yl)-8-(5-methoxy-6-methoxymethyl-pyridin-3-yl)-3-methyl-1,3-dihydro-imidazo[4,5-c]quinolin-2-one

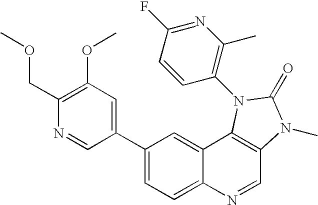

The title compound was synthesized in a similar manner as described for Example 1.1 using 3-methoxy-2-methoxymethyl-5-(4,4,5,5-tetramethyl-[1,3,2]dioxaborolan-2-yl)-pyridine and intermediate G.

(HPLC: t_{R} 3.883 min (Method A); M+H=460.1 MS−ES; ¹H NMR (600 MHz, DMSO-d₆) δ ppm 9.04-9.16 (m, 1H), 8.32 (s, 1H), 8.15-8.23 (m, 2H), 8.05 (dd, 1H), 7.41 (br. s., 1H), 7.31 (d, 1H), 7.15 (d, 1H), 4.48 (s, 2H), 3.88 (s, 3H), 3.57-3.70 (m, 3H), 3.28-3.32 (m, 3H), 2.27-2.32 (m, 3H)).

### Example 7.8

### 1-(6-Fluoro-2-methyl-pyridin-3-yl)-8-[5-(1-hydroxy-1-methyl-ethyl)-pyridin-3-yl]-3-methyl-1,3-dihydro-imidazo[4,5-c]quinolin-2-one

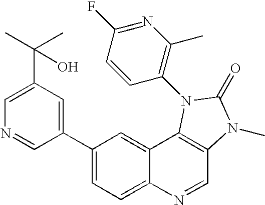

The title compound was synthesized in a similar manner as described for Example 1.1 using 2-[5-(4,4,5,5-Tetramethyl-[1,3,2]dioxaborolan-2-yl)-pyridin-3-yl]-propan-2-ol and intermediate G.

(HPLC: t_{R} 3.667 min (Method A); M+H=444.1 MS−ES; ¹H-NMR (d₆-DMSO, 600 MHz) 9.09 (s, 1H), 8.70 (m, 1H), 8.53 (m, 1H), 8.33-8.32 (m, 1H), 8.19-8.18 (m, 1H), 8.00-7.99 (m, 1H), 7.72-7.71 (m, 1H), 7.39-6.37 (m, 1H), 7.13-7.12 (m, 1H), 5.28 (s, 1H), 3.64 (s, 3H), 2.28 (s, 3H), 1.48 (s, 3H), 1.47 (s, 3H)).

### Stage 7.8.1 2-[5-(4,4,5,5-Tetramethyl-[1,3,2]dioxaborolan-2-yl)-pyridin-3-yl]-propan-2-ol

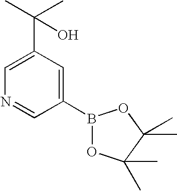

The title compound was synthesized in a similar manner as described for example 2.27, stage 2.27.1 using 2-(5-bromo-pyridin-3-yl)-propan-2-ol (stage 7.8.2) to give the title compound as a crude black sticky oil. (degrading under the HPLC condition: t_{R} 1.87, 3.68 min (Method C); M+H=264 MS−ES).

### Stage 7.8.2 2-(5-Bromo-pyridin-3-yl)-propan-2-ol

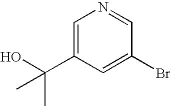

To a solution of 3,5-dibromopyridine (Aldrich, Buchs, Switzerland, 4.6 mmol) in dry THF under Ar was added slowly a ˜2 M solution of isopropylmagnesium chloride in THF (2.76 ml). The RM was stirred for 2 h at rt then was added acetone (6.9 mmol) and the RM was stirred for 2 h at rt. The RM was quenched with brine and extracted with EtOAc (2×). The combined organic layers were washed with brine, dried over Na₂SO₄, filtered and evaporated. The residue was absorbed on silica gel and purified by flash chromatography (heptane/EtOAc 0% to 70%). The fractions containing product were evaporated together to give the title compound as an oil. (HPLC: t_{R} 1.82 min (Method C); M+H=216, 218 MS−ES).

### Example 7.9

### 8-(5-Ethylamino-6-hydroxymethyl-pyridin-3-yl)-1-(6-fluoro-2-methyl-pyridin-3-yl)-3-methyl-1,3-dihydro-imidazo[4,5-c]quinolin-2-one

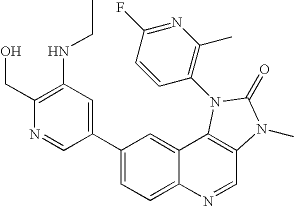

The title compound was synthesized in a similar manner as described for Example 1.1 using acetic acid 3-(tert-butoxycarbonyl-ethyl-amino)-5-(4,4,5,5-tetramethyl-[1,3,2]dioxaborolan-2-yl)-pyridin-2-ylmethyl ester (see example 2.31; stage 2.31.1) and intermediate G. The acetyl protecting group as well as the BOC protecting group were removed together in situ by dissolving the crude product in an aqueous solution of LiOH (1 M, 4 equivalents), stirring for 5 min at RT and the removing the solvent under reduced pressure. (HPLC: t_{R} 3.733 min (Method A); M+H=459.3 MS−ES; ¹H-NMR (d₆-DMSO, 600 MHz) 9.07 (s, 1H), 8.32-8.31 (m, 1H), 8.16-8.14 (m, 1H), 7.98 (m, 1H), 7.87 (m, 1H), 7.42-7.40 (m, 1H), 7.14 (m, 1H), 6.71 (s, 1H), 5.49 (m, 1H), 5.33-5.32 (m, 1H), 4.57-4.56 (m, 2H), 3.64 (s, 3H), 3.12-3.08 (m, 2H), 2.28 (s, 3H), 1.27-1.24 (t, 3H)).

### Example 7.10

### 1-(6-Fluoro-2-methyl-pyridin-3-yl)-8-(6-hydroxymethyl-5-methoxy-pyridin-3-yl)-3-methyl-1,3-dihydro-imidazo[4,5-c]quinolin-2-one

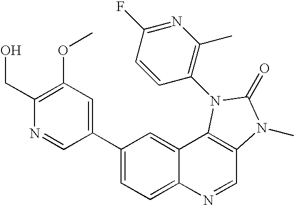

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester and intermediate G. The acetyl protecting group was removed in situ by dissolving the crude product in an aqueous solution of LiOH (1 M, 4 equivalents), stirring for 5 min at RT and the removing the solvent under reduced pressure.

(HPLC: t_{R} 3.650 min (Method A); M+H=446.1 MS−ES; ¹H NMR (600 MHz, DMSO-d₆) δ ppm 9.09 (s, 1H), 8.27-8.38 (m, 1H), 8.18 (dd, 2H), 8.04 (dd, 1H), 7.42 (dd, 1H), 7.28 (d, 1H), 7.14 (d, 1H), 4.94 (t, 1H), 4.55 (d, 2H), 3.87 (s, 3H), 3.64 (s, 3H), 2.29 (s, 3H)).

### Stage 7.10.1. Acetic acid 3-methoxy-5-(4,4,5,5-tetramethyl-[1,3,2]dioxaborolan-2-yl)-pyridin-2-ylmethyl ester

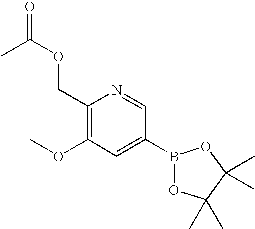

The title compound was prepared as described in example 2.27, stage 27.1 using acetic acid 5-bromo-3-methoxy-pyridin-2-ylmethyl ester (HPLC: t_{R} 3.700 min (Method A); M+H=308.1 MS−ES).

### Stage 7.10.2 Acetic acid 5-bromo-3-methoxy-pyridin-2-ylmethyl ester CR-2607

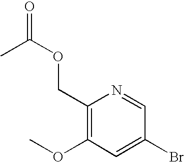

To a mixture of (5-bromo-3-methoxy-pyridin-2-yl)-methanol (Stage 7.10.3, 2.79 mmol) and triethylamine (4.18 mmol) in dichloromethane was added slowly acetyl chloride (3.35 mmol) and the RM was stirred for 1 h at rt. The RM was diluted with dichloromethane, washed with saturated aqueous NaHCO₃, with brine, dried over Na₂SO₄, filtered and evaporated. The residue was absorbed on silica gel and purified by flash chromatography (heptane/EtOAc 0% to 60%). The fractions containing product were evaporated together to give the title compound as a white solid. (HPLC: t_{R} 2.72 min (Method C); M+H=260, 262 MS−ES)

### Stage 7.10.3 (5-Bromo-3-methoxy-pyridin-2-yl)-methanol

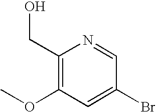

The title compound was synthesized in a similar manner as described for Stage 23.2.3 using methanol as replacement for isopropanol to give the title compound as an off-white solid. (HPLC: t_{R} 1.76 min (Method C); M+H=218, 220 MS−ES).

### Example 7.11

### 1-(6-Fluoro-2-methyl-pyridin-3-yl)-8-(6-hydroxymethyl-5-methylamino-pyridin-3-yl)-3-methyl-1,3-dihydro-imidazo[4,5-c]quinolin-2-one

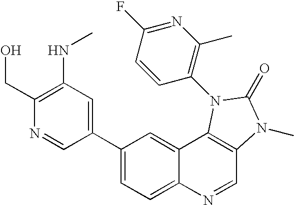

The title compound was synthesized in a similar manner as described for Example 1.1 using acetic acid 3-(tert-butoxycarbonyl-methyl-amino)-5-(4,4,5,5-tetramethyl-[1,3,2]dioxaborolan-2-yl)-pyridin-2-ylmethyl ester (see example 33.2) and intermediate G. The acetyl protecting group as well as the BOC protecting group were removed together in situ by dissolving the crude product in an aqueous solution of LiOH (1 M, 4 equivalents), stirring for 5 min at RT and the removing the solvent under reduced pressure.

(HPLC: t_{R} 3.633 min (Method A); M+H=445.1 MS−ES; ¹H NMR (600 MHz, DMSO-d₆) δ ppm 9.07 (s, 1H), 8.32 (t, 1H), 8.15 (d, 1H), 7.98 (dd, 1H), 7.86 (d, 1H), 7.41 (dd, 1H), 7.14 (d, 1H), 6.66 (d, 1H), 5.68 (d, 1H), 5.23 (t, 1H), 4.54 (d, 2H), 3.64 (s, 3H), 2.77 (d, 3H), 2.28 (s, 3H)).

### Example 7.12

### 8-(5-Fluoro-6-methylamino-pyridin-3-yl)-1-(6-fluoro-2-methyl-pyridin-3-yl)-3-methyl-1,3-dihydro-imidazo[4,5-c]quinolin-2-one

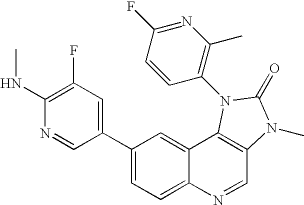

The title compound was synthesized in a similar manner as described for Example 1.1 using [3-fluoro-5-(4,4,5,5-tetramethyl-[1,3,2]dioxaborolan-2-yl)-pyridin-2-yl]-methyl-amine and intermediate G.

(HPLC: t_{R} 3.750 min (Method A); M+H=433.1 MS−ES; ¹H NMR (600 MHz, DMSO-d₆) δ ppm 9.02 (br. s., 1H), 8.29 (t, 1H), 8.08 (d, 1H), 7.79-7.96 (m, 2H), 7.42 (dd, 1H), 7.34 (dd, 1H), 6.87-7.00 (m, 2H), 3.62 (s, 3H), 2.87 (d, 3H), 2.27 (s, 3H)).

### Stage 7.12.1 [3-Fluoro-5-(4,4,5,5-tetramethyl-[1,3,2]dioxaborolan-2-yl)-pyridin-2-yl]-methyl-amine

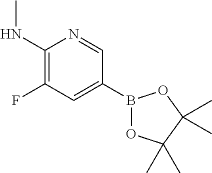

A mixture of (5-bromo-3-fluoro-pyridin-2-yl)-methyl-amine (Stage 7.12.2, 1.255 mmol), bis(pinacolato)-diborane (1.381 mmol), potassium acetate (3.77 mmol) and PdCl₂(dppf) (0.063 mmol) in dioxane (7 ml) was stirred in a closed vial flushed with argon at 90° C. for 15 h. Then the RM was diluted with toluene (7 ml), sonicated and filtered. The solid residue is washed with hot toluene and the filtrated is evaporated to dryness to give the crude title product as a brown oil. (HPLC: t_{R} 3.68 min (Method C); M+H=253 MS−ES).

### Stage 7.12.2 (5-Bromo-3-fluoro-pyridin-2-yl)-methyl-amine

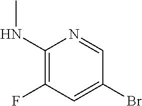

A mixture of 5-bromo-2,3-difluoropyridine (Matrix, Columbia, USA, 2.53 mmol) and 8 M methylamine in EtOH (Aldrich, Buchs, Switzerland, 2 ml, 16 mmol) was sealed in a vial and heated with microwave irradiation at 100° C. for 30 min. The cooled RM was diluted with EtOAc, washed with brine, dried over Na₂SO₄, filtered and evaporated to dryness to give the title product as an off-white solid. (HPLC: t_{R} 1.94 min (Method C); M+H=205, 207, M−H=203, 205 MS−ES).

### Example 7.13

### 1-(6-Fluoro-2-methyl-pyridin-3-yl)-3-methyl-8-(6-methylamino-5-trifluoromethyl-pyridin-3-yl)-1,3-dihydro-imidazo[4,5-c]quinolin-2-one

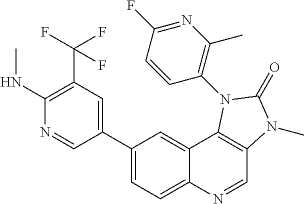

The title compound was synthesized in a similar manner as described for Example 1.1 using Methyl-[5-(4,4,5,5-tetramethyl-[1,3,2]dioxaborolan-2-yl)-3-trifluoromethyl-pyridin-2-yl]-amine and intermediate G.

(HPLC: t_{R} 4.333 min (Method A); M+H=483.1 MS−ES; ¹H NMR (600 MHz, DMSO-d₆) δ ppm 9.03 (s, 1H), 8.50 (d, 1H), 8.29 (t, 1H), 8.10 (d, 1H), 7.97 (dd, 1H), 7.55 (d, 1H), 7.38 (dd, 1H), 6.96 (d, 1H), 6.81 (d, 1H), 3.63 (s, 3H), 2.91 (d, 3H), 2.27 (s, 3H)).

### Stage 7.13.1 Methyl-[5-(4,4,5,5-tetramethyl-[1,3,2]dioxaborolan-2-yl)-3-trifluoromethyl-pyridin-2-yl]-amine

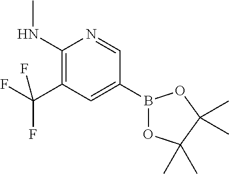

The title compound was prepared as described in example 2.27, stage 27.1 using (5-bromo-3-trifluoromethyl-pyridin-2-yl)-methyl-amine (HPLC: t_{R} 3.458 min (Method A); M+H=303.1 MS−ES).

### Stage 7.13.2. Bromo-3-trifluoromethyl-pyridin-2-yl)-methyl-amine

To a solution of 5-bromo-3-trifluoromethyl-pyridin-2-ylamine (1.928 g, 8 mmol) in DMF anhydrous (10 ml) cooled to 0° C., was added NaH 55% (349 mg, 8 mmol) and stirred at 0° C. for 30 min. Then iodomethane (0.499 ml, 8 mmol) was added, the ice bath was removed and the RM was stirred at rt for 2 h After that, the RM was extracted with EtOAc/H₂O. The organic layers were dried over Na₂SO₄ and evaporated. After filtration, the solvent was removed under reduced pressure to obtain the title compound as a light yellow solid (664 mg). (HPLC: t_{R} 5.625 min (Method A); M+H=257.0 MS−ES).

### Example 7.14

### 2-Amino-5-[1-(6-fluoro-2-methyl-pyridin-3-yl)-3-methyl-2-oxo-2,3-dihydro-1H-imidazo[4,5-c]quinolin-8-yl]-N-methyl-nicotinamide

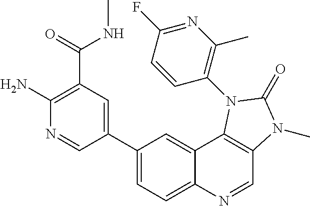

The title compound was synthesized in a similar manner as described for Example 1.1 using 2-amino-N-methyl-5-(4,4,5,5-tetramethyl-[1,3,2]dioxaborolan-2-yl)-nicotinamide and intermediate G.

(HPLC: t_{R} 3.642 min (Method A); M+H=458.1 MS−ES; ¹H NMR (600 MHz, DMSO-d₆) δ ppm 9.03 (br. s., 1H), 8.54 (d, 1H), 8.29 (t, 1H), 8.12 (d, 1H), 7.91-8.07 (m, 3H), 7.38 (d, 1H), 7.26 (br. s., 2H), 7.01 (s, 1H), 3.63 (s, 3H), 2.80 (d, 3H), 2.27 (s, 3H)).

### Stage 7.14.1 2-Amino-N-methyl-5-(4,4,5,5-tetramethyl-[1,3,2]dioxaborolan-2-yl)-nicotinamide

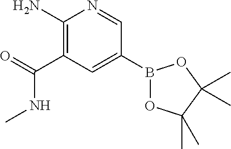

The title compound was synthesized in a similar manner as described for example 2.27, stage 27.1 using 2-amino-5-bromo-N-methyl-nicotinamide (stage 7.14.2, 0.352 mmol) to give the title compound as a crude brown oil. (degrading under the HPLC condition: t_{R} 1.46 min (Method C); M+H=278 MS−ES).

### Stage 7.14.2 2-Amino-5-bromo-N-methyl-nicotinamide

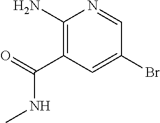

To a solution of 2-amino-5-bromonicotinic acid (Combi-Blocks, San Diego, USA, 1.355 mmol) and triethylamine (0.378 ml) in dichloromethane (10 ml) cooled with an ice-bath was added a solution of trichloromethyl chloroformate (Acros, Basel, Switzerland, 0.677 mmol) in dichloromethane (10 ml). After 40 min stirring at 0° C., was added a solution 8 M of methylamine in EtOH (1.7 ml). The RM was stirred for 30 min at rt then was diluted with dichloromethane, washed with saturated aqueous NaHCO₃, with brine, dried over Na₂SO₄, filtered and evaporated. The residue was taken in DMF and purified by preparative HPLC. The fractions containing product were basified with NaHCO₃, concentrated and extracted with dichloromethane (2×). The combined organic layers were washed with brine, dried over Na₂SO₄, filtered, evaporated and dried under vacuum to give the title compound as a white solid. (HPLC: t_{R} 1.69 min (Method C); M+H=230, 231 MS−ES)

### Example 7.15

### 8-(6-Amino-5-ethoxymethyl-pyridin-3-yl)-1-(6-fluoro-2-methyl-pyridin-3-yl)-3-methyl-1,3-dihydro-imidazo[4,5-c]quinolin-2-one

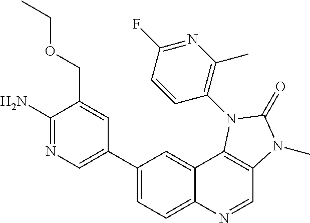

The title compound was synthesized in a similar manner as described for Example 1.1 using 3-ethoxymethyl-5-(4,4,5,5-tetramethyl-[1,3,2]dioxaborolan-2-yl)-pyridin-2-ylamine and intermediate G.

(HPLC: t_{R} 3.792 min (Method A); M+H=459.2 MS−ES; ¹H NMR (600 MHz, DMSO-d₆) δ ppm 9.00 (s, 1H), 8.31 (t, 1H), 8.08 (d, 1H), 7.98 (d, 1H), 7.87 (dd, 1H), 7.41 (dd, 1H), 7.36 (d, 1H), 6.95 (d, 1H), 5.98-6.11 (m, 2H), 4.34 (s, 2H), 3.62 (s, 3H), 3.51 (q, 2H), 2.26 (s, 3H), 1.13-1.26 (m, 3H)).

### Stage 7.15.1 3-Ethoxymethyl-5-(4,4,5,5-tetramethyl-[1,3,2]dioxaborolan-2-yl)-pyridin-2-ylamine

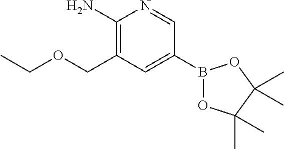

The title compound was synthesized in a similar manner as described for Stage 7.12.1 using 5-bromo-3-ethoxymethyl-pyridin-2-ylamine (stage 7.15.2, 1.082 mmol) to give the title compound as a crude black oil. (degrading under the HPLC condition: t_{R} 1.72 min (Method C); M+H=279 MS−ES).

### Stage 7.15.2 5-Bromo-3-ethoxymethyl-pyridin-2-ylamine

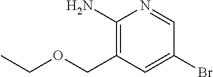

A solution of (2-amino-5-bromo-pyridin-3-yl)-methanol hydrobromide (Apollo, Cheshire, UK, 3.52 mmol) in DMF (15 ml) cooled with an ice-bath was treated with 55% NaH in oil (7.4 mmol) and the RM was stirred for 5 min at 0° C. and 30 min at rt, then was added iodoethane (3.87 mmol) and the RM was stirred for 1 h 15 min at rt. The RM was quenched with water and extracted with EtOAc. The organic layer was washed with saturated aqueous NaHCO₃, with brine (2×), dried over Na₂SO₄, filtered and evaporated. The residue was taken in DMF and purified by preparative HPLC. The fractions containing product were basified with NaHCO₃, concentrated and extracted with CH₂Cl₂ (2×). The combined organic layers were washed with brine, dried over Na₂SO₄, filtered, evaporated and dried under vacuum to give the title compound as a light yellow oil. (HPLC: t_{R} 2.07 min (Method C); M+H=231, 233 MS−ES).

### Example 7.16

### 8-(5-Amino-pyridin-3-yl)-1-(6-fluoro-2-methyl-pyridin-3-yl)-3-methyl-1,3-dihydro-imidazo[4,5-c]quinolin-2-one

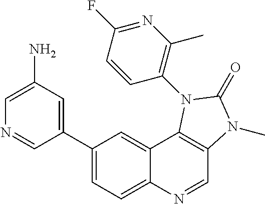

The title compound was synthesized in a similar manner as described for Example 1.1 using 5-(4,4,5,5-tetramethyl-[1,3,2]dioxaborolan-2-yl)-pyridin-3-ylamine and intermediate G.

(HPLC: t_{R} 3.533 min (Method A); M+H=401.1 MS−ES; ¹H NMR (600 MHz, DMSO-d₆) δ ppm 9.07 (s, 1H), 8.25-8.36 (m, 1H), 8.15 (d, 1H), 7.91 (d, 1H), 7.80 (dd, 1H), 7.63 (d, 1H), 7.42 (dd, 1H), 7.05 (d, 1H), 6.96 (t, 1H), 5.47 (s, 2H), 3.64 (s, 3H), 2.27 (s, 3H)).

### Example 7.17

### 8-(6-Amino-5-hydroxymethyl-pyridin-3-yl)-1-(6-fluoro-2-methyl-pyridin-3-yl)-3-methyl-1,3-dihydro-imidazo[4,5-c]quinolin-2-one

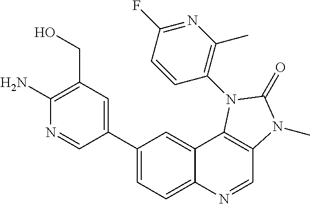

The title compound was synthesized in a similar manner as described for Example 1.1 using acetic acid 2-acetylamino-5-(4,4,5,5-tetramethyl-[1,3,2]dioxaborolan-2-yl)-pyridin-3-ylmethyl ester and intermediate G.

(HPLC: t_{R} 3.517 min (Method A); M+H=431.1 MS−ES; ¹H NMR (600 MHz, DMSO-d₆) δ ppm 9.00 (s, 1H), 8.24-8.37 (m, 1H), 8.08 (d, 1H), 7.90 (d, 1H), 7.87 (dd, 1H), 7.46 (d, 1H), 7.39 (dd, 1H), 6.99 (d, 1H), 6.00 (s, 2H), 5.22 (t, 1H), 4.36 (d, 2H), 3.59-3.67 (m, 3H), 2.26 (s, 3H)).

### Stage 7.17.1 Acetic acid-diacetylamino-5-(4,4,5,5-tetramethyl-[1,3,2]dioxaborolan-2-yl)-pyridin-3-ylmethyl ester

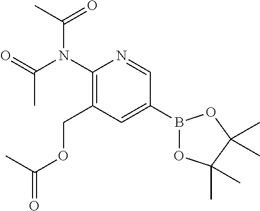

The title compound was synthesized in a similar manner as described for Stage 7.12.1 using acetic acid 2-diacetylamino-5-(4,4,5,5-tetramethyl-[1,3,2]dioxaborolan-2-yl)-pyridin-3-ylmethyl ester (stage 7.17.2, 1.762 mmol) to give the title compound as a crude black oil. (degrading under the HPLC condition: t_{R} 2.05 min (Method C); M+H=377 MS−ES).

### Stage 7.17.2 Acetic acid 2-diacetylamino-5-(4,4,5,5-tetramethyl-[1,3,2]dioxaborolan-2-yl)-pyridin-3-ylmethyl ester

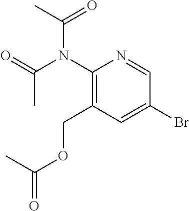

To a suspension of 2-amino-5-bromo-3-hydroxymethylpyridine hydrobromide (Apollo, Cheshire, UK, 1.761 mmol) in dichloromethane (9 ml) were added triethylamine (7.92 mmol) and then acetyl chloride (5.81 mmol). The RM was stirred for 1.5 h at rt then was diluted with dichloromethane, washed with brine (2×), dried over Na₂SO₄, filtered, evaporated and dried under vacuum to give the title compound as an oil. (HPLC: t_{R} 1.69 min (Method C); M+H=329, 331 MS−ES).

### Example 7.18

### 8-(2-Ethylamino-pyrimidin-5-yl)-1-(6-fluoro-2-methyl-pyridin-3-yl)-3-methyl-1,3-dihydro-imidazo[4,5-c]quinolin-2-one

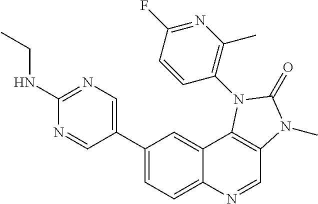

The title compound was synthesized in a similar manner as described for Example 1.1 using ethyl-[5-(4,4,5,5-tetramethyl-[1,3,2]dioxaborolan-2-yl)-pyrimidin-2-yl]-amin and intermediate G.

(HPLC: t_{R} 3.992 min (Method A); M+H=430.1 MS−ES); ¹H NMR (600 MHz, DMSO-d₆) δ ppm 9.02 (s, 1H), 8.21-8.41 (m, 3H), 8.10 (d, 1H), 7.88 (dd, 1H), 7.48 (t, 1H), 7.41 (dd, 1H), 6.93 (d, 1H), 3.63 (s, 3H), 3.24-3.33 (m, 2H), 2.28 (s, 3H), 1.12 (t, 3H)).

### Stage 7.18.1 Ethyl-[5-(4,4,5,5-tetramethyl-[1,3,2]dioxaborolan-2-yl)-pyrimidin-2-yl]-amine

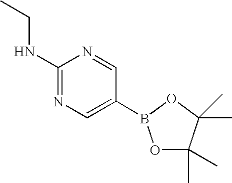

The title compound was synthesized in a similar manner as described for Stage 7.12.1 using (5-bromo-pyrimidin-2-yl)-ethyl-amine (stage 7.18.2, 1.91 mmol) to give the title compound as a crude back oil. (degrading under the HPLC condition: t_{R} 1.54 min (Method C); M+H=250 MS−ES).

### Stage 7.18.2 (5-Bromo-pyrimidin-2-yl)-ethyl-amine

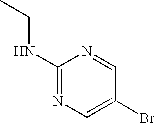

A solution of 5-bromo-2-chloro-pyrimidine (Apollo, Cheshire, UK, 1.37 mmol) and 2 M ethylamine in MeOH (Aldrich, Buchs, Switzerland, 6 ml) was heated with microwave irradiation for 30 min at 150° C. then the reaction mixture was blown off with N₂. The residue was taken in EtOAc, washed with brine, dried over Na₂SO₄, filtered, evaporated and dried under vacuum to give the title compound as a beige solid. (HPLC: t_{R} 2.49 min (Method C);

### M+H=202, 204 MS−ES).

### Example 8.1

### 8-(6-Amino-5-trifluoromethyl-pyridin-3-yl)-1-(2,6-dimethoxy-pyridin-3-yl)-3-methyl-1,3-dihydro-imidazo[4,5-c]quinolin-2-one

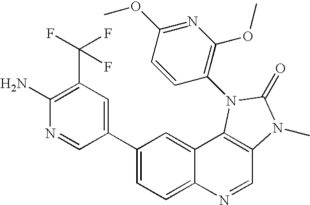

The title compound was synthesized in a similar manner as described for Example 1.1 using 5-(4,4,5,5-tetramethyl-1,3,2-dioxoborolan-2-yl)-1H-pyrrolo[2,3-b]pyridine and intermediate H.

(HPLC: t_{R} 4.31 min (Method A); M+H=497.0 MS−ES; ¹H-NMR (d₆-DMSO, 600 MHz) 8.96 (s, 1H), 8.39 (s, 1H), 8.08-8.07 (m, 1H), 8.02-8.01 (m, 1H), 7.96-7.94 (m, 1H), 7.61 (s, 1H), 7.22 (m, 1H), 6.77 (s, br, 2H), 6.68-6.67 (m, 1H), 3.99 (s, 3H), 3.80 (s, 3H), 3.60 (s, 3H)).

### Example 8.2

### 1-(2,6-Dimethoxy-pyridin-3-yl)-8-(6-ethoxy-pyridin-3-yl)-3-methyl-1,3-dihydro-imidazo[4,5-c]quinolin-2-one

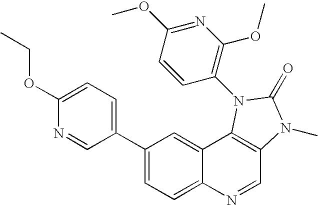

The title compound was synthesized in a similar manner as described for Example 1.1 using 6-ethoxypyridine-3-boronic acid and intermediate H.

(HPLC: t_{R} 4.73 min (Method A); M+H=458.2 MS−ES; ¹H-NMR (d₆-DMSO, 600 MHz) 8.99 (s, 1H), 8.20 (m, 1H), 8.11-8.09 (m, 1H), 8.02-8.01 (m, 1H), 7.90-7.88 (m, 1H), 7.73-7.71 (m, 1H), 7.22-7.21 (m, 1H), 6.88-6.87 (m, 1H), 6.71-6.70 (m, 1H), 4.35-4.32 (m, 2H), 4.02 (s, 3H) 3.79 (s, 3H), 3.60 (s, 3H) 1.35-1.32 (m, 3H)).

### Example 8.3

### 1-(2,6-Dimethoxy-pyridin-3-yl)-3-methyl-8-(6-methylamino-pyridin-3-yl)-1,3-dihydro-imidazo[4,5-c]quinolin-2-one

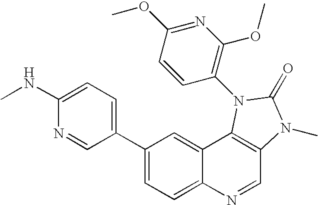

The title compound was synthesized in a similar manner as described for Example 1.1 using 6-(boc-methylamino)pyridine-3-boronic acid pinacol ester and intermediate H. Boc was removed in situ (0.5 mL for 5 min at RT) before purification by dissolving the crude product in TFA (0.5 ml) during 5 min.

(HPLC: t_{R} 3.74 min (Method A); M+H=443.1 MS−ES; ¹H-NMR (d₆-DMSO, 600 MHz) 8.94 (s, 1H), 8.06-8.01 (m, 3H), 7.85-7.83 (m, 1H), 7.43 (m, 1H), 7.16 (m, 1H), 6.72-6.70 (m, 1H), 6.53 (m, 1H), 4.03 (s, 3H), 3.80 (s, 3H), 3.60 (s, 3H), 2.81 (m, 3H)).

### Example 8.4

### 1-(2,6-Dimethoxy-pyridin-3-yl)-3-methyl-8-(2-methylamino-pyrimidin-5-yl)-1,3-dihydro-imidazo[4,5-c]quinolin-2-one

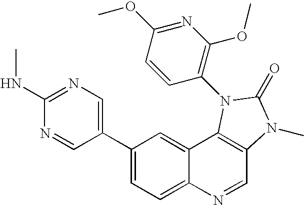

The title compound was synthesized in a similar manner as described for Example 1.1 using methyl-[5-(4,4,5,5-tetramethyl-[1,3,2]dioxaborolan-2-yl)-pyrimidin-2-yl]-amine and intermediate H.

(HPLC: t_{R} 4.03 min (Method A); M+H=444.1 MS−ES, ¹H-NMR (d₆-DMSO, 600 MHz) 8.99 (s, 1H), 8.32 (m, 2H), 8.08-8.07 (m, 1H), 8.03-8.01 (m, 1H), 7.89-7.87 (m, 1H), 7.42-7.41 (m, 1H), 7.17 (m, 1H), 6.71-6.70 (m, 1H), 4.01 (s, 3H), 3.79 (s, 3H), 3.60 (s, 3H), 2.84-2.83 (m, 3H)).

### Example 8.5

### 1-(2,6-Dimethoxy-pyridin-3-yl)-8-(2-dimethylamino-pyrimidin-5-yl)-3-methyl-1,3-dihydro-imidazo[4,5-c]quinolin-2-one

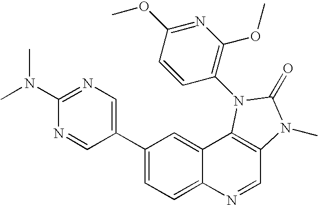

The title compound was synthesized in a similar manner as described for Example 1.1 using di methyl-[5-(4,4,5,5-tetramethyl-[1,3,2]dioxaborolan-2-yl)-pyrimidin-2-yl]-amine and intermediate H.

(HPLC: t_{R} 4.37 min (Method A); M+H=458.1 MS−ES, ¹H-NMR (d₆-DMSO, 600 MHz) 8.96 (s, 1H), 8.41 (m, 2H), 8.09-8.07 (m, 1H), 8.02-8.01 (m, 1H), 7.89-7.87 (m, 1H), 7.16 (m, 1H), 6.72-6.71 (m, 1H), 4.03 (s, 3H), 3.79 (s, 3H), 3.60 (s, 3H), 3.16 (s, 6H)).

### Example 8.6

### 1-(2,6-Dimethoxy-pyridin-3-yl)-3-methyl-8-(1-methyl-1H-pyrrolo[2,3-b]pyridin-5-yl)-1,3-dihydro-imidazo[4,5-c]quinolin-2-one

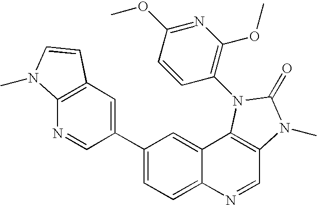

The title compound was synthesized in a similar manner as described for Example 1.1 using 1-methyl-5-(4,4,5,5-tetramethyl-[1,3,2]dioxaborolan-2-yl)-1H-pyrrolo[2,3-b]pyridine and intermediate H.

(HPLC: t_{R} 4.45 min (Method A); M+H=466.8 MS−ES, ¹H-NMR (d₆-DMSO, 600 MHz) 8.98 (s, 1H), 8.31 (m, 1H), 8.13-8.12 (m, 1H), 8.04-8.03 (m, 1H), 7.98-7.97 (m, 2H), 7.60 (m, 1H), 7.31 (m, 1H), 6.71-6.70 (m, 1H), 6.49 (m, 1H), 4.04 (s, 3H), 3.85 (s, 3H), 3.81 (s, 3H), 3.61 (s, 3H)).

### Example 8.7

### 1-(2,6-Dimethoxy-pyridin-3-yl)-8-(5-ethylamino-6-methyl-pyridin-3-yl)-3-methyl-1,3-dihydro-imidazo[4,5-c]quinolin-2-one

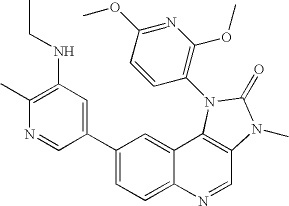

The title compound was synthesized in a similar manner as described for Example 1.1 using ethyl-[2-methyl-5-(4,4,5,5-tetramethyl-[1,3,2]dioxaborolan-2-yl)-pyridin-3-yl]-amine and intermediate H.

(HPLC: t_{R} 3.89 min (Method A); M+H=471.1 MS−ES, ¹H-NMR (d₆-DMSO, 600 MHz) 8.99 (s, 1H), 8.11-8.09 (m, 1H), 8.03-8.02 (m, 1H), 7.94-7.92 (m, 1H), 7.86 (m, 1H), 7.39 (m, 1H), 6.69-6.67 (m, 1H), 6.64 (m, 1H), 5.23-5.22 (m, 1H), 3.99 (s, 3H), 3.80 (s, 3H), 3.60 (s, 3H), 3.08-3.05 (m, 2H), 2.32 (s, 3H), 1.3-1.2 (m, 3H).

### Example 8.8

### 1-(2,6-Dimethoxy-pyridin-3-yl)-8-[5-(2-methoxy-ethoxy)-pyridin-3-yl]-3-methyl-1,3-dihydro-imidazo[4,5-c]quinolin-2-one

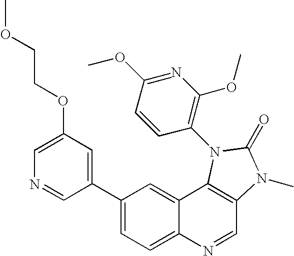

The title compound was synthesized in a similar manner as described for Example 1.1 using 3-(2-Methoxy-ethoxy)-5-(4,4,5,5-tetramethyl-[1,3,2]dioxaborolan-2-yl)-pyridine and intermediate H.

(HPLC: t_{R} 3.98 min (Method A); M+H=488.1 MS−ES, ¹H-NMR (d₆-DMSO, 600 MHz) 9.13 (s, 1H), 8.33-8.32 (m, 1H), 8.28 (m, 1H), 8.20-8.19 (m, 1H), 8.11-8.10 (m, 1H), 8.05-8.04 (m, 1H), 7.41 (m, 1H), 7.36 (s, 1H), 6.72-6.71 (m, 1H), 4.22-4.20 (m, 2H), 4.01 (s, 3H), 3.81 (s, 3H), 3.73-3.71 (m, 2H), 3.63 (s, 3H), 3.35 (s, 3H)).

### Stage 8.8.1. 3-(2-Methoxy-ethoxy)-5-(4,4,5,5-tetramethyl-[1,3,2]dioxaborolan-2-yl)-pyridine

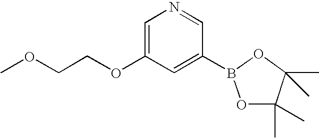

The title compound was synthesized in a similar manner as described for stage 1.3.1 using 3-bromo-5-(2-methoxy-ethoxy)-pyridine (Stage 8.1.2) to give the title compound as a brown oil. (HPLC: t_{R} 2.10 min (Method C)).

### Stage 8.1.2 3-Bromo-5-(2-methoxy-ethoxy)-pyridine

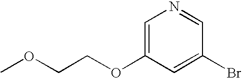

A mixture of 3-bromo-5-hydroxypyridine (Aldrich, Buchs, Switzerland, 611 mg, 3.51 mmol), potassium carbonate (971 mg, 7.02 mmol) and 2-bromoethyl methyl ether (537 mg, 3.86 mmol) in 30 ml DMF was stirred for 14 h at rt and for 2 h at 80° C. The reaction mixture was quenched with water and extracted with EtOAc (2×). The organic layers were washed with brine (3×), dried over Na₂SO₄, filtered and evaporated. The residue was purified by flash chromatography (dichloromethane/MeOH 0% to 3%) to give the title compound as an oil. (HPLC: t_{R} 2.38 min (Method C); M+H=232, 234 MS−ES).

### Example 8.9

### 1-(2,6-Dimethoxy-pyridin-3-yl)-8-[5-(2-methoxy-ethoxy)-6-methyl-pyridin-3-yl]-3-methyl-1,3-dihydro-imidazo[4,5-c]quinolin-2-one

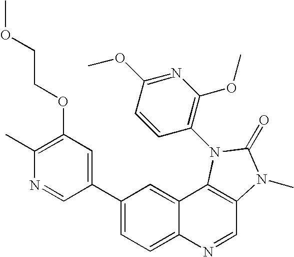

The title compound was synthesized in a similar manner as described for Example 1.1 using 3-(2-methoxy-ethoxy)-2-methyl-5-(4,4,5,5-tetramethyl-[1,3,2]dioxaborolan-2-yl)-pyridine and intermediate H.

(HPLC: t_{R} 3.90 min (Method A); M+H=502.1 MS−ES, ¹H-NMR (d₆-DMSO, 600 MHz) 9.25 (s, 1H), 8.28-8.27 (m, 2H), 8.23 (m, 1H), 8.07-8.06 (m, 1H), 7.48 (s, 1H), 7.38 (s, 1H), 6.74-6.73 (m, 1H), 4.22-4.16 (m, 2H), 4.00 (s, 3H), 3.82 (s, 3H), 3.78-3.75 (m, 2H), 3.64 (s, 3H), 3.38 (s, 3H), 2.44 (s, 3H)).

### Stage 8.9.1. 3-(2-Methoxy-ethoxy)-2-methyl-5-(4,4,5,5-tetramethyl-[1,3,2]dioxaborolan-2-yl)-pyridine

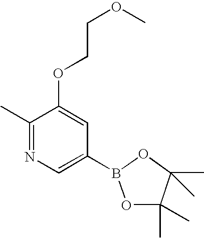

The title compound was synthesized in a similar manner are described for stage 2.19.1-2 using 2-methoxyethanol instead of isopropanol. (HPLC: tR 2.05 min (Method C); M+H=294 MS−ES)

### Example 8.10

### 1-(2,6-Dimethoxy-pyridin-3-yl)-8-imidazo[1,2-a]pyridin-6-yl-3-methyl-1,3-dihydro-imidazo[4,5-c]quinolin-2-one

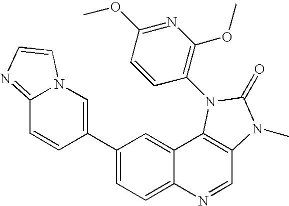

The title compound was synthesized in a similar manner as described for Example 1.1 using imidazo[1,2-A]pyridine-6-boronic acid and intermediate H.

(HPLC: t_{R} 3.68 min (Method A); M+H=453.1 MS−ES).

### Example 8.11

### 8-(5-Azetidin-1-yl-pyridin-3-yl)-1-(2,6-dimethoxy-pyridin-3-yl)-3-methyl-1,3-dihydro-imidazo[4,5-c]quinolin-2-one

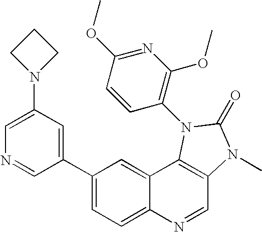

The title compound was synthesized in a similar manner as described for Example 1.1 using 3-azetidin-1-yl-5-(4,4,5,5-tetramethyl-[1,3,2]dioxaborolan-2-yl)-pyridine (see example 7.6; stage 7.6.1) and intermediate H.

(HPLC: t_{R} 4.033 min (Method A); M+H=469.2 MS−ES)

### Example 8.12

### 1-(2,6-Dimethoxy-pyridin-3-yl)-8-(5-methoxy-6-methoxymethyl-pyridin-3-yl)-3-methyl-1,3-dihydro-imidazo[4,5-c]quinolin-2-one

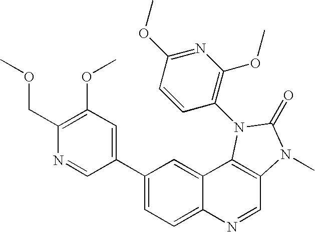

The title compound was synthesized in a similar manner as described for Example 1.1 using 3-methoxy-2-methoxymethyl-5-(4,4,5,5-tetramethyl-[1,3,2]dioxaborolan-2-yl)-pyridine (see example 2.30; stage 2.30.1) and intermediate H.

(HPLC: t_{R} 4.033 min (Method A); M+H=488.1 MS−ES).

### Example 8.13

### 8-(6-Dimethoxymethyl-5-methoxy-pyridin-3-yl)-1-(2,6-dimethoxy-pyridin-3-yl)-3-methyl-1,3-dihydro-imidazo[4,5-c]quinolin-2-one

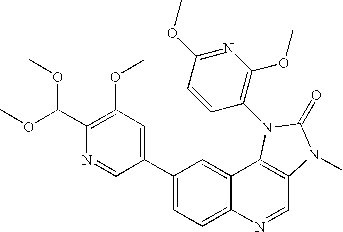

The title compound was synthesized in a similar manner as described for Example 1.1 using 2-dimethoxymethyl-3-methoxy-5-(4,4,5,5-tetramethyl-[1,3,2]dioxaborolaN-2-yl)-pyridine (see example 2.27; stage 2.27.1) and intermediate H.

(HPLC: t_{R} 4.142 min (Method A); M+H=518.2 MS−ES).

### Example 8.14

### 1-(2,6-Dimethoxy-pyridin-3-yl)-8-(6-hydroxymethyl-5-methoxy-pyridin-3-yl)-3-methyl-1,3-dihydro-imidazo[4,5-c]quinolin-2-one

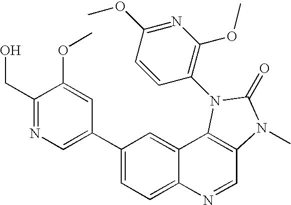

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester (see example 7.10; stage 7.10.1) and intermediate H. The acetyl protecting group was removed in situ by dissolving the crude product in an aqueous solution of LiOH (1 M, 4 equivalents), stirring for 5 min at RT and the removing the solvent under reduced pressure.

(HPLC: t_{R} 3.808 min (Method A); M+H=474.1 MS−ES).

### Example 9.1

### 8-(6-Ethoxy-pyridin-3-yl)-1-(2-methoxy-6-piperazin-1-yl-pyridin-3-yl)-3-methyl-1,3-dihydro-imidazo[4,5-c]quinolin-2-one

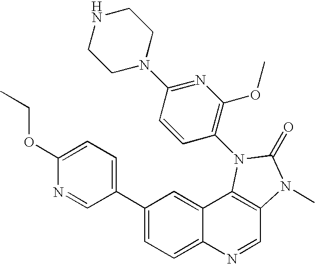

The title compound was synthesized in a similar manner as described for Example 1.1 using 6-ethoxypyridine-3-boronic acid and intermediate I. Boc was removed in situ (0.5 mL for 5 min at RT) before purification by dissolving the crude product in TFA (0.5 ml) during 5 min.

(HPLC: t_{R} 3.83 min (Method A); M+H=512.1 MS−ES; ¹H-NMR (d₆-DMSO, 600 MHz) 8.96 (s, 1H), 8.28 (m, 1H), 8.10-8.08 (m, 1H), 7.92-7.91 (m, 1H), 7.83-7.82 (m, 1H), 7.70-7.69 (m, 1H), 7.33 (m, 1H), 6.88-6.86 (m, 1H), 6.68-6.66 (m, 1H), 4.36-4.34 (m, 2H), 3.77-3.74 (m, 4H), 3.71 (s, 3H), 3.59 (s, 3H), 3.14-3.12 (m, 4H), 1.36-1.33 (m, 3H)).

### Stage 9.1.1 4-(5-Amino-6-methoxy-pyridin-2-yl)-piperazine-1-carboxylic acid tert-butyl ester

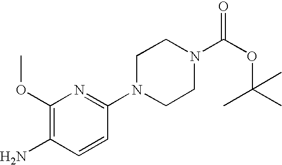

To a solution of 4-(6-Methoxy-5-nitro-pyridin-2-yl)-piperazine-1-carboxylic acid tert-butyl ester (Stage 9.1.2, 857 mg, 2.53 mmol) in MeOH/THF=1:1 (20 ml total) was added Ra-Ni (400 mg, 2.53 mmol) and the RM was stirred under H₂ atmosphere at rt for 9 h. The RM was then filtered over Celite and washed several times with MeOH. The filtrate was evaporated to dryness to give the title compound as a dark purple powder.

(HPLC: t_{R} 4.27 min (Method A); M+H=309.2.

### Stage 9.1.2 4-(6-Methoxy-5-nitro-pyridin-2-yl)-piperazine-1-carboxylic acid tert-butyl ester

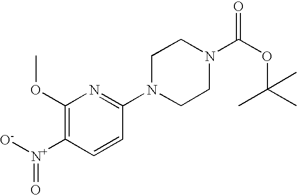

Under an atmosphere of Argon was introduced Na metal (131 mg; 5.7 mMol) into 2 ml of MeOH. After dissolving of the metal, this solution was added dropwise over a period of 15 min into a solution of 2,6-dichloro-3-nitropyridine (1.0 g; 5.18 mMol) in MeOH (3 ml) at 0° C. The reaction mixture was kept stirring for 1 h at 0° C. then for 2 h at RT. To this mixture piperazine-1-carboxylic acid tert-butyl ester (2.316 g; 12.44 mMol) was added and the reaction stirred for 2 h at RT. The reaction mixture was poured onto water (20 mL), the yellow precipitate filtered off and washed with water. Purification was done by chromatography on silica gel (combiflash 40 g; eluting with heptane/EtOAc 0% to 80%) to obtain the desired product as a yellow solid plus the undesired regioisomer. Title compound: (HPLC: t_{R} 3.55 min (Method C). ¹H-NMR (d₆-DMSO, 600 MHz) 8.25-8.24 (m, 1H), 6.53-6.51 (m, 1H), 3.96 (s, 3H), 3.82-3.69 (m, 4H), 3.52-3.37 (m, 4H), 1.41 (s, 9H)).

### Example 9.2

### 8-(6-Amino-5-trifluoromethyl-pyridin-3-yl)-1-(2-methoxy-6-piperazin-1-yl-pyridin-3-yl)-3-methyl-1,3-dihydro-imidazo[4,5-c]quinolin-2-one

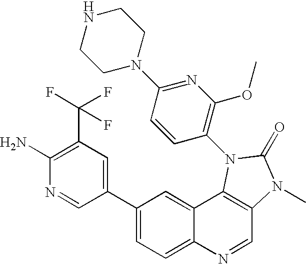

The title compound was synthesized in a similar manner as described for Example 1.1 using 5-(4,4,5,5-tetramethyl-1,3,2-dioxoborolan-2-yl)-1H-pyrrolo[2,3-b]pyridine and intermediate I. Boc was removed in situ (0.5 mL for 5 min at RT) before purification by dissolving the crude product in TFA (0.5 ml) during 5 min.

(HPLC: t_{R} 3.70 min (Method A); M+H=551.1 MS−ES; ¹H-NMR (d₆-DMSO, 600 MHz) 8.94 (s, 1H), 8.36 (s, 1H), 8.07-8.06 (m, 1H), 7.95 (m, 1H), 7.83-7.81 (m, 1H), 7.69 (s, 1H), 7.32 (s, 1H), 6.77 (s, br, 2H), 6.66-6.64 (m, 1H), 3.82-3.81 (m, 4H), 3.72 (s, 3H), 3.59 (s, 3H), 3.17 (m, 4H)).

### Example 9.3

### 1-(2-Methoxy-6-piperazin-1-yl-pyridin-3-yl)-3-methyl-8-(1-methyl-1H-pyrrolo[2,3-b]pyridin-5-yl)-1,3-dihydro-imidazo[4,5-c]quinolin-2-one

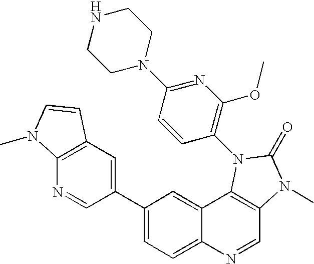

The title compound was synthesized in a similar manner as described for Example 1.1 using 1-methyl-5-(4,4,5,5-tetramethyl-[1,3,2]dioxaborolan-2-yl)-1H-pyrrolo[2,3-b]pyridine and intermediate I. Boc was removed in situ (0.5 mL for 5 min at RT) before purification by dissolving the crude product in TFA (0.5 ml) during 5 min.

(HPLC: t_{R} 3.77 min (Method A); M+H=521.0 MS−ES; ¹H-NMR (d₆-DMSO, 600 MHz) 8.97 (s, 1H), 8.68 (s, br, 1H), 8.33 (m, 1H), 8.13-8.11 (m, 1H), 8.02 (m, 1H), 7.99-7.97 (m, 1H), 7.89-7.88 (m, 1H), 7.61-7.60 (m, 1H), 7.45 (m, 1H), 6.73-6.72 (m, 1H), 6.52-6.51 (m, 1H), 3.88-3.86 (m, 4H), 3.86 (s, 3H) 3.75 (s, 3H), 3.60 (s, 3H), 3.27-3.26 (m, 4H)).

### Example 9.4

### 8-(2-Dimethylamino-pyrimidin-5-yl)-1-(2-methoxy-6-piperazin-1-yl-pyridin-3-yl)-3-methyl-1,3-dihydro-imidazo[4,5-c]quinolin-2-one

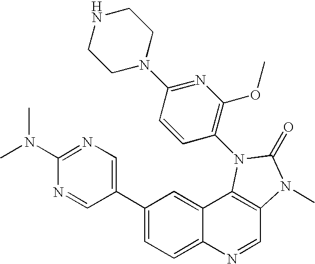

The title compound was synthesized in a similar manner as described for Example 1.1 using 2-dimethylamino-pyrimidin-5-boronic acid pinacol ester. Boc was removed in situ (0.5 mL for 5 min at RT) before purification by dissolving the crude product in TFA (0.5 ml) during 5 min.

(HPLC: t_{R} 3.667 min (Method A); M+H=512.0 MS−ES; ¹H NMR (600 MHz, DMSO-d₆) δ ppm 8.94 (s, 1H), 8.44 (s, 2H), 8.07 (d, 1H), 7.88 (dd, 1H), 7.81 (d, 1H), 7.27 (d, 1H), 6.67 (d, 1H), 3.73-3.89 (m, 4H), 3.71 (s, 3H), 3.59 (s, 3H), 3.17 (s, 6H), 3.06-3.15 (m, 4H)).

### Example 10.1

### 8-(2-Dimethylamino-pyrimidin-5-yl)-1-[2-methoxy-6-(4-methyl-piperazin-1-yl)-pyridin-3-yl]-3-methyl-1,3-dihydro-imidazo[4,5-c]quinolin-2-one

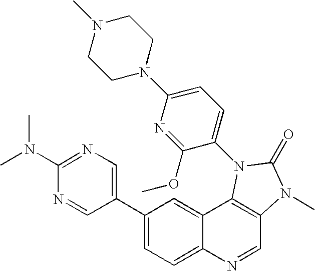

The title compound was synthesized in a similar manner as described for Example 1.1 using Dimethyl-[5-(4,4,5,5-tetramethyl-[1,3,2]dioxaborolan-2-yl)-pyrimidin-2-yl]-amine and intermediate J.

(HPLC: t_{R} 3.71 min (Method A); M+H=526.1 MS−ES; ¹H-NMR (d₆-DMSO, 600 MHz) 10.69 (s, 1H; NH+), 9.11 (s, 1H), 8.46 (s, 2H), 8.17 (m, 1H), 8.03-8.02 (m, 1H), 7.90-7.89 (m, 1H), 7.37 (m, 1H), 6.79-6.77 (m, 1H), 4.57 (m, 2H), 3.74 (s, 3H), 3.61 (s, 3H), 3.59-3.55 (m, 2H), 3.40 (m, 2H), 3.18 (s, 6H), 3.15 (m, 2H), 2.85 (m, 3H)).

### Stage 10.1.1 8-Bromo-1-[2-methoxy-6-(4-methyl-piperazin-1-yl)-pyridin-3-yl]-3-methyl-1,3-dihydro-imidazo[4,5-c]quinolin-2-one

4-[5-(8-Bromo-3-methyl-2-oxo-2,3-dihydro-imidazo[4,5-c]quinolin-1-yl)-6-methoxy-pyridin-2-yl]-piperazine-1-carboxylic acid tert-butyl ester (Intermediate I) (342 mg; 0.6 mMol) was dissolved in TFA (0.919 mL; 12 mMol) and kept at RT for 5 min. To this solution was added a solution of NaHCO₃ to adjust to pH 8-9 and the aqueous solution was extracted with EtOAc. The combined organic layers were washed with water (2×) and the solvent was removed under reduced pressure. The crude product was dried over night at high vacuum. The free amine was dissolved in anhydrous DMF (10 mL) and cooled to 0° C. NaH (79 mg; 1.8 mMol) was added and the mixture kept stirring for 30 min. at 0° C. CH₃I (170 mg; 1.2 mmol) was added, the ice bath was removed, and the mixture kept stirring for 15 min. The reaction mixture was taken up into EtOAc (100 mL), and extracted with water (2×). After removal of the organic under reduced pressure, the crude product was purified by chromatography over silicagel (eluting with DCM/MeOH; 94/6). Fractions containing product were evaporated together to lyophilized under high vacuum to give the title compound as a bright yellow solid. (HPLC: t_{R} 3.38 min (Method A); M+H=484.8 MS−ES).

### Example 10.2

### 1-[2-Methoxy-6-(4-methyl-piperazin-1-yl)-pyridin-3-yl]-3-methyl-8-(6-methylamino-pyridin-3-yl)-1,3-dihydro-imidazo[4,5-c]quinolin-2-one

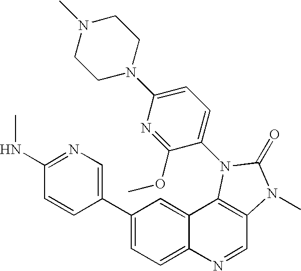

The title compound was synthesized in a similar manner as described for Example 1.1 using 6-(boc-methylamino)pyridine-3-boronic acid pinacol ester and intermediate J. Boc was removed in situ (0.5 mL for 5 min at RT) before purification by dissolving the crude product in TFA (0.5 ml) during 5 min.

(HPLC: t_{R} 3.44 min (Method A); M+H=511.1 MS−ES).

### Example 11.1

### 8-(6-Ethoxy-pyridin-3-yl)-1-(6-methoxy-2-methyl-pyridin-3-yl)-3-methyl-1,3-dihydro-imidazo[4,5-c]quinolin-2-one

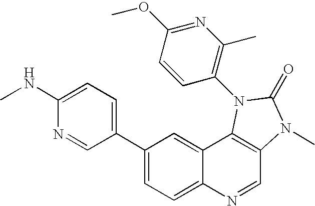

The title compound was synthesized in a similar manner as described for Example 1.1 using 6-ethoxypyridine-3-boronic acid and intermediate K.

(HPLC: t_{R} 4.67 min (Method A); M+H=442.1 MS−ES; ¹H-NMR (d₆-DMSO, 600 MHz) 9.03 (s, 1H), 8.17 (m, 1H), 8.13-8.11 (m, 1H), 7.93-7.92 (m, 1H), 7.90-7.88 (m, 1H), 7.70-7.68 (m, 1H), 7.06 (m, 1H), 6.99-6.97 (m, 1H), 6.87-6.85 (m, 1H), 4.35-4.33 (m, 2H), 3.99 (s, 3H), 3.63 (s, 3H), 2.21 (s, 3H), 1.34-1.32 (m, 3H)).

### Example 11.2

### 1-(6-Methoxy-2-methyl-pyridin-3-yl)-3-methyl-8-(6-methylamino-pyridin-3-yl)-1,3-dihydro-imidazo[4,5-c]quinolin-2-one

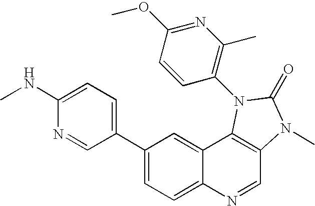

The title compound was synthesized in a similar manner as described for Example 1.1 using 6-(boc-methylamino)pyridine-3-boronic acid pinacol ester and intermediate K. Boc was removed in situ (1.0 mL for 5 min at RT) before purification by dissolving the crude product in TFA (0.5 ml) during 5 min.

(HPLC: t_{R} 3.64 min (Method A); M+H=427.1 MS−ES; ¹H-NMR (d₆-DMSO, 600 MHz) 12.58 (s, br, 1H) 8.99 (s, 1H), 8.07-8.04 (m, 2H), 7.95-7.92 (m, 1H), 7.85-7.84 (m, 1H), 7.40 (m, 1H) 7.01-6.98 (m, 2H), 6.52-6.51 (m, 1H), 4.02-4.01 (m, 3H), 3.63-3.62 (m, 3H), 2.81-2.80 (m, 3H), 2.21 (s, 3H)).

### Example 11.3

### 1-(6-Methoxy-2-methyl-pyridin-3-yl)-3-methyl-8-(2-methylamino-pyrimidin-5-yl)-1,3-dihydro-imidazo[4,5-c]quinolin-2-one

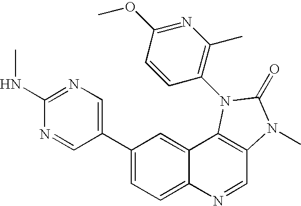

The title compound was synthesized in a similar manner as described for Example 1.1 using 2-methylaminopyrimidin-5-boronic acid PE and intermediate K.

(HPLC: t_{R} 3.90 min (Method A); M+H=428.2 MS−ES; ¹H-NMR (d₆-DMSO, 600 MHz) 9.00 (s, 1H), 8.37 (m, 1H), 8.27 (m, 1H), 8.10-8.08 (m, 1H), 7.93-7.92 (m, 1H), 7.89-7.87 (m, 1H), 7.43-7.40 (m, 1H) 7.03 (m, 1H), 6.99-6.97 (m, 1H), 3.98 (s, 3H), 3.62 (s, 3H), 2.83-2.81 (m, 3H), 2.21 (s, 3H)).

### Example 11.4

### 8-(2-Dimethylamino-pyrimidin-5-yl)-1-(6-methoxy-2-methyl-pyridin-3-yl)-3-methyl-1,3-dihydro-imidazo[4,5-c]quinolin-2-one

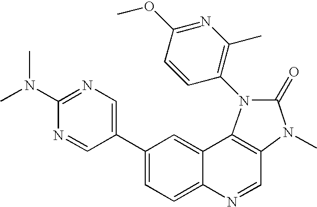

The title compound was synthesized in a similar manner as described for Example 1.1 using 6-dimethyl-[5-(4,4,5,5-tetramethyl-[1,3,2]dioxaborolan-2-yl)-pyrimidin-2-yl]-amine and intermediate K.

(HPLC: t_{R} 4.26 min (Method A); M+H=442.1 MS−ES; ¹H-NMR (d₆-DMSO, 600 MHz) 9.00 (s, 1H), 8.38 (s, 2H), 8.10-8.09 (m, 1H), 7.92-7.91 (m, 1H), 7.89-7.87 (m, 1H), 7.02 (s, 1H), 6.99-6.98 (m, 1H), 3.99 (s, 3H), 3.62 (s, 3H), 3.16 (s, 6H), 2.21 (s, 3H)).

### Example 11.5

### 1-(6-Methoxy-2-methyl-pyridin-3-yl)-8-(6-methoxy-pyridin-3-yl)-3-methyl-1,3-dihydro-imidazo[4,5-c]quinolin-2-one

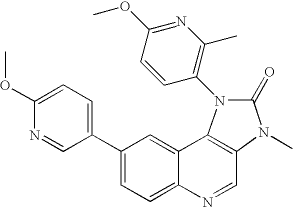

The title compound was synthesized in a similar manner as described for Example 1.1 using 6-2-methoxy-5-pyridine boronic acid and intermediate K.

(HPLC: t_{R} 4.45 min (Method A); M+H=428.1 MS−ES; ¹H-NMR (d₆-DMSO, 600 MHz) 9.03 (s, 1H), 8.19 (m, 1H), 8.13-8.12 (m, 1H), 7.94-7.92 (m, 1H), 7.91-7.89 (m, 1H), 7.71-7.70 (m, 1H), 7.08 (m, 1H), 6.99-6.97 (m, 1H), 6.90-6.89 (m, 1H), 3.99 (s, 3H), 3.89 (s, 3H), 3.63 (s, 3H), 2.21 (s, 3H)).

### Example 11.6

### 8-(6-Amino-5-trifluoromethyl-pyridin-3-yl)-1-(6-methoxy-2-methyl-pyridin-3-yl)-3-methyl-1,3-dihydro-imidazo[4,5-c]quinolin-2-one

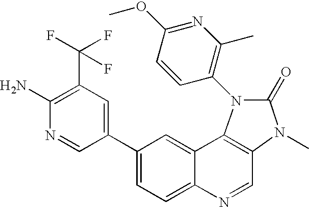

The title compound was synthesized in a similar manner as described for Example 1.1 using 5-(4,4,5,5-tetramethyl-1,3,2-dioxoborolan-2-yl)-1H-pyrrolo[2,3-b]pyridine and intermediate K.

(HPLC: t_{R} 4.217 min (Method A); M+H=481.0 MS−ES; ¹H-NMR (d₆-DMSO, 600 MHz) 9.01 (s, 1H), 8.36 (m, 1H), 8.10-8.08 (m, 1H), 7.94-7.93 (m, 2H), 7.58-7.57 (m, 1H), 7.07 (m, 1H), 6.96-6.95 (m, 1H), 6.76 (s, br, 2H), 3.96 (s, 3H), 3.62 (s, 3H), 2.20 (s, 3H)).

### Example 11.7

### 8-(5-Ethylamino-6-methyl-pyridin-3-yl)-1-(6-methoxy-2-methyl-pyridin-3-yl)-3-methyl-1,3-dihydro-imidazo[4,5-c]quinolin-2-one

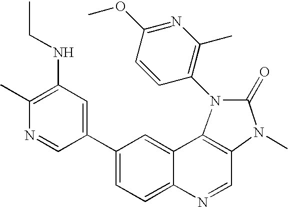

The title compound was synthesized in a similar manner as described for Example 1.1 using ethyl-[2-methyl-5-(4,4,5,5-tetramethyl-[1,3,2]dioxaborolan-2-yl)-pyridin-3-yl]-amine and intermediate K.

(HPLC: t_{R} 3.825 min (Method A); M+H=455.1 MS−ES; ¹H-NMR (d₆-DMSO, 600 MHz) 9.03 (s, 1H), 8.13-8.11 (m, 1H), 7.95-7.93 (m, 2H), 7.84 (m, 1H), 7.26 (m, 1H), 6.96-6.95 (m, 1H), 6.60 (s, 1H), 5.22 (m, 1H), 3.96 (s, 3H), 3.62 (s, 3H), 3.04-3.03 (m, 2H), 2.32 (s, 3H), 2.22 (s, 3H), 1.26-1.23 (m, 3H)).

### Example 11.8

### 8-(6-Hydroxymethyl-5-methoxy-pyridin-3-yl)-1-(6-methoxy-2-methyl-pyridin-3-yl)-3-methyl-1,3-dihydro-imidazo[4,5-c]quinolin-2-one

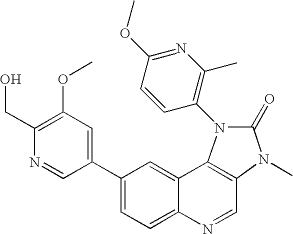

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester (see example 7.10; stage 7.10.1) and intermediate K. The acetyl protecting group was removed in situ by dissolving the crude product in an aqueous solution of LiOH (1 M, 4 equivalents), stirring for 5 min at RT and the removing the solvent under reduced pressure.

(HPLC: t_{R} 3.792 min (Method A); M+H=458.2 MS−ES; ¹H NMR (600 MHz, DMSO-d₆) δ ppm 9.06 (s, 1H), 8.22 (d, 1H), 8.17 (d, 1H), 8.03 (dd, 1H), 7.96 (d, 1H), 7.28 (d, 1H), 7.24 (d, 1H), 6.99 (d, 1H), 4.93 (t, 1H), 4.55 (d, 2H), 3.91-3.99 (m, 3H), 3.85 (s, 3H), 3.63 (s, 3H), 2.23 (s, 3H)).

### Example 12.1

### 1-[6-(Ethyl-methyl-amino)-2-methyl-pyridin-3-yl]-3-methyl-8-(6-methylamino-pyridin-3-yl)-1,3-dihydro-imidazo[4,5-c]quinolin-2-one

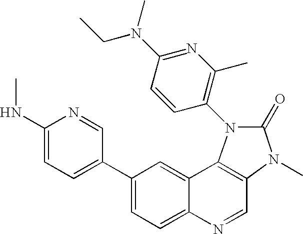

The title compound was synthesized in a similar manner as described for Example 1.1 using 6-(boc-methylamino)pyridine-3-boronic acid pinacol ester and intermediate L. Boc was removed in situ (1.0 mL for 5 min at RT) before purification by dissolving the crude product in TFA (0.5 ml) during 5 min.

(HPLC: t_{R} 3.85 min (Method B); M+H=454.2 MS−ES, ¹H-NMR (d₆-DMSO, 600 MHz) 8.93 (s, 1H), 8.13 (s, 1H), 8.03-8.02 (m, 1H), 7.84-7.83 (m, 1H), 7.62-7.60 (m, 1H), 7.36-7.35 (m, 1H), 7.15 (s, 1H), 6.76 (m, 1H), 6.73-6.72 (m, 1H), 6.44-6.43 (m, 1H), 3.74-3.72 (m, 1H), 3.64-3.62 (m, 1H), 3.60 (s, 3H), 3.11 (s, 3H), 2.79 (m, 3H), 2.07 (s, 3H), 1.18-1.16 (m, 3H)).

### Intermediate L 8-Bromo-1-[6-(ethyl-methyl-amino)-2-methyl-pyridin-3-yl]-3-methyl-1,3-dihydro-imidazo[4,5-c]quinolin-2-one

The title compound was synthesized in analogy as described for intermediate A, stage A.1-A.4 using N*2*-ethyl-6,N*2*-dimethyl-pyridine-2,5-diamine instead.

### Stage 12.1.1 N*2*-Ethyl-6,N*2*-dimethyl-pyridine-2,5-diamine

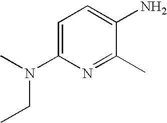

The title compound was prepared from ethyl-methyl-(6-methyl-5-nitro-pyridin-2-yl)-amine (Stage 12.1.2) by catalytic hydrogenation using H2/Raney-Ni in MeOH/THF 1:1 at RT.

Ethyl-methyl-(6-methyl-5-nitro-pyridin-2-yl)-amine (Stage 12.1.2) (554 mg) was dissolved in a mixture of MeOH/THF (17.5 mL each) and subjected to catalytic hydrogenation using H₂/Raney-Ni (120 mg) for 19 h at RT. The catalyst was filtered off carefully, washed with MeOH/THF and the solvent was removed under reduced pressure to afford the title compound as a crude product (505 mg). The product was used without further purification in the next step.

Title compound: (HPLC: t_{R} 3.28 min (Method B); M+H=166.1 MS−ES)

### Stage 12.1.2 Ethyl-methyl-(6-methyl-5-nitro-pyridin-2-yl)-amine

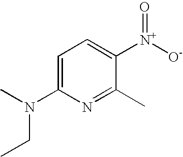

6-Chloro-3-nitro-2-picoline (Acros Organics) (500 mg; 2.84 mMol) and N-methylethylamine (Aldrich) (1.132 mL; 12.78 mMol) was dissolved in CH₃OH (7 mL) and stirred at 40° C. for 1 h. The reaction mixture was diluted with EtOAc and washed with NaHCO₃ (sat. soln.) and with brine (1×). The organic layer was dried on anhydrous Na₂SO₄ and after filtration, the solvent was removed under reduced pressure, and dried under vacuum (50° C.) to give 642 mg of crude product as a yellow solid. The product was used without further purification in the next step.

Title compound: (HPLC: t_{R} 6.85 min (Method B); M+H=196.1 MS−ES)

### Example 12.2

### 8-(5-Ethylamino-6-methyl-pyridin-3-yl)-1-[6-(ethyl-methyl-amino)-2-methyl-pyridin-3-yl]-3-methyl-1,3-dihydro-imidazo[4,5-c]quinolin-2-one

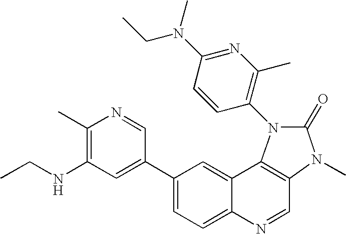

The title compound was synthesized in a similar manner as described for Example 1.1 using ethyl-[2-methyl-5-(4,4,5,5-tetramethyl-[1,3,2]dioxaborolan-2-yl)-pyridin-3-yl]-amine and intermediate L.

(HPLC: t_{R} 4.06 min (Method B); M+H=482 MS−ES).

### Example 13.1

### 8-(6-Ethoxy-pyridin-3-yl)-1-[6-(2-methoxy-ethoxy)-2-methyl-pyridin-3-yl]-3-methyl-1,3-dihydro-imidazo[4,5-c]quinolin-2-one

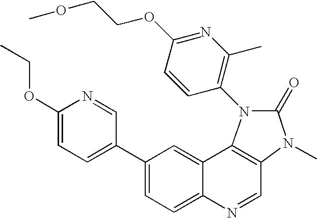

The title compound was synthesized in a similar manner as described for Example 1.1 using 6-Ethoxypyridine-3-boronic acid and intermediate M.

(HPLC: t_{R} 5.70 min (Method B); M+H=486.1 MS−ES, ¹H-NMR (d₆-DMSO, 600 MHz) 9.02 (s, 1H), 8.17 (m, 1H), 8.12-8.11 (m, 1H), 7.93-7.92 (m, 1H), 7.90-7.88 (m, 1H), 7.69-7.67 (m, 1H), 7.06 (s, 1H), 6.99-6.98 (m, 1H), 6.86-6.84 (m, 1H), 4.53-4.51 (m, 2H), 4.35-4.31 (m, 2H), 3.73 (m, 2H), 3.63 (s, 3H), 3.34 (s, 3H), 2.20 (s, 3H), 1.34-1.32 (m, 3H)).

### Intermediate M 8-Bromo-1-[6-(2-methoxy-ethoxy)-2-methyl-pyridin-3-yl]-3-methyl-1,3-dihydro-imidazo[4,5-c]quinolin-2-one

The title compound was synthesized in analogy as described for intermediate L; see example 12.1.1 using 6-(2-methoxy-ethoxy)-2-methyl-3-nitro-pyridine instead.

### Stage 13.1.1 6-(2-Methoxy-ethoxy)-2-methyl-3-nitro-pyridine

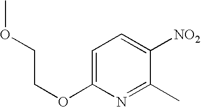

6-Chloro-3-nitro-2-picoline (500 mg, 2.90 mmol) was dissolved in 2-methoxyethanol (20 ml, 254 mmol) and stirred for 5 min under Ar and molecular sieves. NaH 55-65% in mineral oil (579 mg, 14.49 mmol) was added slowly to the light brown solution at rt (careful: strong H₂ evolution and exothermic !!! Ctrl of temperature with a water/ice bath). The black mixture was stirred for 1.5 h at rt. Water and a phosphate buffer solution (pH=7.0) were added to the reaction mixture (neutral pH), which was then extracted three times with EtOAc. The combined organic layers were dried (Na₂SO₄), and after filtration the solvent was removed under reduced pressure. After drying under vacuum (40° C.) the title compound was obtained as a light brown solid (151 mg) and used without further purification in the next step.

Title compound: (HPLC: t_{R} 6.46 min (Method B); M+H=213.1 MS−ES)

### Example 13.2

### 8-(6-Amino-5-trifluoromethyl-pyridin-3-yl)-1-[6-(2-methoxy-ethoxy)-2-methyl-pyridin-3-yl]-3-methyl-1,3-dihydro-imidazo[4,5-c]quinolin-2-one

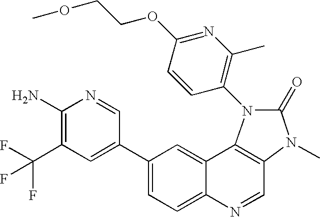

The title compound was synthesized in a similar manner as described for Example 1.1 using 5-(4,4,5,5-tetramethyl-[1,3,2]dioxaborolan-2-yl)-3-trifluoro-methyl-pyridin-2-ylamine and intermediate M.

(HPLC: t_{R} 5.07 min (Method B); M+H=525.0 MS−ES, ¹H-NMR (d₆-DMSO, 600 MHz) 9.01 (s, 1H), 8.36-8.35 (m, 1H), 8.10-8.08 (m, 1H), 7.95-7.93 (m, 2H), 7.59 (s, 1H), 7.09 (s, 1H), 6.96-6.95 (m, 1H), 6.75 (s, br, 2H), 4.55-4.53 (m, 1H), 4.41-4.39 (m, 1H), 3.72-3.71 (m, 2H), 3.62 (s, 3H), 3.34 (s, 3H), 2.19 (s, 3H)).

### Example 13.3

### 1-[6-(2-Methoxy-ethoxy)-2-methyl-pyridin-3-yl]-3-methyl-8-(6-methylamino-pyridin-3-yl)-1,3-dihydro-imidazo[4,5-c]quinolin-2-one

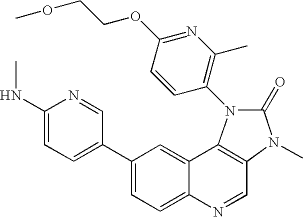

The title compound was synthesized in a similar manner as described for Example 1.1 using 6-(boc-methylamino)pyridine-3-boronic acid pinacol ester and intermediate M. Boc was removed in situ (1.0 mL for 5 min at RT) before purification by dissolving the crude product in TFA (0.5 ml) during 5 min.

(HPLC: t_{R} 4.23 min (Method B); M+H=471.1 MS−ES, ¹H-NMR (d₆-DMSO, 600 MHz) 8.97 (s, 1H), 8.06-8.05 (m, 2H), 7.93-7.92 (m, 1H), 7.84-7.82 (m, 1H), 7.38-7.37 (m, 1H), 7.01-6.98 (m, 2H), 6.76 (m, 1H), 6.47-6.46 (m, 1H), 4.54-4.52 (m, 2H), 3.75-3.73 (m, 2H), 3.61 (s, 3H), 3.34 (s, 3H), 2.80-2.79 (m, 3H), 2.19 (s, 3H)).

### Example 13.4

### 8-(5-Ethylamino-6-methyl-pyridin-3-yl)-1-[6-(2-methoxy-ethoxy)-2-methyl-pyridin-3-yl]-3-methyl-1,3-dihydro-imidazo[4,5-c]quinolin-2-one

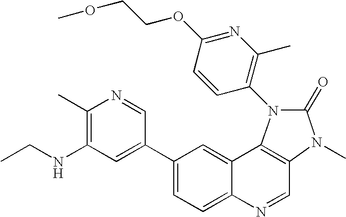

The title compound was synthesized in a similar manner as described for Example 1.1 using ethyl-[2-methyl-5-(4,4,5,5-tetramethyl-[1,3,2]dioxaborolan-2-yl)-pyridin-3-yl]-amine and intermediate M.

(HPLC: t_{R} 4.49 min (Method B); M+H=499 MS−ES).

### Example 13.5

### 1-[6-(2-Methoxy-ethoxy)-2-methyl-pyridin-3-yl]-3-methyl-8-(2-methylamino-pyrimidin-5-yl)-1,3-dihydro-imidazo[4,5-c]quinolin-2-one

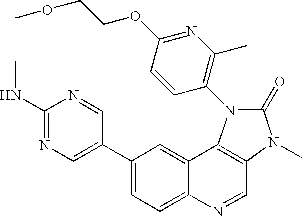

The title compound was synthesized in a similar manner as described for Example 1.1 using methyl-[5-(4,4,5,5-tetramethyl-[1,3,2]dioxaborolan-2-yl)-pyrimidin-2-yl]-amine and intermediate M.

(HPLC: t_{R} 4.61 min (Method B); M+H=472 MS−ES; ¹H NMR (400 MHz, d₆-DMSO) δ ppm 8.98 (s, 1H), 8.30 (br. s., 2H), 8.07 (d, 1H), 7.90 (d, 1H), 7.86 (s, 1H), 7.33 (d, 1H), 7.02 (s, 1H), 6.96 (d, 1H), 4.42-4.58 (m, 2H), 3.65-3.78 (m, 2H), 3.60 (s, 3H), 3.31 (d, 3H), 2.81 (d, 3H), 2.18 (s, 3H)).

### Example 14.1

### 1-(2,6-Dimethyl-pyridin-3-yl)-3-methyl-8-(6-methylamino-pyridin-3-yl)-1,3-dihydro-imidazo[4,5-c]quinolin-2-one

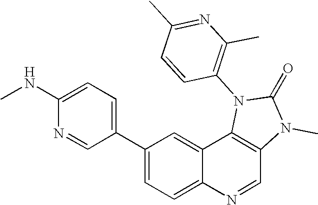

The title compound was synthesized in a similar manner as described for Example 1.1 using 6-(boc-methylamino)pyridine-3-boronic acid pinacol ester and intermediate N. Boc was removed in situ (1.0 mL for 5 min at RT) before purification by dissolving the crude product in TFA (0.5 ml) during 5 min.

(HPLC: t_{R} 3.41 min (Method A); M+H=411.2 MS−ES).

### Example 14.2

### 1-(2,6-Dimethyl-pyridin-3-yl)-8-(6-ethoxy-pyridin-3-yl)-3-methyl-1,3-dihydro-imidazo[4,5-c]quinolin-2-one

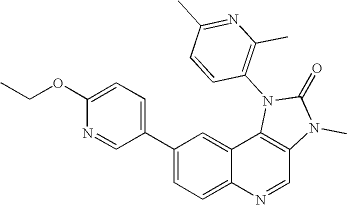

The title compound was synthesized in a similar manner as described for Example 1.1 using 6-ethoxypyridine-3-boronic acid and intermediate N.

(HPLC: t_{R} 4.03 min (Method A); M+H=426.2 MS−ES; ¹H-NMR (d₆-DMSO, 600 MHz) 9.03 (s, 1H), 8.14 (m, 1H), 8.12-8.11 (m, 1H), 7.93-7.91 (m, 1H), 7.90-7.88 (m, 1H), 7.62-7.60 (m, 1H), 7.45-7.44 (m, 1H), 6.91 (m, 1H), 6.87-6.86 (m, 1H), 4.35-4.31 (m, 2H), 3.63 (s, 3H), 2.62 (s, 3H), 2.24 (s, 3H), 1.34-1.32 (m, 3H).

### Example 14.3

### 8-(2-Dimethylamino-pyrimidin-5-yl)-1-(2,6-dimethyl-pyridin-3-yl)-3-methyl-1,3-dihydro-imidazo[4,5-c]quinolin-2-one

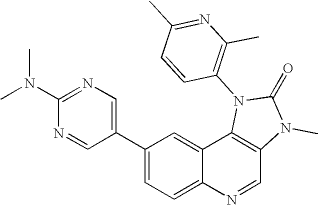

The title compound was synthesized in a similar manner as described for Example 1.1 using dimethyl-[5-(4,4,5,5-tetramethyl-[1,3,2]dioxaborolan-2-yl)-pyrimidin-2-yl]-amine and intermediate N.

(HPLC: t_{R} 3.73 min (Method A); M+H=426.2 MS−ES; ¹H-NMR (d₆-DMSO, 600 MHz) 9.01 (s, 1H), 8.33 (s, 2H), 8.10-8.09 (m, 1H), 7.91-7.90 (m, 1H), 7.88-7.87 (m, 1H), 7.46-7.45 (m, 1H), 6.87 (m, 1H), 3.63 (s, 3H), 3.15 (s, 6H), 2.62 (s, 3H), 2.25 (s, 3H).

### Example 14.4

### 1-(2,6-Dimethyl-pyridin-3-yl)-3-methyl-8-(2-methylamino-pyrimidin-5-yl)-1,3-dihydro-imidazo[4,5-c]quinolin-2-one

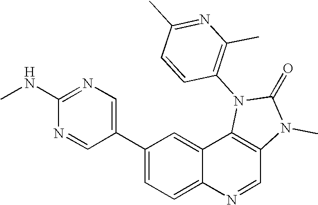

The title compound was synthesized in a similar manner as described for Example 1.1 using methyl-[5-(4,4,5,5-tetramethyl-[1,3,2]dioxaborolan-2-yl)-pyrimidin-2-yl]-amine and intermediate N. Boc was removed in situ (1.0 mL for 5 min at RT) before purification by dissolving the crude product in TFA (0.5 ml) during 5 min.

(HPLC: t_{R} 3.52 min (Method A); M+H=412.2 MS−ES; ¹H-NMR (d₆-DMSO, 600 MHz) 9.01 (s, 1H), 8.33 (br, 1H), 8.10-8.08 (m, 1H), 7.92-7.91 (m, 1H), 7.88 (m, 1H), 7.87-7.86 (m, 1H), 7.46-7.43 (m, 1H), 7.42-7.40 (m, 1H), 6.88-6.87 (m, 1H), 3.62 (s, 3H), 2.83-2.82 (m, 3H), 2.62 (s, 3H), 2.24 (s, 3H).

### Example 14.5

### 8-(6-Amino-5-trifluoromethyl-pyridin-3-yl)-1-(2,6-dimethyl-pyridin-3-yl)-3-methyl-1,3-dihydro-imidazo[4,5-c]quinolin-2-one

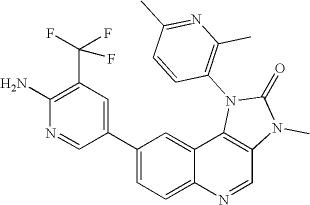

The title compound was synthesized in a similar manner as described for Example 1.1 using 5-(4,4,5,5-tetramethyl-[1,3,2]dioxaborolan-2-yl)-3-trifluoro-methyl-pyridin-2-ylamine and intermediate N.

(HPLC: t_{R} 3.77 min (Method A); M+H=465.1 MS−ES; ¹H-NMR (d₆-DMSO, 600 MHz) 9.01 (s, 1H), 8.35 (s, 1H), 8.10-8.08 (m, 1H), 7.95-7.94 (m, 2H), 7.50 (s, 1H), 7.43-7.42 (m, 1H), 6.93 (s, 1H), 6.76 (s, br, 2H), 3.63 (s, 3H), 2.59 (s, 3H), 2.23 (s, 3H).

### Example 14.6

### 1-(2,6-Dimethyl-pyridin-3-yl)-8-(5-ethylamino-6-methyl-pyridin-3-yl)-3-methyl-1,3-dihydro-imidazo[4,5-c]quinolin-2-one

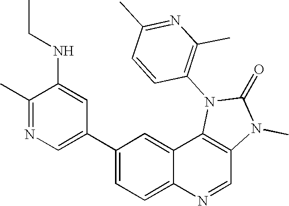

The title compound was synthesized in a similar manner as described for Example 1.1 using ethyl-[2-methyl-5-(4,4,5,5-tetramethyl-[1,3,2]dioxaborolan-2-yl)-pyridin-3-yl]-amine and intermediate N.

(HPLC: t_{R} 3.533 min (Method A); M+H=439.2 MS−ES; ¹H-NMR (d₆-DMSO, 600 MHz) 9.04 (s, 1H), 8.13-8.11 (m, 1H), 7.94-7.91 (m, 2H), 7.78 (s, 1H), 7.43-7.42 (m, 1H), 7.13 (s, 1H), 6.58 (s, 1H), 5.24 (m, 1H), 3.63 (s, 3H), 3.09-3.06 (m, 2H), 2.58 (s, 3H), 2.31 (s, 3H), 2.25 (s, 3H), 1.26-1.24 (m, 3H).

### Example 14.7

### 1-(2,6-Dimethyl-pyridin-3-yl)-3-methyl-8-(1-methyl-1H-pyrrolo[2,3-b]pyridin-5-yl)-1,3-dihydro-imidazo[4,5-c]quinolin-2-one

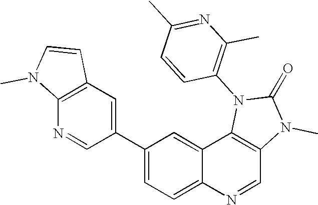

The title compound was synthesized in a similar manner as described for Example 1.1 using 1-methyl-5-(4,4,5,5-tetramethyl-[1,3,2]dioxaborolan-2-yl)-1H-pyrrolo[2,3-b]pyridine and intermediate N.

(HPLC: t_{R} 3.86 min (Method A); M+H=435.2 MS−ES.

### Example 14.8

### 1-(2,6-Dimethyl-pyridin-3-yl)-8-(5-isopropylamino-pyridin-3-yl)-3-methyl-1,3-dihydro-imidazo[4,5-c]quinolin-2-one

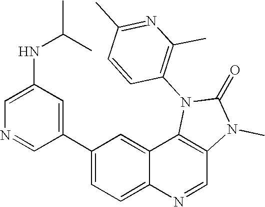

The title compound was synthesized in a similar manner as described for Example 1.1 using isopropyl-[5-(4,4,5,5-tetramethyl-[1,3,2]dioxaborolan-2-yl)-pyridin-3-yl]-amine and intermediate N.

(HPLC: t_{R} 3.59 min (Method A); M+H=439.2 MS−ES).

### Example 15.1

### 8-(6-Amino-5-trifluoromethyl-pyridin-3-yl)-1-{6-[(2-methoxy-ethyl)-methyl-amino]-2-methyl-pyridin-3-yl}-3-methyl-1,3-dihydro-imidazo[4,5-c]quinolin-2-one

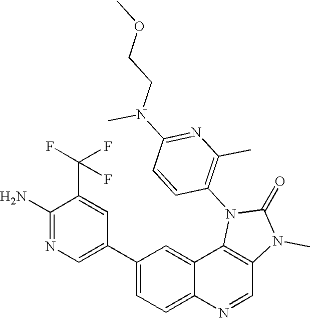

The title compound was synthesized in a similar manner as described for Example 1.1 using 5-(4,4,5,5-tetramethyl-[1,3,2]dioxaborolan-2-yl)-3-trifluoro-methyl-pyridin-2-ylamine and intermediate O.

(HPLC: t_{R} 3.91 min (Method A); M+H=538.0 MS−ES, ¹H-NMR (d₆-DMSO, 600 MHz) 8.98 (s, 1H), 8.40 (s, 1H), 8.08-8.07 (m, 1H), 7.94-7.92 (m, 1H), 7.64-7.63 (m, 1H), 7.58 (s, 1H), 7.25 (s, 1H), 6.72 (s, br, 2H), 6.71-6.69 (m, 1H), 3.87-3.84 (m, 1H), 3.70-3.67 (m, 1H), 3.61 (s, 3H), 3.55-3.53 (m, 2H), 3.26 (s, 3H), 3.12 (s, 3H), 2.06 (s, 3H)).

### Stage 15.1.1. Intermediate O 8-Bromo-1-{6-[(2-methoxy-ethyl)-methyl-amino]-2-methyl-pyridin-3-yl}-3-methyl-1,3-dihydro-imidazo[4,5-c]quinolin-2-one

The title compound was synthesized in analogy as described for intermediate A, stage A.1-A.4 using N*2*-(2-Methoxy-ethyl)-6,N*2*-dimethyl-pyridine-2,5-diamine instead (synthesis from 6-chloro-2-methyl-3-nitropyridine and 2-methoxy-N-methylethanamine; see example 12.1.1 and 12.1.2).

### Example 15.2

### 1-{6-[(2-Methoxy-ethyl)-methyl-amino]-2-methyl-pyridin-3-yl}-3-methyl-8-(2-methylamino-pyrimidin-5-yl)-1,3-dihydro-imidazo[4,5-c]quinolin-2-one

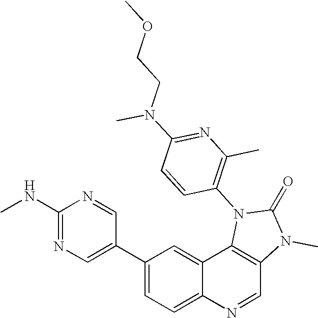

The title compound was synthesized in a similar manner as described for Example 1.1 using methyl-[5-(4,4,5,5-tetramethyl-[1,3,2]dioxaborolan-2-yl)-pyrimidin-2-yl]-amine and intermediate O.

(HPLC: t_{R} 3.67 min (Method A); M+H=485.1 MS−ES, ¹H-NMR (d₆-DMSO, 600 MHz) 8.97 (s, 1H), 8.34 (s, br, 2H), 8.08-8.06 (m, 1H), 7.87-7.86 (m, 1H), 7.63-7.61 (m, 1H), 7.36-7.35 (m, 1H), 7.17 (m, 1H), 6.74-6.73 (m, 1H), 3.82-3.78 (m, 2H), 3.61 (s, 3H), 3.57-3.55 (m, 2H), 3.27 (s, 3H), 3.14 (s, 3H), 2.83-2.81 (m, 3H), 2.08 (s, 3H)).

### Example 15.3

### 8-(6-Ethoxy-pyridin-3-yl)-1-{6-[(2-methoxy-ethyl)-methyl-amino]-2-methyl-pyridin-3-yl}-3-methyl-1,3-dihydro-imidazo[4,5-c]quinolin-2-one

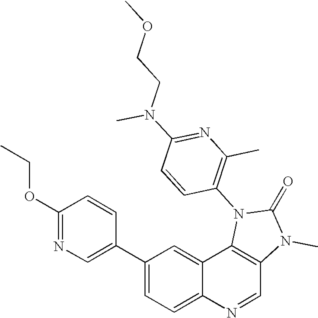

The title compound was synthesized in a similar manner as described for Example 1.1 using 6-ethoxypyridine-3-boronic acid and intermediate O.

(HPLC: t_{R} 4.25 min (Method A); M+H=499.1 MS−ES, ¹H-NMR (d₆-DMSO, 600 MHz) 8.99 (s, 1H), 8.24-8.23 (m, 1H), 8.10-8.09 (m, 1H), 7.91-7.89 (m, 1H), 7.69-7.68 (m, 1H), 7.64-7.62 (m, 1H), 7.21 (s, 1H), 6.83-6.82 (m, 1H), 6.75-6.73 (m, 1H), 4.36-4.32 (m, 2H), 3.86-3.84 (m, 1H), 3.78-3.76 (m, 1H), 3.62 (s, 3H), 3.57-3.55 (m, 2H), 3.26 (s, 3H), 3.15 (s, 3H), 2.07 (s, 3H), 1.35-1.32 (m, 3H)).

### Example 15.4

### 1-{6-[(2-Methoxy-ethyl)-methyl-amino]-2-methyl-pyridin-3-yl}-3-methyl-8-(6-methylamino-pyridin-3-yl)-1,3-dihydro-imidazo[4,5-c]quinolin-2-one

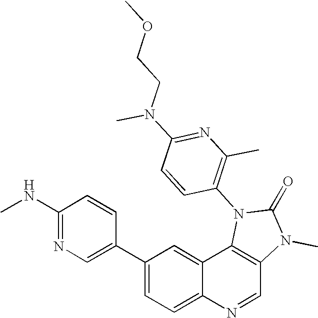

The title compound was synthesized in a similar manner as described for Example 1.1 using 6-(boc-methylamino)pyridine-3-boronic acid pinacol ester and intermediate 0. Boc was removed in situ (1.0 mL for 5 min at RT) before purification by dissolving the crude product in TFA (0.5 ml) during 5 min.

(HPLC: t_{R} 3.533 min (Method A); M+H=484.1 MS−ES, ¹H-NMR (d₆-DMSO, 600 MHz) 8.93 (s, 1H), 8.13 (s, 1H), 8.04-8.02 (m, 1H), 7.84-7.83 (m, 1H), 7.63-7.61 (m, 1H), 7.35 (m, 1H), 7.15 (s, 1H), 6.75-6.73 (m, 2H), 6.47-6.45 (m, 1H), 3.83 (m, 1H), 3.80 (m, 1H), 3.60 (s, 3H), 3.59-3.57 (m, 2H), 3.28 (s, 3H), 3.16 (s, 3H), 2.80-2.78 (s, 3H), 2.07 (s, 3H)).

### Example 15.5

### 8-(2-Dimethylamino-pyrimidin-5-yl)-1-{6-[(2-methoxy-ethyl)-methyl-amino]-2-methyl-pyridin-3-yl}-3-methyl-1,3-dihydro-imidazo[4,5-c]quinolin-2-one

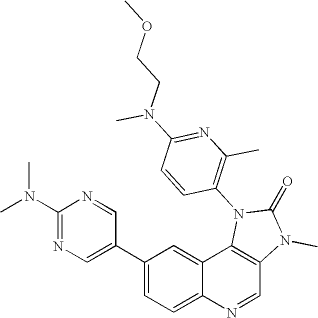

The title compound was synthesized in a similar manner as described for Example 1.1 using di methyl-[5-(4,4,5,5-tetramethyl-[1,3,2]dioxaborolan-2-yl)-pyrimidin-2-yl]-amine and intermediate O.

(HPLC: t_{R} 3.933 min (Method A); M+H=499.2 MS−ES, ¹H-NMR (d₆-DMSO, 600 MHz) 8.97 (s, 1H), 8.41 (s, 2H), 8.08-8.07 (m, 1H), 7.88-7.87 (m, 1H), 7.63-7.61 (m, 1H), 7.16-7.15 (m, 1H), 6.75-6.74 (m, 1H), 3.85 (m, 1H), 3.77-3.74 (m, 1H), 3.61 (s, 3H), 3.58-3.57 (m, 2H), 3.34 (s, 3H), 3.26 (s, 3H), 3.16 (s, 3H), 3.15 (s, 3H), 2.08 (s, 3H)).

### Example 15.6

### 8-(5-Ethylamino-6-methyl-pyridin-3-yl)-1-{6-[(2-methoxy-ethyl)-methyl-amino]-2-methyl-pyridin-3-yl}-3-methyl-1,3-dihydro-imidazo[4,5-c]quinolin-2-one

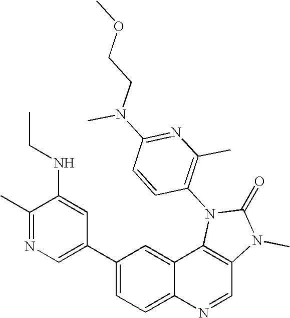

The title compound was synthesized in a similar manner as described for Example 1.1 using ethyl-[2-methyl-5-(4,4,5,5-tetramethyl-[1,3,2]dioxaborolan-2-yl)-pyridin-3-yl]-amine and intermediate O.

(HPLC: t_{R} 3.675 min (Method A); M+H=512.2 MS−ES, ¹H-NMR (d₆-DMSO, 600 MHz) 9.00 (s, 1H), 8.11-8.10 (m, 1H), 7.91-7.89 (m, 1H), 7.81 (s, 1H), 7.63-7.62 (m, 1H), 7.44-7.43 (m, 1H), 6.71-6.68 (m, 2H), 5.24 (m, 1H), 3.84-3.82 (m, 1H), 3.74-3.72 (m, 1H), 3.61 (s, 3H), 3.54-3.52 (m, 2H), 3.26 (s, 3H), 3.11 (s, 3H), 3.08-3.05 (m, 2H), 2.31 (s, 3H), 2.08 (s, 3H), 1.23-1.20 (m, 3H)).

### Example 15.7

### 1-{6-[(2-Methoxy-ethyl)-methyl-amino]-2-methyl-pyridin-3-yl}-3-methyl-8-(1-methyl-1H-pyrrolo[2,3-b]pyridin-5-yl)-1,3-dihydro-imidazo[4,5-c]quinolin-2-one

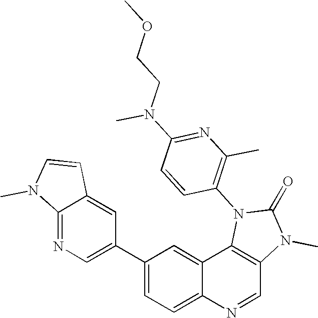

The title compound was synthesized in a similar manner as described for Example 1.1 using 1-Methyl-5-(4,4,5,5-tetramethyl-[1,3,2]dioxaborolan-2-yl)-1H-pyrrolo[2,3-b]pyridine and intermediate O.

(HPLC: t_{R} 4.03 min (Method A); M+H=508 MS−ES).

### Example 15.8

### 8-(5-Amino-pyridin-3-yl)-1-{6-[(2-methoxy-ethyl)-methyl-amino]-2-methyl-pyridin-3-yl}-3-methyl-1,3-dihydro-imidazo[4,5-c]quinolin-2-one

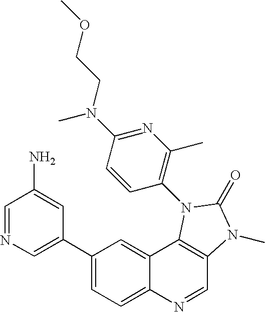

The title compound was synthesized in a similar manner as described for Example 1.1 using 3-aminopyridin-5-boronic acid pinacolester and intermediate O.

(HPLC: t_{R} 3.517 min (Method A); M+H=470.2 MS−ES).

### Example 15.9

### 8-(6-Hydroxymethyl-5-methoxy-pyridin-3-yl)-1-{6-[(2-methoxy-ethyl)-methyl-amino]-2-methyl-pyridin-3-yl}-3-methyl-1,3-dihydro-imidazo[4,5-c]quinolin-2-one

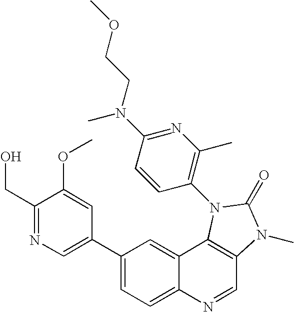

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester and intermediate 0. The acetyl protecting group was removed in situ by dissolving the crude product in an aqueous solution of LiOH (1 M, 4 equivalents), stirring for 5 min at RT and the removing the solvent under reduced pressure.

(HPLC: t_{R} 3.625 (method B); M+H=515.2 MS−ES).

### Example 16.1

### 8-(6-Amino-5-trifluoromethyl-pyridin-3-yl)-1-(6-fluoro-4-methyl-pyridin-3-yl)-3-methyl-1,3-dihydro-imidazo[4,5-c]quinolin-2-one

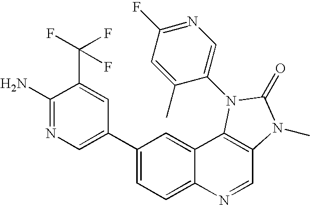

The title compound was synthesized in a similar manner as described for Example 1.1 using 5-(4,4,5,5-tetramethyl-[1,3,2]dioxaborolan-2-yl)-3-trifluoromethyl-pyridin-2-ylamine and intermediate P.

(HPLC: t_{R} 4.09 min (Method A); M+H=469.1 MS−ES).

### Example 16.2

### 8-(5-Ethylamino-6-methyl-pyridin-3-yl)-1-(6-fluoro-4-methyl-pyridin-3-yl)-3-methyl-1,3-dihydro-imidazo[4,5-c]quinolin-2-one

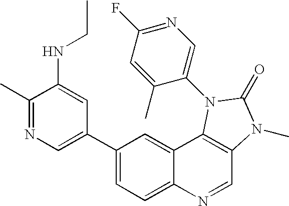

The title compound was synthesized in a similar manner as described for Example 1.1 using ethyl-[2-methyl-5-(4,4,5,5-tetramethyl-[1,3,2]dioxaborolan-2-yl)-pyridin-3-yl]-amine and intermediate P.

(HPLC: t_{R} 3.72 min (Method A); M+H=443.2 MS−ES).

### Example 16.3

### 1-(6-Fluoro-4-methyl-pyridin-3-yl)-3-methyl-8-(2-methylamino-pyrimidin-5-yl)-1,3-dihydro-imidazo[4,5-c]quinolin-2-one

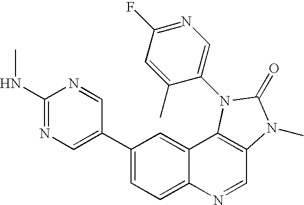

The title compound was synthesized in a similar manner as described for Example 1.1 using methyl-[5-(4,4,5,5-tetramethyl-[1,3,2]dioxaborolan-2-yl)-pyrimidin-2-yl]-amine and intermediate P.

(HPLC: t_{R} 3.78 min (Method A); M+H=416.1 MS−ES, ¹H NMR (600 MHz, DMSO-d₆) δ ppm 8.97-9.10 (m, 1H), 8.47-8.58 (m, 1H), 8.21-8.41 (m, 2H), 8.11 (d, 1H), 7.89 (dd, 1H), 7.56 (s, 1H), 7.42 (t, 1H), 6.93 (d, 1H), 3.60-3.71 (m, 3H), 2.76-2.90 (m, 3H), 2.20 (s, 3H)).

### Example 16.4

### 1-(6-Fluoro-4-methyl-pyridin-3-yl)-3-methyl-8-(6-methylamino-pyridin-3-yl)-1,3-dihydro-imidazo[4,5-c]quinolin-2-one

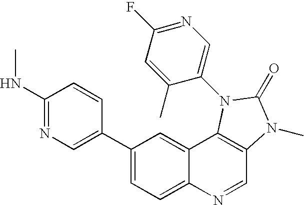

The title compound was synthesized in a similar manner as described for Example 1.1 using 6-(boc-methylamino)pyridine-3-boronic acid pinacol ester and intermediate P. Boc was removed in situ (1.0 mL for 5 min at RT) before purification by dissolving the crude product in TFA (0.5 ml) during 5 min.

(HPLC: t_{R} 3.55 min (Method A); M+H=415.1 MS−ES, ¹H-NMR (d₆-DMSO, 600 MHz) 9.01 (s, 1H), 8.53 (s, 1H), 8.09-8.08 (m, 1H), 8.01 (m, 1H), 7.86-7.84 (m, 1H), 7.57 (m, 1H), 7.41 (m, 1H), 6.92 (m, 1H), 6.53-6.52 (m, 1H), 3.63 (s, 3H), 3.57 (m, 1H), 2.81-2.80 (m, 3H), 2.20 (s, 3H).

### Example 16.5

### 1-(6-Fluoro-4-methyl-pyridin-3-yl)-3-methyl-8-(1-methyl-1H-pyrrolo[2,3-b]pyridin-5-yl)-1,3-dihydro-imidazo[4,5-c]quinolin-2-one

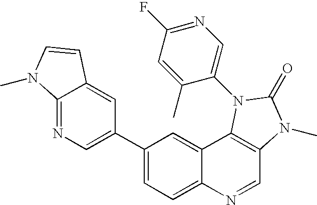

The title compound was synthesized in a similar manner as described for Example 1.1 using 1-methyl-5-(4,4,5,5-tetramethyl-[1,3,2]dioxaborolan-2-yl)-1H-pyrrolo[2,3-b]pyridine and intermediate P.

(HPLC: t_{R} 4.20 min (Method A); M+H=439.1 MS−ES).

### Example 17.1

### 8-(6-Ethoxy-pyridin-3-yl)-3-methyl-1-(5-methyl-pyridin-3-yl)-1,3-dihydro-imidazo[4,5-c]quinolin-2-one

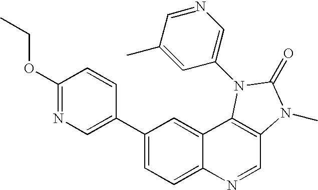

The title compound was synthesized in a similar manner as described for Example 1.1 using 6-ethoxypyridine-3-boronic acid and intermediate Q.

(HPLC: t_{R} 4.22 min (Method A); M+H=412.1 MS−ES, ¹H-NMR (d₆-DMSO, 600 MHz) 9.03 (s, 1H), 8.72-8.71 (m, 2H), 8.18 (m, 1H), 8.13-8.12 (m, 1H), 8.04 (s, 1H), 7.90-7.89 (m, 1H), 7.69-7.67 (m, 1H), 7.17 (m, 1H), 6.87-6.86 (m, 1H), 4.34-4.31 (m, 2H), 3.62 (s, 3H), 2.46 (s, 3H), 1.34-1.31 (m, 3H)).

### Example 17.2

### 3-Methyl-8-(2-methylamino-pyrimidin-5-yl)-1-(5-methyl-pyridin-3-yl)-1,3-dihydro-imidazo[4,5-c]quinolin-2-one

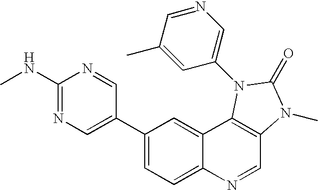

The title compound was synthesized in a similar manner as described for Example 1.1 using methyl-[5-(4,4,5,5-tetramethyl-[1,3,2]dioxaborolan-2-yl)-pyrimidin-2-yl]-amine and intermediate Q.

(HPLC: t_{R} 3.61 min (Method A); M+H=398.1 MS−ES).

### Example 18.1

### 1-(2-Chloro-6-methoxy-pyridin-3-yl)-8-(5-ethylamino-6-methyl-pyridin-3-yl)-3-methyl-1,3-dihydro-imidazo[4,5-c]quinolin-2-one

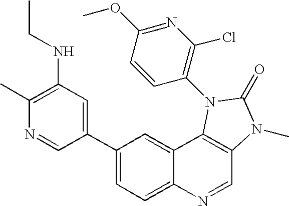

The title compound was synthesized in a similar manner as described for Example 1.1 using ethyl-[2-methyl-5-(4,4,5,5-tetramethyl-[1,3,2]dioxaborolan-2-yl)-pyridin-3-yl]-amine and intermediate R.

(HPLC: t_{R} 3.92 min (Method A); M+H=475.1 MS−ES).

### Example 18.2

### 1-(2-Chloro-6-methoxy-pyridin-3-yl)-3-methyl-8-(2-methylamino-pyrimidin-5-yl)-1,3-dihydro-imidazo[4,5-c]quinolin-2-one

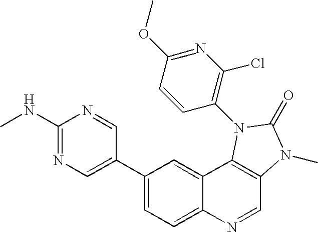

The title compound was synthesized in a similar manner as described for Example 1.1 using methyl-[5-(4,4,5,5-tetramethyl-[1,3,2]dioxaborolan-2-yl)-pyrimidin-2-yl]-amine and intermediate R.

(HPLC: t_{R} 4.03 min (Method A); M+H=448.0 MS−ES).

### Example 18.3

### 1-(2-Chloro-6-methoxy-pyridin-3-yl)-8-(2-dimethylamino-pyrimidin-5-yl)-3-methyl-1,3-dihydro-imidazo[4,5-c]quinolin-2-one

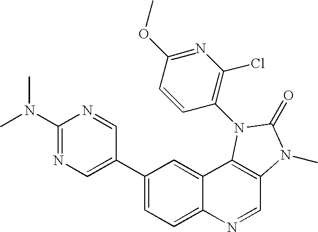

The title compound was synthesized in a similar manner as described for Example 1.1 using (N-dimethylamino)pyrimidin-3-boronic acid pinacolester and intermediate R.

(HPLC: t_{R} 4.200 min (Method A); M+H=439.1 MS−ES, ¹H-NMR (d₆-DMSO, 600 MHz) 9.02 (s, 1H), 8.41 (s, 2H), 8.28-8.27 (m, 1H), 8.12-8.11 (m, 1H), 7.91-7.89 (m, 1H), 7.26-7.24 (m, 1H), 7.05 (m, 1H), 4.02 (s, 3H), 3.63 (s, 3H), 3.16 (s, 6H)).

### Example 19.1

### 1-(6-Dimethylamino-2-methyl-pyridin-3-yl)-3-methyl-8-(2-methylamino-pyrimidin-5-yl)-1,3-dihydro-imidazo[4,5-c]quinolin-2-one

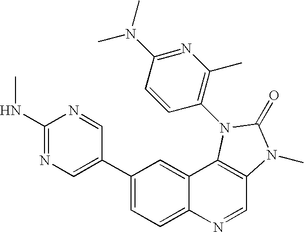

The title compound was synthesized in a similar manner as described for Example 1.1 using 2-methylaminopyrimidin-5-boronic acid pinacolester and intermediate S.

(HPLC: t_{R} 3.525 min (Method A); M+H=441.2 MS−ES).

### Stage 19.1.1 6,N*2*,N*2*-Trimethyl-pyridine-2,5-diamine

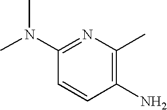

To a solution of dimethyl-(6-methyl-5-nitro-pyridin-2-yl)-amine (782 mg; 4.32 mmol) (example 104, step 104.1) in MeOH/THF=1:1 (20 ml total) was added Ra-Ni (400 mg) and the reaction mixture was stirred under H₂ atmosphere at rt for 10 h. The reaction mixture was then filtered and washed several times with MeOH. The filtrate was evaporated to dryness to give the title compound as a dark yellow powder (642 mg; quant.). HPLC: t_{R}=3.075 min (method D); ESI-MS: [M+H]⁺152.1 (method A).

### Stage 19.1.2 Dimethyl-(6-methyl-5-nitro-pyridin-2-yl)-amine

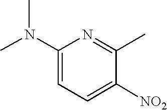

6-methyl-5-nitropyridin-2-amine (1.225 g; 8 mmol) was dissolved in anhydrous DMF (15 mL), cooled to 0° C. and treated with NaH (419 mg; 9.6 mmol) for 30 min. Methyliodide (1136 mg; 8 mmol) was added, followed by removal of the ice bath and the reaction was continued at RT for 2 h. After addition of EtOAc (100 mL), as a side product, methyl-(6-methyl-5-nitro-pyridin-2-yl)-amine, precipitated and was filtered off. After washing the organic layer with water (2×), the solvent was evaporated to dryness to obtain the crude product. Purification was done over silica gel (solvent system: CH₂Cl₂/MeOH=95:5) to yield the title compound as a bright yellow solid. HPLC: t_{R}=4.992 min (method A); ESI-MS: [M+H]⁺ 182.1.

### Example 19.2

### 1-(6-Dimethylamino-2-methyl-pyridin-3-yl)-3-methyl-8-(6-methyl-5-methylamino-pyridin-3-yl)-1,3-dihydro-imidazo[4,5-c]quinolin-2-one

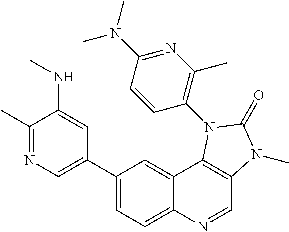

The title compound was synthesized in a similar manner as described for Example 1.1 using methyl-[2-methyl-5-(4,4,5,5-tetramethyl-[1,3,2]dioxaborolan-2-yl)-pyridin-3-yl]-amine (see example 2.32; stage 2.32.1) and intermediate S.

(HPLC: t_{R} 3.525 min (Method A); M+H=454.2 MS−ES, ¹H-NMR (d₆-DMSO, 600 MHz) 8.99 (s, 1H), 8.10-8.09 (m, 1H), 7.95 (m, 1H), 7.90 (m, 1H), 7.65-7.63 (m, 1H), 7.47 (m, 1H), 6.71-6.70 (m, 1H), 6.55 (m, 1H), 5.51-5.50 (m, 1H), 3.61 (s, 3H), 3.11 (s, 6H), 2.68-2.67 (m, 3H), 2.29 (s, 3H), 2.09 (s, 3H)).

### Example 19.3

### 1-(6-Dimethylamino-2-methyl-pyridin-3-yl)-8-(6-ethoxy-pyridin-3-yl)-3-methyl-1,3-dihydro-imidazo[4,5-c]quinolin-2-one

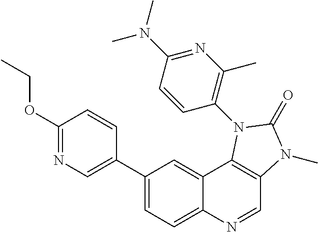

The title compound was synthesized in a similar manner as described for Example 1.1 using 6-ethoxypyridin-3-ylboronic acid and intermediate S.

(HPLC: t_{R} 4.067 min (Method A); M+H=455.2 MS−ES).

### Example 19.4

### 1-(6-Dimethylamino-2-methyl-pyridin-3-yl)-8-(6-hydroxymethyl-5-methoxy-pyridin-3-yl)-3-methyl-1,3-dihydro-imidazo[4,5-c]quinolin-2-one

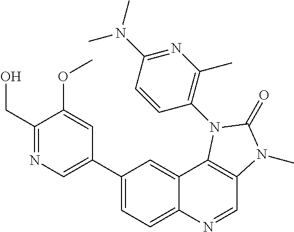

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester (see example 7.10; stage 7.10.1) and intermediate S. The acetyl protecting group was removed in situ by dissolving the crude product in an aqueous solution of LiOH (1 M, 4 equivalents), stirring for 5 min at RT and the removing the solvent under reduced pressure.

(HPLC: t_{R} 3.450 min (Method A); M+H=471.2 MS−ES).

### Example 19.5

### 8-(5-Amino-pyridin-3-yl)-1-(6-dimethylamino-2-methyl-pyridin-3-yl)-3-methyl-1,3-dihydro-imidazo[4,5-c]quinolin-2-one

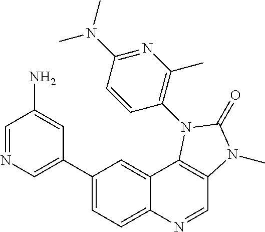

The title compound was synthesized in a similar manner as described for Example 1.1 using 3-aminopyridin-5-boronic acid pinacolester and intermediate S.

(HPLC: t_{R} 3.392 min (Method A); M+H=426.2 MS−ES, ¹H NMR (600 MHz, DMSO-d₆) δ ppm 9.00 (s, 1H), 8.11 (d, 1H), 7.90 (d, 1H), 7.78 (dd, 1H), 7.69 (d, 1H), 7.65 (d, 1H), 7.29 (d, 1H), 6.99 (t, 1H), 6.76 (d, 1H), 5.42 (s, 2H), 3.57-3.67 (m, 3H), 3.05-3.18 (m, 6H), 2.08 (s, 3H)).

### Example 20.1

### N-{5-[8-(5-Amino-pyridin-3-yl)-3-methyl-2-oxo-2,3-dihydro-imidazo[4,5-c]quinolin-1-yl]-6-methyl-pyridin-2-yl}-2-methoxy-acetamide

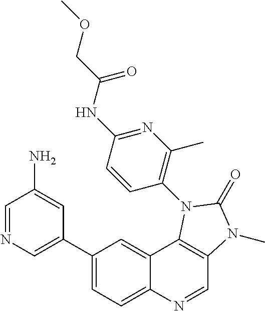

The title compound was synthesized in a similar manner as described for Example 1.1 using 3-aminopyridin-5-boronic acid pinacolester and intermediate T.

(HPLC: t_{R} 3.592 min (Method A); M+H=470.2 MS−ES, ¹H-NMR (d₆-DMSO, 600 MHz) 10.51 (s, 1H), 9.05 (s, 1H), 8.21-8.19 (m, 1H), 8.14-8.13 (m, 1H), 8.06-8.05 (m, 1H), 7.89 (m, 1H), 7.80-7.98 (m, 1H), 7.68 (m, 1H), 7.11 (s, 1H), 6.89 (s, 1H), 5.38 (s, 2H), 4.14 (s, 2H), 3.63 (s, 3H), 3.40 (s, 3H), 2.23 (s, 3H)).

### Stage 20.1.1 N-(5-Amino-6-methyl-pyridin-2-yl)-2-methoxy-acetamide

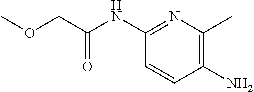

The title compound was prepared from 2-methoxy-N-(6-methyl-5-nitro-pyridin-2-yl)-acetamide as described in example 19., stage 19.1.1. (HPLC: t_{R} 3.367 min (Method A); M+H=196.1 MS−ES.

### Stage 20.1.2 2-Methoxy-N-(6-methyl-5-nitro-pyridin-2-yl)-acetamide

6-Methyl-5-nitropyridin-2-amine (938 mg; 6.0 mmol) was dissolved in THF (10 mL) at RT, followed by portionwise addition of CDI (1,1′-carbonyl-diimidazol) and kept stirring for 5 days. After removal of the solvent under reduced pressure the crude product was purified by chromatography over silica gel (eluting with DCM/MeOH; 98/2) to obtain the title compound as a bright beige powder (320 mg). (HPLC: t_{R} 4.825 min (Method A); M+H=226.1 MS−ES).

### Example 20.2

### N-{5-[8-(6-Ethoxy-pyridin-3-yl)-3-methyl-2-oxo-2,3-dihydro-imidazo[4,5-c]quinolin-1-yl]-6-methyl-pyridin-2-yl}-2-methoxy-acetamide

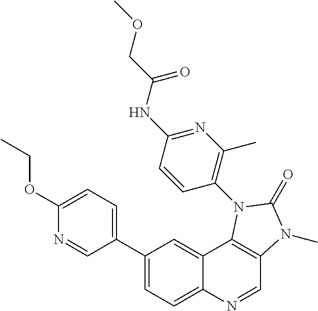

The title compound was synthesized in a similar manner as described for Example 1.1 using 6-ethoxypyridin-3-ylboronic acid and intermediate T.

(HPLC: t_{R} 4.350 min (Method A); M+H=499.2 MS−ES).

### Example 20.3

### 2-Methoxy-N-{6-methyl-5-[3-methyl-8-(2-methylamino-pyrimidin-5-yl)-2-oxo-2,3-dihydro-imidazo[4,5-c]quinolin-1-yl]-pyridin-2-yl}-acetamide

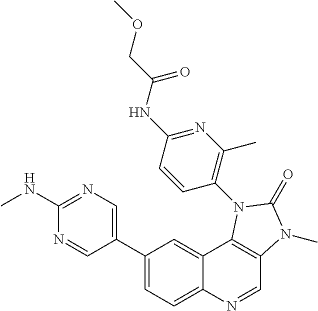

The title compound was synthesized in a similar manner as described for Example 1.1 using 2-Methylaminopyrimidin-5-boronic acid pinacolester and intermediate T.

(HPLC: t_{R} 3.775 min (Method A); M+H=485.2 MS−ES).

### Example 20.4

### 2-Methoxy-N-{6-methyl-5-[3-methyl-8-(6-methylamino-pyridin-3-yl)-2-oxo-2,3-dihydro-imidazo[4,5-c]quinolin-1-yl]-pyridin-2-yl}-acetamide

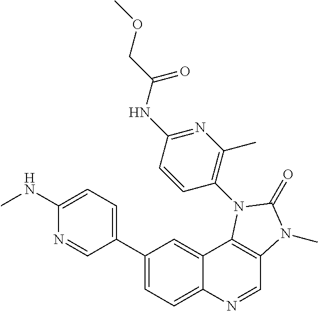

The title compound was synthesized in a similar manner as described for Example 1.1 using 6-(N-BOC-methylamino)pyridin-3-boronicacid pinacolester and intermediate T. Boc was removed in situ (0.5 mL for 5 min at RT) before purification by dissolving the crude product in TFA (0.5 ml) during 5 min.

(HPLC: t_{R} 3.600 min (Method A); M+H=485.2 MS−ES).

### Example 20.5

### N-{5-[8-(5-Isopropoxy-pyridin-3-yl)-3-methyl-2-oxo-2,3-dihydro-imidazo[4,5-c]quinolin-1-yl]-6-methyl-pyridin-2-yl}-2-methoxy-acetamide

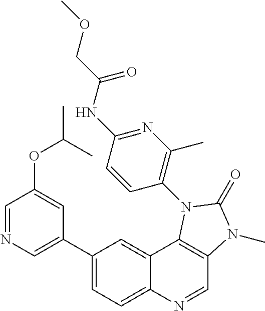

The title compound was synthesized in a similar manner as described for Example 1.1 using 3-isopropoxy-5-(4,4,5,5-tetramethyl-[1,3,2]dioxaborolan-2-yl)-pyridine and intermediate T.

(HPLC: t_{R} 3.950 min (Method A); M+H=513.2 MS−ES, ¹H NMR (600 MHz, DMSO-d₆) δ ppm 10.46 (s, 1H), 9.08 (s, 1H), 8.19-8.25 (m, 3H), 8.16 (d, 1H), 8.07 (d, 1H), 7.98 (dd, 1H), 7.19-7.29 (m, 2H), 4.64 (s, 1H), 4.13 (d, 2H), 3.64 (s, 3H), 3.40 (s, 3H), 2.23 (s, 3H), 1.28 (dd, 6H)).

### Stage 20.5.1 3-Isopropoxy-5-(4,4,5,5-tetramethyl-[1,3,2]dioxaborolan-2-yl)-pyridine

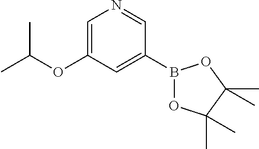

The title compound was synthesized in a similar manner as described in example 2.27, stage 2.27.1 using 3-bromo-5-isopropoxypyridine (Stage 20.5.2) to give the title compound as a brown sticky solid. (HPLC: t_{R} 2.12 min (Method A); M+H=264 MS−ES).

### Stage 20.5.2 3-Bromo-5-isopropoxypyridine

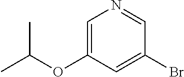

To a mixture of 3-bromo-5-hydroxypyridine (Aldrich, Buchs, Switzerland, 300 mg; 1.672 mmol), triphenylphosphine (658 mg, 2.509 mmol) and isopropanol (0.154 ml, 2.007 mmol) in THF (8 ml) under Argon was added dropwise diisopropyl azodicarboxylate (0.518 ml, 2.509 mmol). The RM was stirred at rt for 15 h. Then the RM was diluted with EtOAc and the organic layer was washed with NaHCO₃ (satd. soln.) and brine (2×), before being dried over Na₂SO₄, filtered and evaporated. The residue was absorbed on silica gel and purified by chromatography (Combiflash; solvent system: heptan/EtOAc 0%-100%) to obtain the title compound (330 mg) as a colorless oil. (HPLC: t_{R} 2.92 min (Method C); M+H=216, 218 MS−ES).

### Example 21.1

### 8-(6-Amino-5-trifluoromethyl-pyridin-3-yl)-3-methyl-1-[2-methyl-6-(4-methyl-3-oxo-piperazin-1-yl)-pyridin-3-yl]-1,3-dihydro-imidazo[4,5-c]quinolin-2-one

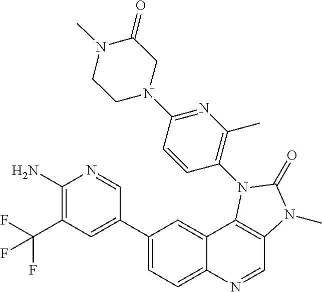

The title compound was synthesized in a similar manner as described for Example 1.1 using 5-(4,4,5,5-tetramethyl-[1,3,2]dioxaborolan-2-yl)-3-trifluoromethyl-pyridin-2-ylamine and intermediate U.

(HPLC: t_{R} 4.582 (method B); M+H=563 MS−ES, ¹H-NMR (d₆-DMSO, 600 MHz) 8.98 (s, 1H), 8.40 (s, 1H), 8.09-8.07 (m, 1H), 7.95-7.94 (m, 1H), 7.76-7.75 (m, 1H), 7.56 (s, 1H), 7.20 (s, 1H), 6.94-6.93 (m, 1H), 6.73 (m, 2H), 4.16 (m, 2H), 3.94-3.92 (m, 2H), 3.61 (s, 3H), 3.48-3.47 (m, 2H), 2.95 (s, 3H), 2.11 (s, 3H)).

### Stage 21.1.1 4-(5-Amino-6-methyl-pyridin-2-yl)-1-methyl-piperazin-2-one

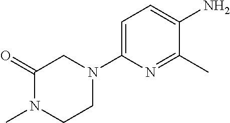

The title compound was prepared from 1-methyl-4-(6-methyl-5-nitro-pyridin-2-yl)-piperazin-2-one as described in example 19., stage 19.1.1. (HPLC: t_{R} 3.160 min (Method B); M+H=221 MS−ES.

### Stage 21.1.2 1-Methyl-4-(6-methyl-5-nitro-pyridin-2-yl)-piperazin-2-one

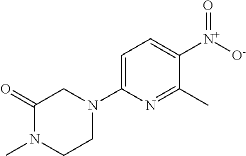

To a mixture of 6-chloro-3-nitro-2-picoline (500 mg, 2.84 mmol) and 1-methyl-piperazin-2-one hydrochloride (535 mg, 3.55 mmol) in dioxane (15 ml) were added Cs₂CO₃ (1215 mg, 3.69 mmol) and xantphos (25.4 mg, 0.043 mmol). The mixture was degassed with Argon for 5 min, then Pd(OAc)₂ (6.37 mg, 0.028 mmol) was added. The light brown suspension was heated up to 110° C. and stirred for 18 h. The reaction mixture was concentrated then diluted in EtOAc/NaHCO₃ aq. sat. sol°. The aqueous layer was extracted twice with EtOAc. The combined organics were washed with brine, dried (Na₂SO₄) and after filtration, the solvent was removed under reduced pressure and the crude product was dried under high vacuum (50° C.). Purification was done by chromatography on silica gel (eluting with DCM/MeOH; 99/1 then 98/2) to obtain the title compound as a pink solid (347 mg). (HPLC: t_{R} 5.319 min (Method B); M+H=251.2 MS−ES)

### Example 22.1

### 8-(5-Ethoxy-6-methoxymethyl-pyridin-3-yl)-1-[6-(2-methoxy-ethoxy)-2-methyl-pyridin-3-yl]-3-methyl-1,3-dihydro-imidazo[4,5-c]quinolin-2-one

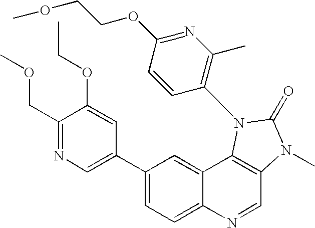

The title compound was synthesized in a similar manner as described for Example 1.1 using 3-ethoxy-2-methoxymethyl-5-(4,4,5,5-tetramethyl-[1,3,2]dioxaborolan-2-yl)-pyridine and intermediate V.

(HPLC: t_{R} 4.869 (method B); M+H=530 MS−ES, ¹H-NMR (d₆-DMSO, 600 MHz) 9.06 (s, 1H), 8.21 (s, 1H), 8.17-8.15 (m, 1H), 8.00 (m, 1H), 7.97-7.95 (m, 1H), 7.26 (s, 2H), 6.99-6.98 (m, 1H), 4.50-4.46 (m, 4H), 4.3-4.1 (m, 2H), 3.71-3.69 (m, 2H), 3.63 (s, 3H), 3.33-3.30 (m, 6H), 2.21 (s, 3H), 1.42-1.40 (m, 3H)).

### Stage 22.1.1 6-(2-Methoxy-ethoxy)-2-methyl-pyridin-3-ylamine

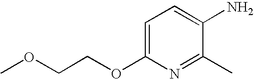

The title compound was prepared from 6-(2-methoxy-ethoxy)-2-methyl-3-nitro-pyridine as described in example 19., stage 19.1.1. (HPLC: t_{R} 3.377 min (Method B); M+H=183.1 MS−ES.

### Stage 22.1.2 6-(2-Methoxy-ethoxy)-2-methyl-3-nitro-pyridine

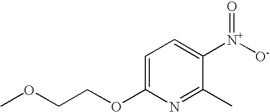

6-Chloro-3-nitro-2-picoline (500 mg, 2.90 mmol) was diluted in 2-methoxyethanol (20 mL, 254 mmol) and stirred for 5 min under Ar. NaH 55-65% in mineral oil (579 mg, 14.49 mmol) was slowly added portionwise over 15 min to the light brown solution at RT (Control of temperature with a water/ice bath). The reaction mixture was stirred for 45 min at 0° C. then for 30 min at RT. The reaction mixture was poured into a mix of water/ice/phosphate buffer solution (pH=7.0). The aqueous layer was then extracted three times with EtOAc. The combined organic layers were dried (Na₂SO₄) and after filtration, the solvent was removed under reduced pressure and the crude product was dried under high vacuum (40/50° C.) to obtain the title compound (778 mg) as a brown solid. The product was used in the next step without further purification. (HPLC: t_{R} 6.371 min (Method B); M+H=213.1 MS−ES.

### Example 22.2

### 8-(5-Azetidin-1-yl-pyridin-3-yl)-1-[6-(2-methoxy-ethoxy)-2-methyl-pyridin-3-yl]-3-methyl-1,3-dihydro-imidazo[4,5-c]quinolin-2-one

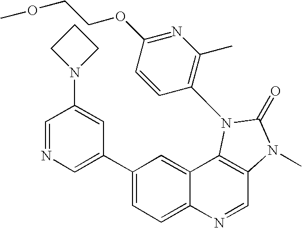

The title compound was synthesized in a similar manner as described for Example 1.1 using 3-azetidin-1-yl-5-(4,4,5,5-tetramethyl-[1,3,2]dioxaborolan-2-yl)-pyridine and intermediate V.

(HPLC: t_{R} 4.526 (method B); M+H=497 MS−ES).

### Example 22.3

### 1-[6-(2-Methoxy-ethoxy)-2-methyl-pyridin-3-yl]-8-(5-methoxy-6-methoxymethyl-pyridin-3-yl)-3-methyl-1,3-dihydro-imidazo[4,5-c]quinolin-2-one

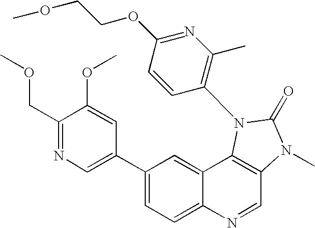

The title compound was synthesized in a similar manner as described for Example 1.1 using 3-methoxy-2-methoxymethyl-5-(4,4,5,5-tetramethyl-[1,3,2]dioxaborolan-2-yl)-pyridine and intermediate V.

(HPLC: t_{R} 4.649 (method B); M+H=516 MS−ES).

### Example 23.1

### 1-(6-Azetidin-1-yl-2-methyl-pyridin-3-yl)-8-(5-azetidin-1-yl-pyridin-3-yl)-3-methyl-1,3-dihydro-imidazo[4,5-c]quinolin-2-one

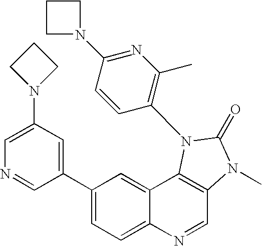

The title compound was synthesized in a similar manner as described for Example 1.1 using 3-azetidin-1-yl-5-(4,4,5,5-tetramethyl-[1,3,2]dioxaborolan-2-yl)-pyridine and intermediate W.

(HPLC: t_{R} 3.533 (method A); M+H=478.2 MS−ES).

### Stage 23.1.1 6-Azetidin-1-yl-2-methyl-pyridin-3-ylamine

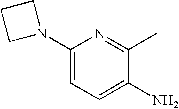

The title compound was prepared from 6-azetidin-1-yl-2-methyl-3-nitro-pyridine as described in example 19., stage 19.1.1. (HPLC: t_{R} 3.208 min (Method A); M+H=164.1 MS−ES.

### Stage 23.1.2 6-Azetidin-1-yl-2-methyl-3-nitro-pyridine

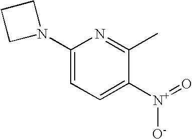

6-Chloro-2-methyl-3-nitropyridine (690 mg; 4.0 mmol) was dissolved in MeOH (10 mL), followed by addition of azetidine (283 mg; 4.8 mmol) and the mixture was stirred for 24 h at 40° C. After cooling to RT, EtOAc (100 mL) was added and the organic layer extracted with water (2×). The organic layer was freed from the solvent under reduced pressure to obtain the title compound as a bright beige powder which was used in the next step without further purification (705 mg). (HPLC: t_{R} 4.242 min (Method A); M+H=194.1 MS−ES.

### Example 23.2

### 1-(6-Azetidin-1-yl-2-methyl-pyridin-3-yl)-8-(5-isopropoxy-6-methoxymethyl-pyridin-3-yl)-3-methyl-1,3-dihydro-imidazo[4,5-c]quinolin-2-one

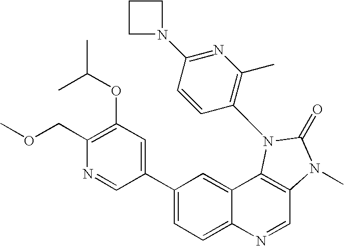

The title compound was synthesized in a similar manner as described for Example 1.1 using 3-isopropoxy-2-methoxymethyl-5-(4,4,5,5-tetramethyl-[1,3,2]dioxaborolan-2-yl)-pyridine and intermediate W.

(HPLC: t_{R} 3.783 (method A); M+H=525.2 MS−ES, ¹H-NMR (d₆-DMSO, 600 MHz) 9.03 (s, 1H), 8.17-8.14 (m, 3H), 8.01-8.00 (m, 1H). 7.67-7.66 (m, 1H), 7.36 (m, 3H), 6.45-6.44 (m, 1H), 4.68 (m, 1H), 4.48 (s, 2H), 4.06-4.01 (m, 4H), 3.62 (s, 3H), 2.38-2.37 (m, 2H), 2.08 (s, 3H), 1.35-1.32 (m, 6H)).

### Stage 23.2.1 3-Isopropoxy-2-methoxymethyl-5-(4,4,5,5-tetramethyl-[1,3,2]dioxaborolan-2-yl)-pyridine

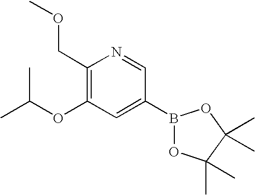

The title compound was synthesized in a similar manner as described for Stage 7.12.1 using 5-bromo-3-isopropoxy-2-methoxymethyl-pyridine (stage 23.2.2, 0.142 mmol) to give the title compound as a crude brown oil. (degrading under the HPLC condition: t_{R} 2.40 min (Method C); M+H=308 MS−ES).

### Stage 23.2.2 5-Bromo-3-isopropoxy-2-methoxymethyl-pyridine

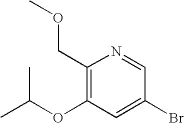

To a solution of (5-bromo-3-isopropoxy-pyridin-2-yl)-methanol (stage 23.2.3, 0.61 mmol) in DMF (3 ml) was added 55% NaH in oil (0.688 mmol) was added. The RM was stirred for 30 min at rt then was added iodomethane (0.684 mmol). The RM was stirred for 2.5 h at rt then was quenched with MeOH and purified by preparative HPLC. The fractions containing product were combined, basified with NaHCO₃, concentrated and extracted with dichloromethane (2×). The combined organic layers were washed with brine, dried over Na₂SO₄, filtered and evaporated to dryness to give the title compound as a yellow oil. (HPLC: t_{R} 2.83 min (Method C); M+H=260, 262 MS−ES).

### Stage 23.2.3 (5-Bromo-3-isopropoxy-pyridin-2-yl)-methanol

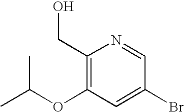

To a solution of 5-bromo-3-isopropoxy-2-methyl-pyridine 1-oxide (Stage 23.2.4, 2.054 mmol) in THF (13 ml) was added trifluoroacetic anhydride (10.27 mmol). The RM was stirred for 4 h at rt. The RM was evaporated to dryness. The residue was treated with saturated aqueous NaHCO₃ (20 ml), stirred for 15 h at rt and then extracted with EtOAc (2×). The combined organic layers were washed with brine, dried over Na₂SO₄, filtered and evaporated. The residue was taken in DMA and purified by preparative HPLC. The fractions containing the product are basified with NaHCO₃, concentrated and extracted with dichloromethane (2×). The combined organic layers were washed with brine, dried over Na₂SO₄, filtered and evaporated to give a yellowish slowly crystallizing solid. (HPLC: t_{R} 2.21 min (Method C); M+H=246, 248 MS−ES).

### Stage 23.2.4 5-Bromo-3-isopropoxy-2-methyl-pyridine 1-oxide

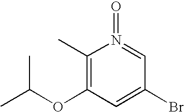

The title compound was synthesized in a similar manner as described for Stage 2.31.3. using 5-bromo-3-isopropoxy-2-methyl-pyridine (Stage 23.2.5, 2.108 mmol) as replacement for (5-bromo-2-methyl-pyridin-3-yl)-ethyl-carbamic acid tert-butyl ester (stage 2.31.4) to give the title compound as a slowly crystallizing orange solid. (HPLC: t_{R} 2.75 min (Method C); M+H=246, 248 MS−ES).

### Stage 23.2.5 5-Bromo-3-isopropoxy-2-methyl-pyridine

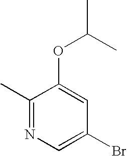

To a solution of 5-bromo-2-methyl-pyridin-3-ylamine (Stage 2.28.6., 400 mg, 2.13 mmol) in isopropanol (33 ml) were added 4 M HCl in dioxane (0.535 ml, 2.13 mmol) and isoamyl nitrite (1.25 g, 10.7 mmol). The reaction mixture was heated at 80° C. for 2.5 h, before being evaporated to dryness. The residue was dissolved in EtOAc and washed with saturated aqueous NaHCO₃. The aqueous layer was extracted with EtOAc and the combined organic layers were dried over Na₂SO₄, filtered and evaporated. The residue was dry loaded on silica gel and purified by MPLC (heptane/EtOAc 0% to 30%) to give the title compound as an orange oil (HPLC: t_{R} 2.47 min (Method C); M+H=230, 232 MS−ES).

### Example 23.3

### 1-(6-Azetidin-1-yl-2-methyl-pyridin-3-yl)-8-[5-(1-hydroxy-1-methyl-ethyl)-pyridin-3-yl]-3-methyl-1,3-dihydro-imidazo[4,5-c]quinolin-2-one

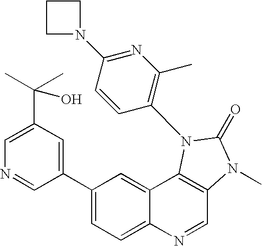

The title compound was synthesized in a similar manner as described for Example 1.1 using 2-[5-(4,4,5,5-tetramethyl-[1,3,2]dioxaborolan-2-yl)-pyridin-3-yl]-propan-2-ol and intermediate W.

(HPLC: t_{R} 3.425 (method A); M+H=481.2 MS−ES, ¹H-NMR (d₆-DMSO, 600 MHz) 9.03 (s, 1H), 8.72 (s, 1H), 8.55 (s, 1H), 8.16-8.14 (m, 1H), 7.98-7.97 (m, 1H), 7.75 (s, 1H), 7.67-7.65 (m, 1H), 7.36 (s, 1H), 6.42-6.41 (m, 1H), 5.29 (s, 1H), 4.06-4.03 (m, 4H), 3.62 (s, 3H), 2.38-2.36 (m, 2H), 2.06 (s, 3H), 1.51-1.50 (m, 6H)).

### Example 23.4

### 1-(6-Azetidin-1-yl-2-methyl-pyridin-3-yl)-3-methyl-8-(6-methylamino-pyridin-3-yl)-1,3-dihydro-imidazo[4,5-c]quinolin-2-one

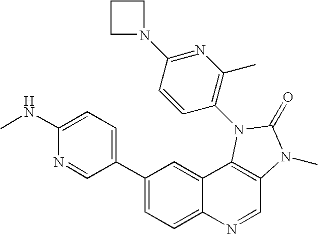

The title compound was synthesized in a similar manner as described for Example 1.1 using 6-(N-BOC-methylamino)pyridin-3-boronicacid pinacolester and intermediate W. Boc was removed in situ (0.5 mL for 5 min at RT) before purification by dissolving the crude product in TFA (0.5 ml) during 5 min.

(HPLC: t_{R} 3.383 (method A); M+H=452.2 MS−ES).

### Example 23.5

### 1-(6-Azetidin-1-yl-2-methyl-pyridin-3-yl)-8-(5-ethoxy-6-methoxymethyl-pyridin-3-yl)-3-methyl-1,3-dihydro-imidazo[4,5-c]quinolin-2-one

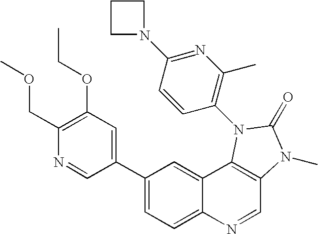

The title compound was synthesized in a similar manner as described for Example 1.1 using 3-ethoxy-2-methoxymethyl-5-(4,4,5,5-tetramethyl-[1,3,2]dioxaborolan-2-yl)-pyridine and intermediate W.

(HPLC: t_{R} 3.700 (method A); M+H=511.2 MS−ES, ¹H NMR (600 MHz, DMSO-d₆) δ ppm 9.03 (s, 1H), 8.21 (d, 1H), 8.14 (d, 1H), 8.01 (dd, 1H), 7.67 (d, 1H), 7.35 (d, 1H), 7.31 (d, 1H), 6.46 (d, 1H), 4.50 (s, 2H), 4.12 (dd, 2H), 4.04 (ddd, 4H), 3.62 (s, 3H), 3.32 (s, 3H), 2.39 (t, 2H), 2.08 (s, 3H), 1.44 (t, 3H)).

### Example 23.6

### 1-(6-Azetidin-1-yl-2-methyl-pyridin-3-yl)-8-(5-methoxy-6-methoxymethyl-pyridin-3-yl)-3-methyl-1,3-dihydro-imidazo[4,5-c]quinolin-2-one

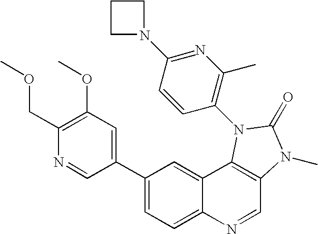

The title compound was synthesized in a similar manner as described for Example 1.1 using 3-methoxy-2-methoxymethyl-5-(4,4,5,5-tetramethyl-[1,3,2]dioxaborolan-2-yl)-pyridine and intermediate W.

(HPLC: t_{R} 3.592 (method A); M+H=497.2 MS−ES).

### Example 23.7

### 1-(6-Azetidin-1-yl-2-methyl-pyridin-3-yl)-8-(6-ethoxy-pyridin-3-yl)-3-methyl-1,3-dihydro-imidazo[4,5-c]quinolin-2-one

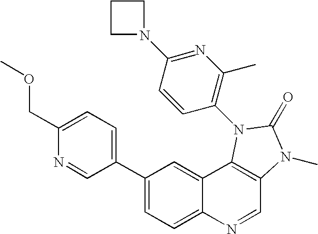

The title compound was synthesized in a similar manner as described for Example 1.1 using 6-ethoxypyridin-3-ylboronic acid and intermediate W.

(HPLC: t_{R} 3.950 (method A); M+H=467.2 MS−ES).

### Example 23.8

### 1-(6-Azetidin-1-yl-2-methyl-pyridin-3-yl)-8-(5-ethylamino-6-hydroxymethyl-pyridin-3-yl)-3-methyl-1,3-dihydro-imidazo[4,5-c]quinolin-2-one

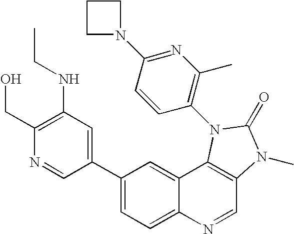

The title compound was synthesized in a similar manner as described for Example 1.1 using acetic acid 3-(tert-butoxycarbonyl-ethyl-amino)-5-(4,4,5,5-tetramethyl-[1,3,2]dioxa-borolan-2-yl)-pyridin-2-ylmethyl ester and intermediate W. The acetyl protecting group as well as the BOC protecting group were removed together in situ by dissolving the crude product in an aqueous solution of LiOH (1 M, 4 equivalents), stirring for 5 min at RT and the removing the solvent under reduced pressure.

(HPLC: t_{R} 3.450 (method A); M+H=496.2 MS−ES).

### Example 23.9

### 1-(6-Azetidin-1-yl-2-methyl-pyridin-3-yl)-8-(5-isopropoxy-pyridin-3-yl)-3-methyl-1,3-dihydro-imidazo[4,5-c]quinolin-2-one

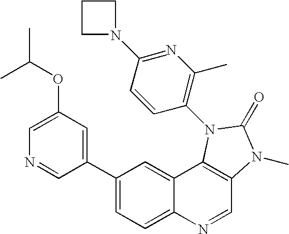

The title compound was synthesized in a similar manner as described for Example 1.1 using 3-isopropoxy-5-(4,4,5,5-tetramethyl-[1,3,2]dioxaborolan-2-yl)-pyridine and intermediate W.

(HPLC: t_{R} 3.717 (method A); M+H=481.2 MS−ES).

### Example 24.1

### 1-[6-(3,3-Difluoro-azetidin-1-yl)-2-methyl-pyridin-3-yl]-3-methyl-8-(2-methylamino-pyrimidin-5-yl)-1,3-dihydro-imidazo[4,5-c]quinolin-2-one

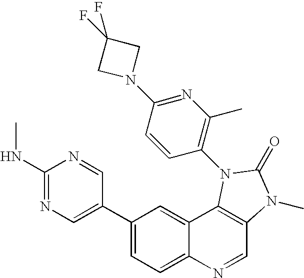

The title compound was synthesized in a similar manner as described for Example 1.1 using 2-methylaminopyrimidin-5-boronic acid pinacolester and intermediate X.

(HPLC: t_{R} 4.68 (method B); M+H=489.2 MS−ES, ¹H-NMR (d₆-DMSO, 600 MHz) 8.98 (s, 1H), 8.34 (s, 2H), 8.09-8.07 (m, 1H), 7.88-7.86 (m, 1H), 7.81-7.80 (m, 1H), 7.38-7.37 (m, 1H), 7.08 (s, 1H), 6.73-6.71 (m, 1H), 4.54-4.50 (m, 4H), 3.61 (s, 3H), 2.83 (m, 3H), 2.14 (s, 3H)).

### Stage 24.1.1 6-(3,3-Difluoro-azetidin-1-yl)-2-methyl-pyridin-3-ylamine

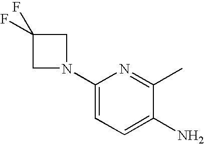

The title compound was prepared from 6-(3,3-difluoro-azetidin-1-yl)-2-methyl-3-nitro-pyridine as described in example 19., stage 19.1.1. (HPLC: t_{R} 3.517 min (Method B); M+H=200.1 MS−ES.)

### Stage 24.1.2 6-(3,3-Difluoro-azetidin-1-yl)-2-methyl-3-nitro-pyridine

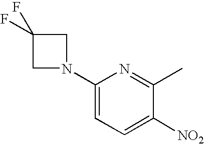

6-Chloro-2-methyl-3-nitropyridine (1 g, 5.79 mmol) and 3,3-difluoroazetidine hydrochloride salt (0.948 g, 6.95 mmol) were dissolved in MeOH (15 ml) followed by addition of TEA (1.22 mL; 8.69 mMol) and stirred at 40° C. for 2 h, then at 60° C. for 17 h. At RT, K₂CO₃ (0.801 g, 5.79 mmol) was added, followed by 3,3-difluoroazetidine hydrochloridride salt (0.395 g, 2.9 mmol) and the solution was stirred at 60° C. for 16 h. The reaction mixture was diluted with EtOAc and washed with NaHCO₃ aq. sat. sol° and brine. The combined organic layers were dried (Na₂SO₄) and after filtration, the solvent was removed under reduced pressure and the crude product was dried under high vacuum (50° C.) to obtain the title compound (1.256 g) as a brown solid. The product was used in the next step without further purification. (HPLC: t_{R} 6.468 min (Method B); M+H=230 MS−ES.)

### Example 24.2

### 1-[6-(3,3-Difluoro-azetidin-1-yl)-2-methyl-pyridin-3-yl]-8-[5-(1-hydroxy-1-methyl-ethyl)-pyridin-3-yl]-3-methyl-1,3-dihydro-imidazo[4,5-c]quinolin-2-one

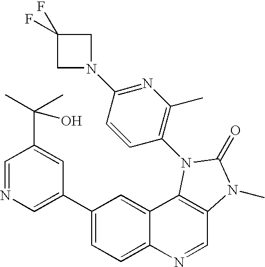

The title compound was synthesized in a similar manner as described for Example 1.1 using 2-[5-(4,4,5,5-tetramethyl-[1,3,2]dioxaborolan-2-yl)-pyridin-3-yl]-propan-2-ol and intermediate X.

(HPLC: t_{R} 4.345 (method B); M+H=517.2 MS−ES).

### Example 24.3

### 1-[6-(3,3-Difluoro-azetidin-1-yl)-2-methyl-pyridin-3-yl]-8-(5-ethylamino-6-hydroxymethyl-pyridin-3-yl)-3-methyl-1,3-dihydro-imidazo[4,5-c]quinolin-2-one

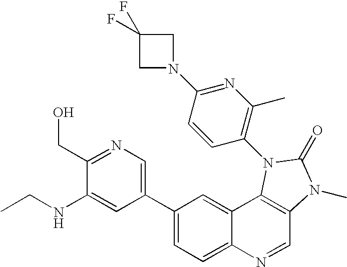

The title compound was synthesized in a similar manner as described for Example 1.1 using acetic acid 3-(tert-butoxycarbonyl-ethyl-amino)-5-(4,4,5,5-tetramethyl-[1,3,2]dioxa-borolan-2-yl)-pyridin-2-ylmethyl ester and intermediate X. The acetyl protecting group as well as the BOC protecting group were removed together in situ by dissolving the crude product in an aqueous solution of LiOH (1 M, 4 equivalents), stirring for 5 min at RT and the removing the solvent under reduced pressure.

(HPLC: t_{R} 4.471 (method B); M+H=532 MS−ES).

### Example 24.4

### 1-[6-(3,3-Difluoro-azetidin-1-yl)-2-methyl-pyridin-3-yl]-8-(6-hydroxymethyl-5-methoxy-pyridin-3-yl)-3-methyl-1,3-dihydro-imidazo[4,5-c]quinolin-2-one

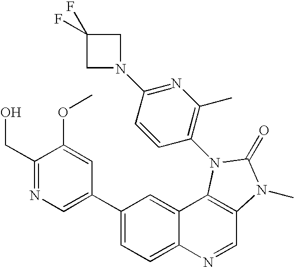

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester and intermediate X. The acetyl protecting group was removed in situ by dissolving the crude product in an aqueous solution of LiOH (1 M, 4 equivalents), stirring for min at RT and the removing the solvent under reduced pressure.

(HPLC: t_{R} 4.369 (method B); M+H=519.2 MS−ES, ¹H NMR (600 MHz, DMSO-d₆) δ ppm 9.04 (s, 1H), 8.23 (d, 1H), 8.16 (d, 1H), 8.03 (dd, 1H), 7.83 (d, 1H), 7.35 (d, 1H), 7.24 (s, 1H), 6.70 (d, 1H), 4.90 (t, 1H), 4.41-4.60 (m, 6H), 3.87 (s, 3H), 3.62 (s, 3H), 2.15 (s, 3H)).

### Example 25.1

### 3-Methyl-8-(6-methylamino-pyridin-3-yl)-1-(2-methyl-6-pyrrolidin-1-yl-pyridin-3-yl)-1,3-dihydro-imidazo[4,5-c]quinolin-2-one

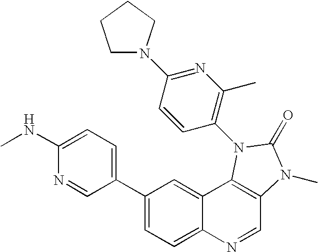

The title compound was synthesized in a similar manner as described for Example 1.1 using 6-(N-BOC-methylamino)pyridin-3-boronicacid pinacolester and intermediate Y. Boc was removed in situ (0.5 mL for 5 min at RT) before purification by dissolving the crude product in TFA (0.5 ml) during 5 min.

(HPLC: t_{R} 3.483 (method A); M+H=466.2 MS−ES).

### Stage 25.1.1 2-Methyl-6-pyrrolidin-1-yl-pyridin-3-ylamine

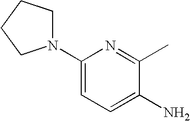

The title compound was prepared from 6-chloro-2-methyl-3-nitropyridine as described in example 23., stage 23.1.1. and 23.1.2., using pyrrolidine instead of aziridine. (HPLC: t_{R} 3.292 min (Method A); M+H=178.1 MS−ES.

### Example 25.2

### 8-(5-Ethoxy-6-methoxymethyl-pyridin-3-yl)-3-methyl-1-(2-methyl-6-pyrrolidin-1-yl-pyridin-3-yl)-1,3-dihydro-imidazo[4,5-c]quinolin-2-one

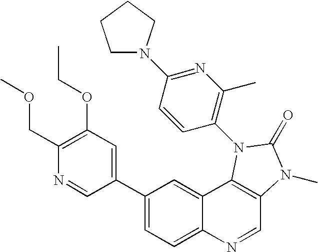

The title compound was synthesized in a similar manner as described for Example 1.1 using 3-ethoxy-2-methoxymethyl-5-(4,4,5,5-tetramethyl-[1,3,2]dioxaborolan-2-yl)-pyridine and intermediate Y.

(HPLC: t_{R} 3.775 (method A); M+H=525.2 MS−ES, ¹H-NMR (d₆-DMSO, 600 MHz) 9.03 (s, 1H), 8.20 (s, 1H), 8.15-8.14 (m, 1H), 8.01-7.99 (m, 1H), 7.64-7.63 (m, 1H), 7.43 (m, 1H), 7.31 (m, 1H), 6.55-6.53 (m, 1H), 4.48 (s, 2H), 4.09-4.06 (m, 2H), 3.62 (s, 3H), 3.47-3.46 (m, 4H), 3.31 (s, 3H), 2.08 (s, 3H), 2.00-1.99 (m, 4H), 1.39-1.36 (m, 3H)).

### Example 25.3

### 8-[5-(1-Hydroxy-1-methyl-ethyl)-pyridin-3-yl]-3-methyl-1-(2-methyl-6-pyrrolidin-1-yl-pyridin-3-yl)-1,3-dihydro-imidazo[4,5-c]quinolin-2-one

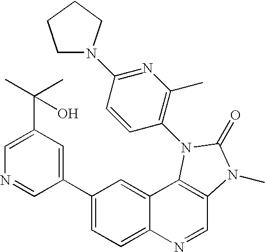

The title compound was synthesized in a similar manner as described for Example 1.1 using 2-[5-(4,4,5,5-tetramethyl-[1,3,2]dioxaborolan-2-yl)-pyridin-3-yl]-propan-2-ol and intermediate Y.

(HPLC: t_{R} 3.533 (method A); M+H=495.2 MS−ES; ¹H NMR (600 MHz, DMSO-d₆) δ ppm 9.02 (s, 1H), 8.70 (s, 1H), 8.61 (s, 1H), 8.15 (d, 1H), 7.99 (dd, 1H), 7.57-7.72 (m, 2H), 7.46 (s, 1H), 6.51 (d, 1H), 5.26 (s, 1H), 3.62 (s, 3H), 3.46 (br. s., 4H), 2.06 (s, 3H), 1.99 (br. s., 4H), 1.42 (d, 6H)).

### Example 25.4

### 8-(6-Hydroxymethyl-5-methoxy-pyridin-3-yl)-3-methyl-1-(2-methyl-6-pyrrolidin-1-yl-pyridin-3-yl)-1,3-dihydro-imidazo[4,5-c]quinolin-2-one

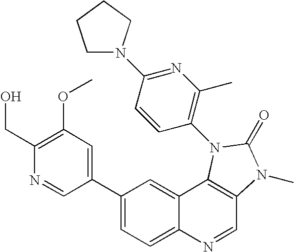

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester and intermediate Y. The acetyl protecting group was removed in situ by dissolving the crude product in an aqueous solution of LiOH (1 M, 4 equivalents), stirring for 5 min at RT and the removing the solvent under reduced pressure.

(HPLC: t_{R} 3.475 (method A); M+H=497.2 MS−ES).

### Example 25.5

### 8-(5-Ethylamino-6-hydroxymethyl-pyridin-3-yl)-3-methyl-1-(2-methyl-6-pyrrolidin-1-yl-pyridin-3-yl)-1,3-dihydro-imidazo[4,5-c]quinolin-2-one

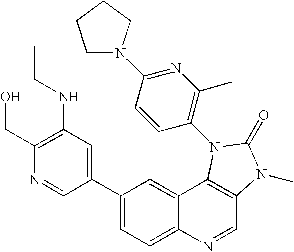

The title compound was synthesized in a similar manner as described for Example 1.1 using acetic acid 3-(tert-butoxycarbonyl-ethyl-amino)-5-(4,4,5,5-tetramethyl-[1,3,2]dioxa-borolan-2-yl)-pyridin-2-ylmethyl ester and intermediate Y. The acetyl protecting group as well as the BOC protecting group were removed together in situ by dissolving the crude product in an aqueous solution of LiOH (1 M, 4 equivalents), stirring for 5 min at RT and the removing the solvent under reduced pressure.

(HPLC: t_{R} 3.533 (method A); M+H=510.4 MS−ES).

### Example 26.1

### 8-(6-Amino-5-trifluoromethyl-pyridin-3-yl)-1-{6-[(2-hydroxy-ethyl)-methyl-amino]-2-methyl-pyridin-3-yl}-3-methyl-1,3-dihydro-imidazo[4,5-c]quinolin-2-one

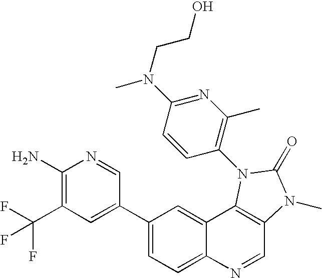

The title compound was synthesized in a similar manner as described for Example 1.1 using 5-(4,4,5,5-tetramethyl-[1,3,2]dioxaborolan-2-yl)-3-trifluoromethyl-pyridin-2-ylamine and intermediate Z1. The silyl protecting group was removed as described below.

(HPLC: t_{R} 4.174 (method B); M+H=524 MS−ES).

Removal of Silyl protecting group:

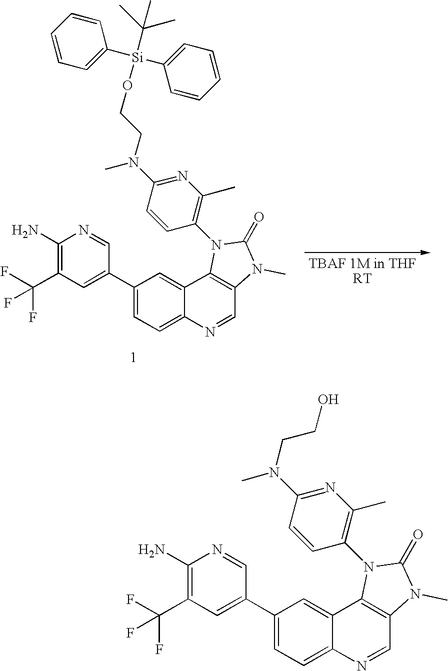

(8-(6-Amino-5-trifluoromethyl-pyridin-3-yl)-1-(6-{[2-(tert-butyl-diphenyl-silanyloxy)-ethyl]-methyl-amino}-2-methyl-pyridin-3-yl)-3-methyl-1,3-dihydro-imidazo[4,5-c]quinolin-2-one (107 mg, 0.098 mmol) was diluted in dry THF (1 ml). The solution was degassed with Ar, then TBAF 1M in THF (0.098 ml, 0.098 mmol) was added. The brown solution was stirred for 1 h at rt under Ar. The solvent was removed under reduced pressure and dried under vacuum (40° C.). Purification was done by chromatography on silica gel (eluting with DCM/MeOH; 99/1 then 97/3) to obtain the title compound as a white solid 1.28 g). (HPLC: t_{R} 4.174 min (Method B); M+H=524.2 MS−ES)).

### Stage 26.1.1 N*2*-[2-(tert-Butyl-diphenyl-silanyloxy)-ethyl]-6,N*2*-dimethyl-pyridine-2,5-diamine

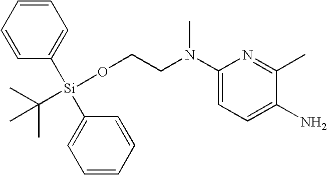

The title compound was prepared from [2-(tert-butyl-diphenyl-silanyloxy)-ethyl]-methyl-(6-methyl-5-nitro-pyridin-2-yl)-amine as described in example 19., stage 19.1.1. (HPLC: t_{R} 6.869 min (Method B); M+H=420.3 MS−ES).

### Stage 26.1.2 [2-(tert-Butyl-diphenyl-silanyloxy)-ethyl]-methyl-(6-methyl-5-nitro-pyridin-2-yl)-amine

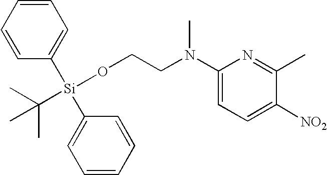

The title compound was prepared from 6-chloro-2-methyl-3-nitropyridine and [2-(tert-butyl-diphenyl-silanyloxy)-ethyl]-methyl-amine as described in example 24., stage 14.1.2. (HPLC: t_{R} 6.403 min (Method B); M+H=450 MS−ES).

### Stage 26.1.3 [2-(tert-Butyl-diphenyl-silanyloxy)-ethyl]methyl-amine

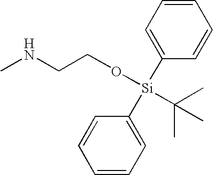

To a colorless solution of 2-(methylamino)ethanol (1 ml, 12.25 mmol) and imidazole (2.085 g, 30.6 mmol) in dry DMF (35 ml) previously degassed with argon, was added slowly dropwise a colorless solution of tert-butyldiphenylchlorosilane (3.88 ml, 14.70 mmol) in dry DMF (15 ml) previously degassed with argon. The colorless solution was stirred for 24 h and then poured onto ice/water. This aqueous layer was extracted twice with CH₂Cl₂. The combined organics were washed with brine, dried (Na₂SO₄) and after filtration, the solvent was removed under reduced pressure and the crude product was dried under high vacuum (50° C.). Purification was done by chromatography on silica gel (eluting with CH₂Cl₂/MeOH/NH₃ 95:5:0.5 DCM+MeOH 95:5+0.5% NH₃) to obtain the title compound as a yellow oil (1.28 g). (HPLC: t_{R} 6.609 min (Method B); M+H=314.2 MS−ES).

### Example 26.2

### 1-{6-[(2-Hydroxy-ethyl)-methyl-amino]-2-methyl-pyridin-3-yl}-3-methyl-8-(2-methylamino-pyrimidin-5-yl)-1,3-dihydro-imidazo[4,5-c]quinolin-2-one

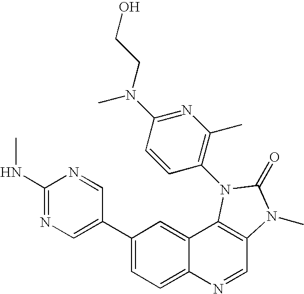

The title compound was synthesized in a similar manner as described for Example 1.1 using 2-methylaminopyrimidin-5-boronic acid pinacolester and intermediate Z1. The silyl protecting group was removed as the very last step as described in example 26.1.

(HPLC: t_{R} 3.855 (method B); M+H=471.2 MS−ES; ¹H NMR (600 MHz, DMSO-d₆) δ ppm 8.96 (s, 1H), 8.35 (br. s., 2H), 8.07 (d, 1H), 7.88 (dd, 1H), 7.61 (d, 1H), 7.40 (d, 1H), 7.17 (d, 1H), 6.73 (d, 1H), 4.78 (t, 1H), 3.63-3.77 (m, 4H), 3.61 (s, 3H), 3.10-3.21 (m, 3H), 2.74-2.87 (m, 3H), 2.07 (s, 3H)).

### Example 26.3

### 8-(5-Ethylamino-6-hydroxymethyl-pyridin-3-yl)-1-{6-[(2-hydroxy-ethyl)-methyl-amino]-2-methyl-pyridin-3-yl}-3-methyl-1,3-dihydro-imidazo[4,5-c]quinolin-2-one

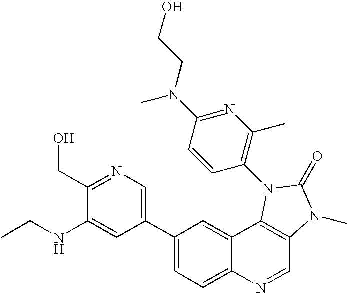

The title compound was synthesized in a similar manner as described for Example 1.1 using acetic acid 3-(tert-butoxycarbonyl-ethyl-amino)-5-(4,4,5,5-tetramethyl-[1,3,2]dioxa-borolan-2-yl)-pyridin-2-ylmethyl ester and intermediate Z1. The acetyl protecting group as well as the BOC protecting group were removed together in situ by dissolving the crude product in an aqueous solution of LiOH (1 M, 4 equivalents), stirring for 5 min at RT and the removing the solvent under reduced pressure. The silyl protecting group was removed as the very last step as described in example 26.1.

(HPLC: t_{R} 3.807 (method B); M+H=514.2 MS−ES).

### Example 27.1

### 1-[6-(3-Hydroxy-pyrrolidin-1-yl)-2-methyl-pyridin-3-yl]-8-(5-isopropoxy-pyridin-3-yl)-3-methyl-1,3-dihydro-imidazo[4,5-c]quinolin-2-one

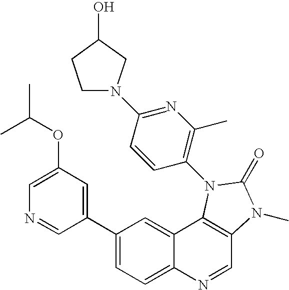

The title compound was synthesized in a similar manner as described for Example 1.1 using 3-isopropoxy-5-(4,4,5,5-tetramethyl-[1,3,2]dioxaborolan-2-yl)-pyridine and intermediate Z2.

(HPLC: t_{R} 3.650 (method A); M+H=511.2 MS−ES; ¹H NMR (600 MHz, DMSO-d₆) δ ppm 9.03 (s, 1H), 8.23 (br. s., 2H), 8.14 (d, 1H), 7.97 (d, 1H), 7.64 (dd, 1H), 7.45 (s, 1H), 7.30 (br. s., 1H), 6.52 (d, 1H), 5.04 (dd, 1H), 4.63-4.77 (m, 1H), 4.43 (br. s., 1H), 3.47-3.66 (m, 7H), 2.00-2.12 (m, 5H), 1.22-1.31 (m, 6H)).

### Stage 27.1.1 1-(5-Amino-6-methyl-pyridin-2-yl)-pyrrolidin-3-ol

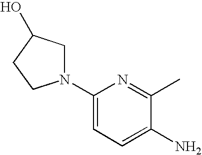

The title compound was prepared from 6-chloro-2-methyl-3-nitropyridine as described in example 23., stage 23.1.1. and 23.1.2., using pyrrolidin-3-ol instead of aziridine. (HPLC: t_{R} 3.042 min (Method A); M+H=194.1 MS−ES).

### Example 27.2

### 1-[6-(3-Hydroxy-pyrrolidin-1-yl)-2-methyl-pyridin-3-yl]-3-methyl-8-(2-methylamino-pyrimidin-5-yl)-1,3-dihydro-imidazo[4,5-c]quinolin-2-one

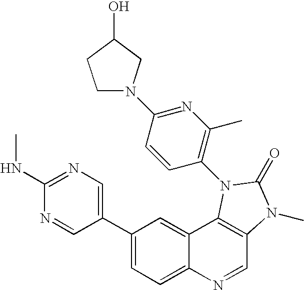

The title compound was synthesized in a similar manner as described for Example 1.1 using 2-methylaminopyrimidin-5-boronic acid pinacolester and intermediate Z2.

(HPLC: t_{R} 3.492 (method A); M+H=483.2 MS−ES, ¹H-NMR (d₆-DMSO, 600 MHz) 8.96 (s, 1H), 8.35 (s, br, 2H), 8.07-8.06 (m, 1H), 7.87-7.85 (m, 1H), 7.62-7.60 (m, 1H), 7.37-7.36 (m, 1H), 7.18 (m, 1H), 6.54-6.53 (m, 1H), 5.04-5.02 (m, 1H), 4.45 (s, 1H), 3.63-3.56 and 3.37-3.30 (m, 7H), 2.83-2.82 (m, 3H), 2.07 (s, 3H), 2.11-1.96 (m, 2H)).

### Example 27.3

### 8-(6-Amino-5-trifluoromethyl-pyridin-3-yl)-1-[6-(3-hydroxy-pyrrolidin-1-yl)-2-methyl-pyridin-3-yl]-3-methyl-1,3-dihydro-imidazo[4,5-c]quinolin-2-one

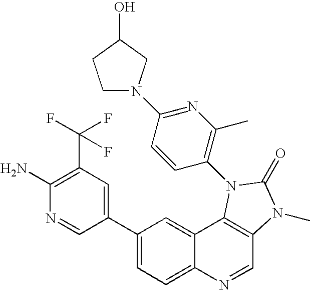

The title compound was synthesized in a similar manner as described for Example 1.1 using 5-(4,4,5,5-tetramethyl-[1,3,2]dioxaborolan-2-yl)-3-trifluoromethyl-pyridin-2-ylamine and intermediate Z2.

(HPLC: t_{R} 3.725 (method A); M+H=536.2 MS−ES).

### Example 27.4

### 8-(6-Ethoxy-pyridin-3-yl)-1-[6-(3-hydroxy-pyrrolidin-1-yl)-2-methyl-pyridin-3-yl]-3-methyl-1,3-dihydro-imidazo[4,5-c]quinolin-2-one

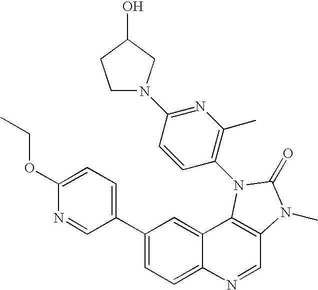

The title compound was synthesized in a similar manner as described for Example 1.1 using 6-ethoxypyridin-3-ylboronic acid and intermediate Z2.

(HPLC: t_{R} 3.900 (method A); M+H=497.2 MS−ES).

### Example 27.5

### 8-[5-(1-Hydroxy-1-methyl-ethyl)-pyridin-3-yl]-1-[6-(3-hydroxy-pyrrolidin-1-yl)-2-methyl-pyridin-3-yl]-3-methyl-1,3-dihydro-imidazo[4,5-c]quinolin-2-one

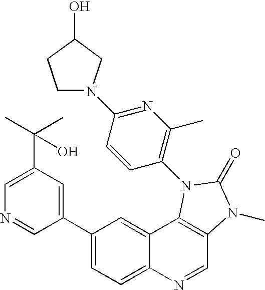

The title compound was synthesized in a similar manner as described for Example 1.1 using 2-[5-(4,4,5,5-tetramethyl-[1,3,2]dioxaborolan-2-yl)-pyridin-3-yl]-propan-2-ol and intermediate Z2.

(HPLC: t_{R} 3.425 (method A); M+H=511.2 MS−ES, ¹H NMR (600 MHz, DMSO-d₆) δ ppm 9.03 (s, 1H), 8.69 (t, 1H), 8.61 (t, 1H), 8.07-8.20 (m, 2H), 7.99 (dd, 1H), 7.66-7.77 (m, 1H), 7.64 (dd, 1H), 7.48 (s, 1H), 6.51 (dd, 1H), 5.26 (s, 1H), 5.10 (s, 1H), 4.44 (br. s., 1H), 3.55-3.63 (m, 7H), 1.8-2.2 (m, 5H), 1.43 (s, 3H), 1.42 (s, 3H)).

### Example 28.1

### 8-[5-(1-Hydroxy-1-methyl-ethyl)-pyridin-3-yl]-1-[6-(3-methoxy-pyrrolidin-1-yl)-2-methyl-pyridin-3-yl]-3-methyl-1,3-dihydro-imidazo[4,5-c]quinolin-2-one

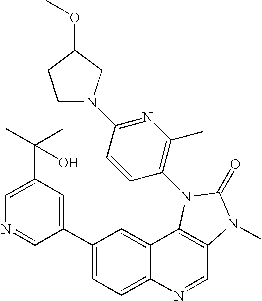

The title compound was synthesized in a similar manner as described for Example 1.1 using 2-[5-(4,4,5,5-tetramethyl-[1,3,2]dioxaborolan-2-yl)-pyridin-3-yl]-propan-2-ol and intermediate Z3.

(HPLC: t_{R} 3.542 (method A); M+H=525.2 MS−ES, ¹H-NMR (d₆-DMSO, 600 MHz) 9.03 (s, 1H), 8.70 (s, 1H), 8.62 (s, 1H), 8.16-8.15 (m, 1H), 8.00 (m, 1H), 7.68-7.65 (m, 2H), 7.47 (m, 1H), 6.54-6.52 (m, 1H), 5.24 (m, 1H), 4.12 (s, 1H), 3.62 (s, 3H), 3.62-3.55 (m, 4H), 3.30 (s, 3H), 2.11-2.08 (m, 2H), 2.08 (s, 3H), 1.43-1.42 (m, 6H)).

Intermediate Z3 was a byproduct in the synthesis of intermediate Z2 at the last stage, the alkylation with methyl iodide (see intermediate A). (HPLC: t_{R} 3.833 (method A); M+H=468.1 MS−ES).

### Example 28.2

### 1-[6-(3-Methoxy-pyrrolidin-1-yl)-2-methyl-pyridin-3-yl]-3-methyl-8-(2-methylamino-pyrimidin-5-yl)-1,3-dihydro-imidazo[4,5-c]quinolin-2-one

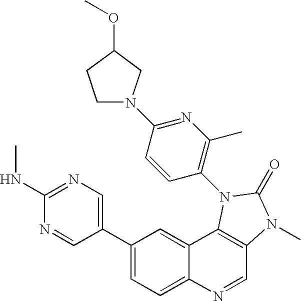

The title compound was synthesized in a similar manner as described for Example 1.1 using 2-methylaminopyrimidin-5-boronic acid pinacolester and intermediate Z3.

(HPLC: t_{R} 3.650 (method A); M+H=497.2 MS−ES).

### Example 28.3

### 8-(6-Hydroxymethyl-5-methoxy-pyridin-3-yl)-1-[6-(3-methoxy-pyrrolidin-1-yl)-2-methyl-pyridin-3-yl]-3-methyl-1,3-dihydro-imidazo[4,5-c]quinolin-2-one

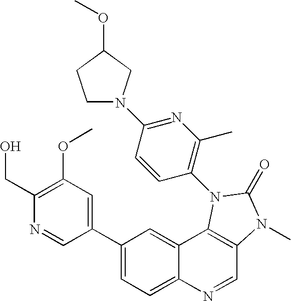

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester and intermediate Z3. The acetyl protecting group was removed in situ by dissolving the crude product in an aqueous solution of LiOH (1 M, 4 equivalents), stirring for 5 min at RT and the removing the solvent under reduced pressure.

(HPLC: t_{R} 3.508 (method A); M+H=527.2 MS−ES).

### Example 29.1

### 1-[6-(3-Hydroxy-azetidin-1-yl)-2-methyl-pyridin-3-yl]-8-[5-(1-hydroxy-1-methyl-ethyl)-pyridin-3-yl]-3-methyl-1,3-dihydro-imidazo[4,5-c]quinolin-2-one

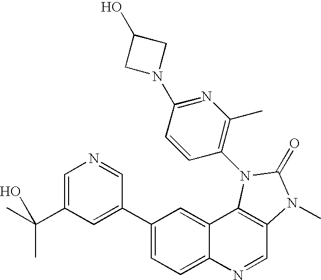

The title compound was synthesized in a similar manner as described for Example 1.1 using 2-[5-(4,4,5,5-tetramethyl-[1,3,2]dioxaborolan-2-yl)-pyridin-3-yl]-propan-2-ol and intermediate Z4. The silyl protecting group was removed as described in example 26.1.

(HPLC: t_{R} 3.53 (method B); M+H=497 MS−ES, ¹H-NMR (d₆-DMSO, 600 MHz) 9.03 (s, 1H), 8.71 (s, 1H), 8.53 (s, 1H), 8.16-8.14 (m, 1H), 7.99-7.97 (m, 1H), 7.76 (s, 1H), 7.68-7.66 (m, 1H), 7.37 (m, 1H), 6.46-6.44 (m, 1H), 5.81-5.80 (m, 1H), 5.30 (s, 1H), 4.63-4.62 (m, 1H), 4.26-4.23 (m, 2H), 3.82-3.78 (m, 2H), 3.62 (s, 3H), 2.08 (s, 3H), 1.49 (s, s, 6H)).

### Stage 29.1.1 6-[3-(tert-Butyl-diphenyl-silanyloxy)-azetidin-1-yl]-2-methyl-pyridin-3-ylamine

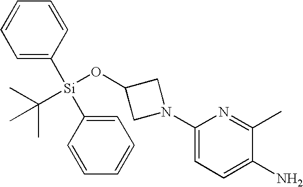

The title compound was prepared as described for example 26.1, stage 261.1-26.1.3 using 3-Hydroxyazetidine hydrochloride instead of 2-(methylamino)ethanol. (HPLC: t_{R} 7.057 (method B); M+H=218.3 MS−ES).

### Example 29.2

### 8-(5-Ethylamino-6-hydroxymethyl-pyridin-3-yl)-1-[6-(3-hydroxy-azetidin-1-yl)-2-methyl-pyridin-3-yl]-3-methyl-1,3-dihydro-imidazo[4,5-c]quinolin-2-one

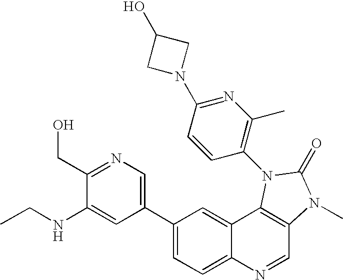

The title compound was synthesized in a similar manner as described for Example 1.1 using acetic acid 3-(tert-butoxycarbonyl-ethyl-amino)-5-(4,4,5,5-tetramethyl-[1,3,2]dioxa-borolan-2-yl)-pyridin-2-yl methyl ester and intermediate Z4. The acetyl protecting group as well as the BOC protecting group were removed together in situ by dissolving the crude product in an aqueous solution of LiOH (1 M, 4 equivalents), stirring for 5 min at RT and the removing the solvent under reduced pressure. The silyl protecting group was removed as described in example 26.1.

(HPLC: t_{R} 3.671 (method B); M+H=512.2 MS−ES, ¹H NMR (600 MHz, DMSO-d₆) δ ppm 9.01 (s, 1H), 8.11 (d, 1H), 7.93 (d, 1H), 7.84 (s, 1H), 7.66 (d, 1H), 7.38 (s, 1H), 6.78 (s, 1H), 6.47 (d, 1H), 5.72-5.86 (m, 1H), 5.46-5.56 (m, 1H), 5.34 (t, 1H), 4.64 (d, 1H), 4.57 (d, 2H), 4.27 (br. s., 1H), 4.23 (br. s., 1H), 3.68-3.88 (m, 2H), 3.61 (s, 3H), 3.12 (dd, 2H), 2.08 (s, 3H), 1.16-1.35 (m, 3H)).

### Example 29.3

### 8-(5-Fluoro-6-methylamino-pyridin-3-yl)-1-[6-(3-hydroxy-azetidin-1-yl)-2-methyl-pyridin-3-yl]-3-methyl-1,3-dihydro-imidazo[4,5-c]quinolin-2-one

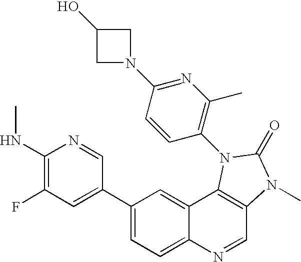

The title compound was synthesized in a similar manner as described for Example 1.1 using [3-fluoro-5-(4,4,5,5-tetramethyl-[1,3,2]dioxaborolan-2-yl)-pyridin-2-yl]-methyl-amine and intermediate Z4. The silyl protecting group was removed as described in example 26.1.

(HPLC: t_{R} 3.691 (method B); M+H=486.3 MS−ES).

### Example 29.4

### 1-[6-(3-Hydroxy-azetidin-1-yl)-2-methyl-pyridin-3-yl]-3-methyl-8-(6-methylamino-5-trifluoromethyl-pyridin-3-yl)-1,3-dihydro-imidazo[4,5-c]quinolin-2-one

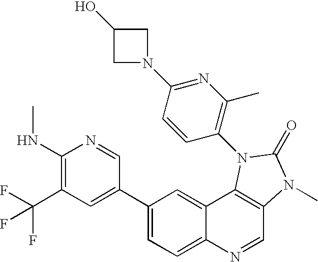

The title compound was synthesized in a similar manner as described for Example 1.1 using methyl-[5-(4,4,5,5-tetramethyl-[1,3,2]dioxaborolan-2-yl)-3-trifluoromethyl-pyridin-2-yl]-amine and intermediate Z4. The silyl protecting group was removed as described in example 26.1.

(HPLC: t_{R} 4.203 (method B); M+H=536.3 MS−ES, ¹H NMR (600 MHz, DMSO-d₆) δ ppm 8.97 (s, 1H), 8.46 (d, 1H), 8.07 (d, 1H), 7.93 (dd, 1H), 7.63-7.72 (m, 2H), 7.20 (s, 1H), 6.80 (d, 1H), 6.44 (d, 1H), 5.71-5.82 (m, 1H), 4.62 (d, 1H), 4.24 (dt, 2H), 3.81 (dd, 2H), 3.60 (s, 3H), 2.93 (d, 3H), 2.06 (s, 3H)).

### Example 29.5

### 1-[6-(3-Hydroxy-azetidin-1-yl)-2-methyl-pyridin-3-yl]-8-(6-hydroxymethyl-5-methoxy-pyridin-3-yl)-3-methyl-1,3-dihydro-imidazo[4,5-c]quinolin-2-one

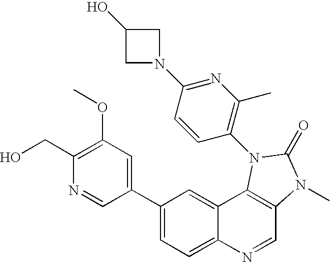

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester and intermediate Z4. The acetyl protecting group was removed in situ by dissolving the crude product in an aqueous solution of LiOH (1 M, 4 equivalents), stirring for 5 min at RT and the removing the solvent under reduced pressure. The silyl protecting group was removed as described in example 26.1.

(HPLC: t_{R} 3.534 (method B); M+H=499.3 MS−ES, ¹H NMR (600 MHz, DMSO-d₆) δ ppm 9.03 (s, 1H), 8.24 (s, 1H), 8.15 (d, 1H), 8.03 (dd, 1H), 7.67 (d, 1H), 7.39 (s, 1H), 7.25 (s, 1H), 6.48 (d, 1H), 5.78 (d, 1H), 4.93 (t, 1H), 4.65 (d, 1H), 4.56 (d, 2H), 4.15-4.32 (m, 2H), 3.88 (s, 3H), 3.70-3.85 (m, 2H), 3.62 (s, 3H), 2.09 (s, 3H)).

### Example 29.6

### 8-(2,4-Dimethoxy-pyrimidin-5-yl)-1-[6-(3-hydroxy-azetidin-1-yl)-2-methyl-pyridin-3-yl]-3-methyl-1,3-dihydro-imidazo[4,5-c]quinolin-2-one

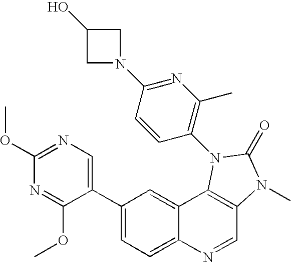

The title compound was synthesized in a similar manner as described for Example 1.1 using 2,4-dimethoxy-pyrimidine-5-boronicic acid and intermediate Z4. The silyl protecting group was removed as described in example 26.1.

(HPLC: t_{R} 4.046 (method B); M+H=500.3 MS−ES, ¹H NMR (600 MHz, DMSO-d₆) δ ppm 9.00 (s, 1H), 8.36 (s, 1H), 8.08 (d, 1H), 7.76 (dd, 1H), 7.59 (d, 1H), 7.45 (d, 1H), 6.44 (d, 1H), 5.74 (d, 1H), 4.62 (d, 1H), 4.16-4.32 (m, 2H), 3.94 (s, 3H), 3.86 (s, 3H), 3.69-3.83 (m, 2H), 3.54-3.65 (m, 3H), 2.06 (s, 3H)).

### Example 29.7

### 8-(2-Ethylamino-pyrimidin-5-yl)-1-[6-(3-hydroxy-azetidin-1-yl)-2-methyl-pyridin-3-yl]-3-methyl-1,3-dihydro-imidazo[4,5-c]quinolin-2-one

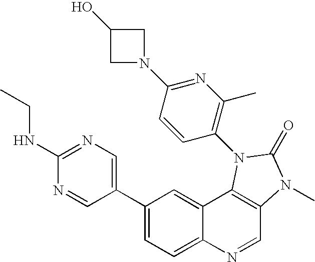

The title compound was synthesized in a similar manner as described for Example 1.1 using id and intermediate Z4. The silyl protecting group was removed as described in example 26.1.

(HPLC: t_{R} 3.897 (method B); M+H=483.3 MS−ES, ¹H NMR (600 MHz, DMSO-d₆) δ ppm 8.96 (s, 1H), 8.34 (br. s., 2H), 8.07 (d, 1H), 7.87 (d, 1H), 7.65 (d, 1H), 7.46 (t, 1H), 7.14 (s, 1H), 6.48 (d, 1H), 5.74 (d, 1H), 4.64 (d, 1H), 4.26 (t, 2H), 3.72-3.91 (m, 2H), 3.60 (s, 3H), 3.32 (d, 2H), 2.07 (s, 3H), 1.13 (t, 3H)).

### Example 30.1

### 1-(6-Amino-2-methyl-pyridin-3-yl)-8-(6-hydroxymethyl-5-methoxy-pyridin-3-yl)-3-methyl-1,3-dihydro-imidazo[4,5-c]quinolin-2-one

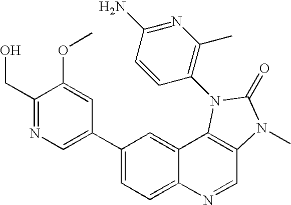

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester and intermediate T. The acetyl protecting group was removed in situ by dissolving the crude product in an aqueous solution of LiOH (1 M, 4 equivalents), stirring for min at RT and the removing the solvent under reduced pressure.

N-[5-(8-Bromo-3-methyl-2-oxo-2,3-dihydro-imidazo[4,5-c]quinolin-1-yl)-6-methyl-pyridin-2-yl]-2-methoxy-acetamide (Intermediate T) (36.5 mg; 0.08 mmol), was dissolved in DMF (5 mL), followed by addition of acetic acid 3-methoxy-5-(4,4,5,5-tetramethyl-[1,3,2]dioxaborolan-2-yl)-pyridin-2-ylmethyl ester (49.1 mg; 0.160 mmol), PdCl₂(PPh₃)₂ (4.49 mg; 6.4 umol) and potassium carbonate (0.32 mL; 1M solution in H₂O; 0.32 mmol) and the mixture was stirred at 100° C. for 2 h. Another acetic acid 3-methoxy-5-(4,4,5,5-tetramethyl-[1,3,2]dioxaborolan-2-yl)-pyridin-2-ylmethyl ester (49.1 mg; 0.160 mmol), PdCl₂(PPh₃)₂ (4.49 mg; 6.4 umol) and potassium carbonate (0.32 mL; 1M solution in H₂O; 0.32 mmol) were added and the mixture was stirred another hour at 100° C. to completion. After cooling to RT, EtOAC (50 mL) was added and the mixture was extracted with water (2×). The organic layer was freed from the solvent under reduced pressure to obtain the still acetylated intermediate. The residue was taken up into a solution of LiOH (mL) and kept for 5 min at RT. After removal of the solvent, purification was done by chromatography on silica gel (eluting with DCM/MeOH; 92/8) to obtain the title compound as a bright beige powder (11 mg). (HPLC: t_{R} 3.342 min (Method A); M+H=443.2 MS−ES) ¹H-NMR (d₆-DMSO, 600 MHz) 9.02 (s, 1H), 8.28 (s, 1H), 8.15-8.14 (m, 1H), 8.03-8.01 (m, 1H), 7.52-7.49 (m, 2H), 7.27-7.26 (m, 1H), 6.51-6.50 (m, 1H), 6.47 (s, 2H), 4.94-4.93 (m, 1H), 4.57-4.56 (m, 2H), 3.90 (s, 3H), 3.61 (s, 3H), 2.01 (3H)).

### Example 31.1

### 8-(5-Fluoro-6-methylamino-pyridin-3-yl)-1-[6-(1-hydroxy-1-methyl-ethyl)-2-methyl-pyridin-3-yl]-3-methyl-1,3-dihydro-imidazo[4,5-c]quinolin-2-one

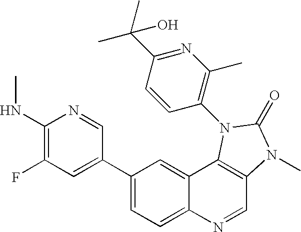

The title compound was synthesized in a similar manner as described for Example 1.1 using [3-Fluoro-5-(4,4,5,5-tetramethyl-[1,3,2]dioxaborolan-2-yl)-pyridin-2-yl]-methyl-amine and intermediate Z5.

(HPLC: t_{R} 4.345 min (Method B); M+H=517.2 MS−ES) ¹H-NMR (d₆-DMSO, 600 MHz) 8.99 (s, 1H), 8.07-8.05 (m, 1H), 7.97 (m, 1H), 7.87 (m, 2H), 7.80-7.79 (m, 1H), 7.20-7.18 (m, 1H), 6.91-6.90 (m, 1H), 6.87-6.86 (m, 1H), 5.41 (s, 1H), 3.63 (s, 3H), 2.84 (m, 3H), 2.28 (s, 3H), 1.58 (s, 3H), 1.56 (s, 3H)).

### Stage 31.1.1 2-(5-Amino-6-methyl-pyridin-2-yl)-propan-2-ol

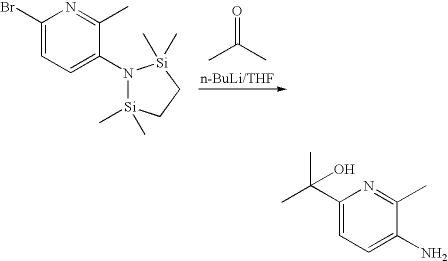

6-Bromo-2-methyl-3-(2,2,5,5-tetramethyl-[1,2,5]azadisilolidin-1-yl)-pyridine (574 mg, 1.743 mmol) was diluted in dry THF (10 ml). The solution was cooled down to −78° C., then n-BuLi (2.396 ml, 3.83 mmol) was slowly added keeping temperature around −78° C. (solution became yellow). The yellow solution was stirred for 1.5 h at −78° C. Then acetone (0.256 ml, 3.49 mmol) was added and the yellow solution was stirred for 1 h at −78° C. The yellow reaction solution was slowly quenched with water (10 mL) at −78° C. The dry ice/acetone bath was removed and the reaction mix was allow to come back at rt (overnight). HCl 1M aq. (−7 mL) was added (acidic pH). The mixture was stirred for 15 min at RT. Diethylether was added to the mixture and both phases were separated. The organic layer was washed twice with water and each aqueous phase was extracted back twice with diethylether. The combined aqueous layers were basified (NaHCO₃ aq. sat. soln) followed by extraction with dichloromethane (3×). The combined organics were washed with brine, dried (Na₂SO₄) and after filtration, the solvent was removed under reduced pressure and the crude product was dried under vacuum (50° C.) to obtain the title compound (240 mg) as a yellow oil which was used in the next step without further purification.

(HPLC: t_{R} 3.180 min (Method B); M+H=167.2 MS−ES)

### Stage 31.1.2 6-Bromo-2-methyl-3-(2,2,5,5-tetramethyl-[1,2,5]azadisilolidin-1-yl)-pyridine

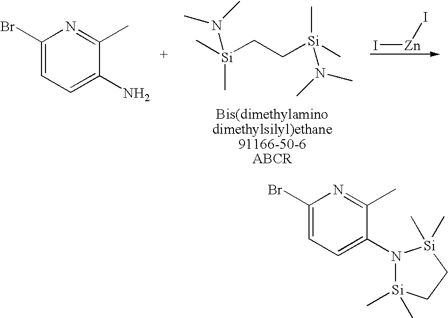

6-Bromo-2-methyl-pyridin-3-ylamine (2.550 g, 12.95 mmol), bis(dimethylaminodimethylsilyl)ethane (5.12 mL, 17.23 mmol) and Zinc Iodide (0.043 g, 0.130 mmol) was heated up to 140° C. and stirred for 25 h under an atmosphere of argon. The reaction mixture was poured onto a water/phosphate buffer solution (pH=7.0) and extracted 3× with diethylether.

The combined organic layers were washed with brine, dried (Na₂SO₄) and after filtration, the solvent was removed under reduced pressure and the crude product was dried under vacuum (RT). Purification was done over silica gel (eluting with EtOAc/heptane 1/5) to obtain the title compound as slightly pinkish solid (931 mg). (HPLC: t_{R} 3.794 min (Method B); M+H=331 MS−ES).

### Stage 31.1.3 6-Bromo-2-methyl-pyridin-3-ylamine

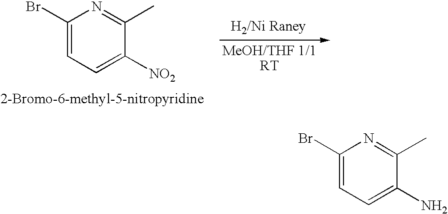

The title compound was prepared from 2-bromo-6-methyl-5-nitropyridine as described in example 19., stage 19.1.1. (HPLC: t_{R} 3.816 min (Method B); M+H=189 MS−ES.

### Example 31.2

### 1-[6-(1-Hydroxy-1-methyl-ethyl)-2-methyl-pyridin-3-yl]-3-methyl-8-(2-methylamino-pyrimidin-5-yl)-1,3-dihydro-imidazo[4,5-c]quinolin-2-one

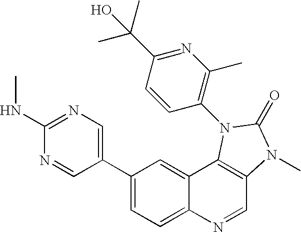

The title compound was synthesized in a similar manner as described for Example 1.1 using methyl-[5-(4,4,5,5-tetramethyl-[1,3,2]dioxaborolan-2-yl)-pyrimidin-2-yl]-amine and intermediate Z5.

(HPLC: t_{R} 4.377 min (Method B); M+H=456.3 MS−ES); ¹H NMR (600 MHz, DMSO-d₆) δ ppm 9.01 (s, 1H), 8.24 (br. s., 2H), 8.08 (d, 1H), 7.98 (d, 1H), 7.88 (dd, 1H), 7.79 (d, 1H), 7.36 (d, 1H), 6.83 (s, 1H), 5.42 (s, 1H), 3.63 (s, 3H), 2.74-2.84 (m, 3H), 2.27 (s, 3H), 1.57 (d, 6H)).

### Example 31.3

### 8-(2,4-Dimethoxy-pyrimidin-5-yl)-1-[6-(1-hydroxy-1-methyl-ethyl)-2-methyl-pyridin-3-yl]-3-methyl-1,3-dihydro-imidazo[4,5-c]quinolin-2-one

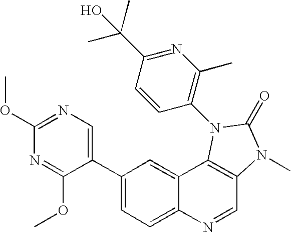

The title compound was synthesized in a similar manner as described for Example 1.1 using 2,4-dimethoxy-pyrimidine-5-boronicic acid and intermediate Z5.

HPLC: t_{R} 4.800 min (Method B); M+H=487.3 MS−ES); ¹H NMR (600 MHz, DMSO-d₆) δ ppm 9.05 (s, 1H), 8.20 (s, 1H), 8.09 (d, 1H), 7.93 (d, 1H), 7.75 (d, 2H), 7.01 (d, 1H), 5.39 (s, 1H), 3.90 (s, 3H), 3.85 (s, 3H), 3.63 (s, 3H), 2.28 (s, 3H), 1.52 (s, 3H), 1.46 (s, 3H)).

### Example 31.4

### 8-(6-Ethoxy-pyridin-3-yl)-1-[6-(1-hydroxy-1-methyl-ethyl)-2-methyl-pyridin-3-yl]-3-methyl-1,3-dihydro-imidazo[4,5-c]quinolin-2-one

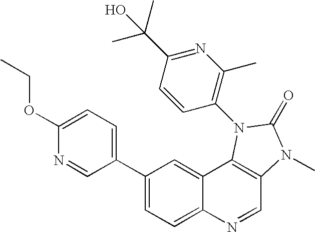

The title compound was synthesized in a similar manner as described for Example 1.1 using 2-ethoxy-5-(4,4,5,5-tetramethyl-[1,3,2]dioxaborolan-2-yl)-pyridine and intermediate Z5.

(HPLC: t_{R} 5.290 min (Method B); M+H=470.3 MS−ES); ¹H NMR (600 MHz, DMSO-d₆) δ ppm 9.03 (s, 1H), 8.07-8.16 (m, 2H), 7.98 (d, 1H), 7.90 (dd, 1H), 7.80 (d, 1H), 7.61 (dd, 1H), 6.90 (s, 1H), 6.77 (d, 1H), 5.43 (s, 1H), 4.30 (q, 2H), 3.64 (s, 3H), 2.28 (s, 3H), 1.58 (s, 3H), 1.54 (s, 3H), 1.30-1.33 (m, 3H)).

### Example 31.5

### 8-(6-Amino-5-trifluoromethyl-pyridin-3-yl)-1-[6-(1-hydroxy-1-methyl-ethyl)-2-methyl-pyridin-3-yl]-3-methyl-1,3-dihydro-imidazo[4,5-c]quinolin-2-one

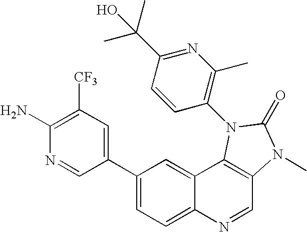

The title compound was synthesized in a similar manner as described for Example 1.1 using 5-(4,4,5,5-Tetramethyl-[1,3,2]dioxaborolan-2-yl)-3-trifluoromethyl-pyridin-2-ylamine and intermediate Z5.

(HPLC: t_{R} 4.769 min (Method B); M+H=509.3 MS−ES); ¹H NMR (600 MHz, DMSO-d₆) δ ppm 9.02 (s, 1H), 8.14 (d, 1H), 8.10 (d, 1H), 7.99 (d, 1H), 7.88 (dd, 1H), 7.76 (d, 1H), 7.67 (d, 1H), 6.94 (s, 1H), 6.69 (s, 2H), 5.36 (s, 1H), 3.63 (s, 3H), 2.28 (s, 3H), 1.55 (s, 3H), 1.49 (s, 3H)).

### Example 31.6

### 2-(5-{1-[6-(1-Hydroxy-1-methyl-ethyl)-2-methyl-pyridin-3-yl]-3-methyl-2-oxo-2,3-dihydro-1H-imidazo[4,5-c]quinolin-8-yl}-pyridin-3-yl)-2-methyl-propionitrile

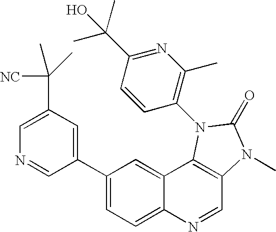

The title compound was synthesized in a similar manner as described for Example 1.1 using 2-methyl-2-[5-(4,4,5,5-tetramethyl-[1,3,2]dioxaborolan-2-yl)-pyridin-3-yl]-propionitrile and intermediate Z5.

(HPLC: t_{R} 4.739 min (Method B); M+H=593.4 MS−ES); ¹H NMR (600 MHz, DMSO-d₆) δ ppm 9.09 (s, 1H), 8.74 (d, 1H), 8.28 (s, 1H), 8.20 (d, 1H), 8.04-8.11 (m, 1H), 7.97-8.04 (m, 2H), 7.79 (d, 1H), 7.09 (s, 1H), 5.39 (s, 1H), 3.65 (s, 3H), 2.29 (s, 3H), 1.73-1.86 (m, 6H), 1.55 (s, 3H), 1.51 (s, 3H)).

### Example 31.7

### 8-(2-Ethylamino-pyrimidin-5-yl)-1-[6-(1-hydroxy-1-methyl-ethyl)-2-methyl-pyridin-3-yl]-3-methyl-1,3-dihydro-imidazo[4,5-c]quinolin-2-one

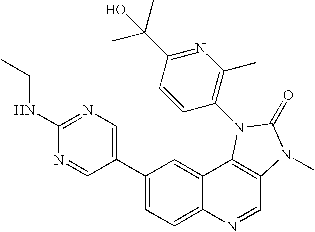

The title compound was synthesized in a similar manner as described for Example 1.1 using acid and intermediate Z5.

(HPLC: t_{R} 4.600 min (Method B); M+H=470.3 MS−ES); ¹H NMR (600 MHz, DMSO-d₆) δ ppm 9.01 (s, 1H), 8.23 (s, 2H), 8.08 (d, 1H), 7.98 (d, 1H), 7.84-7.94 (m, 1H), 7.79 (d, 1H), 7.44 (t, 1H), 6.83 (d, 1H), 5.42 (s, 1H), 3.63 (s, 3H), 3.21-3.32 (m, 2H), 2.27 (s, 3H), 1.57 (d, 6H), 1.08-1.17 (m, 3H)).

### Example 31.8

### 1-(5-{1-[6-(1-Hydroxy-1-methyl-ethyl)-2-methyl-pyridin-3-yl]-3-methyl-2-oxo-2,3-dihydro-1H-imidazo[4,5-c]quinolin-8-yl}-pyridin-3-yl)-cyclobutanecarbonitrile

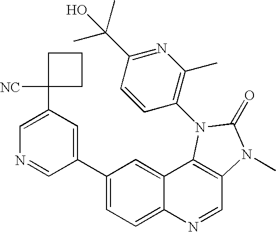

The title compound was synthesized in a similar manner as described for Example 1.1 using 1-[5-(4,4,5,5-Tetramethyl-[1,3,2]dioxaborolan-2-yl)-pyridin-3-yl]cyclobutane-carbonitrile and intermediate Z5.

(HPLC: t_{R} 4.847 min (Method B); M+H=505.3 MS−ES); ¹H NMR (600 MHz, DMSO-d₆) δ ppm 9.09 (s, 1H), 8.67 (d, 1H), 8.30 (s, 1H), 8.19 (d, 1H), 7.96-8.07 (m, 3H), 7.79 (d, 1H), 7.09 (s, 1H), 5.40 (s, 1H), 3.65 (s, 3H), 2.65-2.84 (m, 4H), 2.21-2.37 (m, 4H), 2.04 (dd, 1H), 1.55 (s, 3H), 1.50 (s, 3H)).

### Stage 31.8.1 1-[5-(4,4,5,5-Tetramethyl-[1,3,2]dioxaborolan-2-yl)-pyridin-3-yl]-cyclobutanecarbonitrile

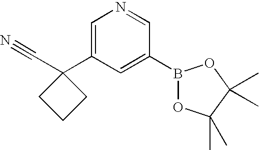

The title compound was synthesized in a similar manner as described for example 2.27, stage 2.27.1 using 1-(5-bromo-pyridin-3-yl)-cyclobutanecarbonitrile (stage 31.8.2, 0.476 mmol) to give the title compound as a crude back oil. (degrading under the HPLC condition: t_{R} 2.69 min (Method C); M+H=285 MS−ES).

### Stage 31.8.2 1-(5-Bromo-pyridin-3-yl)-cyclobutanecarbonitrile

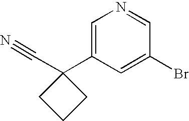

To a solution of (5-bromo-pyridin-3-yl)-acetonitrile (Stage 34.5.3, 1.37 mmol) in dry DMF (10 ml) was added 55% sodium hydride in oil (1.507 mmol). After 30 min stirring at rt was added 1-bromo-3-chloropropane (1.507 mmol). The RM was stirred for 1 h at rt then was added 55% sodium hydride in oil (1.507 mmol). The RM was stirred for 1 h at rt then quenched with saturated aqueous NaHCO₃ and extracted with EtOAc. The organic layer was washed with brine (2×), dried over Na₂SO₄, filtered and evaporated. The residue was taken in DMA and purified by preparative HPLC. The fractions containing product were basified with NaHCO₃, concentrated and extracted with EtOAc (2×). The combined organic layers were washed with brine, dried over Na₂SO₄, filtered, evaporated and dried under vacuum to give the title compound as a red oil. (HPLC: t_{R} 2.86 min (Method C); M+H=237, 239 MS−ES).

### Example 31.9

### 1-[6-(1-Hydroxy-1-methyl-ethyl)-2-methyl-pyridin-3-yl]-8-(5-isopropoxy-pyridin-3-yl)-3-methyl-1,3-dihydro-imidazo[4,5-c]quinolin-2-one

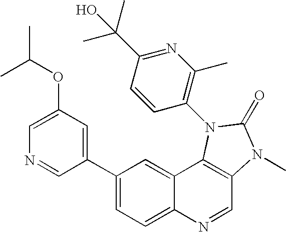

The title compound was synthesized in a similar manner as described for Example 1.1 using 3-Isopropoxy-5-(4,4,5,5-tetramethyl-[1,3,2]dioxaborolan-2-yl)-pyridine and intermediate Z5.

(HPLC: t_{R} 4.632 min (Method B); M+H=484.3 MS−ES); ¹H NMR (600 MHz, DMSO-d₆) δ ppm 9.07 (s, 1H), 8.24 (d, 1H), 8.15 (d, 1H), 7.91-8.06 (m, 3H), 7.78 (d, 1H), 7.31 (d, 1H), 7.05 (d, 1H), 5.39 (s, 1H), 4.69-4.85 (m, 1H), 3.64 (s, 3H), 2.28 (s, 3H), 1.56 (s, 3H), 1.52 (s, 3H), 1.23-1.36 (m, 6H)).

### Example 32.1

### 1-[6-((S)-3-Hydroxy-pyrrolidin-1-yl)-2-methyl-pyridin-3-yl]-3-methyl-8-(2-methylamino-pyrimidin-5-yl)-1,3-dihydro-imidazo[4,5-c]quinolin-2-one

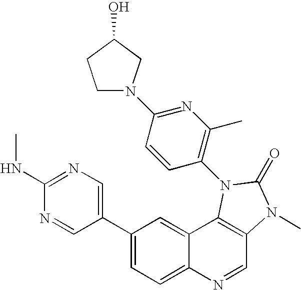

The title compound was synthesized in a similar manner as described for Example 1.1 using methyl-[5-(4,4,5,5-tetramethyl-[1,3,2]dioxaborolan-2-yl)-pyrimidin-2-yl]-amine and intermediate Z6.

(HPLC: t_{R} 3.458 min (Method A); M+H=483.2 MS−ES. ¹H-NMR (d₆-DMSO, 600 MHz) 8.97 (s, 1H), 8.35 (s, br, 2H), 8.07-8.06 (m, 1H), 7.87-7.86 (m, 1H), 7.62-7.60 (m, 1H), 7.37-7.36 (m, 1H), 7.18 (m, 1H), 6.54 (m, 1H), 5.04-5.02 (m, 1H), 4.45 (s, 1H), 3.60 (s, 3H), 3.61-3.56 (m, 4H), 2.83-2.82 (m, 3H), 2.07 (s, 3H), 2.2-2.1 and 5-1.85-1.96 (m, 2H)).

### Stage 32.1.2 (S)-1-(5-Amino-6-methyl-pyridin-2-yl)-pyrrolidin-3-ol

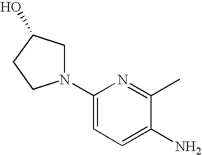

The title compound was prepared from 6-chloro-2-methyl-3-nitropyridine as described in example 23., stage 23.1.1. and 23.1.2., using (S)-pyrrolidin-3-ol instead of aziridine. (HPLC: 3.000 min (Method A); M+H=194.1 MS−ES.

### Example 32.2

### 8-(5-Fluoro-6-methylamino-pyridin-3-yl)-1-[6-((S)-3-hydroxy-pyrrolidin-1-yl)-2-methyl-pyridin-3-yl]-3-methyl-1,3-dihydro-imidazo[4,5-c]quinolin-2-one

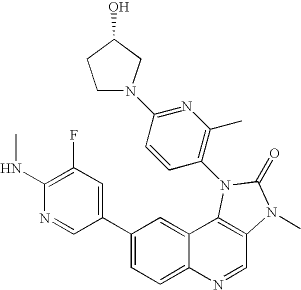

The title compound was synthesized in a similar manner as described for Example 1.1 using [3-Fluoro-5-(4,4,5,5-tetramethyl-[1,3,2]dioxaborolan-2-yl)-pyridin-2-yl]-methyl-amine and intermediate Z6.

(HPLC: t_{R} 3.442 min (Method B); M+H=500.2 MS−ES); ¹H NMR (600 MHz, DMSO-d₆) δ ppm 8.96 (br. s., 1H), 8.03 (d, 1H), 8.00 (br. s., 1H), 7.88 (dd, 1H), 7.62 (dd, 1H), 7.24-7.32 (m, 1H), 7.16 (br. s., 1H), 6.91 (d, 1H), 6.56 (dd, 1H), 5.03 (br. s., 1H), 4.44 (br. s., 1H), 3.56-3.71 (m, 7H), 2.86 (d, 3H), 2.02-2.15 (m, 4H), 1.96 (br. s., 1H)).

### Example 32.3

### 8-(2,4-Dimethoxy-pyrimidin-5-yl)-1-[6-((S)-3-hydroxy-pyrrolidin-1-yl)-2-methyl-pyridin-3-yl]-3-methyl-1,3-dihydro-imidazo[4,5-c]quinolin-2-one

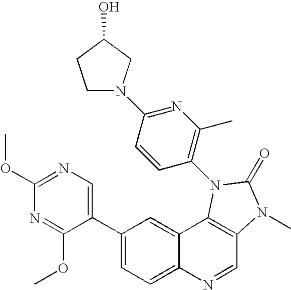

The title compound was synthesized in a similar manner as described for Example 1.1 using 2,4-dimethoxy-pyrimidine-5-boronicic acid and intermediate Z6.

(HPLC: t_{R} 3.633 min (Method B); M+H=514.2 MS−ES); ¹H NMR (600 MHz, DMSO-d₆) δ ppm 9.00 (s, 1H), 8.40 (d, 1H), 8.08 (d, 1H), 7.77 (dd, 1H), 7.49-7.64 (m, 2H), 6.49 (d, 1H), 5.00 (d, 1H), 4.42 (br. s., 1H), 3.86-3.98 (m, 3H), 3.81 (d, 3H), 3.49-3.65 (m, 7H), 1.91-2.10 (m, 5H)).

### Example 32.4

### 8-(5-Chloro-6-methylamino-pyridin-3-yl)-1-[6-((S)-3-hydroxy-pyrrolidin-1-yl)-2-methyl-pyridin-3-yl]-3-methyl-1,3-dihydro-imidazo[4,5-c]quinolin-2-one

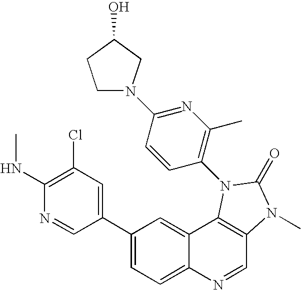

The title compound was synthesized in a similar manner as described for Example 1.1 using [3-Chloro-5-(4,4,5,5-tetramethyl-[1,3,2]dioxaborolan-2-yl)-pyridin-2-yl]-methyl-amine and intermediate Z6.

(HPLC: t_{R} 3.567 min (Method A); M+H=516.2 MS−ES); ¹H NMR (600 MHz, DMSO-d₆) δ ppm 8.95 (s, 1H), 8.22 (br. s., 1H), 8.04 (d, 1H), 7.90 (d, 1H), 7.62 (d, 1H), 7.43 (d, 1H), 7.18 (br. s., 1H), 6.79 (d, 1H), 6.55 (d, 1H), 5.04 (br. s., 1H), 4.45 (br. s., 1H), 3.55-3.8 (m, 7H), 2.88 (d, 3H), 1.8-2.2 (m, 5H)).

### Stage 32.4.1 [3-Chloro-5-(4,4,5,5-tetramethyl-[1,3,2]dioxaborolan-2-yl)-pyridin-2-yl]-methyl-amine

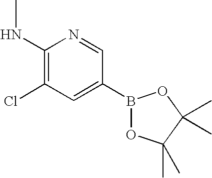

The title compound was synthesized in a similar manner as described for Stage 7.12.1-2 using 5-bromo-2,3-dichloropyridine (Asymchem Laboratories, Morrisville, N.C., USA) and heating to 150° C. as replacement for 5-bromo-2,3-difluoropyridine to give the title compound as a crude brown oil. (HPLC: t_{R} 1.62 min (Method C); M+H=269 MS−ES).

### Example 32.5

### 1-[6-((S)-3-Hydroxy-pyrrolidin-1-yl)-2-methyl-pyridin-3-yl]-3-methyl-8-(6-methylamino-5-trifluoromethyl-pyridin-3-yl)-1,3-dihydro-imidazo[4,5-c]quinolin-2-one

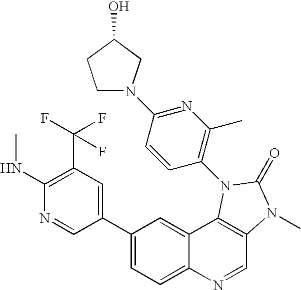

The title compound was synthesized in a similar manner as described for Example 1.1 using methyl-[5-(4,4,5,5-tetramethyl-[1,3,2]dioxaborolan-2-yl)-3-trifluoromethyl-pyridin-2-yl]-amine and intermediate Z6.

(HPLC: t_{R} 3.733 min (Method A); M+H=550.2 MS−ES); ¹H NMR (600 MHz, DMSO-d₆) δ ppm 8.97 (s, 1H), 8.50 (s, 1H), 8.07 (d, 1H), 7.94 (d, 1H), 7.62 (d, 1H), 7.59 (br. s., 1H), 7.25 (s, 1H), 6.77 (q, 1H), 6.50 (dd, 1H), 5.04 (dd, 1H), 4.37-4.48 (m, 1H), 3.49-3.69 (m, 7H), 2.91 (d, 3H), 1.9-2.2 (m, 5H)).

### Example 32.6

### 8-(5-Ethylamino-6-hydroxymethyl-pyridin-3-yl)-1-[6-((S)-3-hydroxy-pyrrolidin-1-yl)-2-methyl-pyridin-3-yl]-3-methyl-1,3-dihydro-imidazo[4,5-c]quinolin-2-one

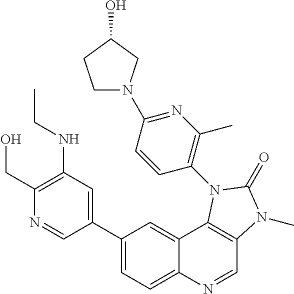

The title compound was synthesized in a similar manner as described for Example 1.1 using acetic acid 3-(tert-butoxycarbonyl-ethyl-amino)-5-(4,4,5,5-tetramethyl-[1,3,2]dioxaborolan-2-yl)-pyridin-2-ylmethyl ester and intermediate Z6. The acetyl protecting group as well as the BOC protecting group were removed together in situ by dissolving the crude product in an aqueous solution of LiOH (1 M, 4 equivalents), stirring for 5 min at RT and the removing the solvent under reduced pressure.

(HPLC: t_{R} 3.433 min (Method A); M+H=526.2 MS−ES); ¹H NMR (600 MHz, DMSO-d₆) δ ppm 9.01 (s, 1H), 8.12 (d, 1H), 7.93 (dd, 1H), 7.87 (dd, 1H), 7.62 (d, 1H), 7.49 (br. s., 1H), 6.77 (br. s., 1H), 6.51 (d, 1H), 5.49 (t, 1H), 5.31 (t, 1H), 5.06 (d, 1H), 4.55 (d, 2H), 4.44 (br. s., 1H), 3.61 (s, 3H), 3.52-3.59 (m, 4H), 3.00-3.15 (m, 2H), 1.9-2.2 (m, 2H), 2.08 (s, 3H), 1.19-1.24 (m, 3H)).

### Example 32.7

### 8-(6-Hydroxymethyl-5-methoxy-pyridin-3-yl)-1-[6-((S)-3-hydroxy-pyrrolidin-1-yl)-2-methyl-pyridin-3-yl]-3-methyl-1,3-dihydro-imidazo[4,5-c]quinolin-2-one

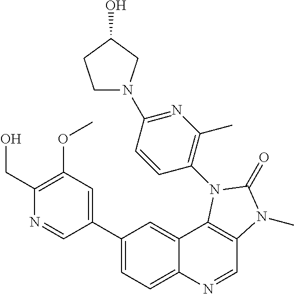

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester and intermediate Z6. The acetyl protecting group was removed in situ by dissolving the crude product in an aqueous solution of LiOH (1 M, 4 equivalents), stirring for 5 min at RT and the removing the solvent under reduced pressure.

(HPLC: t_{R} 3.367 min (Method A); M+H=513.2 MS−ES); ¹H NMR (600 MHz, DMSO-d₆) δ ppm 9.03 (s, 1H), 8.31 (d, 1H), 8.15 (d, 1H), 7.99-8.08 (m, 1H), 7.64 (d, 1H), 7.51 (s, 1H), 7.19 (d, 1H), 6.53 (d, 1H), 5.05 (d, 1H), 4.91 (t, 1H), 4.54 (d, 2H), 4.44 (s, 1H); 3.80 (s, 3H), 3.62 (s, 3H), 3.53-3.60 (m, 4H), 1.96-2.17 (m, 5H)).

### Example 32.8

### 1-[6-((S)-3-Hydroxy-pyrrolidin-1-yl)-2-methyl-pyridin-3-yl]-8-(5-isopropoxy-pyridin-3-yl)-3-methyl-1,3-dihydro-imidazo[4,5-c]quinolin-2-one

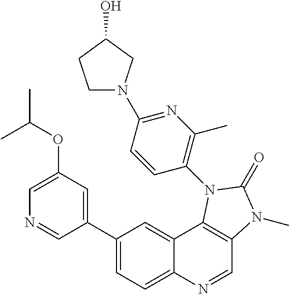

The title compound was synthesized in a similar manner as described for Example 1.1 using 3-Isopropoxy-5-(4,4,5,5-tetramethyl-[1,3,2]dioxaborolan-2-yl)-pyridine and intermediate Z6.

(HPLC: t_{R} 3.608 min (Method A); M+H=511.2 MS−ES); ¹H NMR (600 MHz, DMSO-d₆) δ ppm 9.03 (s, 1H), 8.19-8.28 (m, 2H), 8.14 (d, 1H), 7.97 (d, 1H), 7.64 (dd, 1H), 7.45 (s, 1H), 7.30 (br. s., 1H), 6.52 (d, 1H), 5.04 (dd, 1H), 4.64-4.76 (m, 1H), 4.43 (br. s., 1H), 3.48-3.67 (m, 7H), 2.00-2.12 (m, 5H), 1.29 (d, 6H)).

### Example 32.9

### 8-(2-Ethylamino-4-methoxy-pyrimidin-5-yl)-1-[6-((S)-3-hydroxy-pyrrolidin-1-yl)-2-methyl-pyridin-3-yl]-3-methyl-1,3-dihydro-imidazo[4,5-c]quinolin-2-one

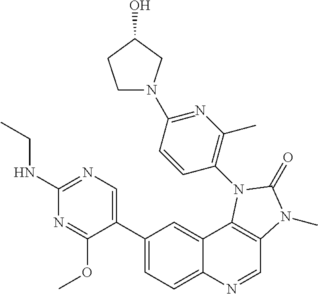

The title compound was synthesized in a similar manner as described for Example 1.1 using ethyl-[4-methoxy-5-(4,4,5,5-tetramethyl-[1,3,2]dioxaborolan-2-yl)-pyrimidin-2-yl]-amine and intermediate Z6.

(HPLC: t_{R} 3.542 min (Method A); M+H=527.2 MS−ES);

### Stage 32.9.1 Ethyl-[4-methoxy-5-(4,4,5,5-tetramethyl-[1,3,2]dioxaborolan-2-yl)-pyrimidin-2-yl]-amine

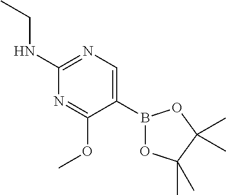

The title compound was synthesized in a similar manner as described for Stage 7.12.1 using (5-bromo-4-methoxy-pyrimidin-2-yl)-ethyl-amine (stage 32.9.2, 0.366 mmol) to give the title compound as a crude back oil. (degrading under the HPLC condition: t_{R} 1.91 min (Method C); M+H=280 MS−ES).

### Stage 32.9.2 (5-Bromo-4-methoxy-pyrimidin-2-yl)-ethyl-amine

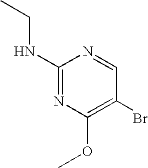

To a solution of 5-bromo-2-chloro-4-methoxypyrimidine (Frontier Scientific, Logan, USA, 0.895 mmol) in THF (2.5 ml) cooled with an ice-bath was added a 2 M solution of ethylamine in MeOH (Aldrich, Buchs, Switzerland, 0.492 ml). The RM was stirred at 0° C. for 1 h and at rt for 66 h then the reaction mixture was diluted with EtOAc, washed with saturated aqueous NaHCO₃, with brine, dried over Na₂SO₄, filtered and evaporated. The residue was absorbed on silica gel and purified by flash chromatography (CH₂Cl₂/iPrOH 0% to 6%) to give after evaporation of the fractions containing the title compound an off-white solid. (HPLC: t_{R} 2.16 min (Method C); M+H=232, 234 MS−ES).

### Example 32.10

### 8-(2-Ethylamino-pyrimidin-5-yl)-1-[6-((S)-3-hydroxy-pyrrolidin-1-yl)-2-methyl-pyridin-3-yl]-3-methyl-1,3-dihydro-imidazo[4,5-c]quinolin-2-one

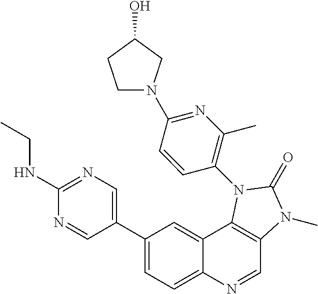

The title compound was synthesized in a similar manner as described for Example 1.1 using ethyl-[5-(4,4,5,5-tetramethyl-[1,3,2]dioxaborolan-2-yl)-pyrimidin-2-yl]-amine and intermediate Z6.

(HPLC: t_{R} 3.542 min (Method A); M+H=497.2 MS−ES);

### Example 33.1

### 8-[5-(1-Hydroxy-1-methyl-ethyl)-pyridin-3-yl]-3-methyl-1-(2-methyl-6-trifluoromethyl-pyridin-3-yl)-1,3-dihydro-imidazo[4,5-c]quinolin-2-one

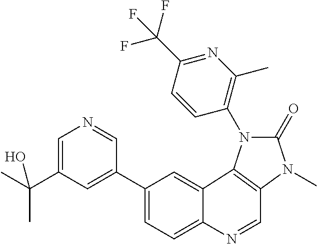

The title compound was synthesized in a similar manner as described for Example 1.1 using 2-[5-(4,4,5,5-Tetramethyl-[1,3,2]dioxaborolan-2-yl)-pyridin-3-yl]-propan-2-ol and intermediate Z7.

(HPLC: t_{R} 4.345 min (Method B); M+H=517.2 MS−ES) ¹H-NMR (d₆-DMSO, 600 MHz) 9.12 (s, 1H), 8.69 (s, 1H), 8.47-8.44 (m, 2H), 8.21-8.19 (m, 1H), 8.13-8.11 (m, 1H), 7.91-7.98 (m, 1H), 7.75 (s, 1H), 7.07 (s, 1H), 5.25 (s, 1H), 3.65 (s, 3H), 2.44 (s, 3H), 1.44 (s, 3H), 1.43 (s, 3H)

### Example 33.2

### 8-(6-Hydroxymethyl-5-methylamino-pyridin-3-yl)-3-methyl-1-(2-methyl-6-trifluoromethyl-pyridin-3-yl)-1,3-dihydro-imidazo[4,5-c]quinolin-2-one

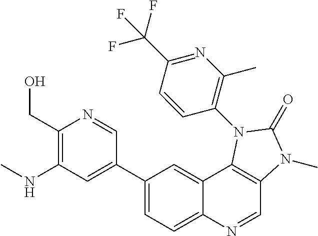

The title compound was synthesized in a similar manner as described for Example 1.1 using Acetic acid 3-(tert-butoxycarbonyl-methyl-amino)-5-(4,4,5,5-tetramethyl-[1,3,2]dioxaborolan-2-yl)-pyridin-2-ylmethyl ester and intermediate Z7. The acetyl protecting group as well as the BOC protecting group were removed together in situ by dissolving the crude product in an aqueous solution of LiOH (1 M, 4 equivalents), stirring for 5 min at RT and the removing the solvent under reduced pressure.

(HPLC: t_{R} 4.406 min (Method B); M+H=495.3 MS−ES); ¹H NMR (600 MHz, DMSO-d₆) δ ppm 9.10 (s, 1H), 8.45 (d, 1H), 8.15 (dd, 2H), 7.96 (dd, 1H), 7.81 (s, 1H), 7.10 (s, 1H), 6.66 (s, 1H), 5.65 (d, 1H), 5.21 (t, 1H), 4.52 (d, 2H), 3.65 (s, 3H), 2.71 (d, 3H), 2.45 (s, 3H)).

### Stage 33.2.1 Acetic acid 3-(tert-butoxycarbonyl-methyl-amino)-5-(4,4,5,5-tetramethyl-[1,3,2]dioxaborolan-2-yl)-pyridin-2-ylmethyl ester

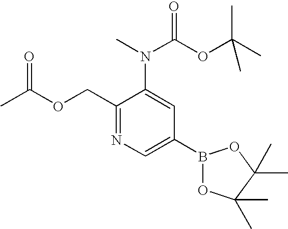

The title compound was prepared as described for acetic acid 3-(tert-butoxycarbonyl-ethyl-amino)-5-(4,4,5,5-tetramethyl-[1,3,2]dioxaborolan-2-yl)-pyridin-2-yl methyl ester (synthesis see example 2.31) except using CH₃I instead of CH₃CH₂I when alkylation of (5-bromo-2-methyl-pyridin-3-yl)-carbamic acid tert-butyl ester.

(HPLC: t_{R} 4.99 min (Method B); M+H=407.2 MS−ES)

### Example 33.3

### 8-(6-Amino-5-methoxymethyl-pyridin-3-yl)-3-methyl-1-(2-methyl-6-trifluoromethyl-pyridin-3-yl)-1,3-dihydro-imidazo[4,5-c]quinolin-2-one

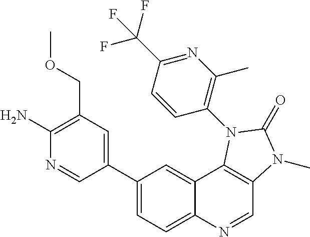

The title compound was synthesized in a similar manner as described for Example 1.1 using 3-methoxymethyl-5-(4,4,5,5-tetramethyl-[1,2]oxaborolan-2-yl)-pyridin-2-ylamine and intermediate Z7.

(HPLC: t_{R} 4.536 min (Method B); M+H=495.3 MS−ES); ¹H NMR (600 MHz, DMSO-d₆) δ ppm 9.02 (s, 1H), 8.45 (d, 1H), 8.17 (d, 1H), 8.09 (d, 1H), 7.99 (s, 1H), 7.88 (d, 1H), 7.26 (s, 1H), 6.87 (s, 1H), 6.05 (s, 2H), 4.25 (s, 2H), 3.63 (s, 3H), 3.29 (s, 3H), 2.43 (s, 3H)).

### Example 33.5

### 8-(6-Amino-5-hydroxymethyl-pyridin-3-yl)-3-methyl-1-(2-methyl-6-trifluoromethyl-pyridin-3-yl)-1,3-dihydro-imidazo[4,5-c]quinolin-2-one

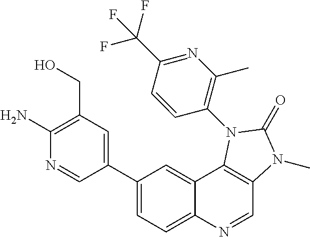

The title compound was synthesized in a similar manner as described for Example 1.1 using [2-amino-5-(4,4,5,5-tetramethyl-[1,3,2]dioxaborolan-2-yl)-pyridin-3-yl]-methanol and intermediate Z7.

(HPLC: t_{R} 4.188 min (Method B); M+H=481.3 MS−ES); ¹H NMR (600 MHz, DMSO-d₆) δ ppm 9.02 (s, 1H), 8.44 (d, 1H), 8.14 (d, 1H), 8.09 (d, 1H), 7.74-7.92 (m, 2H), 7.36 (s, 1H), 6.88 (s, 1H), 6.00 (s, 2H), 5.16 (s, 1H), 4.32 (d, 2H), 3.64 (s, 3H), 2.43 (s, 3H)).

### Example 33.6

### 8-(5-Methoxymethyl-6-methylamino-pyridin-3-yl)-3-methyl-1-(2-methyl-6-trifluoromethyl-pyridin-3-yl)-1,3-dihydro-imidazo[4,5-c]quinolin-2-one

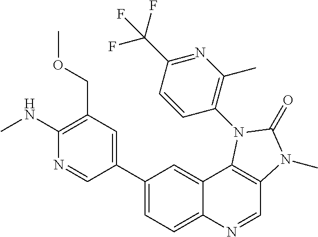

The title compound was synthesized in a similar manner as described for Example 1.1 using [3-methoxymethyl-5-(4,4,5,5-tetramethyl-[1,3,2]dioxaborolan-2-yl)-pyridin-2-yl]-methyl-amine and intermediate Z7.

(HPLC: t_{R} 4.615 min (Method B); M+H=509.3 MS−ES); ¹H NMR (600 MHz, DMSO-d₆) δ ppm 9.02 (s, 1H), 8.44 (d, 1H), 8.17 (d, 1H), 8.12 (s, 1H), 8.09 (d, 1H), 7.89 (d, 1H), 7.19 (s, 1H), 6.85 (s, 1H), 6.18 (d, 1H), 4.24 (s, 2H), 3.64 (s, 3H), 3.29 (s, 3H), 2.85 (d, 3H), 2.43 (s, 3H)).

### Example 34.1

### 1-[6-((R)-3-Hydroxy-pyrrolidin-1-yl)-2-methyl-pyridin-3-yl]-3-methyl-8-(2-methylamino-pyrimidin-5-yl)-1,3-dihydro-imidazo[4,5-c]quinolin-2-one

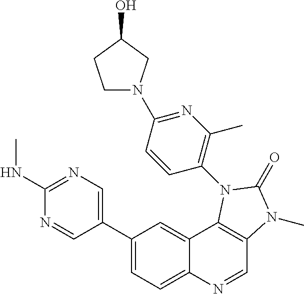

The title compound was synthesized in a similar manner as described for Example 1.1 using methyl-[5-(4,4,5,5-tetramethyl-[1,3,2]dioxaborolan-2-yl)-pyrimidin-2-yl]-amine and intermediate Z8.

(HPLC: t_{R} 3.483 min (Method B); M+H=483.2 MS−ES); ¹H NMR (600 MHz, DMSO-d₆) δ ppm 8.96 (s, 1H), 8.32 (m, 2H), 8.07 (d, 1H), 7.86 (dd, 1H), 7.61 (dd, 1H), 7.37 (d, 1H), 7.18 (s, 1H), 6.53 (dd, 1H), 5.03 (dd, 1H), 4.45 (br. s., 1H), 3.52-3.67 (m, 7H), 2.75-2.88 (m, 3H), 1.8-2.1 (m, 5H)).

### Stage 34.1.2 (R)-1-(5-Amino-6-methyl-pyridin-2-yl)-pyrrolidin-3-ol

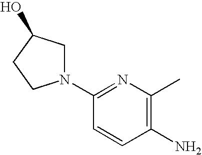

The title compound was prepared from 6-chloro-2-methyl-3-nitropyridine as described in example 23., stage 23.1.1. and 23.1.2., using (R)-pyrrolidin-3-ol instead of aziridine. (HPLC: t_{R} 3.00 min (Method A); M+H=194.1 MS−ES.

### Example 34.2

### 8-(6-Ethylamino-5-trifluoromethyl-pyridin-3-yl)-1-[6-((R)-3-hydroxy-pyrrolidin-1-yl)-2-methyl-pyridin-3-yl]-3-methyl-1,3-dihydro-imidazo[4,5-c]quinolin-2-one

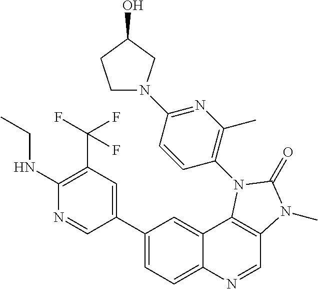

The title compound was synthesized in a similar manner as described for Example 1.1 using ine and intermediate Z8.

(HPLC: t_{R} 3.900 min (Method A); M+H=564.2 MS−ES); ¹H NMR (600 MHz, DMSO-d₆) δ ppm 8.97 (s, 1H), 8.48 (s, 1H), 8.07 (d, 1H), 7.94 (dd, 1H), 7.62 (dd, 1H), 7.59 (br. s., 1H), 7.25 (br. s., 1H), 6.70-6.83 (m, 1H), 6.51 (dd, 1H), 4.97-5.09 (m, 1H), 4.44 (br. s., 1H), 3.53-3.60 (m, 7H), 3.47 (m, 2H), 2.01-2.13 (m, 5H), 1.09-1.18 (m, 3H)).

### Stage 34.2.1 Ethyl-[5-(4,4,5,5-tetramethyl-[1,3,2]dioxaborolan-2-yl)-3-trifluoromethyl-pyridin-2-yl]-amine

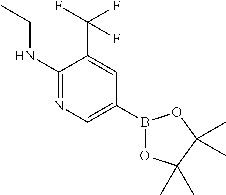

The title compound was synthesized in a similar manner as described for Example 7.13; stage 7.13.1-2 using iodoethane as instead of iodomethane to give the title compound as a crude back oil. (degrading under the HPLC condition: t_{R} 1.87, 3.68 min (Method C); M+H=317 MS−ES).

### Example 34.3

### 8-(2-Ethylamino-pyrimidin-5-yl)-1-[6-((R)-3-hydroxy-pyrrolidin-1-yl)-2-methyl-pyridin-3-yl]-3-methyl-1,3-dihydro-imidazo[4,5-c]quinolin-2-one

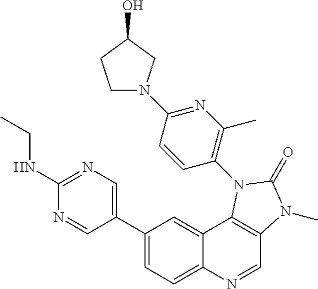

The title compound was synthesized in a similar manner as described for Example 1.1 using ethyl-[5-(4,4,5,5-tetramethyl-[1,3,2]dioxaborolan-2-yl)-pyrimidin-2-yl]-amine and intermediate Z8.

(HPLC: t_{R} 3.558 min (Method A); M+H=497.2 MS−ES); ¹H NMR (600 MHz, DMSO-d₆) δ ppm 8.96 (s, 1H), 8.28-8.42 (m, 2H), 8.06 (d, 1H), 7.86 (d, 1H), 7.61 (d, 1H), 7.44 (t, 1H), 7.18 (br. s., 1H), 6.53 (d, 1H), 5.03 (dd, 1H), 4.45 (br. s., 1H), 3.53-3.58 (m, 6H), 3.25-3.33 (m, 2H), 2.01-2.17 (m, 4H), 1.95 (d, 1H), 1.01-1.21 (m, 3H)).

### Example 34.4

### 8-(5-Ethoxymethyl-6-ethylamino-pyridin-3-yl)-1-[6-((R)-3-hydroxy-pyrrolidin-1-yl)-2-methyl-pyridin-3-yl]-3-methyl-1,3-dihydro-imidazo[4,5-c]quinolin-2-one

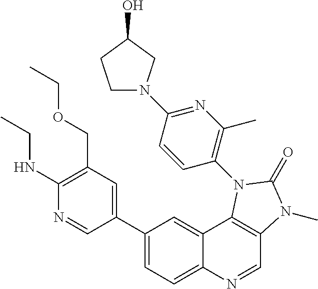

The title compound was synthesized in a similar manner as described for Example 1.1 using [3-ethoxymethyl-5-(4,4,5,5-tetramethyl-[1,3,2]dioxaborolan-2-yl)-pyridin-2-yl]-ethyl-amine and intermediate Z8.

(HPLC: t_{R} 3.567 min (Method A); M+H=554.2 MS−ES); ¹H NMR (600 MHz, DMSO-d₆) δ ppm 8.93 (s, 1H), 8.15 (t, 1H), 8.04 (d, 1H), 7.85 (dd, 1H), 7.62 (d, 1H), 7.31 (s, 1H), 7.26 (d, 1H), 6.55 (d, 1H), 6.06 (t, 1H), 5.00-5.11 (m, 1H), 4.38-4.51 (m, 1H), 3.52-3.66 (m, 7H), 3.37-3.51 (m, 6H), 2.00-2.16 (m, 5H), 1.10-1.21 (m, 6H)).

### Example 34.5

### 2-(5-{1-[6-((R)-3-Hydroxy-pyrrolidin-1-yl)-2-methyl-pyridin-3-yl]-3-methyl-2-oxo-2,3-dihydro-1H-imidazo[4,5-c]quinolin-8-yl}-pyridin-3-yl)-2-methyl-propionitrile

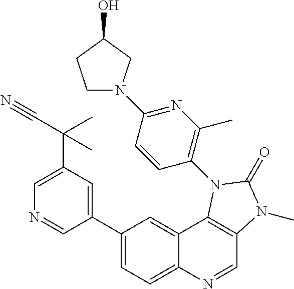

The title compound was synthesized in a similar manner as described for Example 1.1 using 2-methyl-2-[5-(4,4,5,5-tetramethyl-[1,3,2]dioxaborolan-2-yl)-pyridin-3-yl]-propionitrile and intermediate Z8.

(HPLC: t_{R} 3.625 min (Method A); M+H=520.2 MS−ES); ¹H NMR (600 MHz, DMSO-d₆) δ ppm 9.05 (s, 1H), 8.68-8.81 (m, 2H), 8.17 (d, 1H), 8.03 (d, 1H), 7.75 (ddd, 1H), 7.65 (dd, 1H), 7.47 (s, 1H), 6.52 (d, 1H), 5.03 (d, 1H), 4.43 (br. s., 1H), 3.53-3.62 (m, 7H), 1.91-2.07 (m, 5H), 1.72 (d, 6H)).

### Stage 34.5.1 2-Methyl-2-[5-(4,4,5,5-tetramethyl-[1,3,2]dioxaborolan-2-yl)-pyridin-3-yl]-propionitrile

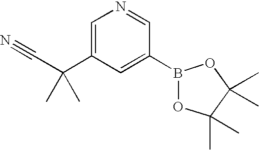

The title compound was synthesized in a similar manner as described for example 2.27, stage 2.27.1 using 2-(5-bromo-pyridin-3-yl)-2-methyl-propionitrile (stage 34.5.2, 1.111 mmol) to give the title compound as a crude brown soft solid. (degrading under the HPLC condition (Method C); M+H=273 MS−ES).

### Stage 34.5.2 2-(5-Bromo-pyridin-3-yl)-2-methyl-propionitrile

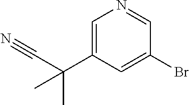

To a solution of (5-bromo-pyridin-3-yl)-acetonitrile (Stage 34.5.3, 4.61 mmol) in dry DMF (15 ml) cooled with an ice-bath was added portionwise 55% sodium hydride in oil (9.97 mmol). Was then added iodomethane (13.56 mmol) and DMF (5 ml). The RM was stirred for 3 h at 0° C. then was quenched with saturated aqueous NH₄Cl, diluted with water and extracted with EtOAc(3×). The combined organic layers were washed with brine, dried over Na₂SO₄, filtered and evaporated. The residue was purified by flash chromatography (heptane/EtOAc 25%). The fractions containing product were evaporated together to give the title compound as an oil. (HPLC: t_{R} 2.72 min (Method C); M+H=225, 227 MS−ES)

### Stage 34.5.3 (5-Bromo-pyridin-3-yl)-acetonitrile

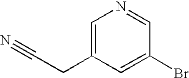

To SOCl₂ (26.7 ml) cooled with an ice-bath was added portionwise (5-bromo-pyridin-3-yl)-methanol (ABCR, Karlsruhe, Germany, 26.6 mmol). The RM was refluxed for 1 h then was cooled at 0° C. and quenched with diethyl ether. The resulting precipitate was filtered, washed with cooled diethyl ether and dried under vacuum at 50° C. The solid was mixed with potassium cyanide (64.5 mmol), MeOH (35 ml) and water (14 ml) and the RM was refluxed for 2 h. The RM was cooled, quenched with aqueous K₂CO₃ and extracted with diethyl ether (3×). The combined organic layers dried over Na₂SO₄, filtered and evaporated to give the title compound as a purple solid. (HPLC: t_{R} 2.16 min (Method C); M+H=197, 199 MS−ES).

### Example 35.1

### 8-[5-(1-Hydroxy-1-methyl-ethyl)-pyridin-3-yl]-3-methyl-1-[2-methyl-6-(3,3,4,4-tetrafluoro-pyrrolidin-1-yl)-pyridin-3-yl]-1,3-dihydro-imidazo[4,5-c]quinolin-2-one

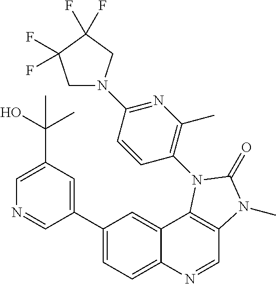

The title compound was synthesized in a similar manner as described for Example 1.1 using 2-[5-(4,4,5,5-Tetramethyl-[1,3,2]dioxaborolan-2-yl)-pyridin-3-yl]-propan-2-ol and intermediate Z9.

(HPLC: t_{R} 4.158 min (Method A); M+H=567.2 MS−ES); ¹H NMR (600 MHz, DMSO-d₆) δ ppm 9.05 (s, 1H), 8.67 (d, 1H), 8.64 (d, 1H), 8.16 (d, 1H), 8.00 (dd, 1H), 7.88 (d, 1H), 7.68 (t, 1H), 7.38 (d, 1H), 6.76 (d, 1H), 5.29 (s, 1H), 4.15-4.35 (m, 4H), 3.59-3.69 (m, 3H), 2.16 (s, 3H), 1.43 (d, 6H)).

### Stage 35.1.2 2-Methyl-6-(3,3,4,4-tetrafluoro-pyrrolidin-1-yl)-pyridin-3-ylamine

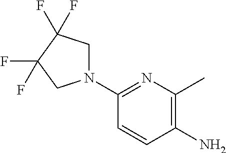

The title compound was prepared from 6-chloro-2-methyl-3-nitropyridine as described in example 23., stage 23.1.1. and 23.1.2., using 3,3,4,4-Tetrafluoro-pyrrolidine instead of aziridine. (HPLC: t_{R} 4.075 min (Method A); M+H=250.1 MS−ES.

### Example 35.2

### 8-(5-Ethylamino-6-hydroxymethyl-pyridin-3-yl)-3-methyl-1-[2-methyl-6-(3,3,4,4-tetrafluoro-pyrrolidin-1-yl)-pyridin-3-yl]-1,3-dihydro-imidazo[4,5-c]quinolin-2-one

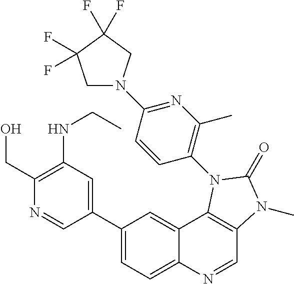

The title compound was synthesized in a similar manner as described for Example 1.1 using acetic acid 3-(tert-butoxycarbonyl-ethyl-amino)-5-(4,4,5,5-tetramethyl-[1,3,2]dioxa-borolan-2-yl)-pyridin-2-ylmethyl ester and intermediate Z9. The acetyl protecting group as well as the BOC protecting group were removed together in situ by dissolving the crude product in an aqueous solution of LiOH (1 M, 4 equivalents), stirring for 5 min at RT and the removing the solvent under reduced pressure.

(HPLC: t_{R} 4.167 min (Method A); M+H=582.2 MS−ES); ¹H NMR (600 MHz, DMSO-d₆) δ ppm 9.03 (s, 1H), 8.13 (d, 1H), 7.93 (dd, 1H), 7.79-7.89 (m, 2H), 7.36 (d, 1H), 6.79 (d, 1H), 6.74 (s, 1H), 5.52 (s, 1H), 5.29 (s, 1H), 4.54 (d, 2H), 4.16-4.33 (m, 4H), 3.62 (s, 3H), 3.07 (d, 2H), 2.17 (s, 3H), 1.20 (t, 3H)).

### Example 35.3

### 8-(6-Hydroxymethyl-5-methoxy-pyridin-3-yl)-3-methyl-1-[2-methyl-6-(3,3,4,4-tetrafluoro-pyrrolidin-1-yl)-pyridin-3-yl]-1,3-dihydro-imidazo[4,5-c]quinolin-2-one

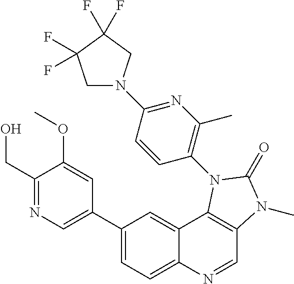

The title compound was synthesized in a similar manner as described for Example 1.1 using acetic acid 3-methoxy-5-(4,4,5,5-tetramethyl-[1,3,2]dioxaborolan-2-yl)-pyridin-2-ylmethyl ester and intermediate Z9. The acetyl protecting group was removed in situ by dissolving the crude product in an aqueous solution of LiOH (1 M, 4 equivalents), stirring for 5 min at RT and the removing the solvent under reduced pressure.

(HPLC: t_{R} 4.183 min (Method A); M+H=569.1 MS−ES).

### Example 35.4

### 8-(6-Amino-5-hydroxymethyl-pyridin-3-yl)-3-methyl-1-[2-methyl-6-(3,3,4,4-tetrafluoro-pyrrolidin-1-yl)-pyridin-3-yl]-1,3-dihydro-imidazo[4,5-c]quinolin-2-one

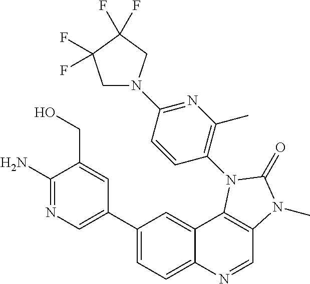

The title compound was synthesized in a similar manner as described for Example 1.1 using acetic acid 2-diacetylamino-5-(4,4,5,5-tetramethyl-[1,3,2]dioxaborolan-2-yl)-pyridin-3-ylmethyl ester and intermediate Z9. The acetyl protecting groups were removed in situ by dissolving the crude product in an aqueous solution of LiOH (1 M, 4 equivalents), stirring for 5 min at RT and the removing the solvent under reduced pressure.

(HPLC: t_{R} 4.067 min (Method A); M+H=554.1 MS−ES).

### Physicochemical Properties

The physicochemical properties of relevance for the absorption of the compound, such as the solubility and the membrane permeability, especially at a pH close to neutral pH can be measured using the procedure as described by L. ZHOU et al. in JOURNAL OF PHARMACEUTICAL SCIENCES, VOL. 96, NO. 11, p. 3052-3071 (2007) for the solubility at pH 6.8 and the procedure as described by F. Wohnsland and B. Faller in Journal of Medicinal Chemistry Vol 44, p. 923-930 (2001) for the Parallel Artificial Membrane Permeability Assay (PAMPA) at pH 6.8.

The following solubility in g/l was measured at pH 6.8 for compounds of formula (I) as exemplified below:

| example | HT-Eq Sol/pH 6.8 |
|---|---|
| number | (g/l) |
| 1.2 | 0.003 |
| 1.6 | 0.002 |
| 1.7 | 0.003 |
| 1.8 | 0.004 |
| 1.9 | 0.003 |
| 2.1 | 0.003 |
| 2.2 | 0.014 |
| 2.3 | 0.015 |
| 2.5 | 0.048 |
| 2.6 | 0.125 |
| 2.7 | 0.017 |
| 2.9 | 0.012 |
| 2.10 | >0.469 |
| 2.11 | 0.013 |
| 2.12 | 0.027 |
| 2.13 | 0.056 |
| 2.15 | 0.011 |
| 2.16 | 0.017 |
| 2.17 | >0.441 |
| 3.1 | 0.039 |
| 3.2 | 0.014 |
| 3.3 | 0.005 |
| 3.4 | 0.011 |
| 3.5 | 0.01 |
| 3.6 | 0.02 |
| 3.7 | >0.469 |
| 3.9 | >0.397 |
| 3.10 | 0.195 |
| 3.11 | 0.039 |
| 3.12 | 0.012 |
| 4.1 | 0.075 |
| 4.2 | 0.050 |
| 4.3 | 0.129 |
| 4.4 | 0.002 |
| 5.1 | 0.015 |
| 5.3 | 0.004 |
| 6.1 | 0.002 |
| 6.2 | 0.006 |
| 7.1 | 0.047 |
| 7.2 | 0.003 |
| 7.3 | 0.002 |
| 7.4 | 0.011 |
| 9.1 | 0.061 |
| 9.2 | 0.013 |
| 9.3 | 0.021 |
| 2.24 | 0.196 |
| 2.25 | 0.228 |
| 2.26 | 0.115 |
| 2.27 | >0.472 |
| 2.28 | 0.051 |
| 2.29 | 0.008 |
| 2.31 | 0.027 |
| 2.32 | 0.127 |
| 7.5 | 0.094 |
| 7.8 | 0.372 |
| 7.9 | 0.299 |
| 7.11 | 0.207 |
| 7.12 | 0.003 |
| 7.13 | 0.02 |
| 7.14 | 0.003 |
| 7.15 | 0.0675 |
| 7.16 | 0.187 |
| 7.17 | >0.43 |
| 8.10 | 0.047 |
| 8.12 | 0.013 |
| 9.4 | >0.512 |
| 11.7 | 0.003 |
| 11.8 | 0.026 |
| 12.2 | 0.005 |
| 13.1 | 0.003 |
| 13.2 | 0.027 |
| 13.3 | 0.01 |
| 13.4 | 0.007 |
| 13.5 | 0.015 |
| 14.2 | 0.004 |
| 14.3 | 0.007 |
| 14.4 | 0.008 |
| 14.6 | 0.175 |
| 14.8 | 0.012 |
| 15.2 | 0.081 |
| 15.6 | 0.14 |
| 15.7 | 0.004 |
| 15.8 | 0.056 |
| 16.1 | 0.003 |
| 16.2 | 0.07 |
| 16.3 | 0.027 |
| 16.4 | 0.055 |
| 16.5 | 0.002 |
| 18.1 | 0.01 |
| 18.2 | 0.003 |
| 18.3 | 0.037 |
| 19.1 | 0.007 |
| 19.2 | 0.034 |
| 19.3 | 0.005 |
| 19.4 | 0.11 |
| 19.5 | 0.01 |
| 20.1 | 0.028 |
| 21.1 | 0.022 |
| 22.1 | 0.007 |
| 23.1 | 0.022 |
| 23.2 | 0.264 |
| 23.3 | 0.204 |
| 23.4 | 0.009 |
| 23.5 | 0.069 |
| 23.6 | 0.021 |
| 23.8 | 0.057 |
| 23.9 | 0.035 |
| 24.1 | 0.059 |
| 25.2 | 0.187 |
| 25.3 | 0.099 |
| 26.1 | 0.004 |
| 26.2 | 0.023 |
| 27.2 | 0.067 |
| 28.1 | 0.427 |
| 29.2 | 0.057 |
| 29.3 | 0.056 |
| 29.4 | 0.044 |
| 29.5 | 0.256 |
| 29.6 | 0.015 |
| 31.1 | 0.003 |
| 31.4 | 0.005 |
| 31.5 | 0.003 |
| 31.6 | 0.003 |
| 31.7 | 0.014 |
| 31.8 | 0.111 |
| 32.1 | 0.064 |
| 32.2 | 0.007 |
| 32.3 | 0.03 |
| 32.5 | 0.049 |
| 32.8 | 0.12 |
| 33.1 | 0.423 |
| 34.2 | 0.023 |
| 34.4 | 0.172 |

The following permeability log Pe was measured for compound of formula (I) as exemplified below:

| example | |
|---|---|
| number | |
| | HT-perm/ |
| | logPe pH6.8 |
| 1.1 | −3.6 |
| 1.2 | −4.5 |
| 1.3 | −3.8 |
| 1.4 | −3.9 |
| 1.5 | −3.6 |
| 1.7 | −3.8 |
| 1.8 | −3.7 |
| 1.9 | −3.3 |
| 2.1 | −3.7 |
| 2.2 | −4.7 |
| 2.5 | −4.0 |
| 2.9 | −3.3 |
| 2.11 | −4.0 |
| 2.12 | −3.3 |
| 2.13 | −4.7 |
| 2.14 | −3.9 |
| 2.15 | −3.7 |
| 2.16 | −3.8 |
| 2.17 | −4.8 |
| 3.1 | −3.8 |
| 3.2 | −5.0 |
| 3.4 | −4.7 |
| 3.8 | −3.9 |
| 3.10 | −3.8 |
| 3.11 | −3.4 |
| 3.12 | −4.0 |
| 3.13 | −3.7 |
| 4.1 | −6.1 |
| 4.2 | −3.5 |
| 4.3 | −3.7 |
| 4.4 | −3.8 |
| 5.2 | −3.9 |
| 5.3 | −3.9 |
| 5.4 | −3.7 |
| 6.1 | −3.9 |
| 6.2 | −3.8 |
| 7.1 | −3.8 |
| 7.2 | −3.7 |
| 7.3 | −3.7 |
| 7.4 | −4.3 |
| 8.1 | −4.1 |
| 8.2 | −4.3 |
| 8.3 | −3.8 |
| 11.1 | −3.8 |
| | HT-Perm |
| | logPe pH6.8 |
| 2.22 | −3.7 |
| 2.23 | −5.3 |
| 2.24 | −4.5 |
| 2.25 | −7.7 |
| 2.26 | −5.3 |
| 2.27 | −5.3 |
| 2.28 | −4.3 |
| 2.29 | −4.3 |
| 2.31 | −5.8 |
| 2.32 | −4.8 |
| 7.5 | −3.7 |
| 7.8 | −5.5 |
| 7.9 | −5.2 |
| 7.11 | −5.4 |
| 7.12 | −3.7 |
| 7.13 | −3.7 |
| 7.14 | −5.9 |
| 7.15 | −4 |
| 7.16 | −6 |
| 8.8 | −3.8 |
| 8.9 | −3.9 |
| 8.10 | −3.9 |
| 8.12 | −4 |
| 9.4 | −6.1 |
| 11.7 | −3.7 |
| 11.8 | −3.9 |
| 12.1 | −4.1 |
| 12.2 | −4 |
| 13.1 | −4 |
| 13.2 | −3.7 |
| 13.3 | −4 |
| 13.4 | −4.1 |
| 13.5 | −4.3 |
| 14.2 | −3.8 |
| 14.3 | −3.8 |
| 14.4 | −4.6 |
| 14.5 | −3.8 |
| 14.6 | −4.1 |
| 14.7 | −4.3 |
| 14.8 | −4.1 |
| 15.1 | −4 |
| 15.2 | −3.9 |
| 15.3 | −3.8 |
| 15.4 | −3.7 |
| 15.5 | −3.7 |
| 15.6 | −3.6 |
| 15.7 | −3.9 |
| 15.8 | −5.1 |
| 16.1 | −3.7 |
| 16.2 | −3.8 |
| 16.3 | −4.4 |
| 16.4 | −4.2 |
| 16.5 | −3.7 |
| 17.1 | −4.5 |
| 18.1 | −3.6 |
| 18.2 | −3.5 |
| 18.3 | −3.5 |
| 19.1 | −3.9 |
| 19.2 | −4.1 |
| 19.3 | −4.4 |
| 19.4 | −4.1 |
| 19.5 | −5.2 |
| 20.1 | −6.1 |
| 21.1 | −6.1 |
| 22.1 | −3.8 |
| 23.2 | −3.7 |
| 23.3 | −5 |
| 23.5 | −3.8 |
| 23.6 | −4.2 |
| 23.7 | |
| 23.8 | −5.7 |
| 23.9 | −3.7 |
| 24.1 | −3.9 |
| 24.2 | −5 |
| 25.1 | −3.9 |
| 25.2 | −3.9 |
| 25.3 | −4.5 |
| 26.1 | −4.2 |
| 26.2 | −4.9 |
| 27.1 | −4.9 |
| 27.2 | −5.1 |
| 28.1 | −5.1 |
| 29.2 | −7.1 |
| 29.3 | −5.1 |
| 29.4 | −5 |
| 29.5 | −7.8 |
| 31.1 | −4.1 |
| 31.3 | −4.1 |
| 31.4 | −3.7 |
| 31.5 | −4.3 |
| 31.6 | −4.4 |
| 31.7 | −4.6 |
| 31.8 | −4.3 |
| 31.9 | −3.8 |
| 32.1 | −5 |
| 32.2 | −4.8 |
| 32.3 | −5.3 |
| 32.5 | −4.5 |
| 32.8 | −3.8 |
| 33.1 | −4.5 |
| 33.2 | −4.8 |
| 34.2 | −3.9 |
| 34.4 | −4.1 |
| 35.1 | −4.3 |

### Biological Activity

The efficacy of the compounds of formula I as PI3 kinase inhibitors can be demonstrated as follows:

The kinase reaction is performed in a final volume of 50 μL per well of a half area COSTAR, 96 well plate. The final concentrations of ATP and phosphatidyl inositol in the assay are 5 μM and 6 μg/mL respectively. The reaction is started by the addition of PI3 kinase, e.g. PI3 kinase.

p110β The components of the assay are added per well as follows:
10 μL test compound in 5% DMSO per well in columns 2-1.
Total activity is determined by addition 10 μL of 5% vol/vol DMSO in the first 4 wells of column 1 and the last 4 wells of column 12.
The background is determined by addition of 10 μM control compound to the last 4 wells of column 1 and the first 4 wells of column 12.
2 mL ‘Assay mix’ are prepared per plate:
   1.912 mL of HEPES assay buffer
   8.33 μL of 3 mM stock of ATP giving a final concentration of 5 μM per well
   1 μL of [³³P]ATP on the activity date giving 0.05 μCi per well
   30 μL of 1 mg/mL PI stock giving a final concentration of 6 μg/mL per well
   5 μL of 1 M stock MgCl₂ giving a final concentration of 1 mM per well
20 μL of the assay mix are added per well.
2 mL ‘Enzyme mix’ are prepared per plate (x* μL PI3 kinase p110β in 2 mL of kinase buffer). The ‘Enzyme mix’ is kept on ice during addition to the assay plates.
20 μl ‘Enzyme mix’ are added/well to start the reaction.
The plate is then incubated at room temperature for 90 minutes.
The reaction is terminated by the addition of 50 μL WGA-SPA bead (wheat germ agglutinin-coated Scintillation Proximity Assay beads) suspension per well.
The assay plate is sealed using TopSeal-S) heat seal for polystyrene microplates, PerkinElmer LAS (Deutschland) GmbH, Rodgau, Germany) and incubated at room temperature for at least 60 minutes.
The assay plate is then centrifuged at 1500 rpm for 2 minutes using the Jouan bench top centrifuge (Jouan Inc., Nantes, France).
The assay plate is counted using a Packard TopCount, each well being counted for 20 seconds.
* The volume of enzyme is dependent on the enzymatic activity of the batch in use.

In a more preferred assay, the kinase reaction is performed in a final volume of 10 μL per well of a low volume non binding CORNING, 384 well black plate (Cat. No. #3676). The final concentrations of ATP and phosphatidyl inositol (PI) in the assay are 1 μM and 10 μg/mL respectively. The reaction is started by the addition of ATP.

The components of the assay are added per well as follows:

50 nL test compounds in 90% DMSO per well, in columns 1-20, 8 concentrations (⅓ and 1/3.33 serial dilution step) in single.
Low control: 50 mL of 90% DMSO in half the wells of columns 23-24 (0.45% in final).
High control: 50 nL of reference compound (e.g. compound of Example 7 in WO 2006/122806) in the other half of columns 23-24 (2.5 μM in final).
Standard: 50 nL of reference compound as just mentioned diluted as the test compounds in columns 21-22
20 mL ‘buffer’ are prepared per assay:
   200 μL of 1M TRIS HCl pH7.5 (10 mM in final)
   60 μL of 1M MgCl₂ (3 mM in final)
   500 μL of 2M NaCl (50 mM in final)
   100 μL of 10% CHAPS (0.05% in final)
   200 μL of 100 mM DTT (1 mM in final)
   18.94 mL of nanopure water
10 mL ‘PI’ are prepared per assay:
   200 μL of 1 mg/ml L-alpha-Phosphatidylinositol (Liver Bovine, Avanti Polar Lipids Cat. No. 840042C MW=909.12) prepared in 3% OctylGlucoside (10 μg/ml in final)
   9.8 mL of ‘buffer’
10 mL ‘ATP’ are prepared per assay:
   6.7 μL of 3 mM stock of ATP giving a final concentration of 1 μM per well
   10 mL of ‘buffer’
2.5 mL of each PI3K construct are prepared per assay in ‘PI’ with the following final concentration:
   10 nM PI3K alfa EMV B1075
   25 nM beta EMV BV949
   10 nM delta EMV BV1060
   150 nM gamma EMV BV950
5 μL of ‘PI/PI3K’ are added per well.
5 μl ‘ATP’ are added per well to start the reaction.
The plates are then incubated at room temperature for 60 minutes (alfa, beta, delta) or 120 minutes (gamma).
The reaction is terminated by the addition of 10 μL Kinase-Glo (Promega Cat. No. #6714).
The assay plates are read after 10 minutes in Synergy 2 reader (BioTek, Vermont USA) with an integration time of 100 milliseconds and sensitivity set to 191.
Output: The High control is around 60′000 counts and the Low control is 30′000 or lower
This luminescence assay gives a useful Z′ ratio between 0.4 and 0.7

The Z′ value is a universal measurement of the robustness of an assay. A Z′ between 0.5 and 1.0 is considered an excellent assay.

For this assay, the PI3K constructs mentioned are prepared as follows:

### Molecular Biology:

Two different constructs, BV 1052 and BV 1075, are used to generate the PI3 Kinase α proteins for compound screening.

PI3Kα BV-1052 p85(iSH2)-Gly Linker-p110a(D20aa)-C-Term His taq

PCR products for the inter SH2 domain (iSH2) of the p85 subunit and for the p110-a subunit (with a deletion of the first 20 amino acids) are generated and fused by overlapping PCR.

The iSH2 PCR product is generated from first strand cDNA using initially primers

(SEQ ID NO: 1)
gwG130-p01
(5′-CGAGAATATGATAGATTATATGAAGAAT-3′)
and
(SEQ ID NO: 2)
gwG130-p02
(5′-TGGTTT-AATGCTGTTCATACGTTTGTCAAT-3′).

Subsequently in a secondary PCR reaction, Gateway (Invitrogen AG, Basel, Switzerland) recombination AttB1 sites and linker sequences are added at the 5′end and 3′end of the p85 iSH2 fragment respectively, using primers

(SEQ ID NO: 3)
gwG130-p03
(5′- GGGACAAGTTTGTACAAAAAAGCAGGCTACGAAGGAGATATACAT
AT-GCGAGAATATGATAGATTATATGAAGAAT -3′)
and
(SEQ ID NO: 4)
gwG152-p04
(5′- TACCATAATTCCACCACCACCACCGGAAATTCCCCCTGGTTT-
AATGCTGTTCATACGTTTGTCAAT-3′).

The p110-a fragment is also generated from first strand cDNA, initially using primers

(SEQ ID NO: 5)
gwG152-p01
(5′- CTAGTGGAATGTTTACTACCAAATGG-3′)
and
(SEQ ID NO: 6)
gwG152-p02
(5′- GTTCAATG-CATGCTGTTTAATTGTGT -3′).

In a subsequent PCR reaction, linker sequence and a Histidine tag are added at the 5′end and 3′end of the p110-a fragment respectively, using primers

(SEQ ID NO: 7)
gw152-p03
(5′-GGGGGAATTTCCGGTGGTGGTGGTGGAATTATGGTAC-
TAGTGGAATGTTTACTACC-AAATGGA-3′)
and
(SEQ ID NO: 8)
gwG152-p06
(5′-AGCTCCGTGATGGTGATGGTGATGTGCTCCGTTCAATG-
CATGCTGTTTAATTGTGT-3′).

The p85-iSH2/p110-a fusion protein is assembled in a third PCR reaction by the overlapping linkers at the 3′end of the iSH2 fragment and the 5′end of the p110-a fragment, using the above mentioned gwG130-p03 primer and a primer containing an overlapping Histidine tag and the AttB2 recombination sequences

(SEQ ID NO: 9)
(5′-GGGACCACTTTGTACAAGAAAGCTGGGTTTAAGCTCCGTGATGGTG
ATGGTGAT-GTGCTCC-3′).

This final product is recombined in a (Invitrogen) OR reaction into the donor vector pDONR201 to generate the ORF318 entry clone. This clone is verified by sequencing and used in a Gateway LR reaction to transfer the insert into the Gateway adapted pBlueBac4.5 (Invitrogen) vector for generation of the baculovirus expression vector LR410.

PI3Kα BV-1075 p85(iSH2)-12 XGly linker-p110a(D20aa)-C-Term H is taq

The construct for Baculovirus BV-1075 is generated by a three-part ligation comprised of a p85 fragment and a p110-a fragment cloned into vector pBlueBac4.5. The p85 fragment is derived from plasmid p1661-2 digested with Nhe/Spe. The p110-a fragment derived from LR410 (see above) as a SpeI/HindIII fragment. The cloning vector pBlueBac4.5 (Invitrogen) is digested with Nhe/HindIII. This results in the construct PED 153.8

The p85 component (iSH2) is generated by PCR using ORF 318 (described above) as a template and one forward primer

(SEQ ID NO: 10)
KAC1028
(5′- GCTAGCATGCGAGAATATGATAGATTATATGAAGAATATACC)

and two reverse primers,

(SEQ ID NO: 11)
KAC1029
(5′- GCCTCCACCACCTCCGCCTGGTTTAATGCTGTTCATACGTTTGT
C)
and
(SEQ ID NO: 12)
KAC1039
(5′-TACTAGTCCGCCTCCACCACCTCCGCCTCCACCACCTCCGCC).

The two reverse primers overlap and incorporate the 12×Gly linker and the N-terminal sequence of the p110a gene to the SpeI site. The 12×Gly linker replaces the linker in the BV1052 construct. The PCR fragment is cloned into pCR2.1 TOPO (Invitrogen). Of the resulting clones, p1661-2 is determined to be correct. This plasmid is digested with Nhe and SpeI and the resulting fragment is gel-isolated and purified for sub-cloning.

The p110-a cloning fragment is generated by enzymatic digest of clone LR410 (see above) with Spe I and HindIII. The SpeI site is in the coding region of the p110a gene. The resulting fragment is gel-isolated and purified for sub-cloning.

The cloning vector, pBlueBac4.5 (Invitrogen) is prepared by enzymatic digestion with Nhe and HindIII. The cut vector is purified with Qiagen (Quiagen N.V, Venlo, Netherlands) column and then dephosphorylated with Calf Intestine alkaline phosphatase (CIP) (New England BioLabs, Ipswich, Mass.). After completion of the CIP reaction the cut vector is again column purified to generate the final vector. A 3 part ligation is performed using Roche Rapid ligase and the vendor specifications.

PI3Kβ BV-949 p85(iSH2)-Gly linker-r 110b(full-length)-C-term His Tag

PCR products for the inter SH2 domain (iSH2) of the p85 subunit and for the full-length p110-b subunit are generated and fused by overlapping PCR.

The iSH2 PCR product is generated from first strand cDNA initially using primers

(SEQ ID NO: 1)
gwG130-p01
(5′-CGAGAATATGATAGATTATATGAAGAAT-3′)
and
(SEQ ID NO: 2)
gwG130-p02
(5′-TGGTTT-AATGCTGTTCATACGTTTGTCAAT-3′).

Subsequently, in a secondary PCR reaction Gateway (Invitrogen) recombination AttB1 sites and linker sequences are added at the 5′end and 3′end of the p85 iSH2 fragment respectively, using primers

(SEQ ID NO: 3)
gwG130-p03
(5′- GGGACAAGTTTGTACAAAAAAGCAGGCTACGAAGGAGATA-
TACATATGCGAGAATATGATAGATTATATGAAGAAT -3′)
and
(SEQ ID NO: 13)
gwG130-p05
(5′-ACTGAAGCATCCTCCTCCTCCTCCTCCTGGTTTAAT-
GCTGTTCATACGTTTGTC-3′).

The p110-b fragment is also generated from first strand cDNA initially using primers

(SEQ ID NO: 4)
gwG130-p04
(5′- ATTAAACCAGGAGGAGGAGGAGGAGGATGCTTCAGTTTCATAATG
CC-TCCTGCT -3′)

which contains linker sequences and the 5′end of p110-b and

(SEQ ID NO: 14)
gwG130-p06
(5′-AGCTCCGTGATGGTGATGGTGATGTGCTCCAGATCTGTAGTCTTT-
CCGAACTGTGTG -3′)

which contains sequences of the 3′end of p110-b fused to a Histidine tag.

The p85-iSH2/p110-b fusion protein is assembled by an overlapping PCR a reaction of the linkers at the 3′end of the iSH2 fragment and the 5′end of the p110-b fragment, using the above mentioned gwG130-p03 primer and a primer containing an overlapping Histidine tag and the AttB2 recombination sequences (5′-GGGACCACTTTGTACAAGAAAGCTGGGTTT-AAGCTCCGTGATGGTGATGGTGATGTGCTCC-3′) (SEQ ID NO: 9).

This final product is recombined in a Gateway (Invitrogen) OR reaction into the donor vector pDONR201 to generate the ORF253 entry clone. This clone is verified by sequencing and used in a Gateway LR reaction to transfer the insert into the Gateway adapted pBlueBac4.5 (Invitrogen) vector for generation of the baculovirus expression vector LR280.

PI3Kδ BV-1060 p85(iSH2)-Gly Linker-p110d(Full-Length)-C-Term His Tag

PCR products for the inter SH2 domain (iSH2) of the p85 subunit and for the full-length p110-d subunit are generated and fused by overlapping PCR.

The iSH2 PCR product is generated from first strand cDNA using initially primers

(SEQ ID NO: 1)
gwG130-p01
(5′-CGAGAATATGATAGATTATATGAAGAAT-3′)
and
(SEQ ID NO: 2)
gwG130-p02
(5′-TGGTTT-AATGCTGTTCATACGTTTGTCAAT-3′).

Subsequently, in a secondary PCR reaction Gateway (Invitrogen) recombination AttB1 sites and linker sequences are added at the 5′end and 3′end of the p85 iSH2 fragment respectively, using primers

(SEQ ID NO: 3)
gwG130-p03
(5′- GGGACAAGTTTGTACAAAAAAGCAGGCTACGAAGGAGATATACA
T-ATGCGAGAATATGATAGATTATATGAAGAAT -3′)
and
(SEQ ID NO: 15)
gwG154-p04
(5′- TCCTCCTCCTCCTCCTCCTGGTTTAATGCTGTTCATACGTTTGT
C -3′).

The p110-a fragment is also generated from first strand cDNA using initially primers

(SEQ ID NO: 16)
gwG154-p01
(5′- ATGCCCCCTGGGGTGGACTGCCCCAT -3′)
and
(SEQ ID NO: 17)
gwG154-p02
(5′- CTACTG-CCTGTTGTCTTTGGACACGT -3′).

In a subsequent PCR reaction linker sequences and a Histidine tag is added at the 5′end and 3′end of the p110-d fragment respectively, using primers

(SEQ ID NO: 18)
gw154-p03
(5′- ATTAAACCAGGAGGAGGAGGAGGAGGACCCCCTGGGGTGGAC-
TGCCCCATGGA -3′)
and
(SEQ ID NO: 19)
gwG154-p06
(5′-AGCTCCGTGATGGTGAT-GGTGATGTGCT-
CCCTGCCTGTTGTCTTTGGACACGTTGT -3′).

The p85-iSH2/p110-d fusion protein is assembled in a third PCR reaction by the overlapping linkers at the 3′end of the iSH2 fragment and the 5′end of the p110-d fragment, using the above mentioned gwG130-p03 primer and a primer containing an overlapping Histidine tag and the Gateway (Invitrogen) AttB2 recombination sequences (5′-GGGACCACTTTGTA-CAAGAAAGCTGGGTTT-AAGCTCCGTGATGGTGATGGTGATGTGCTCC-3′) (SEQ ID NO: 09).

This final product is recombined in a Gateway (Invitrogen) OR reaction into the donor vector pDONR201 to generate the ORF319 entry clone. This clone is verified by sequencing and used in a Gateway LR reaction to transfer the insert into the Gateway adapted pBlueBac4.5 (Invitrogen) vector for generation of the baculovirus expression vector LR415.

PI3Kγ BV-950 p110g(D144aa)-C-term His Tag

This construct is obtained from Roger Williams lab, MRC Laboratory of Molecular Biology, Cambridge, UK (November, 2003). Description of the construct in: Pacold M. E. et al. (2000) Cell 103, 931-943.

### Expression:

Methods to generate recombinant baculovirus and protein for PI3K isoforms:

The pBlue-Bac4.5 (for a, b, and d isoforms) or pVL1393 (for g) plasmids containing the different PI3 kinase genes are co-transfected with BaculoGold WT genomic DNA (BD Biosciences, Franklin Lakes, N.J., USA) using methods recommended by the vendor. Subsequently, the recombinant baculovirus obtained from the transfection is plaque-purified on Sf9 insect cells to yield several isolates expressing recombinant protein. Positive clones are selected by anti-HIS or anti-isoform antibody western. For PI3K alpha and delta isoforms, a secondary plaque-purification is performed on the first clonal virus stocks of PI3K. Amplification of all baculovirus isolates is performed at low multiplicity of infection (moi) to generate high-titer, low passage stock for protein production. The baculoviruses are designated BV1052 (α) and BV1075 (α), BV949 (β), BV1060 (δ) and BV950 (γ).

Protein production involves infection (passage 3 or lower) of suspended Tn5 (Trichoplusia ni) or TiniPro (Expression Systems, LLC, Woodland, Calif., USA) cells in protein-free media at moi of 2-10 for 39-48 hours in 2 L glass Erlenmyer flasks (110 rpm) or wave-bioreactors (22-25 rpm). Initially, 10 L working volume wave-bioreactors are seeded at a density of 3e5 cells/ml at half capacity (5 L). The reactor is rocked at 15 rpm during the cell growth phase for 72 hours, supplemented with 5% oxygen mixed with air (0.2 L per minute). Immediately prior to infection, the wave-reactor cultures are analyzed for density, viability and diluted to approximately 1.5e6 cell/ml. 100-500 ml of high titer, low passage virus is added following 2-4 hours of additional culture. Oxygen is increased to 35% for the 39-48 hour infection period and rocking platform rpm increased to 25. During infection, cells are monitored by ViceII viability analyzer (Beckman Coulter, Inc, Fullerton, Calif., USA) bioprocess for viability, diameter and density. Nova Bioanalyzer (NOVA Biomedical Corp., Waltham, Mass., USA) readings of various parameters and metabolites (pH, O₂ saturation, glucose, etc.) are taken every 12-18 hours until harvest. The wave-bioreactor cells are collected within 40 hours post infection. Cells are collected by centrifugation (4 degrees C. at 1500 rpm), and subsequently maintained on ice during pooling of pellets for lysis and purification. Pellet pools are made with small amounts of cold, un-supplemented Grace's media (w/o protease inhibitors).

### PI3K Alpha Purification Protocol for HTS (BV1052)

PI3K alpha is purified in three chromatographic steps: immobilized metal affinity chromatography on a Ni Sepharose resin (GE Healthcare, belonging to General Electric Company, Fairfield, Conn., USA), gel filtration utilizing a Superdex 200 26/60 column (GE Healthcare), and finally a cation exchange step on a SP-XL column (GE Healthcare). All buffers are chilled to 4° C. and lysis is performed chilled on ice. Column fractionation is performed rapidly at room temperature.

Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The pool from the GFC column is diluted into a low salt buffer and applied to a prepared SP-XL column. The column is washed with low salt buffer until a stable A280 baseline absorbance is achieved, and eluted using a 20 column volume gradient from 0 mM NaCl to 500 mM NaCl. Again, fractions from the SP-XL column are analyzed by Coomassie stained SDS-PAGE gels, and fractions containing the target protein are pooled. The final pool is dialyzed into a storage buffer containing 50% glycerol and stored at −20° C. The final pool is assayed for activity in a phosphoinosititol kinase assay.

### PI3K Beta Purification Protocol for HTS (BV949)

PI3K beta is purified in two chromatographic steps: immobilized metal affinity chromatography (IMAC) on a Ni Sepharose resin (GE Healthcare) and gel filtration (GFC) utilizing a Superdex 200 26/60 column (GE Healthcare). All buffers are chilled to 4° C. and lysis is performed chilled on ice. Column fractionation is performed rapidly at room temperature.

Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The final pool is dialyzed into a storage buffer containing 50% glycerol and stored at −20° C. The final pool is assayed for activity in the phosphoinostitol kinase assay.

### PI3K Gamma Purification Protocol for HTS (BV950)

PI3K gamma is purified in two chromatographic steps: immobilized metal affinity chromatography (IMAC) on a Ni Sepharose resin (GE Healthcare) and gel filtration (GFC) utilizing a Superdex 200 26/60 column (GE Healthcare). All buffers are chilled to 4° C. and lysis is performed chilled on ice. Column fractionation is performed rapidly at room temperature. Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The final pool is dialyzed into a storage buffer containing 50% glycerol and stored at −20° C. The final pool is assayed for activity in the phosphoinostitol kinase assay.

### PI3K Delta Purification Protocol for HTS (BV1060)

PI3K delta is purified in three chromatographic steps: immobilized metal affinity chromatography on a Ni Sepharose resin (GE Healthcare), gel filtration utilizing a Superdex 200 26/60 column (GE Healthcare), and finally a anion exchange step on a Q-HP column (GE Healthcare). All buffers are chilled to 4° C. and lysis is performed chilled on ice. Column fractionation is performed rapidly at room temperature. Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing the target protein are pooled and applied to a prepared GFC column. Fractions from the GFC column are analyzed by Coomassie stained SDS-PAGE gels, and fractions containing the target protein are pooled. The pool from the GFC column is diluted into a low salt buffer and applied to a prepared Q-HP column. The column is washed with low salt buffer until a stable A280 baseline absorbance is achieved, and eluted using a 20 column volume gradient from 0 mM NaCl to 500 mM NaCl. Again, fractions from the Q-HP column are analyzed by Coomassie stained SDS-PAGE gels, and fractions containing the target protein are pooled. The final pool is dialyzed into a storage buffer containing 50% glycerol and stored at −20° C. The final pool is assayed for activity in the phosphoinostitol kinase assay.

IC50 is determined by a four parameter curve fitting routine that comes along with “excel fit”. A 4 Parameter logistic equation is used to calculate IC₅₀ values (IDBS XLfit) of the percentage inhibition of each compound at 8 concentrations (usually 10, 3.0, 1.0, 0.3, 0.1, 0.030, 0.010 and 0.003 μM). Alternatively, IC₅₀ values are calculated using idbsXLfit model 204, which is a 4 parameter logistic model.

Yet alternatively, for an ATP depletion assay, compounds of the formula I to be tested are dissolved in DMSO and directly distributed into a white 384-well plate at 0.5 μL per well. To start the reaction, 10 μL of 10 nM PI3 kinase and 5 μg/mL 1-alpha-phosphatidylinositol (PI) are added into each well followed by 10 μL of 2 μM ATP. The reaction is performed until approx 50% of the ATP is depleted, and then stopped by the addition of 20 μL of Kinase-Glo solution (Promega Corp., Madison, Wis., USA). The stopped reaction is incubated for 5 minutes and the remaining ATP is then detected via luminescence. IC50 values are then determined.

Some of the compounds of formula (I) show a certain level of selectivity against the different paralogs PI3K alpha, beta, gamma and delta.

The range of activity, expressed as IC₅₀, in these assays is preferably between 1 nM and 5000 nM, more preferably between 1 nM and about 1000 nM

| example number | |
|---|---|
| | PI3Kalfa/IC50 |
| | [umol] |
| 1.1 | 0.023 |
| 1.2 | 0.015 |
| 1.3 | 0.010 |
| 1.4 | 0.048 |
| 1.5 | 0.040 |
| 1.6 | 0.017 |
| 1.7 | 0.134 |
| 1.8 | 0.058 |
| 1.9 | 0.071 |
| 2.1 | 0.056 |
| 2.2 | 0.026 |
| 2.3 | 0.020 |
| 2.4 | 0.029 |
| 2.5 | 0.119 |
| 2.6 | 0.100 |
| 2.7 | 0.018 |
| 2.8 | 0.026 |
| 2.9 | 0.012 |
| 2.11 | 0.007 |
| 2.12 | 0.026 |
| 2.13 | 0.014 |
| 2.14 | 0.034 |
| 2.15 | 0.035 |
| 3.1 | 0.410 |
| 3.3 | 0.051 |
| 3.4 | 0.080 |
| 3.5 | 0.053 |
| 3.6 | 0.206 |
| 3.8 | 0.070 |
| 3.9 | 0.383 |
| 3.10 | 0.057 |
| 3.11 | 0.321 |
| 3.12 | 0.016 |
| 3.13 | 0.090 |
| 4.1 | 0.230 |
| 4.2 | 0.206 |
| 4.3 | 0.073 |
| 5.1 | 0.074 |
| 5.2 | 0.029 |
| 5.3 | 0.047 |
| 5.4 | 0.019 |
| 6.1 | 0.021 |
| 6.2 | 0.009 |
| 6.3 | 0.041 |
| 7.1 | 0.004 |
| 7.2 | 0.032 |
| 7.3 | 0.067 |
| 7.4 | 0.027 |
| 8.1 | 0.011 |
| 9.1 | 0.058 |
| 9.2 | 0.018 |
| | EPK_PI3Kalfa/ |
| | IC50 [umol] |
| 2.22 | 0.081 |
| 2.24 | 0.028 |
| 2.25 | 0.064 |
| 2.26 | 0.014 |
| 2.27 | 0.080 |
| 2.28 | 0.267 |
| 2.29 | 0.179 |
| 2.30 | 0.075 |
| 2.31 | 0.014 |
| 2.32 | 0.031 |
| 7.5 | 0.094 |
| 7.6 | 0.068 |
| 7.7 | 0.127 |
| 7.8 | 0.021 |
| 7.9 | 0.020 |
| 7.10 | 0.024 |
| 7.11 | 0.017 |
| 7.12 | 0.044 |
| 7.13 | 0.011 |
| 7.14 | 0.011 |
| 7.15 | 0.059 |
| 7.16 | 0.101 |
| 7.17 | 0.064 |
| 7.18 | 0.065 |
| 8.9 | 0.011 |
| 8.10 | 0.080 |
| 8.11 | 0.365 |
| 8.12 | 0.041 |
| 8.13 | 0.247 |
| 8.14 | 0.059 |
| 9.4 | 0.015 |
| 11.7 | <0.003 |
| 11.8 | 0.016 |
| 12.1 | 0.036 |
| 12.2 | 0.015 |
| 13.1 | 0.158 |
| 13.2 | 0.036 |
| 13.3 | 0.031 |
| 13.4 | 0.013 |
| 13.5 | 0.043 |
| 14.2 | 0.043 |
| 14.3 | 0.140 |
| 14.4 | 0.030 |
| 14.5 | 0.005 |
| 14.6 | 0.019 |
| 14.7 | 0.016 |
| 14.8 | 0.037 |
| 15.1 | 0.007 |
| 15.2 | 0.039 |
| 15.3 | 0.103 |
| 15.4 | 0.046 |
| 15.5 | 0.031 |
| 15.6 | 0.012 |
| 15.7 | 0.018 |
| 15.8 | 0.049 |
| 15.9 | 0.023 |
| 16.1 | 0.014 |
| 16.2 | 0.033 |
| 16.3 | 0.071 |
| 16.4 | 0.047 |
| 16.5 | 0.027 |
| 17.1 | 0.078 |
| 18.1 | 0.040 |
| 18.3 | 0.005 |
| 19.1 | 0.011 |
| 19.2 | 0.014 |
| 19.3 | 0.055 |
| 19.4 | 0.009 |
| 19.5 | 0.065 |
| 20.1 | 0.050 |
| 20.2 | 0.162 |
| 20.3 | 0.123 |
| 20.4 | 0.039 |
| 20.5 | 0.040 |
| 21.1 | 0.005 |
| 22.1 | 0.188 |
| 22.2 | 0.171 |
| 22.3 | 0.164 |
| 23.1 | 0.014 |
| 23.2 | 0.033 |
| 23.3 | 0.007 |
| 23.4 | 0.027 |
| 23.5 | 0.021 |
| 23.6 | 0.044 |
| 23.7 | 0.385 |
| 23.8 | 0.014 |
| 23.9 | 0.033 |
| 24.1 | 0.049 |
| 24.2 | 0.010 |
| 24.3 | 0.011 |
| 24.4 | 0.056 |
| 25.1 | 0.035 |
| 25.2 | 0.067 |
| 25.3 | <0.003 |
| 25.4 | 0.027 |
| 25.5 | 0.028 |
| 26.1 | 0.012 |
| 26.2 | 0.063 |
| 26.3 | 0.084 |
| 27.1 | 0.021 |
| 27.2 | 0.032 |
| 27.3 | 0.005 |
| 27.4 | 0.039 |
| 27.5 | 0.010 |
| 28.1 | 0.018 |
| 28.2 | 0.034 |
| 28.3 | 0.023 |
| 29.1 | 0.019 |
| 29.2 | 0.009 |
| 29.3 | 0.040 |
| 29.4 | 0.017 |
| 29.5 | 0.031 |
| 29.6 | 0.023 |
| 29.7 | 0.049 |
| 30.1 | 0.054 |
| 31.1 | 0.023 |
| 31.2 | 0.026 |
| 31.4 | 0.084 |
| 31.5 | 0.006 |
| 31.6 | 0.002 |
| 31.7 | 0.075 |
| 31.8 | 0.031 |
| 31.9 | 0.051 |
| 32.1 | 0.017 |
| 32.2 | 0.044 |
| 32.3 | 0.032 |
| 32.4 | 0.018 |
| 32.5 | 0.009 |
| 32.7 | 0.026 |
| 32.8 | 0.071 |
| 32.9 | 0.027 |
| 32.10 | 0.044 |
| 33.1 | 0.013 |
| 33.2 | 0.015 |
| 33.3 | 0.012 |
| 33.5 | 0.041 |
| 33.6 | 0.027 |
| 34.1 | 0.097 |
| 34.2 | 0.028 |
| 34.3 | 0.045 |
| 34.4 | 0.0655 |
| 35.1 | 0.007 |
| 35.2 | 0.027 |
| 35.3 | 0.014 |
| 35.4 | 0.039 |
| | PI3Kbeta/IC50 |
| | [umol] |
| 1.1 | 0.202 |
| 1.2 | 0.115 |
| 1.3 | 0.023 |
| 1.4 | 0.374 |
| 1.5 | 0.505 |
| 1.6 | 0.491 |
| 2.1 | 0.580 |
| 2.2 | 0.165 |
| 2.3 | 0.577 |
| 2.4 | 0.682 |
| 2.5 | 0.749 |
| 2.6 | 0.706 |
| 2.7 | 0.133 |
| 2.8 | 0.173 |
| 2.9 | 0.077 |
| 2.11 | 0.014 |
| 2.12 | 0.132 |
| 2.13 | 0.226 |
| 2.14 | 0.315 |
| 2.15 | 0.360 |
| 3.12 | 0.109 |
| 4.3 | 0.601 |
| 5.1 | 0.801 |
| 5.2 | 0.295 |
| 5.4 | 0.263 |
| 6.1 | 0.223 |
| 6.2 | 0.007 |
| 6.3 | 0.112 |
| 7.1 | 0.007 |
| 7.2 | 0.309 |
| 7.3 | 0.452 |
| 7.4 | 0.263 |
| 8.1 | 0.023 |
| 9.1 | 0.267 |
| 9.2 | 0.036 |
| | EPK_PI3Kbeta/ |
| | IC50 [umol] |
| 2.22 | 0.201 |
| 2.24 | 0.040 |
| 2.25 | 0.143 |
| 2.26 | 0.031 |
| 2.27 | 1.238 |
| 2.28 | 0.563 |
| 2.29 | 0.782 |
| 2.30 | 0.395 |
| 2.31 | 0.067 |
| 2.32 | 0.061 |
| 7.5 | 0.152 |
| 7.6 | 0.965 |
| 7.7 | 0.359 |
| 7.8 | 0.045 |
| 7.9 | 0.069 |
| 7.10 | 0.020 |
| 7.11 | 0.041 |
| 7.12 | 0.265 |
| 7.13 | 0.042 |
| 7.14 | 0.014 |
| 7.15 | 0.194 |
| 7.16 | 0.2365 |
| 7.17 | 0.188 |
| 7.18 | 0.321 |
| 8.9 | 0.043 |
| 8.10 | 1.340 |
| 8.11 | 0.730 |
| 8.12 | 0.212 |
| 8.13 | 0.265 |
| 8.14 | 1.549 |
| 9.4 | 0.142 |
| 11.7 | 0.006 |
| 11.8 | 2.587 |
| 12.1 | 0.154 |
| 12.2 | 0.049 |
| 13.1 | 1.954 |
| 13.2 | 0.020 |
| 13.3 | 0.502 |
| 13.4 | 0.062 |
| 13.5 | 0.658 |
| 14.2 | 0.358 |
| 14.3 | 0.396 |
| 14.4 | 0.282 |
| 14.5 | 0.009 |
| 14.6 | 0.050 |
| 14.7 | 1.102 |
| 14.8 | 0.053 |
| 15.1 | 0.015 |
| 15.2 | 0.541 |
| 15.3 | 0.793 |
| 15.4 | 0.407 |
| 15.5 | 0.448 |
| 15.6 | 0.028 |
| 15.7 | 0.279 |
| 15.8 | 0.098 |
| 15.9 | 0.015 |
| 16.1 | 0.047 |
| 16.2 | 0.150 |
| 16.3 | 1.176 |
| 16.4 | 1.327 |
| 16.5 | 0.261 |
| 17.1 | 3.947 |
| 18.1 | 0.041 |
| 18.3 | 0.045 |
| 19.1 | 0.099 |
| 19.2 | 0.068 |
| 19.3 | 1.974 |
| 19.4 | <0.003 |
| 19.5 | 0.300 |
| 20.1 | 0.038 |
| 20.2 | 0.891 |
| 20.3 | 2.764 |
| 20.4 | 0.592 |
| 20.5 | 0.542 |
| 21.1 | 0.009 |
| 22.1 | 0.429 |
| 22.2 | 0.746 |
| 22.3 | 0.122 |
| 23.1 | 1.160 |
| 23.2 | 0.193 |
| 23.3 | 0.019 |
| 23.4 | 0.441 |
| 23.5 | 0.208 |
| 23.6 | 0.353 |
| 23.7 | >9.1 |
| 23.8 | 0.036 |
| 23.9 | 0.171 |
| 24.1 | 1.009 |
| 24.2 | 0.019 |
| 24.3 | 0.027 |
| 24.4 | 2.268 |
| 25.1 | 1.522 |
| 25.2 | 0.291 |
| 25.3 | <0.003 |
| 25.4 | 0.269 |
| 25.5 | 0.012 |
| 26.1 | <0.003 |
| 26.2 | 0.099 |
| 26.3 | 2.014 |
| 27.1 | 0.013 |
| 27.2 | 0.124 |
| 27.3 | 0.036 |
| 27.4 | 0.904 |
| 27.5 | 0.017 |
| 28.1 | 0.048 |
| 28.2 | 0.299 |
| 28.3 | 0.175 |
| 29.1 | 0.060 |
| 29.2 | 0.677 |
| 29.3 | 0.301 |
| 29.4 | 0.069 |
| 29.5 | 0.277 |
| 29.6 | 0.059 |
| 29.7 | 0.339 |
| 30.1 | 0.426 |
| 31.1 | 0.217 |
| 31.2 | 0.105 |
| 31.4 | 0.342 |
| 31.5 | 0.009 |
| 31.6 | 0.025 |
| 31.7 | 0.625 |
| 31.8 | 0.030 |
| 31.9 | 0.062 |
| 32.1 | 0.103 |
| 32.2 | 1.074 |
| 32.3 | 0.192 |
| 32.4 | 0.106 |
| 32.5 | 0.037 |
| 32.7 | 0.198 |
| 32.8 | 0.216 |
| 32.9 | 0.385 |
| 32.10 | 0.401 |
| 33.1 | 0.069 |
| 33.2 | 0.038 |
| 33.3 | 0.022 |
| 33.5 | 0.056 |
| 33.6 | 0.062 |
| 34.1 | 1.096 |
| 34.2 | 0.134 |
| 34.3 | 0.416 |
| 34.4 | 0.4545 |
| 35.1 | 0.034 |
| 35.2 | 0.0785 |
| 35.3 | 0.0487 |
| 35.4 | 0.207 |
| | PI3Kgamma/IC50 |
| | [umol] |
| 1.1 | 0.237 |
| 1.2 | 0.168 |
| 1.3 | 0.131 |
| 1.4 | 0.227 |
| 1.5 | 0.211 |
| 1.6 | 0.120 |
| 2.1 | 0.587 |
| 2.2 | 0.331 |
| 2.3 | 0.230 |
| 2.4 | 0.455 |
| 2.6 | 0.913 |
| 2.7 | 0.388 |
| 2.8 | 0.361 |
| 2.9 | 0.126 |
| 2.11 | 0.124 |
| 2.12 | 0.242 |
| 2.13 | 0.195 |
| 3.3 | 0.322 |
| 3.4 | 0.386 |
| 3.5 | 0.434 |
| 3.6 | 0.522 |
| 3.8 | 0.404 |
| 3.10 | 0.307 |
| 3.11 | 0.846 |
| 3.12 | 0.202 |
| 3.13 | 0.517 |
| 4.2 | 0.546 |
| 4.3 | 0.439 |
| 5.1 | 0.943 |
| 5.2 | 0.365 |
| 5.3 | 0.362 |
| 5.4 | 0.377 |
| 6.1 | 0.206 |
| 6.2 | 0.139 |
| 6.3 | 0.464 |
| | PI3Kdelta/IC50 |
| | [umol] |
| 1.1 | 0.026 |
| 1.2 | 0.013 |
| 1.3 | 0.008 |
| 1.4 | 0.081 |
| 1.5 | 0.166 |
| 1.6 | 0.163 |
| 1.7 | 0.101 |
| 1.8 | 0.364 |
| 1.9 | 0.085 |
| 2.1 | 0.094 |
| 2.2 | 0.022 |
| 2.3 | 0.093 |
| 2.4 | 0.221 |
| 2.5 | 0.058 |
| 2.6 | 0.059 |
| 2.7 | 0.035 |
| 2.8 | 0.035 |
| 2.9 | 0.037 |
| 2.10 | 0.855 |
| 2.11 | 0.012 |
| 2.12 | 0.032 |
| 2.13 | 0.020 |
| 2.14 | 0.057 |
| 2.15 | 0.032 |
| 3.1 | 0.851 |
| 3.3 | 0.834 |
| 3.4 | 0.236 |
| 3.8 | 0.264 |
| 3.9 | 0.583 |
| 3.10 | 0.151 |
| 3.12 | 0.021 |
| 3.13 | 0.249 |
| 4.1 | 0.406 |
| 4.2 | 0.401 |
| 4.3 | 0.120 |
| 5.1 | 0.088 |
| 5.2 | 0.072 |
| 5.3 | 0.193 |
| 5.4 | 0.039 |
| 6.1 | 0.064 |
| 6.2 | 0.012 |
| 6.3 | 0.078 |
| 7.1 | 0.005 |
| 7.2 | 0.031 |
| 7.3 | 0.071 |
| 7.4 | 0.021 |
| 8.1 | 0.012 |
| 9.1 | 0.066 |
| 9.2 | 0.015 |
| | EPK_PI3Kdelta/ |
| | IC50 [umol] |
| 2.22 | 0.061 |
| 2.24 | 0.031 |
| 2.25 | 0.057 |
| 2.26 | 0.046 |
| 2.27 | 0.406 |
| 2.28 | 0.054 |
| 2.29 | 0.504 |
| 2.30 | 0.053 |
| 2.31 | 0.025 |
| 2.32 | 0.031 |
| 7.5 | 0.078 |
| 7.6 | 0.148 |
| 7.7 | 0.079 |
| 7.8 | 0.038 |
| 7.9 | 0.036 |
| 7.10 | 0.186 |
| 7.11 | 0.026 |
| 7.12 | 0.012 |
| 7.13 | 0.003 |
| 7.14 | 0.005 |
| 7.15 | 0.037 |
| 7.16 | 0.0565 |
| 7.17 | 0.020 |
| 7.18 | 0.028 |
| 8.9 | 0.014 |
| 8.10 | 0.192 |
| 8.11 | 0.126 |
| 8.12 | 0.042 |
| 8.13 | 0.167 |
| 8.14 | 0.311 |
| 9.4 | 0.017 |
| 11.7 | 0.003 |
| 11.8 | <0.003 |
| 12.1 | 0.015 |
| 12.2 | 0.013 |
| 13.1 | 0.742 |
| 13.2 | 0.014 |
| 13.3 | 0.026 |
| 13.4 | 0.015 |
| 13.5 | 0.060 |
| 14.2 | 0.030 |
| 14.3 | 0.049 |
| 14.4 | 0.044 |
| 14.5 | 0.007 |
| 14.6 | 0.020 |
| 14.7 | 0.028 |
| 14.8 | 0.046 |
| 15.1 | 0.007 |
| 15.2 | 0.032 |
| 15.3 | 0.117 |
| 15.4 | 0.036 |
| 15.5 | 0.044 |
| 15.6 | 0.009 |
| 15.7 | 0.021 |
| 15.8 | 0.045 |
| 15.9 | 0.040 |
| 16.1 | 0.027 |
| 16.2 | 0.027 |
| 16.3 | 0.113 |
| 16.4 | 0.207 |
| 16.5 | 0.039 |
| 17.1 | 0.275 |
| 18.1 | 0.022 |
| 18.3 | 0.004 |
| 19.1 | 0.012 |
| 19.2 | 0.028 |
| 19.3 | 0.395 |
| 19.4 | 0.024 |
| 19.5 | 0.067 |
| 20.1 | 0.034 |
| 20.2 | 0.247 |
| 20.3 | 0.403 |
| 20.4 | 0.078 |
| 20.5 | 0.142 |
| 21.1 | 0.005 |
| 22.1 | 0.060 |
| 22.2 | 0.101 |
| 22.3 | 0.056 |
| 23.1 | 0.084 |
| 23.2 | 0.031 |
| 23.3 | 0.016 |
| 23.4 | 0.048 |
| 23.5 | 0.025 |
| 23.6 | 0.046 |
| 23.7 | 0.772 |
| 23.8 | 0.017 |
| 23.9 | 0.032 |
| 24.1 | 0.164 |
| 24.2 | 0.018 |
| 24.3 | 0.055 |
| 24.4 | 0.248 |
| 25.1 | 0.024 |
| 25.2 | 0.043 |
| 25.3 | <0.003 |
| 25.4 | 0.272 |
| 25.5 | 0.033 |
| 26.1 | <0.003 |
| 26.2 | 0.088 |
| 26.3 | 0.287 |
| 27.1 | 0.009 |
| 27.2 | 0.055 |
| 27.3 | <0.003 |
| 27.4 | 0.193 |
| 27.5 | 0.148 |
| 28.1 | 0.059 |
| 28.2 | 0.159 |
| 28.3 | 0.042 |
| 29.1 | 0.019 |
| 29.2 | 0.019 |
| 29.3 | 0.023 |
| 29.4 | 0.013 |
| 29.5 | 0.050 |
| 29.6 | 0.010 |
| 29.7 | 0.024 |
| 30.1 | 0.118 |
| 31.1 | 0.003 |
| 31.2 | 0.011 |
| 31.4 | 0.036 |
| 31.5 | <0.003 |
| 31.6 | 0.007 |
| 31.7 | 0.027 |
| 31.8 | 0.007 |
| 31.9 | 0.014 |
| 32.1 | 0.009 |
| 32.2 | 0.036 |
| 32.3 | 0.011 |
| 32.4 | 0.005 |
| 32.5 | 0.004 |
| 32.7 | 0.035 |
| 32.8 | 0.039 |
| 32.9 | 0.021 |
| 32.10 | 0.020 |
| 33.1 | 0.006 |
| 33.2 | 0.01 |
| 33.3 | 0.007 |
| 33.5 | 0.025 |
| 33.6 | 0.010 |
| 34.1 | 0.042 |
| 34.2 | 0.012 |
| 34.3 | 0.018 |
| 34.4 | 0.026 |
| 35.1 | 0.005 |
| 35.2 | 0.01 |
| 35.3 | 0.01 |
| 35.4 | 0.021 |

The efficacy of the compounds of formula I and salts thereof as mTor kinase inhibitors can be demonstrated as follows

Biochemical Assay for m-TOR by TR-FRET.

Assay components are purchased from Invitrogen Corporation (Carlsbad/Calif., USA): GFP-4EBP1 (Cat. No. PV4759), Tb3+-α-p4EBP1 [pThr46] Antibody (Cat. No. PV4757), proprietary TR-FRET dilution buffer (Cat. No. PV3574), mTOR protein (Cat. No. PV4753).

50 nL of compound dilutions (final concentration of 10, 3.0, 1.0, 0.3, 0.1, 0.03, 0.01 and 0.003 μM) are dispensed onto black 384-well low volume non-binding polystyrene (Cat. No. NBS#3676, Corning, Lowell/Mass., USA). Then 5 μL of ATP (8 μM final concentration) and GFP-4EBP1 (400 nM final concentration) with 5 μL mTOR proteins (0.5 nM final concentration) are incubated at rt. The standard reaction buffer for the TR-FRET mTOR assay contained 50 mM HEPES pH 7.5, 10 mM MnCl2, 50 mM NaCl, 1 mM EGTA, 1 mM DTT. Reactions were stopped with 10 μL of a mixture of EDTA containing the Tb3+-α-p4EBP1[pT46] detection Ab (0.5 nM) in TR-FRET dilution buffer (proprietary to IVG). Plates are read 15 mins later in a Synergy2 reader using an integration time of 0.2 seconds and a delay of 0.1 seconds. Control for the 100% inhibition of the kinase reaction is performed by replacing the mTOR kinase by the standard reaction buffer. The control for the 0% inhibition is given by the solvent vehicle of the compounds (90% DMSO in H2O). A standard compound is used as a reference compound and included in all assay plates in the form of 16 dilution points in duplicate.

The range of activity, expressed as IC₅₀, in this assay is preferably between 1 nM and 5000 nM, more preferably between 1 nM and about 1000 nM.

| example | |
|---|---|
| number | |
| | mTOR/IC50 [umol] |
| 1.1 | 0.036 |
| 1.2 | 0.004 |
| 1.3 | 0.009 |
| 1.4 | 0.025 |
| 1.5 | 0.107 |
| 1.6 | 0.010 |
| 1.7 | 0.016 |
| 1.8 | 0.087 |
| 1.9 | 0.014 |
| 2.1 | 0.042 |
| 2.2 | 0.008 |
| 2.3 | 0.011 |
| 2.4 | 0.012 |
| 2.5 | 0.166 |
| 2.6 | 0.104 |
| 2.7 | 0.018 |
| 2.9 | 0.023 |
| 2.10 | 0.600 |
| 2.11 | 0.016 |
| 2.12 | 0.074 |
| 2.13 | 0.019 |
| 2.14 | 0.024 |
| 2.15 | 0.053 |
| 3.3 | 0.345 |
| 3.4 | 0.607 |
| 3.5 | 0.331 |
| 3.8 | 0.544 |
| 3.12 | 0.389 |
| 4.3 | 0.442 |
| 5.1 | 0.351 |
| 5.2 | 0.232 |
| 5.3 | 0.253 |
| 5.4 | 0.115 |
| 6.1 | 0.107 |
| 6.2 | 0.015 |
| 6.3 | 0.047 |
| 7.1 | 0.008 |
| 7.2 | 0.017 |
| 7.3 | 0.059 |
| 7.4 | 0.008 |
| | EPK_mTOR/ |
| | IC50 [umol] |
| 2.22 | 0.080 |
| 2.24 | 0.029 |
| 2.25 | 0.012 |
| 2.26 | 0.022 |
| 2.27 | 0.169 |
| 2.28 | 0.058 |
| 2.29 | 0.043 |
| 2.30 | 0.077 |
| 2.31 | <0.003 |
| 2.32 | 0.007 |
| 7.5 | 0.088 |
| 7.6 | 0.059 |
| 7.7 | 0.447 |
| 7.8 | 0.086 |
| 7.9 | <0.003 |
| 7.10 | 0.301 |
| 7.11 | 0.011 |
| 7.12 | 0.066 |
| 7.13 | 0.067 |
| 7.14 | 0.251 |
| 7.15 | 0.426 |
| 7.16 | 0.11 |
| 7.17 | 0.080 |
| 7.18 | 0.036 |
| 8.9 | 0.010 |
| 8.10 | 0.470 |
| 8.11 | 0.005 |
| 8.12 | 0.017 |
| 8.13 | 0.086 |
| 8.14 | 0.115 |
| 9.4 | 0.009 |
| 11.7 | 0.003 |
| 11.8 | 0.046 |
| 12.1 | 0.010 |
| 12.2 | 0.007 |
| 13.1 | 0.049 |
| 13.2 | 0.018 |
| 13.3 | 0.014 |
| 13.4 | 0.006 |
| 13.5 | 0.039 |
| 14.2 | 0.026 |
| 14.3 | 0.048 |
| 14.4 | 0.014 |
| 14.5 | 0.017 |
| 14.6 | 0.006 |
| 14.7 | 0.013 |
| 14.8 | 0.028 |
| 15.1 | 0.008 |
| 15.2 | 0.007 |
| 15.3 | 0.120 |
| 15.4 | 0.011 |
| 15.5 | 0.071 |
| 15.6 | 0.006 |
| 15.7 | 0.075 |
| 15.8 | 0.011 |
| 15.9 | 0.119 |
| 16.1 | 0.110 |
| 16.2 | 0.053 |
| 16.3 | 0.151 |
| 16.4 | 0.704 |
| 16.5 | 0.622 |
| 17.1 | 0.484 |
| 18.1 | 0.013 |
| 18.3 | 0.215 |
| 19.1 | 0.003 |
| 19.2 | 0.009 |
| 19.4 | 0.042 |
| 19.5 | 0.009 |
| 20.1 | 0.007 |
| 20.2 | 0.154 |
| 20.3 | 0.059 |
| 20.4 | 0.011 |
| 20.5 | 0.900 |
| 21.1 | <0.003 |
| 22.1 | 0.098 |
| 22.2 | 0.021 |
| 22.3 | 0.140 |
| 23.1 | 0.009 |
| 23.2 | 0.044 |
| 23.3 | 0.007 |
| 23.4 | 0.006 |
| 23.5 | 0.025 |
| 23.6 | 0.137 |
| 23.7 | 0.423 |
| 23.8 | 0.004 |
| 23.9 | 0.037 |
| 24.1 | 0.021 |
| 24.2 | 0.048 |
| 24.3 | <0.003 |
| 24.4 | 0.449 |
| 25.1 | 0.034 |
| 25.2 | 0.076 |
| 25.3 | 0.012 |
| 25.4 | 0.655 |
| 25.5 | 0.022 |
| 26.1 | <0.006 |
| 26.2 | 0.003 |
| 26.3 | 0.009 |
| 27.1 | 0.025 |
| 27.2 | 0.006 |
| 27.3 | 0.007 |
| 27.4 | 0.023 |
| 27.5 | 0.015 |
| 28.1 | 0.022 |
| 28.2 | 0.008 |
| 28.3 | 0.172 |
| 29.1 | 0.005 |
| 29.2 | 0.005 |
| 29.3 | 0.008 |
| 29.4 | 0.012 |
| 29.5 | 0.061 |
| 29.6 | 0.011 |
| 29.7 | 0.008 |
| 30.1 | 0.118 |
| 31.1 | 0.021 |
| 31.2 | 0.006 |
| 31.4 | 0.075 |
| 31.5 | 0.006 |
| 31.6 | 0.095 |
| 31.7 | 0.021 |
| 31.8 | 0.122 |
| 31.9 | 0.139 |
| 32.1 | 0.004 |
| 32.2 | 0.010 |
| 32.3 | 0.015 |
| 32.4 | 0.007 |
| 32.5 | 0.009 |
| 32.7 | 0.05 |
| 32.8 | 0.0425 |
| 32.9 | 0.021 |
| 32.10 | 0.015 |
| 33.1 | 0.092 |
| 33.2 | 0.003 |
| 33.3 | 0.060 |
| 33.5 | 0.036 |
| 33.6 | 0.070 |
| 34.1 | 0.028 |
| 34.2 | 0.036 |
| 34.3 | 0.011 |
| 34.4 | 0.214 |
| 35.1 | 0.068 |
| 35.2 | 0.045 |
| 35.3 | 0.175 |
| 35.4 | 0.066 |

The efficacy of the compounds of the invention in blocking the activation of the PI3K/PKB pathway was demonstrated in cellular settings using a reverse protein array assay for sensitive quantification of compound of formula I mediated inhibition of PKB Ser473 phosphorylation in Rat1 cells stably transfected with activated versions of PI3-kinase isoforms alpha, beta or delta:

### Cells and Cell Culture Conditions:

Rat1 cell lines stably expressing a myr-HA-tagged, constitutively active subunit of the catalytic PI3K class I p110 isoform α, β or δ (addition of a myristylation signal at the N-terminus of p110 isoforms has been shown to lead to constitutive activation of PI3K and corresponding downstream signals, such as phosphorylation of PKB at Ser473) were cultivated in Dulbecco's modified Eagle's medium (DMEM high Glucose, GIBCO, cat. No. 41956-039) supplemented with 10% heat inactivated fetal bovine calf serum (Amimed, cat. No. 2-01F16-I), 1% L-Glutamine (Invitrogen, cat. No. 25030-02), 1% penicillin-streptomycin (GIBCO, cat. No. 15140-114) and 10 μg/ml Puromycine (Sigma, cat. No. P9620).

### Compound Treatment of Cells and Preparation of Samples:

The Rat1-myr-HA-p110 alpha, beta and delta cells were trypsinized and counted with a CASY TT cell counter (Schärfe System GmbH, Reutlingen Germany). The cells were diluted in fresh DMEM complete medium and 3×10⁴ cells/150 μl/well seeded in 96-well TPP-tissue culture plates (TPP, cat. No. 92096). The plates were incubated at 37° C. and 5% CO₂ under humidified condition for at least 20 h. Compound of formula I working solutions were prepared freshly as serial 8 step dilutions (vol/vol) in DMSO on the day of the experiment. The working solutions were further diluted 1/500 in cell culture medium to get final compound concentrations of 10, 3.333, 1.111, 0.370, 0.123, 0.041, 0.014, 0.005 μM. The final DMSO concentration was kept constant at 0.2%. cell culture medium containing 0.2% DMSO was used as vehicle treatment control. Post incubation, the cells were treated with the serial compound dilution. 50 μl of the compound medium mixture was added to the wells containing cells and 150 μl DMEM medium and incubated for 30 minutes (37° C., 5% CO₂). After 30 minutes of incubation, the medium mixture was quickly removed by aspiration. For cell lysis, a mixture of 10% CLB1 CeLyA lysis buffer (Zeptosens, cat. No. 9000), 90% CSBL1 CeLyA spotting buffer (Zeptosens, cat. No. 9020) was freshly prepared and supplemented with 1% Octyl β-D-glucopyranoside (SIGMA, cat. No. 08001-5G) and 1 mM Na-Orthovanadate (Sigma, cat No. S-6508). 50 μl of the described lysis buffer mix was added to each well, followed by 10 minutes of incubation on ice. After an additional freeze-thaw cycle, another 50 μl of the lysis buffer mix without Octyl β-D-glucopyranoside was added to the wells, and 90 μl of the cell lysate was transferred to a 96-well V-bottom plate (Fisher Scientific, cat. No. 6067Y), followed by a centrifugation step (5 min, 1500 rpm at 19° C. in an Eppendorf 5810R centrifuge) to remove the unlysed cell debris.

### Spotting of the ZeptoMARK® Chips:

The samples were spotted onto ZeptoMARK® PWG protein microarray chips (Zeptosens, Witterswil, Switzerland) with the piezoelectric microdispense-based, non-contact Nano-Plotter 2.1 (GeSiM, Grosserkmannsdorf, Germany). Each sample was spotted at 4 different sample concentrations (d1=100%, d2=75%, d3=50%, d4=25%) by diluting the cell lysate with the corresponding volume of lysis buffer mix. After spotting the ZeptoMARK® protein microarrays, the chips are incubated for 1 hour at 37° C. To receive a uniform blocking result, the CeLyA blocking buffer BB1 (Zeptosens, cat. No. 9040) is administered via an ultrasonic nebulizer. After 20 minutes of blocking the chips are extensively rinsed with deionized water (Milli-Q quality, 18MΩ×cm) and dried in a nitrogen air flow.

### ZeptoREADER Signal Detection and Data Analysis:

After the sample spotting and blocking procedure, the ZeptoMARK® chips were transferred to the ZeptoCARRIER (Zeptosens, cat. No. 1100), whose six flow cells individually address the six arrays on a chip, and washed twice with 200 μl CAB1 CeLyA assay buffer (Zeptosens, cat. No. 9032). The assay buffer was then aspirated and each compartment incubated with 100 μl of the primary target antibody (pAkt Ser473; CST#9271) at RT over night. Post incubation, the primary antibody was removed, the arrays washed twice with CAB1 buffer and further incubated with 100 μl of Alexa fluor 647-labeled anti rabbit IgG Fab fragments (Invitrogen; #Z25305) for one hour at RT in the dark. After incubation, the arrays were washed twice with 200 μl CAB1 buffer. The fluorescence of the target-bound Fab fragments is read out on the ZeptoReader (Zeptosens, Witterswil, Switzerland) using a laser (excitation wavelength 635 nm) and a CCD camera. The fluorescence signal was assessed with exposure times of 1, 3, 5 and 10 seconds, depending on the intensity of the signal. The resulting four images taken per array are stored as 16-bit TIF files.

The fluorescence images for each array were analyzed with the ZeptoVIEW Pro 2.0 software (Zeptosens, Witterswil, Switzerland) and the RFI for each signal was calculated. The range of activity, expressed as IC₅₀, in this assay is preferably between 5 nM and 10 μM, more preferably between 5 nM and about 1 μM.

| example | |
|---|---|
| number | |
| | p-PKB/IC50_alpha |
| | [nmol] |
| 1.1 | 42.92 |
| 1.2 | 5.70 |
| 1.3 | 6.94 |
| 1.4 | 8.68 |
| 1.5 | 9.35 |
| 1.6 | 6.56 |
| 1.7 | 5.44 |
| 1.8 | 97.63 |
| 1.9 | 57.63 |
| 2.1 | 6.31 |
| 2.2 | 15.86 |
| 2.3 | 20.99 |
| 2.4 | 14.58 |
| 2.5 | 49.00 |
| 2.6 | 38.57 |
| 2.7 | 11.63 |
| 2.8 | 6.84 |
| 2.9 | 8.40 |
| 2.10 | 114.48 |
| 2.11 | 7.02 |
| 2.12 | 29.83 |
| 2.13 | 11.52 |
| 2.14 | 7.38 |
| 2.15 | 72.42 |
| 2.16 | 25.25 |
| 2.17 | 85.62 |
| 3.1 | 631.51 |
| 3.2 | 58.49 |
| 3.3 | 177.92 |
| 3.4 | 226.44 |
| 3.5 | 92.74 |
| 3.6 | 150.35 |
| 3.7 | 398.29 |
| 3.8 | 54.90 |
| 3.9 | 301.14 |
| 3.10 | 78.33 |
| 3.11 | 371.59 |
| 3.12 | 13.74 |
| 3.13 | 308.39 |
| 4.1 | 542.09 |
| 4.2 | 47.29 |
| 4.3 | 202.81 |
| 4.4 | 167.07 |
| 5.1 | 166.93 |
| 5.2 | 18.14 |
| 5.3 | 92.21 |
| 5.4 | 92.43 |
| 6.1 | 31.48 |
| 6.2 | 4.57 |
| 6.3 | 13.68 |
| 7.1 | 11.76 |
| 7.2 | 12.07 |
| 7.3 | 42.22 |
| 7.4 | 7.18 |
| 8.1 | 7.27 |
| 8.2 | 7.36 |
| 8.3 | 4.57 |
| 9.1 | 18.41 |
| 9.2 | 33.91 |
| 9.3 | 6.50 |
| 11.1 | 5.01 |
| 11.2. | 6.97 |
| 11.3 | 8.83 |
| 11.4 | 86.62 |
| 11.5 | 16.86 |
| 11.6 | 6.11 |
| | p-PKB/IC50_alfa |
| | [nmol] |
| 2.22 | 11.0 |
| 2.23 | 64.1 |
| 2.24 | 22.2 |
| 2.25 | 36.3 |
| 2.26 | 6.4 |
| 2.27 | 100.4 |
| 2.28 | 23.7 |
| 2.29 | 8.7 |
| 2.30 | 52.2 |
| 2.31 | <4.57 |
| 2.32 | <4.57 |
| 7.5 | 31.5 |
| 7.6 | 34.4 |
| 7.7 | 96.1 |
| 7.8 | 14.5 |
| 7.9 | <4.57 |
| 7.10 | 123.0 |
| 7.11 | 12.6 |
| 7.12 | 6.4 |
| 7.13 | <4.57 |
| 7.14 | 15.2 |
| 7.15 | 20.1 |
| 7.16 | 30.3 |
| 7.17 | 31.1 |
| 8.8 | 72.6 |
| 8.9 | 24.9 |
| 8.10 | 20.5 |
| 8.11 | <4.57 |
| 8.12 | 27.7 |
| 8.13 | 207.9 |
| 8.14 | 35.0 |
| 9.4 | 18.0 |
| 11.7 | 4.9 |
| 11.8 | 7.8 |
| 12.1 | 5.2 |
| 12.2 | <4.57 |
| 13.1 | 17.0 |
| 13.2 | <4.57 |
| 13.3 | 5.0 |
| 13.4 | <4.57 |
| 13.5 | 6.9 |
| 14.2 | 20.4 |
| 14.3 | 39.0 |
| 14.4 | 16.2 |
| 14.5 | 4.9 |
| 14.6 | 5.2 |
| 14.7 | 11.5 |
| 14.8 | 9.0 |
| 15.1 | <4.57 |
| 15.2 | 5.0 |
| 15.3 | 12.3 |
| 15.4 | 7.2 |
| 15.5 | 40.7 |
| 15.6 | 4.8 |
| 15.7 | 9.6 |
| 15.8 | 5.1 |
| 15.9 | 10.8 |
| 16.1 | 13.3 |
| 16.2 | 21.8 |
| 16.3 | 64.1 |
| 16.4 | 30.7 |
| 16.5 | 18.4 |
| 17.1 | 53.4 |
| 17.2 | 46.8 |
| 18.1 | <4.57 |
| 18.2 | 7.8 |
| 18.3 | 7.1 |
| 19.2 | <4.57 |
| 19.3 | 6.8 |
| 19.4 | 6.8 |
| 19.5 | 6.9 |
| 20.1 | 286.6 |
| 20.2 | 50.0 |
| 20.3 | 6.6 |
| 20.4 | 5.6 |
| 20.5 | 90.2 |
| 21.1 | <4.57 |
| 22.1 | 17.7 |
| 22.2 | 6.4 |
| 22.3 | 24.2 |
| 23.1 | 21.1 |
| 23.2 | 24.8 |
| 23.3 | 5.7 |
| 23.4 | 5.7 |
| 23.5 | 37.3 |
| 23.6 | 37.1 |
| 23.7 | 28.5 |
| 23.8 | 7.3 |
| 23.9 | 26.2 |
| 24.1 | 15.3 |
| 24.2 | 17.5 |
| 24.3 | 5.9 |
| 24.4 | 22.3 |
| 25.1 | 11.5 |
| 25.2 | 48.0 |
| 25.3 | 13.9 |
| 25.4 | 27.5 |
| 25.5 | <4.57 |
| 26.1 | <4.57 |
| 26.2 | 5.1 |
| 26.3 | <4.57 |
| 27.1 | 41.1 |
| 27.2 | 11.3 |
| 27.3 | <4.57 |
| 27.4 | <4.57 |
| 27.5 | 160.4 |
| 28.1 | 22.7 |
| 28.2 | 9.1 |
| 28.3 | 44.5 |
| 29.1 | 335.2 |
| 29.2 | 14.0 |
| 29.3 | 21.9 |
| 29.4 | 8.0 |
| 29.5 | 197.4 |
| 29.6 | 17.0 |
| 30.1 | 53.6 |
| 31.1 | 14.6 |
| 31.3 | 5.3 |
| 32.1 | <4.57 |
| 32.2 | 8.1 |
| 32.3 | 45.0 |
| 32.4 | 8.0 |
| 32.5 | 10.0 |
| 32.6 | 24.3 |
| 32.7 | 61.7 |
| 32.8 | 35.8 |
| 33.1 | 19.0 |
| 33.2 | 5.2 |
| 34.1 | 13.0 |
| 34.2 | 11.7 |
| 34.4 | 35.0 |
| 34.5 | 30.3 |
| 35.1 | 14.6 |
| 35.2 | 4.6 |
| | p-PKB/IC50_beta [nmol] |
| 1.1 | 64.97 |
| 1.2 | <4.57 |
| 1.3 | 13.87 |
| 1.4 | 9.81 |
| 1.5 | 38.77 |
| 1.6 | 16.93 |
| 1.7 | 29.60 |
| 1.8 | 324.36 |
| 1.9 | 60.32 |
| 2.1 | 18.33 |
| 2.2 | 14.68 |
| 2.3 | 15.94 |
| 2.4 | 14.82 |
| 2.5 | 53.78 |
| 2.6 | 72.93 |
| 2.7 | 19.25 |
| 2.8 | 9.49 |
| 2.9 | 15.74 |
| 2.10 | 84.23 |
| 2.11 | 9.92 |
| 2.12 | 19.84 |
| 2.13 | 18.70 |
| 2.14 | 13.10 |
| 2.15 | 101.13 |
| 2.16 | 37.47 |
| 2.17 | 490.70 |
| 3.2 | 74.07 |
| 3.3 | 259.11 |
| 3.4 | 527.09 |
| 3.5 | 130.69 |
| 3.6 | 546.00 |
| 3.8 | 107.97 |
| 3.9 | 689.31 |
| 3.10 | 516.35 |
| 3.12 | 172.11 |
| 3.13 | 945.38 |
| 4.1 | 693.76 |
| 4.2 | 149.34 |
| 4.3 | 362.55 |
| 4.4 | 299.78 |
| 5.1 | 165.86 |
| 5.2 | 50.34 |
| 5.3 | 184.87 |
| 5.4 | 151.95 |
| 6.1 | 56.20 |
| 6.2 | 10.19 |
| 6.3 | 14.09 |
| 7.1 | 13.44 |
| 7.2 | 29.47 |
| 7.3 | 43.82 |
| 7.4 | 8.60 |
| 8.1 | 12.79 |
| 8.2 | 34.60 |
| 8.3 | <4.57 |
| 9.1 | 33.33 |
| 9.2 | 20.58 |
| 9.3 | 9.39 |
| 11.1 | 11.72 |
| 11.2. | 15.17 |
| 11.3 | 18.08 |
| 11.4 | 127.12 |
| 11.5 | 40.80 |
| 11.6 | 13.00 |
| 2.22 | 22.5 |
| 2.23 | 273.2 |
| 2.24 | 16.4 |
| 2.25 | 58.7 |
| 2.26 | 7.4 |
| 2.27 | 246.6 |
| 2.28 | 61.0 |
| 2.29 | 19.7 |
| 2.30 | 139.0 |
| 2.31 | 5.7 |
| 2.32 | 6.0 |
| 7.5 | 82.1 |
| 7.6 | 22.8 |
| 7.7 | 277.8 |
| 7.8 | 68.6 |
| 7.9 | 10.0 |
| 7.10 | 96.6 |
| 7.11 | 15.8 |
| 7.12 | 13.5 |
| 7.13 | 7.6 |
| 7.14 | 50.6 |
| 7.15 | 50.6 |
| 7.16 | 23.3 |
| 7.17 | 212.1 |
| 8.8 | 115.2 |
| 8.9 | 39.5 |
| 8.11 | 17.1 |
| 8.12 | 81.6 |
| 8.13 | 212.2 |
| 8.14 | 56.7 |
| 9.4 | 19.6 |
| 11.7 | 7.5 |
| 11.8 | 31.1 |
| 12.1 | <4.57 |
| 12.2 | <4.57 |
| 13.1 | 15.7 |
| 13.2 | 5.3 |
| 13.3 | <4.57 |
| 13.4 | <4.57 |
| 13.5 | 11.6 |
| 14.2 | 21.7 |
| 14.3 | 40.3 |
| 14.4 | 24.2 |
| 14.5 | 10.8 |
| 14.6 | 8.1 |
| 14.7 | 20.8 |
| 14.8 | 61.4 |
| 15.1 | 6.1 |
| 15.2 | 21.4 |
| 15.3 | 27.9 |
| 15.4 | 15.1 |
| 15.5 | 132.4 |
| 15.6 | 7.0 |
| 15.7 | 24.4 |
| 15.8 | 23.2 |
| 15.9 | 40.4 |
| 16.1 | 65.6 |
| 16.2 | 78.3 |
| 16.3 | 301.9 |
| 16.4 | 140.9 |
| 17.1 | 202.1 |
| 17.2 | 94.6 |
| 18.2 | 7.0 |
| 19.2 | <4.57 |
| 19.3 | 14.2 |
| 19.4 | 40.2 |
| 19.5 | 11.2 |
| 20.1 | 169.9 |
| 20.2 | 210.5 |
| 20.3 | 16.3 |
| 20.4 | 9.5 |
| 20.5 | 167.3 |
| 21.1 | 5.3 |
| 22.1 | 66.7 |
| 22.2 | 32.1 |
| 22.3 | 77.6 |
| 23.1 | 15.1 |
| 23.2 | 53.4 |
| 23.3 | 23.1 |
| 23.4 | 6.8 |
| 23.5 | 98.6 |
| 23.6 | 103.4 |
| 23.7 | 98.4 |
| 23.8 | <4.57 |
| 23.9 | 136.4 |
| 24.1 | 12.7 |
| 24.2 | 30.7 |
| 24.3 | <4.57 |
| 24.4 | 44.1 |
| 25.1 | 10.7 |
| 25.2 | 39.9 |
| 25.3 | 30.6 |
| 25.4 | 17.6 |
| 25.5 | <4.57 |
| 26.1 | <4.57 |
| 26.2 | <4.57 |
| 26.3 | 6.9 |
| 27.1 | 85.7 |
| 27.2 | 11.3 |
| 27.3 | 8.6 |
| 27.4 | 20.4 |
| 27.5 | 197.2 |
| 28.1 | 36.7 |
| 28.2 | 7.4 |
| 28.3 | 44.5 |
| 29.1 | 1096.7 |
| 29.2 | 32.7 |
| 29.3 | 31.9 |
| 29.4 | 21.9 |
| 29.5 | 373.5 |
| 29.6 | 207.8 |
| 30.1 | 237.4 |
| 31.1 | 19.3 |
| 31.3 | 131.7 |
| 32.1 | <4.57 |
| 32.2 | 15.2 |
| 32.3 | 80.0 |
| 32.4 | 13.0 |
| 32.5 | 11.0 |
| 32.6 | 39.7 |
| 32.7 | 200.4 |
| 32.8 | 63.7 |
| 33.1 | 50.0 |
| 33.2 | <4.47 |
| 34.1 | 20.0 |
| 34.2 | 15.2 |
| 34.4 | 27.9 |
| 34.5 | 31.0 |
| 35.1 | 27.89 |
| 35.2 | 5.27 |
| | p-PKB/IC50_delta |
| | [nmol] |
| 1.1 | 53.34 |
| 1.2 | 8.01 |
| 1.3 | 15.47 |
| 1.4 | 29.45 |
| 1.5 | 59.61 |
| 1.6 | 29.28 |
| 1.7 | 71.98 |
| 1.8 | 612.27 |
| 1.9 | 83.86 |
| 2.1 | 95.99 |
| 2.2 | 20.51 |
| 2.3 | 23.57 |
| 2.4 | 29.14 |
| 2.5 | 204.00 |
| 2.6 | 74.74 |
| 2.7 | 12.16 |
| 2.8 | 13.81 |
| 2.9 | 34.10 |
| 2.10 | 226.78 |
| 2.11 | 20.85 |
| 2.12 | 79.76 |
| 2.13 | 36.81 |
| 2.14 | 61.66 |
| 2.15 | 102.40 |
| 2.16 | 146.29 |
| 2.17 | 802.58 |
| 3.2 | 109.08 |
| 3.3 | 619.63 |
| 3.4 | 742.38 |
| 3.5 | 457.92 |
| 3.10 | 478.55 |
| 3.12 | 75.46 |
| 4.3 | 680.62 |
| 4.4 | 894.27 |
| 5.1 | 364.97 |
| 5.2 | 74.33 |
| 5.3 | 250.64 |
| 5.4 | 222.82 |
| 6.1 | 144.45 |
| 6.2 | 11.48 |
| 6.3 | 43.16 |
| 7.1 | 24.68 |
| 7.2 | 69.66 |
| 7.3 | 85.81 |
| 7.4 | 19.28 |
| 8.1 | 10.68 |
| 8.2 | 113.16 |
| 8.3 | 19.53 |
| 9.1 | 32.76 |
| 9.2 | 23.91 |
| 9.3 | 52.90 |
| 11.1 | 92.07 |
| 11.2. | 31.41 |
| 11.3 | 23.25 |
| 11.4 | 136.35 |
| 11.5 | 66.90 |
| 11.6 | 15.51 |
| 2.22 | 75.7 |
| 2.23 | 1057.5 |
| 2.24 | 28.9 |
| 2.25 | 110.1 |
| 2.26 | 67.3 |
| 2.27 | 825.0 |
| 2.28 | 218.0 |
| 2.29 | 121.8 |
| 2.30 | 335.9 |
| 2.31 | 31.4 |
| 2.32 | 33.7 |
| 7.5 | 182.3 |
| 7.6 | 118.7 |
| 7.7 | 597.8 |
| 7.8 | 80.8 |
| 7.9 | 41.5 |
| 7.10 | 315.4 |
| 7.11 | 17.1 |
| 7.12 | 97.8 |
| 7.13 | 37.9 |
| 7.14 | 139.8 |
| 7.15 | 172.4 |
| 7.16 | 78.5 |
| 7.17 | 119.0 |
| 8.8 | 142.6 |
| 8.9 | 74.9 |
| 8.10 | 412.2 |
| 8.11 | 93.1 |
| 8.12 | 134.5 |
| 8.13 | 599.0 |
| 8.14 | 186.5 |
| 9.4 | 40.2 |
| 11.7 | 8.3 |
| 11.8 | 113.9 |
| 12.1 | 45.7 |
| 12.2 | <4.57 |
| 13.1 | 113.2 |
| 13.2 | 29.4 |
| 13.3 | 38.5 |
| 13.4 | <4.57 |
| 13.5 | 37.1 |
| 14.2 | 68.1 |
| 14.3 | 122.3 |
| 14.4 | 32.6 |
| 14.5 | 14.4 |
| 14.6 | <4.57 |
| 14.7 | 45.4 |
| 14.8 | 34.4 |
| 15.1 | 13.3 |
| 15.2 | 53.3 |
| 15.3 | 211.7 |
| 15.4 | 31.3 |
| 15.5 | 229.4 |
| 15.6 | 6.8 |
| 15.7 | 44.3 |
| 15.8 | 59.7 |
| 15.9 | 38.8 |
| 16.1 | 42.9 |
| 16.2 | 63.3 |
| 16.3 | 304.5 |
| 16.4 | 407.8 |
| 16.5 | 407.2 |
| 17.1 | 412.4 |
| 17.2 | 206.1 |
| 18.1 | <4.57 |
| 18.2 | 12.2 |
| 18.3 | 90.7 |
| 19.2 | <4.57 |
| 19.3 | 74.7 |
| 19.4 | 74.5 |
| 19.5 | 46.4 |
| 20.1 | 193.8 |
| 20.2 | 326.0 |
| 20.3 | 218.3 |
| 20.4 | 196.7 |
| 20.5 | 351.9 |
| 21.1 | 10.3 |
| 22.1 | 206.9 |
| 22.2 | 83.9 |
| 22.3 | 210.2 |
| 23.1 | 66.9 |
| 23.2 | 122.9 |
| 23.3 | 42.2 |
| 23.4 | 23.8 |
| 23.5 | 154.8 |
| 23.6 | 215.6 |
| 23.7 | 139.4 |
| 23.8 | 10.2 |
| 23.9 | 80.1 |
| 24.1 | 42.6 |
| 24.2 | 30.0 |
| 24.3 | 8.1 |
| 24.4 | 88.5 |
| 25.1 | 26.0 |
| 25.2 | 124.0 |
| 25.3 | 25.4 |
| 25.4 | 58.1 |
| 25.5 | 7.6 |
| 26.1 | <4.57 |
| 26.2 | 10.4 |
| 26.3 | 12.8 |
| 27.1 | 64.5 |
| 27.2 | 20.7 |
| 27.3 | 8.9 |
| 27.4 | 38.1 |
| 27.5 | 309.4 |
| 28.1 | 39.9 |
| 28.2 | 22.9 |
| 28.3 | 121.2 |
| 29.1 | 350.5 |
| 29.2 | 39.7 |
| 29.3 | 65.3 |
| 29.4 | 18.3 |
| 29.5 | 400.7 |
| 29.6 | 119.1 |
| 30.1 | 714.9 |
| 31.1 | 36.0 |
| 31.3 | 26.4 |
| 32.1 | 17.7 |
| 32.2 | 30.5 |
| 32.3 | 59.0 |
| 32.4 | 16.0 |
| 32.5 | 16.0 |
| 32.6 | 83.2 |
| 32.7 | 178.3 |
| 32.8 | 95.3 |
| 33.1 | 43.0 |
| 33.2 | 28.2 |
| 34.1 | 42.0 |
| 34.2 | 39.9 |
| 34.4 | 253.2 |
| 34.5 | 84.2 |
| 35.1 | 25.48 |
| 35.2 | 16.34 |

The efficacy of the compounds of the invention in displaying an inhibitory effect downstream of mTORC1 complex (mTOR/raptor) was demonstrated in cellular setting using a reverse protein array assay for sensitive quantification of drug-mediated inhibition of S6 Ribosomal Protein phosphorylation Ser235/236 in TSC1 null mouse embryo fibroblast (MEF) cells.

### Cells and Cell Culture Conditions:

TSC1−/− MEFs cells (provided by D. Kwiatkowski), were cultivated in Dulbecco's modified Eagle's medium (DMEM high Glucose, GIBCO, cat. No. 41956-039) supplemented with 10% heat inactivated fetal bovine calf serum (Amimed, cat. No. 2-01F16-I), 1% L-Glutamine (Invitrogen, cat. No. 25030-02), 1% penicillin-streptomycin (GIBCO, cat. No. 15140-114) at 37° C. in a 5% CO2 and 95% relative humidity atmosphere incubator.

### Treatment of Cells and Preparation of Samples:

The TSC1−/− cells were trypsinized and counted with a CASY TT cell counter (Schärfe System GmbH, Reutlingen Germany). The cells were diluted in fresh DMEM complete medium and 0.75×10⁴ cells/150 μl/well seeded in 96-well TPP-tissue culture plates (TPP, cat. No. 92096). The plates were incubated at 37° C. and 5% CO₂ under humidified condition for at least 20 h. Working solutions were prepared freshly as serial 8 step dilutions (vol/vol) in DMSO on the day of the experiment. The working solutions were further diluted 1/5000 in cell culture medium to get final compound concentrations of 1, 0.3333, 0.1111, 0.037, 0.0123, 0.041, 0.0014, 0.005 μM. The final DMSO concentration was kept constant at 0.02%. cell culture medium containing 0.02% DMSO was used as vehicle treatment control. Post incubation, the cells were treated with the serial compound dilution. 50 μl of the compound medium mixture was added to the wells containing cells and 150 μl DMEM medium and incubated for 60 minutes (37° C., 5% CO₂). After 60 minutes of incubation, the medium mixture was quickly removed by aspiration. For cell lysis, a mixture of 10% CLB1 CeLyA lysis buffer (Zeptosens, cat. No. 9000), 90% CSBL1 CeLyA spotting buffer (Zeptosens, cat. No. 9020) was freshly prepared and supplemented with 1% Octyl β-D-glucopyranoside (SIGMA, cat. No. 08001-5G) and 1 mM Na-Orthovanadate (Sigma, cat No. S-6508). 50 μl of the described lysis buffer mix was added to each well, followed by 10 minutes of incubation on ice. After an additional freeze-thaw cycle, another 50 μl of the lysis buffer mix without Octyl β-D-glucopyranoside was added to the wells, and 90 μl of the cell lysate was transferred to a 96-well V-bottom plate (Fisher Scientific, cat. No. 6067Y), followed by a centrifugation step (5 min, 1500 rpm at 19° C. in an Eppendorf 5810R centrifuge) to remove the unlysed cell debris.

### Spotting of the ZeptoMARK® Chips:

The spotting of the chips follows the previous described method.

### ZeptoREADER Signal Detection and Data Analysis:

The signal detection and the data analysis follows the previous described method using the pS6 ribosomal protein Ser235/236 (CST, cat. No. #2211) as a primary antibody.

The range of activity, expressed as IC₅₀, in this assay is preferably between 0.5 nM and 1 μM, more preferably between 0.5 nM and about 0.5 μM.

| example | |
|---|---|
| number | |
| | p-S6 [nmol] |
| 1.1 | 24.53 |
| 1.2 | 2.61 |
| 1.3 | 1.52 |
| 1.4 | 8.24 |
| 1.5 | 48.61 |
| 1.6 | 21.89 |
| 1.7 | 12.09 |
| 1.9 | 32.81 |
| 2.1 | 19.01 |
| 2.2 | 5.33 |
| 2.3 | 56.40 |
| 2.4 | 23.09 |
| 2.5 | 236.76 |
| 2.6 | 66.09 |
| 2.7 | 12.37 |
| 2.10 | 158.84 |
| 2.11 | 2.35 |
| 2.12 | 18.03 |
| 2.13 | 2.78 |
| 2.14 | 8.74 |
| 2.15 | 68.21 |
| 3.3 | 297.06 |
| 3.12 | 13.24 |
| 4.2 | 459.55 |
| 4.3 | 220.17 |
| 6.2 | 1.47 |
| 7.1 | 2.23 |
| 7.2 | 17.87 |
| 7.3 | 84.30 |
| 7.4 | 8.52 |
| 8.1 | 1.07 |
| 8.2 | 7.69 |
| 8.3 | 1.05 |
| 9.1 | 13.46 |
| 9.2 | 3.35 |
| 11.1 | 10.62 |
| | p-S6 IC50 [nmol] |
| 2.22 | 12.0 |
| 2.23 | 37.0 |
| 2.24 | 4.0 |
| 2.26 | 6.0 |
| 2.27 | 54.0 |
| 2.28 | 14.0 |
| 2.29 | 5.0 |
| 2.30 | 34.0 |
| 2.31 | 21.0 |
| 2.32 | 2.0 |
| 7.5 | 21.0 |
| 7.6 | 7.0 |
| 7.7 | 113.0 |
| 7.8 | 10.0 |
| 7.9 | 5.0 |
| 7.10 | 15.0 |
| 7.11 | 7.0 |
| 7.15 | 8.0 |
| 7.16 | 26.0 |
| 7.17 | 5.0 |
| 8.8 | 7.0 |
| 8.9 | 4.0 |
| 8.10 | 6.8 |
| 8.11 | 1.0 |
| 8.12 | 4.0 |
| 8.13 | 15.0 |
| 9.4 | 6.0 |
| 11.7 | 2.0 |
| 12.1 | <0.457 |
| 12.2 | 3.0 |
| 13.1 | 18.0 |
| 13.2 | 1.0 |
| 13.3 | 3.0 |
| 13.4 | 1.0 |
| 13.5 | 1.1 |
| 14.2 | 16.0 |
| 14.3 | 10.0 |
| 14.4 | 7.0 |
| 14.5 | 1.0 |
| 14.6 | 2.0 |
| 14.7 | 3.0 |
| 14.8 | 2.0 |
| 15.1 | 1.0 |
| 15.2 | 7.0 |
| 15.3 | 130.0 |
| 15.4 | 3.0 |
| 15.5 | 19.0 |
| 15.6 | 3.0 |
| 15.7 | 2.0 |
| 15.8 | 6.0 |
| 16.1 | <0.457 |
| 16.2 | 6.0 |
| 16.3 | 22.0 |
| 16.4 | 5.0 |
| 16.5 | 12.6 |
| 17.1 | 29.0 |
| 17.2 | 10.0 |
| 18.1 | <0.457 |
| 18.2 | 1.0 |
| 18.3 | 4.3 |
| 19.2 | 1.1 |
| 19.3 | 2.0 |
| 20.1 | 32.0 |
| 20.2 | 131.0 |
| 20.3 | 44.0 |
| 20.4 | 63.0 |
| 22.1 | 7.0 |
| 22.2 | 2.0 |
| 22.3 | 15.0 |
| 23.1 | 2.0 |
| 23.8 | 6.0 |
| 24.1 | 2.0 |
| 24.2 | 1.0 |
| 24.3 | <0.457 |
| 25.1 | 1.0 |
| 25.2 | 2.0 |
| 25.3 | <0.457 |
| 25.4 | 7.0 |
| 26.1 | <0.457 |
| 26.2 | <0.457 |
| 27.1 | 2.0 |
| 27.2 | 2.0 |
| 27.5 | 33.0 |
| 28.1 | 1.0 |
| 28.2 | 1.0 |
| 28.3 | 8.0 |
| 29.1 | 15.0 |
| 29.3 | 7.8 |
| 29.4 | 1.7 |
| 29.5 | 39.1 |
| 29.6 | 5.0 |
| 31.3 | 1.0 |
| 32.2 | 1.0 |
| 32.3 | 4.0 |
| 32.4 | 4.0 |
| 32.5 | 1.0 |
| 32.6 | 8.6 |
| 32.7 | 21.4 |
| 32.8 | 2.2 |
| 33.1 | 2.0 |
| 33.2 | 2.0 |
| 34.1 | 3.0 |
| 34.2 | 1.9 |
| 34.4 | 8.0 |
| 34.5 | 2.0 |
| 35.1 | 0.79 |
| 35.2 | 1 |

There are also experiments to demonstrate the antitumor activity of compounds of the formula (I) in vivo.

For example, female Harlan (Indianapolis, Ind., USA) athymic nu/nu mice with s.c. transplanted human glioblastoms U87MG tumors can be used to determine the anti-tumor activity of PI3 kinase inhibitors. On day 0, with the animals under peroral Forene® (1-chloro-2,2,2-trifluoroethyldifluormethylether, Abbot, Wiesbaden, Germany) narcosis, a tumor frag-ment of approximately 25 mg is placed under the skin on the animals' left flank and the small incised wound is closed by means of suture clips. When tumors reach a volume of 100 mm³, the mice are divided at random into groups of 6-8 animals and treatment commences. The treatment is carried out for a 2-3 weeks period with peroral, intravenous or intra-peritoneal administration once daily (or less frequently) of a compound of formula (I) in a suitable vehicle at defined doses. The tumors are measured twice a week with a slide gauge and the volume of the tumors is calculated.

As an alternative to cell line U87MG, other cell lines may also be used in the same manner, for example,
the MDA-MB 468 breast adenocarcinoma cell line (ATCC No. HTB 132; see also In Vitro 14, 911-15 [1978]);
the MDA-MB 231 breast carcinoma cell line (ATCC No. HTB-26; see also In Vitro 12, 331 [1976]);
the MDA-MB 453 breast carcinoma cell line (ATCC No. HTB-131);
the Colo 205 colon carcinoma cell line (ATCC No. CCL 222; see also Cancer Res. 38, 1345-55 [1978]);
the DU145 prostate carcinoma cell line DU 145 (ATCC No. HTB 81; see also Cancer Res. 37, 4049-58 [1978]),
the PC-3 prostate carcinoma cell line PC-3 (especially preferred; ATCC No. CRL 1435; see also Cancer Res. 40, 524-34 [1980]) and the PC-3M prostate carcinoma cell line;
the A549 human lung adenocarcinoma (ATCC No. CCL 185; see also Int. J. Cancer 17, 62-70 [1976]),
the NCI-H596 cell line (ATCC No. HTB 178; see also Science 246, 491-4 [1989]);
the pancreatic cancer cell line SUIT-2 (see Tomioka et al., Cancer Res. 61, 7518-24 [2001]).

[symbol]

What is claimed is:

1. A compound of the formula (I) 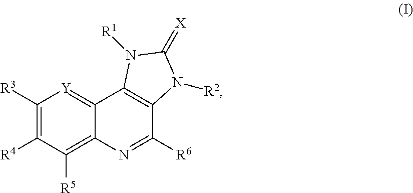
wherein
X is O or S;
Y is CH or N;
R¹ is substituted or unsubstituted pyridyl;
R² is hydrogen or lower alkyl;
R³ is a substituted or unsubstituted aryl or heterocyclyl; and
R⁴, R⁵ and R⁶ are hydrogen;
or a pharmaceutically acceptable salt thereof.

2. A compound according to claim 1,
wherein
R¹ is pyridyl which is unsubstituted or independently substituted by one, two or three substituents selected from halo, unsubstituted or substituted alkyl, unsubstituted or substituted alkenyl, unsubstituted or substituted alkynyl, unsubstituted or substituted lower alkoxy, unsubstituted or substituted alkoxy lower alkyl, unsubstituted or substituted lower alkoxy lower alkoxy, unsubstituted or substituted cycloalkyl, unsubstituted or substituted cycloalkenyl, unsubstituted or substituted carbamoyl lower alkyl, lower mono- and di-alkyl carbamoyl lower alkyl, unsubstituted or substituted cycloaminocarbonyl lower alkyl, unsubstituted or substituted heterocyclylcarbonyl lower alkyl, unsubstituted or substituted hydroxyl lower alkyl, unsubstituted or substituted carboxy lower alkyl, halo-loweralkyl, hydroxycarbonyl-loweralkyl, unsubstituted or substituted pyrazinyl, hydroxyl, amino, mono- or di-loweralkyl substituted amino, unsubstituted or substituted piperazinyl, N-loweralkyl-N-lower alkoxy lower alkyl substituted amino, lower alkoxy lower alkyl carbonyl amino, oxo-piperazinyl, unsubstituted or substituted cycloamino, N-loweralkyl-N-hydroxy lower alkyl substituted amino
or a pharmaceutically acceptable salt thereof.

3. A compound according to claim 1 or claim 2, wherein said substituted pyridinyl is substituted with one, two or three substituents independently selected from halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino.

4. A compound according to any one of claims 1-3, wherein said substituted pyridinyl is substituted with one, two or three of said substituents, at least one of which is in the alpha-position.

5. A compound according to any one of claims 1 to 4, wherein
R¹ is a group selected from: 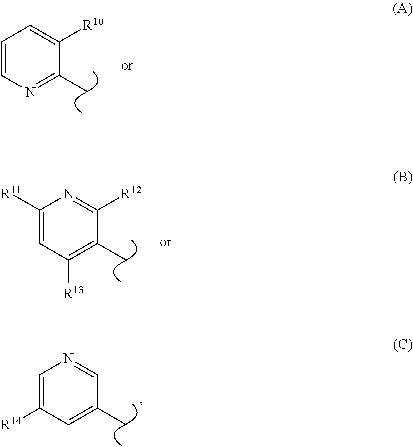
wherein, the curved line indicates the bonding position to the rest of the molecule, and
R¹⁰ is independently selected from hydrogen, halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino;
R¹¹ is independently selected from hydrogen, halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino;
R¹² is independently selected from hydrogen, halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino;
R¹³ is independently selected from hydrogen, halo, unsubstituted or substituted alkyl, unsubstituted or substituted lower alkoxy, unsubstituted or substituted lower alkoxy lower alkoxy, amino, mono- or di-loweralkyl substituted amino, N-loweralkyl-N-lower alkoxy lower alkyl substituted amino, lower alkoxy lower alkyl carbonyl amino, unsubstituted or substituted piperazinyl, oxo-piperazinyl, cycloamino, halo-substituted cycloamino, hydroxy-substituted cycloamino, alkoxy-substituted cycloamino, N-loweralkyl-N-hydroxy lower alkyl substituted amino;
or a pharmaceutically acceptable salt thereof.

6. A compound according to any preceding claim wherein,
R³ is selected from phenyl; hydroxyphenyl; methoxyphenyl; ethoxyphenyl; methoxy ethoxy-phenyl; methoxy-(methoxy ethoxy)-phenyl, hydroxy alkoxy phenyl; halo-hydroxy-phenyl; hydroxy-haloalkyl-phenyl; 2,2-difluoro-benzo[1,3]dioxolo; benzene sulfonamide; 3-(pyrrolidine-1-sulfonyl)-phenyl; N-(phen-3-yl)-methanesulfonamide; N-methyl-N-phen-3-yl-methanesulfonamide; alkyl-sulfonyl phenyl; benzamide; N-methyl-benzamide; N,N-dimethyl-benzamide; pyrazol-phenyl; imidazol-phenyl; pyridyl; alkylpyridyl; alkoxypyridyl; ethoxypyridyl; propoxypyridyl; cycloalkylalkoxypyridyl; alkoxyalkylpyridyl; alkoxyalkoxypyridyl; benzyloxyalkoxypyridyl; hydroxyalkylpyridyl; alkyl-sulfonyl pyridyl; hydroxyalkoxypyridyl; alkoxycarbonylpyridyl; aminopyridyl; alkylaminopyridyl; dialkylaminopyridyl; cycloaminopyridyl; (cyanoloweralkyl)-pyridinyl; (cyanocycloloweralkyl)-pyridinyl; hydroxyalkylaminopyridyl; amino-haloloweralkyl-pyridyl; haloalkylpyridinyl; halopyridyl; halo-alkoxy-pyridyl; carbamoylpyridyl; alkyl-substituted carbamoyl; piperazinylpyridyl; N-alkylpiperazinylpyridyl; alkylsulfonamidopyridyl; dialkylsulfonamidopyridyl; (alkylsulfonamido)(alkyl)pyridyl; dialkylsulfonamido(alkyl)pyridyl; 3H-tetrazol-5-yl pyridyl; (alkoxy)(alkylcarbonylamino)pyridyl; (alkoxy)(alkylcarbonyl-N-alkylamino)pyridyl; (alkoxy)(nitro)pyridyl; (alkoxy)(amino)pyridyl; (alkoxy)(alkylaminocarbonyl)pyridyl; (alkoxy)(hydroxycarbonyl)pyridyl; N,N-di-loweralkyl aminoloweralkoxy; (N,N-dimethylaminopropoxy)pyridyl; (alkyl)(alkoxy)pyridyl; (dialkoxy-alkyl)(alkoxy)pyridyl; (alkoxyalkyl)(alkoxy)pyridyl; (hydroxyalkyl)(alkylamino)pyridyl; (alkyl)(alkylamino)pyridyl; (halo)(alkylamino)pyridyl; (haloalkyl)(alkylamino)pyridyl; (haloalkyl)(amino)pyridyl; (hydroxyalkyl)(alkoxy)pyridyl; (hydroxyalkyl)(amino)pyridyl; (alkoxyalkyl)(amino)pyridyl; (alkyl)(alkoxyalkoxy)pyridyl; (alkoxyalkyl)(alkylamino)pyridyl; (amino)(alkylaminocarbonyl)pyridyl; pyrimidinyl; loweralkylaminopyrimidinyl; di-loweralkylaminopyrimidinyl; alkoxypyrimidinyl; di-loweralkoxypyrimidinyl; (alkylamino)(alkoxy)pyrimidinyl; 1H-pyrrolo[2,3-b]pyridinyl; 1-methyl-1H-pyrrolo[2,3-b]pyridinyl; pyrazinyl; pyrazolyl; substituted pyrazolyl; quinolinyl; 2-oxo-2,3-dihydro-1H-indol-5-yl; 1-methyl-2,3-dihydro-1H-indol-5-yl; 1H-imidazo[4,5-b]pyridin-2(3H)-one-6-yl; 3H-imidazo[4,5-b]pyridin-6-yl; 3H-[1,2,3]triazolo[4,5-b]pyridin-6-yl; imidazo[1,2-a]pyridinyl,
or a pharmaceutically acceptable salt thereof.

7. A compound according to claim 1, selected from the group consisting of:
1-(2-Methoxy-pyridin-3-yl)-8-(6-methoxy-pyridin-3-yl)-3-methyl-1,3-dihydro-imidazo[4,5-c]quinolin-2-one;
1-(2-Methoxy-pyridin-3-yl)-3-methyl-8-(1H-pyrrolo[2,3-b]pyridin-5-yl)-1,3-dihydro-imidazo[4,5-c]quinolin-2-one;
8-(6-Amino-5-trifluoromethyl-pyridin-3-yl)-1-(2-methoxy-pyridin-3-yl)-3-methyl-1,3-dihydro-imidazo[4,5-c]quinolin-2-one;
1-(2-Methoxy-pyridin-3-yl)-3-methyl-8-(6-methylamino-pyridin-3-yl)-1,3-dihydro-imidazo[4,5-c]quinolin-2-one;
8-(6-Ethoxy-pyridin-3-yl)-1-(2-methoxy-pyridin-3-yl)-3-methyl-1,3-dihydro-imidazo[4,5-c]quinolin-2-one;
4-[1-(2-Methoxy-pyridin-3-yl)-3-methyl-2-oxo-2,3-dihydro-1H-imidazo[4,5-c]quinolin-8-yl]-benzamide;
1-(2-Methoxy-pyridin-3-yl)-3-methyl-8-(1-methyl-1H-pyrrolo[2,3-b]pyridin-5-yl)-1,3-dihydro-imidazo[4,5-c]quinolin-2-one;
8-(6-Fluoro-pyridin-3-yl)-1-(2-methoxy-pyridin-3-yl)-3-methyl-1,3-dihydro-imidazo[4,5-c]quinolin-2-one;
8-(2-Dimethylamino-pyrimidin-5-yl)-1-(2-methoxy-pyridin-3-yl)-3-methyl-1,3-dihydro-imidazo[4,5-c]quinolin-2-one;
8-(6-Ethoxy-pyridin-3-yl)-3-methyl-1-(2-methyl-pyridin-3-yl)-1,3-dihydro-imidazo[4,5-c]quinolin-2-one;
3-Methyl-8-(2-methylamino-pyrimidin-5-yl)-1-(2-methyl-pyridin-3-yl)-1,3-dihydro-imidazo[4,5-c]quinolin-2-one;
4-[3-Methyl-1-(2-methyl-pyridin-3-yl)-2-oxo-2,3-dihydro-1H-imidazo[4,5-c]quinolin-8-yl]-benzamide;
N-Methyl-4-[3-methyl-1-(2-methyl-pyridin-3-yl)-2-oxo-2,3-dihydro-1H-imidazo[4,5-c]quinolin-8-yl]-benzamide;
8-(2-Ethoxy-pyrimidin-5-yl)-3-methyl-1-(2-methyl-pyridin-3-yl)-1,3-dihydro-imidazo[4,5-c]quinolin-2-one;
8-(6-Hydroxymethyl-pyridin-3-yl)-3-methyl-1-(2-methyl-pyridin-3-yl)-1,3-dihydro-imidazo[4,5-c]quinolin-2-one;
8-(6-Amino-pyridin-3-yl)-3-methyl-1-(2-methyl-pyridin-3-yl)-1,3-dihydro-imidazo[4,5-c]quinolin-2-one;
3-Methyl-1-(2-methyl-pyridin-3-yl)-8-(1H-pyrrolo[2,3-b]pyridin-5-yl)-1,3-dihydro-imidazo[4,5-c]quinolin-2-one;
8-(3,4-Dimethoxy-phenyl)-3-methyl-1-(2-methyl-pyridin-3-yl)-1,3-dihydro-imidazo[4,5-c]quinolin-2-one;
8-[6-(3-Dimethylamino-propoxy)-pyridin-3-yl]-3-methyl-1-(2-methyl-pyri din-3-yl)-1,3-dihydro-imidazo[4,5-c]quinolin-2-one;
8-(6-Amino-5-trifluoromethyl-pyridin-3-yl)-3-methyl-1-(2-methyl-pyridin-3-yl)-1,3-dihydro-imidazo[4,5-c]quinolin-2-one;
8-(6-Methoxy-pyridin-3-yl)-3-methyl-1-(2-methyl-pyridin-3-yl)-1,3-dihydro-imidazo[4,5-c]quinolin-2-one;
3-Methyl-8-(6-methylamino-pyridin-3-yl)-1-(2-methyl-pyridin-3-yl)-1,3-dihydro-imidazo[4,5-c]quinolin-2-one;
3-Methyl-1-(2-methyl-pyridin-3-yl)-8-(1-methyl-1H-pyrrolo[2,3-b]pyridin-5-yl)-1,3-dihydro-imidazo[4,5-c]quinolin-2-one;
8-(2-Dimethylamino-pyrimidin-5-yl)-3-methyl-1-(2-methyl-pyridin-3-yl)-1,3-dihydro-imidazo[4,5-c]quinolin-2-one;
8-(5-Methoxymethyl-pyridin-3-yl)-3-methyl-1-(2-methyl-pyridin-3-yl)-1,3-dihydro-imidazo[4,5-c]quinolin-2-one;
8-[5-(2-Methoxy-ethoxy)-pyridin-3-yl]-3-methyl-1-(2-methyl-pyridin-3-yl)-1,3-dihydro-imidazo[4,5-c]quinolin-2-one;
8-(2-Ethoxy-pyridin-4-yl)-3-methyl-1-(2-methyl-pyridin-3-yl)-1,3-dihydro-imidazo[4,5-c]quinolin-2-one;
8-(5-Isopropoxy-6-methyl-pyridin-3-yl)-3-methyl-1-(2-methyl-pyridin-3-yl)-1,3-dihydro-imidazo[4,5-c]quinolin-2-one;
8-(5-Ethoxy-6-methyl-pyridin-3-yl)-3-methyl-1-(2-methyl-pyridin-3-yl)-1,3-dihydro-imidazo[4,5-c]quinolin-2-one;
8-(5-Ethylamino-6-methyl-pyridin-3-yl)-3-methyl-1-(2-methyl-pyridin-3-yl)-1,3-dihydro-imidazo[4,5-c]quinolin-2-one;
8-(5-Ethoxy-6-methyl-pyridin-3-yl)-3-methyl-1-(2-methyl-pyridin-3-yl)-1,3-dihydro-imidazo[4,5-c]quinolin-2-one;
8-Imidazo[1,2-a]pyridin-6-yl-3-methyl-1-(2-methyl-pyridin-3-yl)-1,3-dihydro-imidazo[4,5-c]quinolin-2-one;
8-(5-Isopropylamino-pyridin-3-yl)-3-methyl-1-(2-methyl-pyridin-3-yl)-1,3-dihydro-imidazo[4,5-c]quinolin-2-one;
8-(5-Amino-pyridin-3-yl)-3-methyl-1-(2-methyl-pyridin-3-yl)-1,3-dihydro-imidazo[4,5-c]quinolin-2-one;
3-Methyl-8-(5-methylamino-pyridin-3-yl)-1-(2-methyl-pyridin-3-yl)-1,3-dihydro-imidazo[4,5-c]quinolin-2-one;
8-(6-Dimethoxymethyl-5-methoxy-pyridin-3-yl)-3-methyl-1-(2-methyl-pyridin-3-yl)-1,3-dihydro-imidazo[4,5-c]quinolin-2-one;
8-(5-Ethoxy-6-methoxymethyl-pyridin-3-yl)-3-methyl-1-(2-methyl-pyridin-3-yl)-1,3-dihydro-imidazo[4,5-c]quinolin-2-one;
8-(5-Azetidin-1-yl-pyridin-3-yl)-3-methyl-1-(2-methyl-pyridin-3-yl)-1,3-dihydro-imidazo[4,5-c]quinolin-2-one;
8-(5-Methoxy-6-methoxymethyl-pyridin-3-yl)-3-methyl-1-(2-methyl-pyridin-3-yl)-1,3-dihydro-imidazo[4,5-c]quinolin-2-one;
8-(5-Ethylamino-6-hydroxymethyl-pyridin-3-yl)-3-methyl-1-(2-methyl-pyridin-3-yl)-1,3-dihydro-imidazo[4,5-c]quinolin-2-one;
3-Methyl-8-(6-methyl-5-methylamino-pyridin-3-yl)-1-(2-methyl-pyridin-3-yl)-1,3-dihydro-imidazo[4,5-c]quinolin-2-one;
8-(2-Ethoxy-pyrimidin-5-yl)-3-methyl-1-(3-methyl-pyridin-2-yl)-1,3-dihydro-imidazo[4,5-c]quinolin-2-one;
3-Methyl-1-(3-methyl-pyridin-2-yl)-8-(1H-pyrrolo[2,3-b]pyridin-5-yl)-1,3-dihydro-imidazo[4,5-c]quinolin-2-one;
4-[3-Methyl-1-(3-methyl-pyridin-2-yl)-2-oxo-2,3-dihydro-1H-imidazo[4,5-c]quinolin-8-yl]-benzamide;
3-Methyl-8-(2-methylamino-pyrimidin-5-yl)-1-(3-methyl-pyridin-2-yl)-1,3-dihydro-imidazo[4,5-c]quinolin-2-one;
N-Methyl-4-[3-methyl-1-(3-methyl-pyridin-2-yl)-2-oxo-2,3-dihydro-1H-imidazo[4,5-c]quinolin-8-yl]-benzamide;
8-(6-Ethoxy-pyridin-3-yl)-3-methyl-1-(3-methyl-pyridin-2-yl)-1,3-dihydro-imidazo[4,5-c]quinolin-2-one;
8-[6-(3-Dimethylamino-propoxy)-pyridin-3-yl]-3-methyl-1-(3-methyl-pyridin-2-yl)-1,3-dihydro-imidazo[4,5-c]quinolin-2-one;
3-Methyl-1-(3-methyl-pyridin-2-yl)-8-quinolin-3-yl-1,3-dihydro-imidazo[4,5-c]quinolin-2-one;
8-(6-Hydroxymethyl-pyridin-3-yl)-3-methyl-1-(3-methyl-pyridin-2-yl)-1,3-dihydro-imidazo[4,5-c]quinolin-2-one;
8-(3,4-Dimethoxy-phenyl)-3-methyl-1-(3-methyl-pyridin-2-yl)-1,3-dihydro-imidazo[4,5-c]quinolin-2-one;
8-(6-Dimethylamino-pyridin-3-yl)-3-methyl-1-(3-methyl-pyridin-2-yl)-1,3-dihydro-imidazo[4,5-c]quinolin-2-one;
8-(6-Amino-5-trifluoromethyl-pyridin-3-yl)-3-methyl-1-(3-methyl-pyridin-2-yl)-1,3-dihydro-imidazo[4,5-c]quinolin-2-one;
8-(6-Methoxy-pyridin-3-yl)-3-methyl-1-(3-methyl-pyridin-2-yl)-1,3-dihydro-imidazo[4,5-c]quinolin-2-one;
8-(6-Hydroxymethyl-pyridin-3-yl)-3-methyl-1-(4-methyl-pyridin-3-yl)-1,3-dihydro-imidazo[4,5-c]quinolin-2-one;
8-(6-Ethoxy-pyridin-3-yl)-3-methyl-1-(4-methyl-pyridin-3-yl)-1,3-dihydro-imidazo[4,5-c]quinolin-2-one;
8-(6-Methoxy-pyridin-3-yl)-3-methyl-1-(4-methyl-pyridin-3-yl)-1,3-dihydro-imidazo[4,5-c]quinolin-2-one;
8-(6-Dimethylamino-pyridin-3-yl)-3-methyl-1-(4-methyl-pyridin-3-yl)-1,3-dihydro-imidazo[4,5-c]quinolin-2-one;
8-(6-Hydroxymethyl-pyridin-3-yl)-3-methyl-1-pyridin-3-yl-1,3-dihydro-imidazo[4,5-c]quinolin-2-one;
8-(3,4-Dimethoxy-phenyl)-3-methyl-1-pyridin-3-yl-1,3-dihydro-imidazo[4,5-c]quinolin-2-one;
8-(6-Dimethylamino-pyridin-3-yl)-3-methyl-1-pyridin-3-yl-1,3-dihydro-imidazo[4,5-c]quinolin-2-one;
8-(6-Methoxy-pyridin-3-yl)-3-methyl-1-pyridin-3-yl-1,3-dihydro-imidazo[4,5-c]quinolin-2-one;
8-(6-Ethoxy-pyridin-3-yl)-1-(2-fluoro-pyridin-3-yl)-3-methyl-1,3-dihydro-imidazo[4,5-c]quinolin-2-one;
8-(6-Amino-5-trifluoromethyl-pyridin-3-yl)-1-(2-fluoro-pyridin-3-yl)-3-methyl-1,3-dihydro-imidazo[4,5-c]quinolin-2-one;
1-(2-Fluoro-pyridin-3-yl)-3-methyl-8-(1H-pyrrolo[2,3-b]pyridin-5-yl)-1,3-dihydro-imidazo[4,5-c]quinolin-2-one;
8-(6-Amino-5-trifluoromethyl-pyridin-3-yl)-1-(6-fluoro-2-methyl-pyridin-3-yl)-3-methyl-1,3-dihydro-imidazo[4,5-c]quinolin-2-one;
1-(6-Fluoro-2-methyl-pyridin-3-yl)-3-methyl-8-(1-methyl-1H-pyrrolo[2,3-b]pyridin-5-yl)-1,3-dihydro-imidazo[4,5-c]quinolin-2-one;
8-(6-Ethoxy-pyridin-3-yl)-1-(6-fluoro-2-methyl-pyridin-3-yl)-3-methyl-1,3-dihydro-imidazo[4,5-c]quinolin-2-one;
1-(6-Fluoro-2-methyl-pyridin-3-yl)-3-methyl-8-(6-methylamino-pyridin-3-yl)-1,3-dihydro-imidazo[4,5-c]quinolin-2-one;
8-(5-Ethoxy-6-methoxymethyl-pyridin-3-yl)-1-(6-fluoro-2-methyl-pyridin-3-yl)-3-methyl-1,3-dihydro-imidazo[4,5-c]quinolin-2-one;
8-(5-Azetidin-1-yl-pyridin-3-yl)-1-(6-fluoro-2-methyl-pyridin-3-yl)-3-methyl-1,3-dihydro-imidazo[4,5-c]quinolin-2-one;
1-(6-Fluoro-2-methyl-pyridin-3-yl)-8-(5-methoxy-6-methoxymethyl-pyridin-3-yl)-3-methyl-1,3-dihydro-imidazo[4,5-c]quinolin-2-one;
1-(6-Fluoro-2-methyl-pyridin-3-yl)-8-[5-(1-hydroxy-1-methyl-ethyl)-pyridin-3-yl]-3-methyl-1,3-dihydro-imidazo[4,5-c]quinolin-2-one;
8-(5-Ethylamino-6-hydroxymethyl-pyridin-3-yl)-1-(6-fluoro-2-methyl-pyridin-3-yl)-3-methyl-1,3-dihydro-imidazo[4,5-c]quinolin-2-one;
1-(6-Fluoro-2-methyl-pyridin-3-yl)-8-(6-hydroxymethyl-5-methoxy-pyridin-3-yl)-3-methyl-1,3-dihydro-imidazo[4,5-c]quinolin-2-one;
1-(6-Fluoro-2-methyl-pyridin-3-yl)-8-(6-hydroxymethyl-5-methylamino-pyridin-3-yl)-3-methyl-1,3-dihydro-imidazo[4,5-c]quinolin-2-one;
8-(5-Fluoro-6-methylamino-pyridin-3-yl)-1-(6-fluoro-2-methyl-pyridin-3-yl)-3-methyl-1,3-dihydro-imidazo[4,5-c]quinolin-2-one;
1-(6-Fluoro-2-methyl-pyridin-3-yl)-3-methyl-8-(6-methylamino-5-trifluoromethyl-pyridin-3-yl)-1,3-dihydro-imidazo[4,5-c]quinolin-2-one;
2-Amino-5-[1-(6-fluoro-2-methyl-pyridin-3-yl)-3-methyl-2-oxo-2,3-dihydro-1H-imidazo[4,5-c]quinolin-8-yl]-N-methyl-nicotinamide;
8-(6-Amino-5-ethoxymethyl-pyridin-3-yl)-1-(6-fluoro-2-methyl-pyridin-3-yl)-3-methyl-1,3-dihydro-imidazo[4,5-c]quinolin-2-one;
8-(5-Amino-pyridin-3-yl)-1-(6-fluoro-2-methyl-pyridin-3-yl)-3-methyl-1,3-dihydro-imidazo[4,5-c]quinolin-2-one;
8-(6-Amino-5-hydroxymethyl-pyridin-3-yl)-1-(6-fluoro-2-methyl-pyridin-3-yl)-3-methyl-1,3-dihydro-imidazo[4,5-c]quinolin-2-one;
8-(2-Ethylamino-pyrimidin-5-yl)-1-(6-fluoro-2-methyl-pyridin-3-yl)-3-methyl-1,3-dihydro-imidazo[4,5-c]quinolin-2-one;
8-(6-Amino-5-trifluoromethyl-pyridin-3-yl)-1-(2,6-dimethoxy-pyridin-3-yl)-3-methyl-1,3-dihydro-imidazo[4,5-c]quinolin-2-one;
1-(2,6-Dimethoxy-pyridin-3-yl)-8-(6-ethoxy-pyridin-3-yl)-3-methyl-1,3-dihydro-imidazo[4,5-c]quinolin-2-one;
1-(2,6-Dimethoxy-pyridin-3-yl)-3-methyl-8-(6-methylamino-pyridin-3-yl)-1,3-dihydro-imidazo[4,5-c]quinolin-2-one;
1-(2,6-Dimethoxy-pyridin-3-yl)-3-methyl-8-(2-methylamino-pyrimidin-5-yl)-1,3-dihydro-imidazo[4,5-c]quinolin-2-one;
1-(2,6-Dimethoxy-pyridin-3-yl)-8-(2-dimethylamino-pyrimidin-5-yl)-3-methyl-1,3-dihydro-imidazo[4,5-c]quinolin-2-one;
1-(2,6-Dimethoxy-pyridin-3-yl)-3-methyl-8-(1-methyl-1H-pyrrolo[2,3-b]pyridin-5-yl)-1,3-dihydro-imidazo[4,5-c]quinolin-2-one;
1-(2,6-Dimethoxy-pyridin-3-yl)-8-(5-ethylamino-6-methyl-pyridin-3-yl)-3-methyl-1,3-dihydro-imidazo[4,5-c]quinolin-2-one;
1-(2,6-Dimethoxy-pyridin-3-yl)-8-[5-(2-methoxy-ethoxy)-pyridin-3-yl]-3-methyl-1,3-dihydro-imidazo[4,5-c]quinolin-2-one;
1-(2,6-Dimethoxy-pyridin-3-yl)-8-[5-(2-methoxy-ethoxy)-6-methyl-pyridin-3-yl]-3-methyl-1,3-dihydro-imidazo[4,5-c]quinolin-2-one;
1-(2,6-Dimethoxy-pyridin-3-yl)-8-imidazo[1,2-a]pyridin-6-yl-3-methyl-1,3-dihydro-imidazo[4,5-c]quinolin-2-one;
8-(5-Azetidin-1-yl-pyridin-3-yl)-1-(2,6-dimethoxy-pyridin-3-yl)-3-methyl-1,3-dihydro-imidazo[4,5-c]quinolin-2-one;
1-(2,6-Dimethoxy-pyridin-3-yl)-8-(5-methoxy-6-methoxymethyl-pyridin-3-yl)-3-methyl-1,3-dihydro-imidazo[4,5-c]quinolin-2-one;
8-(6-Dimethoxymethyl-5-methoxy-pyridin-3-yl)-1-(2,6-dimethoxy-pyridin-3-yl)-3-methyl-1,3-dihydro-imidazo[4,5-c]quinolin-2-one;
1-(2,6-Dimethoxy-pyridin-3-yl)-8-(6-hydroxymethyl-5-methoxy-pyridin-3-yl)-3-methyl-1,3-dihydro-imidazo[4,5-c]quinolin-2-one;
8-(6-Ethoxy-pyridin-3-yl)-1-(2-methoxy-6-piperazin-1-yl-pyridin-3-yl)-3-methyl-1,3-dihydro-imidazo[4,5-c]quinolin-2-one;
4-(6-Methoxy-5-nitro-pyridin-2-yl)-piperazine-1-carboxylic acid tert-butyl ester;
8-(6-Amino-5-trifluoromethyl-pyridin-3-yl)-1-(2-methoxy-6-piperazin-1-yl-pyridin-3-yl)-3-methyl-1,3-dihydro-imidazo[4,5-c]quinolin-2-one;
1-(2-Methoxy-6-piperazin-1-yl-pyridin-3-yl)-3-methyl-8-(1-methyl-1H-pyrrolo[2,3-b]pyridin-5-yl)-1,3-dihydro-imidazo[4,5-c]quinolin-2-one;
8-(2-Dimethylamino-pyrimidin-5-yl)-1-(2-methoxy-6-piperazin-1-yl-pyridin-3-yl)-3-methyl-1,3-dihydro-imidazo[4,5-c]quinolin-2-one;
8-(2-Dimethylamino-pyrimidin-5-yl)-1-[2-methoxy-6-(4-methyl-piperazin-1-yl)-pyridin-3-yl]-3-methyl-1,3-dihydro-imidazo[4,5-c]quinolin-2-one;
1-[2-Methoxy-6-(4-methyl-piperazin-1-yl)-pyridin-3-yl]-3-methyl-8-(6-methylamino-pyridin-3-yl)-1,3-dihydro-imidazo[4,5-c]quinolin-2-one;
8-(6-Ethoxy-pyridin-3-yl)-1-(6-methoxy-2-methyl-pyridin-3-yl)-3-methyl-1,3-dihydro-imidazo[4,5-c]quinolin-2-one;
1-(6-Methoxy-2-methyl-pyridin-3-yl)-3-methyl-8-(6-methylamino-pyridin-3-yl)-1,3-dihydro-imidazo[4,5-c]quinolin-2-one;
1-(6-Methoxy-2-methyl-pyridin-3-yl)-3-methyl-8-(2-methylamino-pyrimidin-5-yl)-1,3-dihydro-imidazo[4,5-c]quinolin-2-one;
8-(2-Dimethylamino-pyrimidin-5-yl)-1-(6-methoxy-2-methyl-pyridin-3-yl)-3-methyl-1,3-dihydro-imidazo[4,5-c]quinolin-2-one;
1-(6-Methoxy-2-methyl-pyridin-3-yl)-8-(6-methoxy-pyridin-3-yl)-3-methyl-1,3-dihydro-imidazo[4,5-c]quinolin-2-one;
8-(6-Amino-5-trifluoromethyl-pyridin-3-yl)-1-(6-methoxy-2-methyl-pyridin-3-yl)-3-methyl-1,3-dihydro-imidazo[4,5-c]quinolin-2-one;
8-(5-Ethylamino-6-methyl-pyridin-3-yl)-1-(6-methoxy-2-methyl-pyridin-3-yl)-3-methyl-1,3-dihydro-imidazo[4,5-c]quinolin-2-one;
8-(6-Hydroxymethyl-5-methoxy-pyridin-3-yl)-1-(6-methoxy-2-methyl-pyridin-3-yl)-3-methyl-1,3-dihydro-imidazo[4,5-c]quinolin-2-one;
1-[6-(Ethyl-methyl-amino)-2-methyl-pyridin-3-yl]-3-methyl-8-(6-methylamino-pyridin-3-yl)-1,3-dihydro-imidazo[4,5-c]quinolin-2-one;
8-(5-Ethylamino-6-methyl-pyridin-3-yl)-1-[6-(ethyl-methyl-amino)-2-methyl-pyridin-3-yl]-3-methyl-1,3-dihydro-imidazo[4,5-c]quinolin-2-one;
8-(6-Ethoxy-pyridin-3-yl)-1-[6-(2-methoxy-ethoxy)-2-methyl-pyridin-3-yl]-3-methyl-1,3-dihydro-imidazo[4,5-c]quinolin-2-one;
8-(6-Amino-5-trifluoromethyl-pyridin-3-yl)-1-[6-(2-methoxy-ethoxy)-2-methyl-pyridin-3-yl]-3-methyl-1,3-dihydro-imidazo[4,5-c]quinolin-2-one;
1-[6-(2-Methoxy-ethoxy)-2-methyl-pyridin-3-yl]-3-methyl-8-(6-methylamino-pyridin-3-yl)-1,3-dihydro-imidazo[4,5-c]quinolin-2-one;
8-(5-Ethylamino-6-methyl-pyridin-3-yl)-1-[6-(2-methoxy-ethoxy)-2-methyl-pyridin-3-yl]-3-methyl-1,3-dihydro-imidazo[4,5-c]quinolin-2-one;
1-[6-(2-Methoxy-ethoxy)-2-methyl-pyridin-3-yl]-3-methyl-8-(2-methylamino-pyrimidin-5-yl)-1,3-dihydro-imidazo[4,5-c]quinolin-2-one;
1-(2,6-Dimethyl-pyridin-3-yl)-3-methyl-8-(6-methylamino-pyridin-3-yl)-1,3-dihydro-imidazo[4,5-c]quinolin-2-one;
1-(2,6-Dimethyl-pyridin-3-yl)-8-(6-ethoxy-pyridin-3-yl)-3-methyl-1,3-dihydro-imidazo[4,5-c]quinolin-2-one;
8-(2-Dimethylamino-pyrimidin-5-yl)-1-(2,6-dimethyl-pyridin-3-yl)-3-methyl-1,3-dihydro-imidazo[4,5-c]quinolin-2-one;
1-(2,6-Dimethyl-pyridin-3-yl)-3-methyl-8-(2-methylamino-pyrimidin-5-yl)-1,3-dihydro-imidazo[4,5-c]quinolin-2-one;
8-(6-Amino-5-trifluoromethyl-pyridin-3-yl)-1-(2,6-dimethyl-pyridin-3-yl)-3-methyl-1,3-dihydro-imidazo[4,5-c]quinolin-2-one;
1-(2,6-Dimethyl-pyridin-3-yl)-8-(5-ethylamino-6-methyl-pyridin-3-yl)-3-methyl-1,3-dihydro-imidazo[4,5-c]quinolin-2-one;
1-(2,6-Dimethyl-pyridin-3-yl)-3-methyl-8-(1-methyl-1H-pyrrolo[2,3-b]pyridin-5-yl)-1,3-dihydro-imidazo[4,5-c]quinolin-2-one;
1-(2,6-Dimethyl-pyridin-3-yl)-8-(5-isopropylamino-pyridin-3-yl)-3-methyl-1,3-dihydro-imidazo[4,5-c]quinolin-2-one;
8-(6-Amino-5-trifluoromethyl-pyridin-3-yl)-1-{6-[(2-methoxy-ethyl)-methyl-amino]-2-methyl-pyridin-3-yl}-3-methyl-1,3-dihydro-imidazo[4,5-c]quinolin-2-one;
1-{6-[(2-Methoxy-ethyl)-methyl-amino]-2-methyl-pyridin-3-yl}-3-methyl-8-(2-methylamino-pyrimidin-5-yl)-1,3-dihydro-imidazo[4,5-c]quinolin-2-one;
8-(6-Ethoxy-pyridin-3-yl)-1-{6-[(2-methoxy-ethyl)-methyl-amino]-2-methyl-pyridin-3-yl}-3-methyl-1,3-dihydro-imidazo[4,5-c]quinolin-2-one;
1-{6-[(2-Methoxy-ethyl)-methyl-amino]-2-methyl-pyridin-3-yl}-3-methyl-8-(6-methylamino-pyridin-3-yl)-1,3-dihydro-imidazo[4,5-c]quinolin-2-one;
8-(2-Dimethylamino-pyrimidin-5-yl)-1-{6-[(2-methoxy-ethyl)-methyl-amino]-2-methyl-pyridin-3-yl}-3-methyl-1,3-dihydro-imidazo[4,5-c]quinolin-2-one;
8-(5-Ethylamino-6-methyl-pyridin-3-yl)-1-{6-[(2-methoxy-ethyl)-methyl-amino]-2-methyl-pyridin-3-yl}-3-methyl-1,3-dihydro-imidazo[4,5-c]quinolin-2-one;
1-{6-[(2-Methoxy-ethyl)-methyl-amino]-2-methyl-pyridin-3-yl}-3-methyl-8-(1-methyl-1H-pyrrolo[2,3-b]pyridin-5-yl)-1,3-dihydro-imidazo[4,5-c]quinolin-2-one;
8-(5-Amino-pyridin-3-yl)-1-{6-[(2-methoxy-ethyl)-methyl-amino]-2-methyl-pyridin-3-yl}-3-methyl-1,3-dihydro-imidazo[4,5-c]quinolin-2-one;
8-(6-Hydroxymethyl-5-methoxy-pyridin-3-yl)-1-{6-[(2-methoxy-ethyl)-methyl-amino]-2-methyl-pyridin-3-yl}-3-methyl-1,3-dihydro-imidazo[4,5-c]quinolin-2-one;
8-(6-Amino-5-trifluoromethyl-pyridin-3-yl)-1-(6-fluoro-4-methyl-pyridin-3-yl)-3-methyl-1,3-dihydro-imidazo[4,5-c]quinolin-2-on;
8-(5-Ethylamino-6-methyl-pyridin-3-yl)-1-(6-fluoro-4-methyl-pyridin-3-yl)-3-methyl-1,3-dihydro-imidazo[4,5-c]quinolin-2-one;
1-(6-Fluoro-4-methyl-pyridin-3-yl)-3-methyl-8-(2-methylamino-pyrimidin-5-yl)-1,3-dihydro-imidazo[4,5-c]quinolin-2-one;
1-(6-Fluoro-4-methyl-pyridin-3-yl)-3-methyl-8-(6-methylamino-pyridin-3-yl)-1,3-dihydro-imidazo[4,5-c]quinolin-2-one;
1-(6-Fluoro-4-methyl-pyridin-3-yl)-3-methyl-8-(1-methyl-1H-pyrrolo[2,3-b]pyridin-5-yl)-1,3-dihydro-imidazo[4,5-c]quinolin-2-one;
8-(6-Ethoxy-pyridin-3-yl)-3-methyl-1-(5-methyl-pyridin-3-yl)-1,3-dihydro-imidazo[4,5-c]quinolin-2-one;
3-Methyl-8-(2-methylamino-pyrimidin-5-yl)-1-(5-methyl-pyridin-3-yl)-1,3-dihydro-imidazo[4,5-c]quinolin-2-one;
1-(2-Chloro-6-methoxy-pyridin-3-yl)-8-(5-ethylamino-6-methyl-pyridin-3-yl)-3-methyl-1,3-dihydro-imidazo[4,5-c]quinolin-2-one;
1-(2-Chloro-6-methoxy-pyridin-3-yl)-3-methyl-8-(2-methylamino-pyrimidin-5-yl)-1,3-dihydro-imidazo[4,5-c]quinolin-2-one;
1-(2-Chloro-6-methoxy-pyridin-3-yl)-8-(2-dimethylamino-pyrimidin-5-yl)-3-methyl-1,3-dihydro-imidazo[4,5-c]quinolin-2-one;
1-(6-Dimethylamino-2-methyl-pyridin-3-yl)-3-methyl-8-(2-methylamino-pyrimidin-5-yl)-1,3-dihydro-imidazo[4,5-c]quinolin-2-one;
1-(6-Dimethylamino-2-methyl-pyridin-3-yl)-3-methyl-8-(6-methyl-5-methylamino-pyridin-3-yl)-1,3-dihydro-imidazo[4,5-c]quinolin-2-one;
1-(6-Dimethylamino-2-methyl-pyridin-3-yl)-8-(6-ethoxy-pyridin-3-yl)-3-methyl-1,3-dihydro-imidazo[4,5-c]quinolin-2-one;
1-(6-Dimethylamino-2-methyl-pyridin-3-yl)-8-(6-hydroxymethyl-5-methoxy-pyridin-3-yl)-3-methyl-1,3-dihydro-imidazo[4,5-c]quinolin-2-one;
8-(5-Amino-pyridin-3-yl)-1-(6-dimethylamino-2-methyl-pyridin-3-yl)-3-methyl-1,3-dihydro-imidazo[4,5-c]quinolin-2-one;
N-{5-[8-(5-Amino-pyridin-3-yl)-3-methyl-2-oxo-2,3-dihydro-imidazo[4,5-c]quinolin-1-yl]-6-methyl-pyridin-2-yl}-2-methoxy-acetamide;
N-{5-[8-(6-Ethoxy-pyridin-3-yl)-3-methyl-2-oxo-2,3-dihydro-imidazo[4,5-c]quinolin-1-yl]-6-methyl-pyridin-2-yl}-2-methoxy-acetamide;
2-Methoxy-N-{6-methyl-5-[3-methyl-8-(2-methylamino-pyrimidin-5-yl)-2-oxo-2,3-dihydro-imidazo[4,5-c]quinolin-1-yl]-pyridin-2-yl}-acetamide;
2-Methoxy-N-{6-methyl-5-[3-methyl-8-(6-methylamino-pyridin-3-yl)-2-oxo-2,3-dihydro-imidazo[4,5-c]quinolin-1-yl]-pyridin-2-yl}-acetamide;
N-{5-[8-(5-Isopropoxy-pyridin-3-yl)-3-methyl-2-oxo-2,3-dihydro-imidazo[4,5-c]quinolin-1-yl]-6-methyl-pyridin-2-yl}-2-methoxy-acetamide;
8-(6-Amino-5-trifluoromethyl-pyridin-3-yl)-3-methyl-1-[2-methyl-6-(4-methyl-3-oxo-piperazin-1-yl)-pyridin-3-yl]-1,3-dihydro-imidazo[4,5-c]quinolin-2-one;
8-(5-Ethoxy-6-methoxymethyl-pyridin-3-yl)-1-[6-(2-methoxy-ethoxy)-2-methyl-pyridin-3-yl]-3-methyl-1,3-dihydro-imidazo[4,5-c]quinolin-2-one;
8-(5-Azetidin-1-yl-pyridin-3-yl)-1-[6-(2-methoxy-ethoxy)-2-methyl-pyridin-3-yl]-3-methyl-1,3-dihydro-imidazo[4,5-c]quinolin-2-one;
1-[6-(2-Methoxy-ethoxy)-2-methyl-pyridin-3-yl]-8-(5-methoxy-6-methoxymethyl-pyridin-3-yl)-3-methyl-1,3-dihydro-imidazo[4,5-c]quinolin-2-one;
1-(6-Azetidin-1-yl-2-methyl-pyridin-3-yl)-8-(5-azetidin-1-yl-pyridin-3-yl)-3-methyl-1,3-dihydro-imidazo[4,5-c]quinolin-2-one;
1-(6-Azetidin-1-yl-2-methyl-pyridin-3-yl)-8-(5-isopropoxy-6-methoxymethyl-pyridin-3-yl)-3-methyl-1,3-dihydro-imidazo[4,5-c]quinolin-2-one;
1-(6-Azetidin-1-yl-2-methyl-pyridin-3-yl)-8-[5-(1-hydroxy-1-methyl-ethyl)-pyridin-3-yl]-3-methyl-1,3-dihydro-imidazo[4,5-c]quinolin-2-one;
1-(6-Azetidin-1-yl-2-methyl-pyridin-3-yl)-3-methyl-8-(6-methylamino-pyridin-3-yl)-1,3-dihydro-imidazo[4,5-c]quinolin-2-one;
1-(6-Azetidin-1-yl-2-methyl-pyridin-3-yl)-8-(5-ethoxy-6-methoxymethyl-pyridin-3-yl)-3-methyl-1,3-dihydro-imidazo[4,5-c]quinolin-2-one;
1-(6-Azetidin-1-yl-2-methyl-pyridin-3-yl)-8-(5-methoxy-6-methoxymethyl-pyridin-3-yl)-3-methyl-1,3-dihydro-imidazo[4,5-c]quinolin-2-one;
1-(6-Azetidin-1-yl-2-methyl-pyridin-3-yl)-8-(6-ethoxy-pyridin-3-yl)-3-methyl-1,3-dihydro-imidazo[4,5-c]quinolin-2-one;
1-(6-Azetidin-1-yl-2-methyl-pyridin-3-yl)-8-(5-ethylamino-6-hydroxymethyl-pyridin-3-yl)-3-methyl-1,3-dihydro-imidazo[4,5-c]quinolin-2-one;
1-(6-Azetidin-1-yl-2-methyl-pyridin-3-yl)-8-(5-isopropoxy-pyridin-3-yl)-3-methyl-1,3-dihydro-imidazo[4,5-c]quinolin-2-one;
1-[6-(3,3-Difluoro-azetidin-1-yl)-2-methyl-pyridin-3-yl]-3-methyl-8-(2-methylamino-pyrimidin-5-yl)-1,3-dihydro-imidazo[4,5-c]quinolin-2-one;
1-[6-(3,3-Difluoro-azetidin-1-yl)-2-methyl-pyridin-3-yl]-8-[5-(1-hydroxy-1-methyl-ethyl)-pyridin-3-yl]-3-methyl-1,3-dihydro-imidazo[4,5-c]quinolin-2-one;
1-[6-(3,3-Difluoro-azetidin-1-yl)-2-methyl-pyridin-3-yl]-8-(5-ethylamino-6-hydroxymethyl-pyridin-3-yl)-3-methyl-1,3-dihydro-imidazo[4,5-c]quinolin-2-one;
1-[6-(3,3-Difluoro-azetidin-1-yl)-2-methyl-pyridin-3-yl]-8-(6-hydroxymethyl-5-methoxy-pyridin-3-yl)-3-methyl-1,3-dihydro-imidazo[4,5-c]quinolin-2-one;
3-Methyl-8-(6-methylamino-pyridin-3-yl)-1-(2-methyl-6-pyrrolidin-1-yl-pyridin-3-yl)-1,3-dihydro-imidazo[4,5-c]quinolin-2-one;
8-(5-Ethoxy-6-methoxymethyl-pyridin-3-yl)-3-methyl-1-(2-methyl-6-pyrrolidin-1-yl-pyridin-3-yl)-1,3-dihydro-imidazo[4,5-c]quinolin-2-one;
8-[5-(1-Hydroxy-1-methyl-ethyl)-pyridin-3-yl]-3-methyl-1-(2-methyl-6-pyrrolidin-1-yl-pyridin-3-yl)-1,3-dihydro-imidazo[4,5-c]quinolin-2-one;
8-(6-Hydroxymethyl-5-methoxy-pyridin-3-yl)-3-methyl-1-(2-methyl-6-pyrrolidin-1-yl-pyridin-3-yl)-1,3-dihydro-imidazo[4,5-c]quinolin-2-one;
8-(5-Ethylamino-6-hydroxymethyl-pyridin-3-yl)-3-methyl-1-(2-methyl-6-pyrrolidin-1-yl-pyridin-3-yl)-1,3-dihydro-imidazo[4,5-c]quinolin-2-one;
8-(6-Amino-5-trifluoromethyl-pyridin-3-yl)-1-{6-[(2-hydroxy-ethyl)-methyl-amino]-2-methyl-pyridin-3-yl}-3-methyl-1,3-dihydro-imidazo[4,5-c]quinolin-2-one;
1-{6-[(2-Hydroxy-ethyl)-methyl-amino]-2-methyl-pyridin-3-yl}-3-methyl-8-(2-methylamino-pyrimidin-5-yl)-1,3-dihydro-imidazo[4,5-c]quinolin-2-one;
8-(5-Ethylamino-6-hydroxymethyl-pyridin-3-yl)-1-{6-[(2-hydroxy-ethyl)-methyl-amino]-2-methyl-pyridin-3-yl}-3-methyl-1,3-dihydro-imidazo[4,5-c]quinolin-2-one;
1-[6-(3-Hydroxy-pyrrolidin-1-yl)-2-methyl-pyridin-3-yl]-8-(5-isopropoxy-pyridin-3-yl)-3-methyl-1,3-dihydro-imidazo[4,5-c]quinolin-2-one;
1-[6-(3-Hydroxy-pyrrolidin-1-yl)-2-methyl-pyridin-3-yl]-3-methyl-8-(2-methylamino-pyrimidin-5-yl)-1,3-dihydro-imidazo[4,5-c]quinolin-2-one;
8-(6-Amino-5-trifluoromethyl-pyridin-3-yl)-1-[6-(3-hydroxy-pyrrolidin-1-yl)-2-methyl-pyridin-3-yl]-3-methyl-1,3-dihydro-imidazo[4,5-c]quinolin-2-one;
8-(6-Ethoxy-pyridin-3-yl)-1-[6-(3-hydroxy-pyrrolidin-1-yl)-2-methyl-pyridin-3-yl]-3-methyl-1,3-dihydro-imidazo[4,5-c]quinolin-2-one;
8-[5-(1-Hydroxy-1-methyl-ethyl)-pyridin-3-yl]-1-[6-(3-hydroxy-pyrrolidin-1-yl)-2-methyl-pyridin-3-yl]-3-methyl-1,3-dihydro-imidazo[4,5-c]quinolin-2-one;
8-[5-(1-Hydroxy-1-methyl-ethyl)-pyridin-3-yl]-1-[6-(3-methoxy-pyrrolidin-1-yl)-2-methyl-pyridin-3-yl]-3-methyl-1,3-dihydro-imidazo[4,5-c]quinolin-2-one;
1-[6-(3-Methoxy-pyrrolidin-1-yl)-2-methyl-pyridin-3-yl]-3-methyl-8-(2-methylamino-pyrimidin-5-yl)-1,3-dihydro-imidazo[4,5-c]quinolin-2-one;
8-(6-Hydroxymethyl-5-methoxy-pyridin-3-yl)-1-[6-(3-methoxy-pyrrolidin-1-yl)-2-methyl-pyridin-3-yl]-3-methyl-1,3-dihydro-imidazo[4,5-c]quinolin-2-one;
1-[6-(3-Hydroxy-azetidin-1-yl)-2-methyl-pyridin-3-yl]-8-[5-(1-hydroxy-1-methyl-ethyl)-pyridin-3-yl]-3-methyl-1,3-dihydro-imidazo[4,5-c]quinolin-2-one;
8-(5-Ethylamino-6-hydroxymethyl-pyridin-3-yl)-1-[6-(3-hydroxy-azetidin-1-yl)-2-methyl-pyridin-3-yl]-3-methyl-1,3-dihydro-imidazo[4,5-c]quinolin-2-one;
8-(5-Fluoro-6-methylamino-pyridin-3-yl)-1-[6-(3-hydroxy-azetidin-1-yl)-2-methyl-pyridin-3-yl]-3-methyl-1,3-dihydro-imidazo[4,5-c]quinolin-2-one;
1-[6-(3-Hydroxy-azetidin-1-yl)-2-methyl-pyridin-3-yl]-3-methyl-8-(6-methylamino-5-trifluoromethyl-pyridin-3-yl)-1,3-dihydro-imidazo[4,5-c]quinolin-2-one;
1-[6-(3-Hydroxy-azetidin-1-yl)-2-methyl-pyridin-3-yl]-8-(6-hydroxymethyl-5-methoxy-pyridin-3-yl)-3-methyl-1,3-dihydro-imidazo[4,5-c]quinolin-2-one;
8-(2,4-Dimethoxy-pyrimidin-5-yl)-1-[6-(3-hydroxy-azetidin-1-yl)-2-methyl-pyridin-3-yl]-3-methyl-1,3-dihydro-imidazo[4,5-c]quinolin-2-one;
8-(2-Ethylamino-pyrimidin-5-yl)-1-[6-(3-hydroxy-azetidin-1-yl)-2-methyl-pyridin-3-yl]-3-methyl-1,3-dihydro-imidazo[4,5-c]quinolin-2-one;
1-(6-Amino-2-methyl-pyridin-3-yl)-8-(6-hydroxymethyl-5-methoxy-pyridin-3-yl)-3-methyl-1,3-dihydro-imidazo[4,5-c]quinolin-2-one;
8-(5-Fluoro-6-methylamino-pyridin-3-yl)-1-[6-(1-hydroxy-1-methyl-ethyl)-2-methyl-pyridin-3-yl]-3-methyl-1,3-dihydro-imidazo[4,5-c]quinolin-2-one;
1-[6-(1-Hydroxy-1-methyl-ethyl)-2-methyl-pyridin-3-yl]-3-methyl-8-(2-methylamino-pyrimidin-5-yl)-1,3-dihydro-imidazo[4,5-c]quinolin-2-one;
8-(2,4-Dimethoxy-pyrimidin-5-yl)-1-[6-(1-hydroxy-1-methyl-ethyl)-2-methyl-pyridin-3-yl]-3-methyl-1,3-dihydro-imidazo[4,5-c]quinolin-2-one;
8-(6-Ethoxy-pyridin-3-yl)-1-[6-(1-hydroxy-1-methyl-ethyl)-2-methyl-pyridin-3-yl]-3-methyl-1,3-dihydro-imidazo[4,5-c]quinolin-2-one;
8-(6-Amino-5-trifluoromethyl-pyridin-3-yl)-1-[6-(1-hydroxy-1-methyl-ethyl)-2-methyl-pyridin-3-yl]-3-methyl-1,3-dihydro-imidazo[4,5-c]quinolin-2-one;
2-(5-{1-[6-(1-Hydroxy-1-methyl-ethyl)-2-methyl-pyridin-3-yl]-3-methyl-2-oxo-2,3-dihydro-1H-imidazo[4,5-c]quinolin-8-yl}-pyridin-3-yl)-2-methyl-propionitrile;
8-(2-Ethylamino-pyrimidin-5-yl)-1-[6-(1-hydroxy-1-methyl-ethyl)-2-methyl-pyridin-3-yl]-3-methyl-1,3-dihydro-imidazo[4,5-c]quinolin-2-one;
1-(5-{1-[6-(1-Hydroxy-1-methyl-ethyl)-2-methyl-pyridin-3-yl]-3-methyl-2-oxo-2,3-dihydro-1H-imidazo[4,5-c]quinolin-8-yl}-pyridin-3-yl)-cyclobutanecarbonitrile;
1-[6-(1-Hydroxy-1-methyl-ethyl)-2-methyl-pyridin-3-yl]-8-(5-isopropoxy-pyridin-3-yl)-3-methyl-1,3-dihydro-imidazo[4,5-c]quinolin-2-one;
1-[6-((S)-3-Hydroxy-pyrrolidin-1-yl)-2-methyl-pyridin-3-yl]-3-methyl-8-(2-methylamino-pyrimidin-5-yl)-1,3-dihydro-imidazo[4,5-c]quinolin-2-one;
8-(5-Fluoro-6-methylamino-pyridin-3-yl)-1-[6-((S)-3-hydroxy-pyrrolidin-1-yl)-2-methyl-pyridin-3-yl]-3-methyl-1,3-dihydro-imidazo[4,5-c]quinolin-2-one;
8-(2,4-Dimethoxy-pyrimidin-5-yl)-1-[6-((S)-3-hydroxy-pyrrolidin-1-yl)-2-methyl-pyridin-3-yl]-3-methyl-1,3-dihydro-imidazo[4,5-c]quinolin-2-one;
8-(5-Chloro-6-methylamino-pyridin-3-yl)-1-[6-((S)-3-hydroxy-pyrrolidin-1-yl)-2-methyl-pyridin-3-yl]-3-methyl-1,3-dihydro-imidazo[4,5-c]quinolin-2-one;
1-[6-((S)-3-Hydroxy-pyrrolidin-1-yl)-2-methyl-pyridin-3-yl]-3-methyl-8-(6-methylamino-5-trifluoromethyl-pyridin-3-yl)-1,3-dihydro-imidazo[4,5-c]quinolin-2-one;
8-(5-Ethylamino-6-hydroxymethyl-pyridin-3-yl)-1-[6-((S)-3-hydroxy-pyrrolidin-1-yl)-2-methyl-pyridin-3-yl]-3-methyl-1,3-dihydro-imidazo[4,5-c]quinolin-2-one;
8-(6-Hydroxymethyl-5-methoxy-pyridin-3-yl)-1-[6-((S)-3-hydroxy-pyrrolidin-1-yl)-2-methyl-pyridin-3-yl]-3-methyl-1,3-dihydro-imidazo[4,5-c]quinolin-2-one;
1-[6-((S)-3-Hydroxy-pyrrolidin-1-yl)-2-methyl-pyridin-3-yl]-8-(5-isopropoxy-pyridin-3-yl)-3-methyl-1,3-dihydro-imidazo[4,5-c]quinolin-2-one;
8-(2-Ethylamino-4-methoxy-pyrimidin-5-yl)-1-[6-((S)-3-hydroxy-pyrrolidin-1-yl)-2-methyl-pyridin-3-yl]-3-methyl-1,3-dihydro-imidazo[4,5-c]quinolin-2-one;
8-(2-Ethylamino-pyrimidin-5-yl)-1-[6-((S)-3-hydroxy-pyrrolidin-1-yl)-2-methyl-pyridin-3-yl]-3-methyl-1,3-dihydro-imidazo[4,5-c]quinolin-2-one;
8-[5-(1-Hydroxy-1-methyl-ethyl)-pyridin-3-yl]-3-methyl-1-(2-methyl-6-trifluoromethyl-pyridin-3-yl)-1,3-dihydro-imidazo[4,5-c]quinolin-2-one;
8-(6-Hydroxymethyl-5-methylamino-pyridin-3-yl)-3-methyl-1-(2-methyl-6-trifluoromethyl-pyridin-3-yl)-1,3-dihydro-imidazo[4,5-c]quinolin-2-one;
8-(6-Amino-5-methoxymethyl-pyridin-3-yl)-3-methyl-1-(2-methyl-6-trifluoromethyl-pyridin-3-yl)-1,3-dihydro-imidazo[4,5-c]quinolin-2-one;
8-(6-Amino-5-hydroxymethyl-pyridin-3-yl)-3-methyl-1-(2-methyl-6-trifluoromethyl-pyridin-3-yl)-1,3-dihydro-imidazo[4,5-c]quinolin-2-one;
8-(5-Methoxymethyl-6-methylamino-pyridin-3-yl)-3-methyl-1-(2-methyl-6-trifluoromethyl-pyridin-3-yl)-1,3-dihydro-imidazo[4,5-c]quinolin-2-one;
1-[6-((R)-3-Hydroxy-pyrrol idin-1-yl)-2-methyl-pyridin-3-yl]-3-methyl-8-(2-methylamino-pyrimidin-5-yl)-1,3-dihydro-imidazo[4,5-c]quinolin-2-one;
8-(6-Ethylamino-5-trifluoromethyl-pyridin-3-yl)-1-[6-((R)-3-hydroxy-pyrrolidin-1-yl)-2-methyl-pyridin-3-yl]-3-methyl-1,3-dihydro-imidazo[4,5-c]quinolin-2-one;
8-(2-Ethylamino-pyrimidin-5-yl)-1-[6-((R)-3-hydroxy-pyrrolidin-1-yl)-2-methyl-pyridin-3-yl]-3-methyl-1,3-dihydro-imidazo[4,5-c]quinolin-2-one;
8-(5-Ethoxymethyl-6-ethylamino-pyridin-3-yl)-1-[6-((R)-3-hydroxy-pyrrolidin-1-yl)-2-methyl-pyridin-3-yl]-3-methyl-1,3-dihydro-imidazo[4,5-c]quinolin-2-one;
2-(5-{1-[6-((R)-3-Hydroxy-pyrrolidin-1-yl)-2-methyl-pyridin-3-yl]-3-methyl-2-oxo-2,3-dihydro-1H-imidazo[4,5-c]quinolin-8-yl}-pyridin-3-yl)-2-methyl-propionitrile;
8-[5-(1-Hydroxy-1-methyl-ethyl)-pyridin-3-yl]-3-methyl-1-[2-methyl-6-(3,3,4,4-tetrafluoro-pyrrolidin-1-yl)-pyridin-3-yl]-1,3-dihydro-imidazo[4,5-c]quinolin-2-one;
8-(5-Ethylamino-6-hydroxymethyl-pyridin-3-yl)-3-methyl-1-[2-methyl-6-(3,3,4,4-tetrafluoro-pyrrolidin-1-yl)-pyridin-3-yl]-1,3-dihydro-imidazo[4,5-c]quinolin-2-one;
8-(6-Hydroxymethyl-5-methoxy-pyridin-3-yl)-3-methyl-1-[2-methyl-6-(3,3,4,4-tetrafluoro-pyrrolidin-1-yl)-pyridin-3-yl]-1,3-dihydro-imidazo[4,5-c]quinolin-2-one;
8-(6-Amino-5-hydroxymethyl-pyridin-3-yl)-3-methyl-1-[2-methyl-6-(3,3,4,4-tetrafluoro-pyrrolidin-1-yl)-pyridin-3-yl]-1,3-dihydro-imidazo[4,5-c]quinolin-2-one.

8. A pharmaceutical composition comprising a compound of formula (I), according to any of claims 1 to 6, or a pharmaceutically acceptable salt thereof, and optionally a further therapeutic agent, together with a pharmaceutically acceptable carrier.

9. A compound of the formula (I), according to any one of claims 1 to 7, or pharmaceutically acceptable salt thereof, for use in the treatment of the animal or human body, especially in the treatment of a lipid and/or protein kinase dependent disease.

10. Use of a compound of formula (I), according to any one of claims 1 to 7, or a pharmaceutically acceptable salt thereof, for the preparation of a pharmaceutical composition for use in the treatment of a lipid and/or protein kinase dependent disease.

11. A method of treatment of a disease that responds to inhibition of a lipid and/or protein kinase, which comprises administering a prophylactically or especially therapeutically effective amount of a compound of formula (I) according to any one of claims 1-7, or a pharmaceutically acceptable salt thereof, to a warm-blooded animal, e.g., a human, in need of such treatment.

12. A compound for use according to claim 9, or the use of a compound according to claim 10, or a method of treatment according to claim 11, wherein the lipid kinase dependent disease is preferably one dependent on a Class I PI3K and the protein kinase dependent disease is preferably dependent on a Class IV PI3K.

13. A compound for use according to claim 9, or the use of a compound according to claim 10, or a method of treatment according to claim 110, wherein the lipid kinase dependent disease is dependent on a Class I PI3K selected from the group consisting of PI3Kalpha, PI3Kbeta, PI3Kdelta, PI3Kgamma and the protein kinase dependent disease is dependent on a Class IV PI3K which is mTOR.

14. A compound for use according to claim 9, or the use of a compound according to claim 10, or a method of treatment according to claim 11, wherein the disease is a proliferative disease, a benign or malignant tumor, a carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, colon carcinoma or colorectal adenoma or a tumor of the neck or head, an epidermal hyperproliferation, psoriasis, prostate hyperplasia, a neoplasia, a neoplasia of epithelial character, lymphomas, a mammary carcinoma or a leukemia, Cowden syndrome, Lhermitte-Dudos disease or Bannayan-Zonana syndrome, basal cell carcinoma, squamous cell carcinoma and actinic keratosis.
